SEQUENCE LISTING (844 sequences)

SEQ ID NO: 1 - protein, 128 aa, synthetic construct
QVQLQESGGGLVQPGGSLRLSCAASGRTFSSYGMGWFRQAPGKKREFVAAISRDARITDYADSVKGRFTISRDNAKNTAYLEMNSLNPEDTAVYFCAAAYTYSGSHYVTARSSDYHSWGQGTQVTVSS

SEQ ID NO: 2 - protein, 128 aa, synthetic construct
QVQLQESGGGLVQAGGSLTLSCAASGMPISFNVLGWFRQIPGKERDFVAAISRDARITDYADSVKGRFTISRDNVKNTVYLQMNSLNPEDTAVYFCAAAYTYSGSHYVTARSSDYHSWGQGTQVTVSS

SEQ ID NO: 3 - protein, 128 aa, synthetic construct
QVQLQESGGGLVQAGGSLRLSCAASGRTFSSYGMGWFRQAPGKEREFVAAISRDARITDYADSVKGRFTISRDNAKNTAYLEMNSLNPEDTAVYFCAAAYTYSGSHYVTARSSDYHSWGQGTQVTVSS

SEQ ID NO: 4 - protein, 128 aa, synthetic construct
QVQLQESGGGLVQAGGSLRLSCAASGRTFSSYGMGWFRQAPGKERDFVAAISRDARITDYADSVKGRFTISRDNVKNTVYLQMNSLNPEDTAVYFCAAAYTYSGSHYVTARSSDYHSWGQGTQVTVSS

SEQ ID NO: 5 - protein, 128 aa, synthetic construct
QVQLQESGGGLMQAGDSLRLSCAASGRTFSSYGMGWFRQAPGKEREFVAAISRGARITDYADSVKGRFTISRDNAKNTAYLQMNSLNPEDTAVYFCAAAYTYSGSHYVTARSSDYHSWGQGTQVTVSS

SEQ ID NO: 6 - protein, 128 aa, synthetic construct
QVQLQESGGGLVQAGGSLRLSCAASGRTFSSYGMGWFRQAPGKERDFVAAISRDARYTDYADSVKGRFTISKDNAKNTAYLQMNSLNPEDTAVYFCAAAYTYSGSHYVTARSGDYNYWGQGTQVTVSS

SEQ ID NO: 7 - protein, 128 aa, synthetic construct
QVQLQESGGGLVQAGGSLRLSCAASGRTFSSRGMGXFRQAPGKERDFVAAISRGARNTDYADSVKGRFTISRDNAKNTAYLQMNSLNPGDTAVYFCAAAYIYSGSHYVTARSSDYEYWGQGTQVTVSS

SEQ ID NO: 8 - protein, 128 aa, synthetic construct
QVQLQESGGGLVQAGGSLRLSCAASGRTFSMHGMGWFRQAPGKEREFVAAISRDARITDYADSVKGRFTISRDNAKNTAYLQMNSLNPEDTAVYFCAAAYTYSGSHYVTARSSDYNYWGQGTQVTVSS

SEQ ID NO: 9 - protein, 128 aa, synthetic construct
QVQLQESGGGLVQAGDSLRLSCAASGRTFSSYGMGWFRQAPGKEREFVAAISRGARITDYADSVKGRFTISRDNAKNTAYLQMNSLNPEDTAVYFCAAAYTYSGSHYVTARSSDYHSWGQGTQVTVSS

SEQ ID NO: 10 - protein, 128 aa, synthetic construct
QVQLQESGGGLVQAGGSLRLSCASSGRTFSSRTVGWFRQAPGKERYFVAAISRDTAYTDYADSVKGRFTISRGNAKNTVYLQMNSLNPEDTAVYYCAASHIYSGSHYISARSSDYMYWGQGTQVTVSS

SEQ ID NO: 11 - protein, 128 aa, synthetic construct
QVQLQESGGGLVQAGGSLRLSCAASGRTFSSYGMGWFRQAPGKEREFVAAISRDASITDYADSVKGRFTISRDNAKNTAYLEMNSLNPEDTAVYFCAAAYTYSGSHYVTARSSDYHSWGQGTQVTVSS

SEQ ID NO: 12 - protein, 128 aa, synthetic construct
QVQLQESGGGLVQPGGSLRLSCAASGFTFSSYGMGWFRQAPGKEREFVAAISRDARITDYADSVKGRFTISRDNAKNTAYLEMNSLNPEDTAVYFCAAAYTYSGSHYVTARSSDYHSWGQGTQVTVSS

SEQ ID NO: 13 - protein, 128 aa, synthetic construct
QVQLQESGGGLVQTGGSLKLSCAASGRTFSSYGMGWFRQAPGKERDFVAAISRDARYTDYADSVKGRFTISKDNAKNTAYLQMNSLNPEDTAVYFCAAAYTYSGSHYVTARSGDYNYWGXGTQVTVSS

SEQ ID NO: 14 - protein, 128 aa, synthetic construct
QVQLQESGGGLVQAGGSLKLSCAATGFILDYVGVGWFRQAPGKERDFVAAISRDARITDYADSVKGRFTISRDNAKNTAYLQMNSLNPEDTAVYFCAAAYTYSGSHYVTARSSDYHTWGQGTQVTVSS

SEQ ID NO: 15 - protein, 128 aa, synthetic construct
QVQLQESGGGSVQAGGSLRLSCAASGRTFSSYGMGWFRQAPGKEREFVAAISRDARITDYADSVKGRFTISRDNAKNTAYLEMNSLNPEDTAVYFCAAAYTYSGSHYVTARSSDYHSWGQGTQVTVSS

SEQ ID NO: 16 - protein, 128 aa, synthetic construct
QVQLQESGGGLVQAGGSLRLSCAAAGRTFSSYGMGWFRQAPGKERDFVAAISRDARITDYADSVKGRFTISRDNAKNTAYLQMNSLNPEDTAVYFCAAAYTYSGSHYVTARSSDYHTWGQGTQDTVSS

SEQ ID NO: 17 - protein, 128 aa, synthetic construct
QVQLQESGGGLVQAGGSLRLSCAASGRTFSSYGMGWFRQAPGKEREFVAAISRGARITDYADSVKGRFTISRDNAKNTAYLQMNSLNPEDTAVYFCAAAYTYSGSHYVTARSSDYHSWGQGTQVTVSS

SEQ ID NO: 18 - protein, 128 aa, synthetic construct
QVQLQESGGGLVQAGESLTLSCTASGRTFSSYGMGWFRQAPGKERDFVAAISRDARYTDYADSVKGRFTISKDNAKNTAYLQMNSLNPEDTAVYFCAAAYTYSGSHYVTARSGDYNYWGQGTQVTVSS

SEQ ID NO: 19 - protein, 128 aa, synthetic construct
QVQLQESGGGLVQAGDSLRLSCIASGPSFSSYGMGWFRQAPGKEREFVAAISRDARITDYADSVKGRFTISRDNAKNTAYLEMNSLNPEDTAVYFCAAAYTYSGSHYVTARSSDYHSWGQGTQVTVSS

SEQ ID NO: 20 - protein, 128 aa, synthetic construct
QVQLQESGGGLVQAGGSLRLSCAASGRTFSSYGMSWFRQAPGKERDFVAAISRDARITDYADSVKGRFTISRDNVKNTVYLQMNSLNPEDTAVYFCAAAYTYSGSHYVTARSSDYHSWGQGTQVTVSS

SEQ ID NO: 21 - protein, 128 aa, synthetic construct
QVQLQESGGGMVQAGGSLRLSCAASGRTFSSYGMGWFRQAPGKEREFVAAISRGARNTDYADSVKGRFTISRDNAKNTAYLQMNSLNPEDTAVYFCAAAYTYSGSHYVTARSSDYPYWGQGTQVTVSS

SEQ ID NO: 22 - protein, 128 aa, synthetic construct
QVQLQESGGGLVQPGDSLRLSCAASGRTFSSYGMGWFRQAPGKEREFVAAISRGARNTDYADSVKGRFTISRDNAKNTAYLQMNSLNPEDTAVYFCAAAYTYSGSHYVTARSSDYPYWGQGTQVTVSS

SEQ ID NO: 23 - protein, 128 aa, synthetic construct
QVQLQESGGGLVQAGGSLRLSCAASGRTFSSYGMGWFRQAPGKEREFVAAISRGARNTDYADSVKGRFTISRDNAKNTAFLQMNSLNPEDTAVYFCAAAYTYSGSHYVTARSSDYHTWGQGTQVTVSS

SEQ ID NO: 24 - protein, 128 aa, synthetic construct
QVQLQESGGGLVQAGDSLRISCAASGRTFSSYGMGWFRQAPGKERDFVAAISRDARYTDYADSVKGRFTISKDNAKNTAYLQMNSLNPEDTAVYFCAAAYTYSGSHYVTARSGDYNYWGQGTQVTVSS

SEQ ID NO: 25 - protein, 128 aa, synthetic construct
QVQLQESGGGLVQAGGSLRLSCAASGRTFSSYGMGWFRQAPGKEREFVAAISRDARITDYADSVKGRFTISRDNAKNTAYLEMNSLNPEDTAVYSCAAAYTYSGSHYVTARSSDYHSWGQGTQVTVSS

SEQ ID NO: 26 - protein, 128 aa, synthetic construct
QVQLQESGGGLVQAGGPLRLSCAASGRTFSSYGMGWFRQAPGKERDFVAAISRDARITDYADSVKGRFTISRDNVKNTVYLQMNSLNPEDTAVYFCAAAYTYSGSHYVTARSSDYHSWGQGTQVTVSS

SEQ ID NO: 27 - protein, 128 aa, synthetic construct
QVQLQESGGGLVQAGGSLRLSCAASGRTFSSYGMGWFRQAPGKERDFVAAISRGARNADYADSVKGRFTISRDNAKNTAYLQMNSLNPEDTAVYFCAAAYTYSGSHYVTARSSDYAYWGQGTQVTVSS

SEQ ID NO: 28 - protein, 128 aa, synthetic construct
QVQLQESGGGLVQAGGSLRLSCADSGRTFSSYGMGWFRQAPGKEREFVAAISRGARITDYADSVKGRFTISRDNAKNTAYLQMNSLNPEDTAVYFCAAAYTYSGSHYVTARSSDYHSWGQGTQVTVSS

SEQ ID NO: 29 - protein, 128 aa, synthetic construct
QVQLQESGGGSVQLGGSLRLSCAASGRTFSSYGMGWFRXAPGKERDFVAAISRDARYTDYADSVKGRFTISKDNAKNTAYLQMNSLNPEDTAVYFCAAAYTYSGSHYVTARSGDYNYWGQGTQVTVSS

SEQ ID NO: 30 - protein, 119 aa, synthetic construct
QVQLQESGGGLVQPGGSLRLSCTASGSIFAIDDMYWYRQAPGKQREFVAYITATNSANYLDSVNGRFTISRDNAKNSVYLQMNSLKPEDTAVYYCNAKQIVGFQIDEYWGQGTQVTVSS

SEQ ID NO: 31 - protein, 119 aa, synthetic construct
QVQLQESGGGLVQAGGSLRLSCTASGSIFAIDDMYWYRQAPGKQREFVAYITATNSANYLDSVNGRFTISRDNAKNSVYLQMNSLKPEDTAVYYCNAKQIVGFQIDEYWGQGTQVTVSS

SEQ ID NO: 32 - protein, 119 aa, synthetic construct
QVQLQESGGGLVQPGGSLRLSCTASGSIFASDDMYWYRQAPGKQREFVAYITATNSANYLDSVNGRFTISRDNAKNSVYLQMNSLKPEDTAVYYCNAKQIVGFQIDEYWGQGTQVTVSS

SEQ ID NO: 33 - protein, 119 aa, synthetic construct
QVQLQESGGGLVQAGDSLRLSCTASGSIFAIDDMYWYRQAPGKQREFVAYITATNSANYLDSVNGRFTISRDNAENSVYLQMNSLKPEDTVVYYCNAKQIVGFQIDEYWGQGTQVTVSS

SEQ ID NO: 34 - protein, 119 aa, synthetic construct
QVQLQESGGGLVQPGGSLRLSCTASGSIFAIDDMYWYRQAPGKQREFVAYITATNSANYLDSVNGRFTISRDNAKNSVYLQMNSLKPEDTAVYYCNTKQIVGFQIDEYWGQGTQVTVSS

SEQ ID NO: 35 - protein, 119 aa, synthetic construct
QVQLQESGGGLVQPGGSLTLSCTASGSIFAIDDMYWYRQAPGKQREFVAYITATNSANYLDSVNGRFTISRDNAKNSVYLQMNSLKPEDTAVYYCNAKQIVGFQIDEYWGQGTQVTVSS

SEQ ID NO: 36 - protein, 119 aa, synthetic construct
QVQLQESGGGLVQAGGSLRLSCTASGSIFAIDDMYWYRQAPGKEREFVAYITATNSANYLDSVNGRFTISRDNAKNSVYLQMNSLKPEDTAVYYCNAKQIVGFQIDEYWGQGTQVTVSS

SEQ ID NO: 37 - protein, 119 aa, synthetic construct
QVQLQESGGGLVQAGGSLRLFCTASGSIFAIDDMYWYRQAPGKQREFVAYITATNSANYLDSVNGRFTISRDNAKNSVYLQMNSLKPEDTAVYYCNAKQIVGFQIDEYWGQGTQVTVSS

SEQ ID NO: 38 - protein, 119 aa, synthetic construct
QVQLQESGGGLAQPGGSLRLSCTASGSIFAIDDMYWYRQAPGKQREFVAYITATNSANYLDSVNGRFTISRDNAKNSVYLQMNSLKPEDTAVYYCNAKQIVGFQIDEYWGQGTQVTVSS

SEQ ID NO: 39 - protein, 119 aa, synthetic construct
QVQLQESGGGLVQPGGSLRLSCTASGSIFAIDDMYWYRQAPGRQREFVAYITATNSANYLDSVNGRFTISRDNAKNSVYLQMNSLKPEDTAVYYCNAKQIVGFQIDEYWGQGTQVTVSS

SEQ ID NO: 40 - protein, 119 aa, synthetic construct
QVQLQESGGGLVQAGESLRLSCTASGSIFAIDDMYWYRQAPGKQREFVAYITATNSANYLDSVNGRFTISRDNAKNSVYLQMNSLKPEDTAVYYCNAKQIVGFQIDEYWGQGTQVTVSS

SEQ ID NO: 41 - protein, 119 aa, synthetic construct
QVQLQESGGGLVQPGGSLRLSCTASGSIFAIDDMYWYRQAPGKQREFVAYITATNSANYLDSVNGRFTISRDNAKNSVYLQMNSLKPEDTAVYYCNAKQIVGFQIDDYWGQGTQVTVSS

SEQ ID NO: 42 - protein, 119 aa, synthetic construct
QVQLQESGGGLVQAGGSLRLSCTASGSIFAIDDMYWYRQAPGKQREFVAYITATNSANYLDPVNGRFTISRDNAKNSVYLQMNSLKPEDTAVYYCNAKQIVGFQIDEYWGQGTQVTVSS

SEQ ID NO: 43 - protein, 119 aa, synthetic construct
QVQLQESGGGLVQAGGSLRLSCAASGSIFAIDDMYWYRQAPGKQREFVAYITATNSANYLDSVNGRFTISRDNAKNSVYLQMNSLKPEDTAVYYCNAKQIVGFQIDEYWGQGTQVTVSS

SEQ ID NO: 44 - protein, 119 aa, synthetic construct
QVQLQESGGGSVQAGDSLRLSCTASGSIFAIDDMYWYRQAPGKQREFVAYITATNSANYLDSVNGRFTISRDNAKNSVYLQMNSLKPEDTAVYYCNAKQIVGFQIDEYWGQGTQVTVSS

SEQ ID NO: 45 - protein, 115 aa, synthetic construct
QVQLQESGGGLVQPGGSLRLSCTASGNIVSIYNMDWYRQAPGNQREWVASITSSGDPYYDGSAKGRFTISRDNAKMTVYLQMNSLRPEDTAVYYCKVPTEKGNYWGQGTQVTVSS

SEQ ID NO: 46 - protein, 115 aa, synthetic construct
QVQLQESGGGLVQAGGSLRLSCTASGSIVSIYNMDWYRQAPGNEREWVASITSSGDPYYDGSAKGRFTISRDNAKMTVYLQMNSLKPEDTAVYYCKVPTEKGNYWGQGTQVTVSS

SEQ ID NO: 47 - protein, 115 aa, synthetic construct
QVQLQESGGGLVQPGGSLRLSCTASGSILSIYNMDWYRQVPGNQREWVASITPSGDPYYDGSVKGRFTISRDNAKMMVYLQMNSLKPEDTAVYYCKVPTEKGNYWGQGTQVTVSS

SEQ ID NO: 48 - protein, 115 aa, synthetic construct
QVQLQESGGGLVQPGGSLRLSCTASGSIFSIYNMDWYRQAPGNQREWVASITSSGDPYYDASVKGRFTISRDSAKMTVYLQMNSLKPEDTAVYYCKLPTEKGNYWGQGTQVTVSS

SEQ ID NO: 49 - protein, 115 aa, synthetic construct
QVQLQESGGGLVQPGGSLRLSCTASGSIVSIYNMDWYRQAPGNEREWVASITSSGDPYYDGSAKGRFTISRDNAKMTVYLQMNSLKPEDTAVYYCKVPTEKGNYWGQGTQVTVSS

SEQ ID NO: 50 - protein, 115 aa, synthetic construct
QVQLQESGGGLVQPGGSLRLSCTASGSIFSIYNMDWYRQAPGNQREWVASITSSGDPYYDGPVKGRFTISRDNAKMTAYLQMNSLKPEDTAVYYCKLPTEKGNYWGXGTQVTVSS

SEQ ID NO: 51 - protein, 115 aa, synthetic construct
QVQLQESGGGLVXPGGSLRLSCTASGSIFSIYLMSWYRQAPGNQREWVASITSSGDPYYDESAKGRFTISRDNAKMTGYLQMNSLKPEDTAVYYCKLPTEKENYWGQGTQVTVSS

SEQ ID NO: 52 - protein, 115 aa, synthetic construct
QVQLQESGGGLVQPGGSLRLSCTASGSIVSIYNMDWYRQAPGNEREWVASITSSGDPYYDGSAKGRFTISRDNAKMTVYLQMNSLEPEDTAVYFCKVPTEKGNYWGQGTQVTVSS

SEQ ID NO: 53 - protein, 115 aa, synthetic construct
QVQLQESGGGLVQAGGSLNLSCTASGSIISIYNMDWYRQAPGNQREWVASITSNGDSYYDGSAKGRFTISRDNAKMTVYLQMNSLKPEDTAVYYCKVPTENGNYWGQGTQVTVSS

SEQ ID NO: 54 - protein, 115 aa, synthetic construct
QVQLQESGGGLVQPGGSLRLSCTASGSIFSIYNMDWYRXAPGNQREWVASITPSGDPYYDGPVKGRFTISRDNARMTVYLQMNSLKPEDTAVYYCKLPTEKGNYWGQGTQVTVSS

SEQ ID NO: 55 - protein, 115 aa, synthetic construct
QVQLQESGGGLVQPGGSLRLSCTASGSIFSIYNMDWYRQAPGNQREWVASITPSGDPYYDDSAKGRFTISRDNAKMTVYLQMNSLKPEDTAVYYCKVPTEKGNYWGQGTQVTVSS

SEQ ID NO: 56 - protein, 115 aa, synthetic construct
QVQLQESGGGLVQPGGSLRLSCIASGSIFSIYNMDWYRQAPGNQREWVASITPSGDPYYDDSAKGRFTISRDNAKMTVYLQMNSLKPEDTAVYYCKVPTEKGNYWGQGTQVTVSS

SEQ ID NO: 57 - protein, 115 aa, synthetic construct
QVQLQESGGGLVQPGGSLRLSCTASGSTVSIYNMDWYRQAPGNEREWVASITSSGDPYYDGSAKGRFTISRDNAKMTVYLQMNSLEPEDTAVYFCKVPTERGNYWGXGTQVTVSS

SEQ ID NO: 58 - protein, 115 aa, synthetic construct
QVQLQESGGGLVXPGGSLRLSCTASGSIFSIYNMDWYRQAPGNQREWVASITSSGDPYYDGSAKGRFTISRDNAKMTVYLQMNSLKPEDTAVYYCKVPTEKGNYWGQGTQVTVSS

SEQ ID NO: 59 - protein, 115 aa, synthetic construct
QVQLQESGGGSVQPGWSLRLTCAASGFTFSTSGMHWFRQAPGKGLEWVSSISSGGGDTYYTDSVKGRITISRDNAKNTLYLQLNDLKSEDTAVYYCSKDPSGATRGQGTQVTVSS

SEQ ID NO: 60 - protein, 115 aa, synthetic construct
QVQLQESGGGSVQPGGSLRLTCAASGFTFSTSGMHWFRQAPGKGLEWVSSISSGGGDTYYTDSVKGRITISRDNAKNTLYLQLNDLKSEDTAVYYCSKDPSGATRGQGTQVTVSS

SEQ ID NO: 61 - protein, 115 aa, synthetic construct
QVQLQESGGGLVQPGGSLRLSCAASGFTFSTSGMHWFRQAPGKGLEWVSSIGIIVGSSYYASSVKGRFTISRDNAKNTLYLHMNSLKSEDTAVYYCSKDVTGSSRGQGTQVTVSS

SEQ ID NO: 62 - protein, 115 aa, synthetic construct
QVQLQESGGGLVQPGGSLRLSCAASGFTFSTSGMHWFRQAPGKGLEWVSSIGIIVDSSYYADSVKGRFTISRDNAKNTLFLQMNSLKSEDTAVYYCSKDPTGSSRGQGTQVTVSS

SEQ ID NO: 63 - protein, 115 aa, synthetic construct
QVQLQESGGGFVXPGGSLRLSCAASGFTFSSSGMHWFRQAPGKGLEWVSTISSGGDVTYYLDSVKGRITISRDNAKNTLYLQMNDLKSEDTAVYYCSKDPTGATRGQGTQVTVSS

SEQ ID NO: 64 - protein, 115 aa, synthetic construct
QVQLQESGGGSVQPGGSLRLSCAASGFTFSTSGMHWFRQAPGKGLEWVSSIGIIVDSSYYASSVKGRFTISRDNAKNIVYLQMNSLKSEDTAVYYCSKDPTGSSRGQGTQVTVSS

SEQ ID NO: 65 - protein, 115 aa, synthetic construct
QVQLQESGGGLVQPGGSLRLSCAASGFTFSTFGMHWFRQAPGKGLEWVSSIGIIDDNSYYASSVKGRFTISRDNAKNTLYLQMNSLKSEDTAVYYCSKDTTGSSRGQGTQVTVSS

SEQ ID NO: 66 - protein, 115 aa, synthetic construct
QVQLQESGGGSVQPGGSLRLSCAASGFTFSTSGMHWFRQAPGKGLEWVSSIGIIVDSSYYASSVKGRFTISRDNAKNTLYLQMNSLKSEDTAVYYCSKDPTGSSRGQGTQVTVSS

SEQ ID NO: 67 - protein, 115 aa, synthetic construct
QVQLQESGGGLVQPGGSLRLSCAASGFTFSTSGMHWFRQAPGKGLEWVSSIGIIVDSSYYASSVKGRFTISRDNAKNTLYLHMNSLKSEDTAVYYCSKDVTGSSRGQGTQVTVSS

SEQ ID NO: 68 - protein, 115 aa, synthetic construct
QVQLQESGGGLVQPGGSLRLSCAASGFTFSTSGMHWFRQAPGKGLEWVSSIGATVDNSYYASSVKGRFTISRDNAKNTLYLLMNSLKSEDTAVYYCSKDPTGSSRGQGTQVTVSS

SEQ ID NO: 69 - protein, 119 aa, synthetic construct
QVQLQESGGGLVQAGGSLVLSCAASGSAALIFLMSWYRQAPGKQRELVAEVSRGGSTTYADSVEGRFHISRDNAKNIVDLQMNSLKPEDTAVYYCKADQNYGDSLRSSWGRGTQVTVSS

SEQ ID NO: 70 - protein, 119 aa, synthetic construct
QVQLQESGGGLVQAGGSLVLSCAASGSAALIFLMSWYRQAPGKQRELVAEVSRYGSTTYADSVEGRFTISRDNAKDIVDLQMNSLKPEDTAVYYCKADQYYGDSLRSSWGRGTQVTVSS

SEQ ID NO: 71 - protein, 119 aa, synthetic construct
QVQLQESGGGLVQAGGSLRLTCTASGSAALIFLMSWYRQAPGKQRELVAEVSRRGSTTYADSVEGRFTISRDNAKNIVDLQMNSLKPEDTAVYYCKADQYYGDSLRSSWGRGTQVTVSS

SEQ ID NO: 72 - protein, 119 aa, synthetic construct
QVQLQESGGGLVQAGGSLVLSCAASGSAALIFLMSWYRQAPGKQRELVAEVSRGGSTTYADSVEGRFTISRDNAKNIVDLQMNSLKPEDTAVYYCKADQNYGDSLRSSWGQGTQVTVSS

SEQ ID NO: 73 - protein, 119 aa, synthetic construct
QVQLQESGGGLVQAGGSLVLSCAASGSAALIFLMSWYRQAPGKQRELVAEVSRYGSTTYADSVEGRFTISRDNAKNIVDLQMNSLKPEDTAVYYCKADQYYGDSLRSSWGRGTQVTVSS

SEQ ID NO: 74 - protein, 119 aa, synthetic construct
QVQLQESGGGLVQAGGSLVLSCAASGSAALIFLMSWYRQAPGKQRELVAEVSRGGSTTYADSVEGRFTISRDNAKNIVDLQMNSLKPEDTAVYYCKADQNYGDSLRSSWGRGTQVTVSS

SEQ ID NO: 75 - protein, 119 aa, synthetic construct
QVQLQESGGGLVQAGGSLVLSCAASGSAALIFLMSWYRQAPGKQRELVAEVSRYGSTTYADSVEGRFTISRDNAKNIVDLQMNSLKPEDTAAYYCKADQYYGDSLRSSWGRGTQVTVSS

SEQ ID NO: 76 - protein, 119 aa, synthetic construct
QVQLQESGGGLVQLGGSLTLSCAASGSAALIFLMSWYRQAPGKQRELVAEVSRYGSTTYADSVEGRFTISRDNAKNIVDLQMNSLKPEDTAVYYCKADQYYGDSLRSSWGRGTQVTVSS

SEQ ID NO: 77 - protein, 119 aa, synthetic construct
QVQLQESGGGLVQAGGSLVLSCAASGSAALIFLMSWYRQAPGKQRELVAEVSRYGSTTYADSVEGRFTISRDNAKNIVDLQMNSLKPEDTAVYYCKADQYYGDSLRSSWGRGTQLTVSS

SEQ ID NO: 78 - protein, 116 aa, synthetic construct
QVQLQESGGGLVQPGGSLRLSCAASGFTFRSYAMSWVRQAPGKGLEWVSSISSSGTDTNYADSVKGRLTISRDNVKNTLYLQMNSLKPEDTAVYYCAKASQNYSPPGQGTQVTVSS

SEQ ID NO: 79 - protein, 116 aa, synthetic construct
QVQLQESGGGLVQPGGSLRLSCAASGFTISNYAMSWVRQAPGKGLEWVSSIGSNIASTNYADSVKGRFTISRDNAKNTLYLQMNSLKPEDTAVYYCAKASQNHSPPGQGTQVTVSS

SEQ ID NO: 80 - protein, 116 aa, synthetic construct
QVQLQESGGGLVQPGGSLRLSCAASGFTFSAYAMSWVRQAPGKGLEWVSSISSTGTNPNYADSVKGRFTISRDNAKNTLYLQMNSLKPEDTAVYYCAEAYRNYSPPGQGTQVTVSS

SEQ ID NO: 81 - protein, 116 aa, synthetic construct
QVQLQESGGGLVQAGGSLRLSCAASGFAFSSVAMSWVRQAPGKGLEWVSSISSGGHDTNYANSVKGRFTISRDNAKNTLYLQMNSLKPEDTAVYYCAKSSRNYSPPGQGTQVTVSS

SEQ ID NO: 82 - protein, 116 aa, synthetic construct
QVQLQESGGGLVQAGGSLRLSCAASGFTLGSYAMSWVRQAPGKGLEWVSSISSSGTNTNYADSVKGRFTISRDNAKNTLYLQMNSLKPEDTAVYYCAKASQNHSPPGQGTPVTVSS

SEQ ID NO: 83 - protein, 116 aa, synthetic construct
QVQLQESGGGLVQAGGSLRLSCAASGFTLSTYAMSWVRQAPGKGLEWVSSISSSGASTNYADSVKGRFTISRDNAKNTLYLQMNSLKPEDTAVYYCAKASQNYSPPGQGTQVTVSS

SEQ ID NO: 84 - protein, 116 aa, synthetic construct
QVQLQESGGGLVQPGGSLRLSCAASGFTFRSYAMSWVRXAPGKGLEWVSSISSSGTDTNYADSVKGRFTISRDNVKNTLYLQMNSLKPEDTAVYYCAKASQNYSPPGQGTQVTVSS

SEQ ID NO: 85 - protein, 126 aa, synthetic construct
QVQLQESGGGLVQAGDSLRLSCAPSGRTFSTYAMGWFRQAPGKEREFVAGISWSGGRTYYADSVKGRFTISRDNAKNTVYLQMNSLKPEDTAVYYCAADPMDYSGSLYAQRSYDYWGQGTQVTVSS

SEQ ID NO: 86 - protein, 126 aa, synthetic construct
QVQLQESGGGLVQAGGSLRLSCAASGRTFVTYAMGWFRQAPGKEREFVAAISWSGGRLYYADSAKGRFTISRDNAKNTVYLQMNSLKPEDTAVYYCAADPQDYTGRDYQRRRYDYWGQGTQVTVSS

SEQ ID NO: 87 - protein, 126 aa, synthetic construct
QVQLQESGGGLVQAGGSLRLSCAASGRTFSTYAMGWFRQAPGKEREFVAAISWSGGRTYYADSVKGRFAISRDNAKNTAYLQMNSLKPEDTAVYYCAADPSDYMGSYYGARRYDYWGQGTQVTVSS

SEQ ID NO: 88 - protein, 126 aa, synthetic construct
QVQLQESGGGLVQAGGSLRLSCAASGRTFVTYAMGWFRQAPGKEREFVAAISWSGGRLYYADSVKGRFTISRDNAKNTVYLQMNSLKPEDTAVYYCAADPQDYTGRDYQRRRYDYWGQGTQVTVSS

SEQ ID NO: 89 - protein, 126 aa, synthetic construct
QVQLQESGGGLVQAGGSLRLSCAASGRTFSTYAMGWFRQAPGKEREFVAAISWSGGRTYYADSVKGRFTISRDNAKNTAYLQMSSLIPEDTAVYYCAADPADYTGSYYYQRSYDYWGQGTQVTVSS

SEQ ID NO: 90 - protein, 126 aa, synthetic construct
QVQLQESGGGLVQPGGSLRLSCAASGRTFSTYAMGWFRQAPGKEREFVAAISWSGGRTYYADSVKGRFTISRDNAKNTAYLQMSSLIPEDTAVYYCAADPADYTGSYYYQRSYDYWGQGTQVTVSS

SEQ ID NO: 91 - protein, 126 aa, synthetic construct
QVQLQESGGGLVQAGGSLRLSCAASGRTFSTYAMGWFRQAPGKEREFVAAISWSGGRLYYADSVKGRFTISRDNAKNTAYLQMNSLKPEDTAVYYCAADEEDYSGTYYYSRKYNYWGQGTQVTVSS

SEQ ID NO: 92 - protein, 127 aa, synthetic construct
QVQLQESGGGLVQAGGSLRLSCAASGFTFDDYTIGWFRQAPGKEREFVAALSWSAGSTFFTDSVKDRFSISRDNAKNTVLLQMNSLKPEDTAVYYCAAKNPPHRWSDITKSLQYAYWGQGTQVTVSS

SEQ ID NO: 93 - protein, 127 aa, synthetic construct
QVQLQESGGGLVQTGGSLRLSCAASGRTFSNSVMGWFRQAPGKEREFVAALMWSAGSEFYTDSVKGRFSISGDGAKNTVYLQMNSLKAEDTAVYYCAAKNPPHRWSDIGKSPHYEYWGQGTQVTVSS

SEQ ID NO: 94 - protein, 127 aa, synthetic construct
QVQLQESGGGLVQAGGSLRLSCAASGRTFSNSVIGWFRQAPGKEREFVAALSWSAGSEFYVDSFKGRFSISRDNAKNTVYLQMNSLKPEDTAVYYCAAKNPPHRWSDIGKSPQYAYWGQGTQVTVSS

SEQ ID NO: 95 - protein, 127 aa, synthetic construct
QVQLQESGGGLVQPGGSLRLSCAASGRTFSNSVMGWFRQAPGKEREFVAALMWSAGSEFYTDSVKGRFSISGDGAKNTVYLQMNSLKAEDTAVYYCAAKNPPHRWSDIGKSPHYEYWGQGTQVTVSS

SEQ ID NO: 96 - protein, 127 aa, synthetic construct
QVQLQESGGGLVQAGGSLRLSCAASGRTLSNSVMGWFRQAPGKEREFVAALMWSAGSEFYTDSVKGRFSISGDGAKNTVYLQMNSLKAEDTAVYYCAAKNPPHRWSDIGKSPHYEYWGQGTQVTVSS

SEQ ID NO: 97 - protein, 127 aa, synthetic construct
QVQLQESGGGLVXAGGSLRLSCAASGPTFSNSVIGWFRQAPGKEREFVAALSWSADTIFYADSVKDRFSISRDNAKNTVHLQMNSLKPEDTAVYYCAAKNPPHRYSDINNAPHYAYWAQGTQVTVSS

SEQ ID NO: 98 - protein, 124 aa, synthetic construct
QVQLQESGGGLVQAGGSLRLSCAASGRTINAYIMGWFRQAPGKEREFVAAVSGSGGSTYYADSVKGRFTISRDNAKNTVNLQMDNLRSEDTDVYYCAATYESVYLQNPGQYDYWGQGTQVTVSS

SEQ ID NO: 99 - protein, 124 aa, synthetic construct
QVQLQESGGGSVQAGGSLRLSCAASGRTINAYIMGWFRQAPGKEREFVAAVSGSGGSTYYADSVKGRFTISRDNAKNTVNLQMDNLRSEDTDVYYCAATYESVYLRNPGQYDYWGQGTQVTVSS

SEQ ID NO: 100 - protein, 124 aa, synthetic construct
QVQLQESGGGSVQAGGSLRLSCAASGRTINAYIMGWFRQAPGKEREFVAAVSGSGGSTYYADSVKGRFTISRDNAKNTVNLQMDNLRSEDTDVYYCAATYESVYLQNPGQYDYWGQGTQVTVSS

SEQ ID NO: 101 - protein, 124 aa, synthetic construct
QVQLQESGGGLVEPGGSLRLSCAASGRTINAYIMGWFRQAPGKEREFVAAVSGSGGSTYYADSVKGRFTISRDSAKNTVNLQMDNLRSEDTDVYYCAATYESVYLQNPGQYDYWGQGTQVTVSS

SEQ ID NO: 102 - protein, 124 aa, synthetic construct
QVQLQESGGGLVQPGGSLRLSCAASGRTINAYIMGWFRQAPGKEREFVAAVSGSGGSTYYADSVKGRFTISRDNAKNTVNLQMDNLRSEDTDVYYCAATYESVYLQNPGQYDYWGQGTQVTVSS

SEQ ID NO: 103 - protein, 124 aa, synthetic construct
QVQLQESGGGSVQAGGSLRLSCAASGRTINAYVMGWFRQAPGKEREFVAAISGSGGSTYYADSVKGRFTISRDNAKNTVNLQMDNLRSEDTDVYYCAATYESVYLQNPGQYDYWGQGTQVTVSS

SEQ ID NO: 104 - protein, 131 aa, synthetic construct
QVQLQESGGGLVQAGGSLRLSCVASGRTFSSYGMGWFRQAPGKEREFVTGISSAGSWIGDITSYADSVKGRFTISRDNAKNILYLQMNSLKPEDTAVYYCAAAYHGSRYYVGSTANEYTTWGQGTQVTVSS

SEQ ID NO: 105 - protein, 131 aa, synthetic construct
QVQLQESGGGSVQAGGSLRLSCAVFGLSFKDYSMSWFRQAPGKEREFVAGISSGGSWIGDITSYADSVKGRFTISRDNAKNMIYLQMNSLKPDDTGVYYCAAASYGTRYYVGSTANEYTTWGQGTQVTVSS

SEQ ID NO: 106 - protein, 131 aa, synthetic construct
QVQLQESGGGLVQAEGSLRLSCVASGRTFSSYGMGWFRQAPGKEREFVTGISSAGSWIGDITSYADSVKGRFTISRDNAKNILYLQMNSLKPEDTAVYYCAAAYHGSRYYVGSTANEYTTWGQGTQVTVSS

SEQ ID NO: 107 - protein, 131 aa, synthetic construct
QVQLQESGGGLVQAGDSLRLSCAASGRTFSNYNMGWLRQAPGKEREFVTGISSAGSWIGDITSYADSVKGRFTISRDNAKNILYLQMNSLKPEDTAVYYCAAAYHGSRYYVGSTANEYTTWGQGTQVTVSS

SEQ ID NO: 108 - protein, 131 aa, synthetic construct
QVQLQESGGGLVQAGGSLTLSCTASGRTFSSYTTGWFRQAPGKEREFVAGISSGGSWIGDITSYADSVKGRFTISRDNAKNMIYLQMNSLKPDDTGVYYCAAASYGTRYYVGSTANEYTTWGQGTQVTVSS

SEQ ID NO: 109 - protein, 128 aa, synthetic construct
QVQLQESGGGSVQSGGSLRLSCVASGRTFSSYAMGWFRQGPGKEREFVAAISWSGVSIYYADSVKGRFTISRDNAKNTVYLQMNSLEPEDTAVYYCAGTEGAYTVAGGSTFGYDMDYWGKGTQVTVSS

SEQ ID NO: 110 - protein, 128 aa, synthetic construct
QVQLQESGGGLVQAGGSLRLSCAASGRTFSSYAMGWFRQGPGKEREFVAAISWSGVSTYYADSVKGRFTISRDNAKNTVYLQMNSLRPEDTAVYYCAATEGAYTVAGGSTFGYDMDYWGKGTQVTVSS

SEQ ID NO: 111 - protein, 127 aa, synthetic construct
QVQLQESGGGLVQAGESLTLSCIASGVIKDYAMGWFRQAPGKEREFVAAISWSGVSIYYADSVKGRFTISRDNAKNTVYLQMNSLEPEDTAVYYCAGTEGAYTVAGGSTFGYDMDYWGKGTQVTVSS

SEQ ID NO: 112 - protein, 128 aa, synthetic construct
QVQLQESGGGLVQAGGSLRLSCVASGRTFSSYAMGWFRQGPGKEREFVAAISXSGVSIYYADSVKGRFTISRDNAKNTVYLQMNSLEPEDTAAYYCAGTEGAYTVAGGSTFGYDMDYWGKGTQVTVSS

SEQ ID NO: 113 - protein, 128 aa, synthetic construct
QVQLQESGGGLVQAGGSLRLSCVASGRTFSSYAMGWFRQGPGKEREFVAAISWSGVSIYYADSVKGRFTISRDNAKNTVYLQMNSLEPEDTAVYYCAGTEGAYTVAGGSTFGYDMDYWGKGTQVTVSS

SEQ ID NO: 114 - protein, 119 aa, synthetic construct
QVQLQESGGGLVQAGGSLRLSCAPSGRTFSTLAMAWFRQAPGKEREVVATISWSGGRTYYADSVKGRFTISRDNAKNTAYLQMNSLRPDDTAVYYCAARGSDYGSYDYWGQGTQVTVSS

SEQ ID NO: 115 - protein, 119 aa, synthetic construct
QVQLQESGGGLVQAGGSLRLSCAPSGRTFSTYAMGWFRQALGKEREVVATISWSGGRIYYTDSVKGRFTISRDNAKNTAYLQMNSLRPDDTAVYYCAARGTDYGAWEYWGQGTQVTVSS

SEQ ID NO: 116 - protein, 119 aa, synthetic construct
QVQLQESGGGLVQAGGSLRLSCAPSGRTFSTLAMAWFRQAPGKEREVVATISWSGGRTYYADSVKGRFTISRDNAKNTAFLQMNSLRPDDTAVYYCAARESDYGSYDYWGQGTQVTVSS

SEQ ID NO: 117 - protein, 119 aa, synthetic construct
QVQLQESGGGLVQPGGPLRLSCAPSGRTFSTLAMAWFRQAPGKEREVVATISWSGGRTYYADSVKGRFTISRDNAKNTAFLQMNSLRPDDTAVYYCAARESDYGSYDYWGQGTQVTVSS

SEQ ID NO: 118 - protein, 119 aa, synthetic construct
QVQLQESGGGLVQVGGSLRLSCAPSGRTFSTYAMGWFRQALGKEREVVATISWSGGRIYYTDSVKGRFTISRDNAKNTAYLQMNSLRPDDADVYYCATRGTDYGAWEYWGQGTQVTVSS

SEQ ID NO: 119 - protein, 119 aa, synthetic construct
QVQLQESGGGLVQAGGSLRLSCTPSGRTFSTLAMGWFRQAPGKEREVVATISWSGGRTYYASSVKSRFTISRDNAKNTAYLQMNSLRPDDTAVYYCAARGTDYGAYDYWGQGTQVTVSS

SEQ ID NO: 120 - protein, 119 aa, synthetic construct
QVQLQESGGGLVQAGGSLRLSCAPSGRTFSTLAMGWFRQAPGKEREVVATISWSGGRTYYADSVKGRFTISRNNAKNTAYLQMNNLRPDDTAVYYCAARETDYGAYDYWGQGTQVTVSS

SEQ ID NO: 121 - protein, 119 aa, synthetic construct
QVQLQESGGGLVQPGGSLRLSCVSSQTLVGIEAMGWYRQAPGKQRELVALITLNGGTFYADSVKGRFTISRDVAGHMLYLEMNSLNPEDTAVYFCVAGVESDNEEPTYWGQGTQVTVSS

SEQ ID NO: 122 - protein, 119 aa, synthetic construct
QVQLQESGGGLVQAGGSLTLSCVSSQTLVGIEAMGWYRQAPGKQRELVALITLNGGTFYADSVKGRFTISRDVAEHMLYLEMNSLNPEDTAVYFCVAGVESDNEEPTYWGQGTQVTVSS

SEQ ID NO: 123 - protein, 119 aa, synthetic construct
QVQLQESGGGLVQAGGSLRLSCVSSQTLVGIEAMGWYRQAPGKQRELVALITLNGGTFYADSVKGRFTISRDVAEHMLYLEMNSLNPEDTAVYFCVAGVESDNEEPTYWGQGTQVTVSS

SEQ ID NO: 124 - protein, 119 aa, synthetic construct
QVQLQESGGGLVQSGGSLRLSCVSSQTLVGIEAMGWYRQAPGKQRELVALITLNGGTFYADSVKGRFAISRDVAEHMLYLEMNSLNPEDTAVYFCVAGVESDNEEPTYWGQGTQVTVSS

SEQ ID NO: 125 - protein, 119 aa, synthetic construct
QVQLQESGGGLVQSGGSLRLSCVSSQTLVGIEAMGWYRQAPGKQRELVALITLNGGTFYADSVKGRFTISRDVAEHMLYLEMNSLNPEDTAVYFCVAGVESDNEEPTYWGQGTQVTVSS

SEQ ID NO: 126 - protein, 122 aa, synthetic construct
QVQLQESGGGLVQPGGSLRLSCTASGFAFSSYWMYWVRQSPEKGFEWVSVVDTDGGITYYADSVKGRFTISRDNAKNTLYLQMNSLKPQDTALYYCARDRTTLEYTRTYYRTGQGTQVTVSS

SEQ ID NO: 127 - protein, 122 aa, synthetic construct
QVQLQESGGGLVQPGGSLRLSCTASGFAFSSYWMYWVRQTPGKGFEWVSVVNPGGEITYYGDSMEDRFTISRDNAKNTLYLQMNSLKPEDTGLYYCARDRTTIDYTRTYYRGGQGTQVTVSS

SEQ ID NO: 128 - protein, 122 aa, synthetic construct
QVQLQESGGGLVQPGGSLRLSCTASGFAFSTYWMYWIRQSPEKGFEWVSVVNTGGDITYYADSVKGRFTISRDNAKNTLYLQMNSLKPEDTALYYCARDRTTMSYTRTYYRGGQGTQVTVSS

SEQ ID NO: 129 - protein, 125 aa, synthetic construct
QVQLQESGGGLVQAGKSLTLACAASGFPFSTYAMSWVRXAPGKGLEWVSAVNSDGSSTDYADSVKGRFTISRDNAKNMVYLQMNSLKPEDTARYYCAKYEDCSDYVCNRDRATYWGQGTQVTVSS

SEQ ID NO: 130 - protein, 125 aa, synthetic construct
QVQLQESGGGLVQPGGSLRLSCKASGFTFSTYAMSWVRQAPGKGLEWVSAINSGGLSTDYADSVEGRFTISRDDAKNMLFLQMNSLKAEDTARYYCAKYEDCSDYMCHRDRAGYWGQGTQVTVSS

SEQ ID NO: 131 - protein, 125 aa, synthetic construct
QVQLQESGGGLVQPGGSLRLSCKASGFTFSTYAMSWVRQAPGKGLEWVSAVNSGGLSTDYADSVEGRFTISRDDAKNMLFLQMNSLKPEDTARYYCAKYEDCSDYMCHRDRAGYWGQGTQVTVSS

SEQ ID NO: 132 - protein, 127 aa, synthetic construct
QVQLQESGGGLVQAGDSLRLSCTASGHTFSRYTTIGWFRQAPGKDREFVAAIRWSGDITDYADAVKGRFTISRDNVKNTVYLQMNSLNPEDTAVYYCARGYSEYGVLSRNRGLYAYWGQGTQVTVSS

SEQ ID NO: 133 - protein, 127 aa, synthetic construct
QVQLQESGGGLVQAGGSLRLSCTASGHTFSRYTTIGWFRQAPGKDREFVAAIRWSGDITDYADAVKGRFTISRDNVKNTVYLQTNSLNPEDTAVYYCARGYSEYGVLSRNRGLYAYWGQGTQVTVSS

SEQ ID NO: 134 - protein, 127 aa, synthetic construct
QVQLQESGGGLVQAGGSLRLSCTASGHTFSSYTTIGWFRQAPGKEREFVAAIGWSGDITDYADAVKGRFTISRHNVENTVYLQMNSLNPEDTAVYYCARGYSEYGVLSRNRGLYAYWGQGTQVTVSS

SEQ ID NO: 135 - protein, 125 aa, synthetic construct
QVQLQESGGGLVQAGDSLRLSCAASGRTFSSYTMGWFRQAPGKEREFVARISQDGGSTYYADSMKGRFTVARDNAKNTVYLQMNSLKPEDTAVYYCAADMIKWGLRVGVDSFKYWGQGTQVTVSS

SEQ ID NO: 136 - protein, 125 aa, synthetic construct
QVQLQESGGGLVPPGDSLRLSCAASGRTFSSYTMGWFRQAPGKEREFVARISQDGGSTYYADSMKGRFTVARDNAKNTVYLQMNSLKPEDTAVYYCAADMIKWGLRVGVDSFKYWGQGTQVTVSS

SEQ ID NO: 137 - protein, 125 aa, synthetic construct
QVQLQESGGGLVXAGDSLRLSCAASGRTFSSYTMGWFRQAPRKEREFVARISQDGGSTYYADSMKGRFTVARDNAKNTVYLQMNSLKPEDTAVYYCAADMIKWGLRVGVDSFKYWGQGTQVTVSS

SEQ ID NO: 138 - protein, 125 aa, synthetic construct
QVQLQESGGGLVQAGGSLRLSCAASGRTFSNYRMGWFRQAPGKEREFVAGINWDGGHTYYTDSVKGRFTISRDNAKNTVYLQMNSLKPEDTAVYQCAVRVREYGSSRYEDQFDYWGQGTQVTVSS

SEQ ID NO: 139 - protein, 125 aa, synthetic construct
QVQLQESGGGLVQPGESLRLSCQASGRTFSNYRMGWFRQAPGKEREFVAGINWDGGHTYYTDSVKGRFTISRDNAKNTVYLQMNSLKPEDTAVYQCAVRVREYGSSRYEDQFDYWGQGTQVTVSS

SEQ ID NO: 140 - protein, 125 aa, synthetic construct
QVQLQESGGGLVQAGGSLRLSCQASGRTFSNYRMGWFRQAPGKEREFVAGINWDGGHTYYTDSVKGRFTISRDNAKNTVYLQMNSLKPEDTAVYQCAVRVREYGSSRYEDQFDYWGQGTQVTVSS

SEQ ID NO: 141 - protein, 120 aa, synthetic construct
QVQLQESGGGLVQAGMSLRLSCAASGLTFSWYRMGWFRQAPGKEREFVAGWYGVLTQYRDSVKGRFTISRDDAKNTVYLQMNSLKPEDTAVYYCAASLGGNYRSYEYDYWGQGTQVTVSS

SEQ ID NO: 142 - protein, 120 aa, synthetic construct
QVQLQESGGGLVQAGGSLRLSCAASGRTFSWYRMGWFRQAPGKEREFVAGWYGVLTQYRDSVKGRFTISRDDAKNTVYLQMNSLKPEDTAVYYCAASLGGNYRSYEYDYWGQGTQVTVSS

SEQ ID NO: 143 - protein, 120 aa, synthetic construct
QVQLQESGGGLVXPGGSLRLSCAASGRTFSWYRMGWFRQAPGKEREFVAGWYGVLTQYRDSVKGRFTISRDDAKNTVYLQMNSLKPEDTAVYYCAASLGGNYRSYEYDYWGQGTQVTVSS

SEQ ID NO: 144 - protein, 120 aa, synthetic construct
QVQLQESGGGLVQAGGSLRLSCAASGSILRINLMGWYRQVPGKQRELVAEMSTGGSTTYVDSVKGRFTISRDNAKNTVYLQMNSLKPEDTAVYYCNAKAWQLGRLENDDWGQGTQVTVSS

SEQ ID NO: 145 - protein, 120 aa, synthetic construct
QVQLQESGGGLVQPGGSLRLSCAASGSILRINLMGWYRQVPGKQRELVAEMSTGGSTTYVDSVKGRFTISRDNAKNTVYLQMNSLKPEDTAVYYCNAKAWQLGRLENDDWGQGTQVTVSS

SEQ ID NO: 146 - protein, 120 aa, synthetic construct
QVQLQESGGGLVQPGGSLRLSCAASGSILRINLMGWYRQVPGKQRELVAEMSTGGSTTYVDSVEGRFTISRDNAKNTVYLQMNSLKPEDTAVYYCNAKAWQLGRLENDDWGQGTQVTVSS

SEQ ID NO: 147 - protein, 123 aa, synthetic construct
QVQLQESGGGLVQPGGSLRLTCVASGFPFSTHAMSWVRQPPGKGREWISAINSGGDSTDYADSVMGRFTISRDNAKNTVYLQMNSLKSEDTAVYYCAKYEDCSDYDCSSRASWGQGTQVTVSS

SEQ ID NO: 148 - protein, 123 aa, synthetic construct
QVQLQESGGGLVQPGGSLRLTCVASGFPFSTHAMSWVRQPPGKGREWISAINSGGDSTDYADSVMGRFTISRDNAKNTVYLQMNSLKSEDTAAYYCAKYEDCSDYDCSSRASWGQGTQVTVSS

SEQ ID NO: 149 - protein, 123 aa, synthetic construct
QVQLQESGGGLVQAGGSLRLSCAASGFTFSTYAMSWVRQAPGKGLEWVSSVNSGGLSTDYADSVEGRFTISRDDAKNMLFLQMNSLKPEDTARYYCAKYEDCSDYVCDRAGYWGQGTQVTVSS

SEQ ID NO: 150 - protein, 123 aa, synthetic construct
QVQLQESGGGLVQPGGSLRLSCAASGFTFSTYAMSWVRQAPGKGLEWVSAVNSGGLSTDYADSVEGRFTISRDDAKNMLFLQMNRLKPEDTARYYCAKYEDCSDYVCDRAGYWGQGTQVTVSS

SEQ ID NO: 151 - protein, 129 aa, synthetic construct
QVQLQESGGGLVQAGDSLRLSCAASGRTFDTYIVGWFRQAPGKEREFVSALSWSDDTTIRDDSLRGRFTISRDNAKNTVYLLMNSLKPEDTAVYYCAAKAKPPAATWSSYRNREWYDYWGQGTQVTVSS

SEQ ID NO: 152 - protein, 129 aa, synthetic construct
QVQLQESGGGLVQAGDSLRLSCAASGRTFDTYIVGWFRQAPGKEREFVSALSWSDDTTIRDDSLRGRFTLSRDNAKNTVYLLMNSLKPEDTAVYYCAAKAKPPAATWSSYRNREWYDYWGQGTQVTVSS

SEQ ID NO: 153 - protein, 123 aa, synthetic construct
QVQLQESGGGLVQTGGSLRLSCAASGRTYAMAWFRQAPGKEREFAAVIIGGDRTYYANSVKDRFTISRDNAKETVYLQMNNLKPEDTAVYYCAANRRLYNSGRGYSQSEYEYWGQGTQVTVSS

SEQ ID NO: 154 - protein, 123 aa, synthetic construct
QVQLQESGGGLVQAGGSLRLSCAASGRTYAMAWFRXAPGKEREFAAVIIGGDRTYYANSVKDRFTISRDNAKETVYLQMNNLKPEDTAVYYCAANRRLYNSGRGYSQSEYEYWGQGTQVTVSS

SEQ ID NO: 155 - protein, 125 aa, synthetic construct
QVQLQESGGGLVQAGGSLRLSCAASGRTFSDYPMAWFRQAPGKEREFVAGISNGGTAYYADSVKGRFTISRDNTRNTVYLQMNRLKVEDTAVYYCAAVHLLSNLRGADPHEADYWGQGTQVTVSS

SEQ ID NO: 156 - protein, 125 aa, synthetic construct
QVQLQESGGGLVQAGGSLRLSCAASGRTFSTYPMAWFRXIPGKEREFVAGTSNGGSAYYADSVKGRFTISRDNTKNTVYLQMNSLKAEDTAVYYCAAVHLLSSLWGADPHEADYWGQGTQVTVSS

SEQ ID NO: 157 - protein, 125 aa, synthetic construct
QVQLQESGGGLVQAGGSLRLSCAASGRTFSNYAMGWFRQAPGEEREFVAAINRSGTSLYHDSVMGRFTISRDNAKNTVALEMNSLKPEDTAVYYCVAGMPFSYSDYSRDSKYDYXGQGTQVTVSS

SEQ ID NO: 158 - protein, 125 aa, synthetic construct
QVQLQESGGGLVQAGGSLRLSCAASGRTFSNYAMGWFRQAPGKEREFVAAINRSGTTLYVDSVKGRFTISRDNAKNTVALQMNSLKPEDTAVYYCVAGMPFSYSDYPRDSKYDYWGQGTQVTVSS

SEQ ID NO: 159 - protein, 125 aa, synthetic construct
QVQLQESGGGLVQAGGSLRLSCAASGRTFNNYAMAWFRQAPGKEREFIAAITRSDTTLYIDSVKGRFTISRDNAKNTVALQMNSLKPEDTAVYYCAAGMPYSYSDYPRDSKYDYWGRGTQVTVSS

SEQ ID NO: 160 - protein, 130 aa, synthetic construct
QVQLQESGGGVVQEGASLRLSCAASGRNLSTYAMGWIRQAPGKEREFVAAISWAGSTTYYADSVKGRFIISRDNAKNTLNLQMNSLKPEDTAVYYCAAPPYVTTTYYATPPSDIRDYHYWGQGTQVTVSS

SEQ ID NO: 161 - protein, 130 aa, synthetic construct
QVQLQESGGGLVQAGGSLRLSCAASGRNLSTYAMGWIRQAPGKEREFVAAISWAGSTTYYADSVKGRFIISRDNAKNTLNLQMNSLKPEDTAVYYCAAPPYVTTTYYATPPSDIRDYHYWGQGTQVTVSS

SEQ ID NO: 162 - protein, 122 aa, synthetic construct
QVQLQESGGGEVQAGGSLRLSCVASGRTFSVYTTGWFRQAPGKERELVAGMSGTRTYYADSVKGRFTISRDNAKNTVYLQMNSLKPDDTAVYYCAERRYGGPSLVWDEYDYRGQGTQVTVSS

SEQ ID NO: 163 - protein, 122 aa, synthetic construct
QVQLQESGGGEVQAGGSLRLSCVASGRTFSVYTTGWFRQAPGKEREFVAGMSGTRTYYADSVKGRFTISRDNAKNTVYLQMNSLKPDDTAVYYCAERRYGGPSLVWDEYDYRGQGTQVTVSS

SEQ ID NO: 164 - protein, 116 aa, synthetic construct
QVQLQESGGGLVQPGGSLTLSCAASGFTFSSNAMSWVRQAPGKGLEWVSSISSGGGTTYYTDSVKGRFTTARDNAKNTLYLRMNSLKPEDTAVYYCSKSSTGSGSRGQGTQVTVSS

SEQ ID NO: 165 - protein, 120 aa, synthetic construct
QVQLQESGGGLVQTGASLRLSCAASGRTFSTYAMGWFRQAPGKEREFVAHISWSGGRTNYADSVKGRFTISRDNAKNTAYLQMNSLKPEDTAVYYCAARIDDYDRGYDYWGQGTQVTVSS

SEQ ID NO: 166 - protein, 120 aa, synthetic construct
QVQLQESGGGLVQPGGSLRLSCAASGRTFSTYAMGWFRQAPGKEREFVAHISWSGGRTYYADSVKGRFTISRDNAXNTAYLQMNSLKPEDTAVYYCAARIDDYDRGYDYWGQGTQVTVSS

SEQ ID NO: 167 - protein, 125 aa, synthetic construct
QVQLQESGGGLVQAGGSLKLSCAASGNTFSINAMAWYRQAPGQELELVAAISDVGNTIYADSMKGRFTISRGNADNTVYLQMNSLKPEDTATYYCNAGTFYTSPGDSYYRGYDFWGQGTQVTVSS

SEQ ID NO: 168 - protein, 125 aa, synthetic construct
QVQLQESGGGLVQAGGSLKLSCAASGNTFSINAMAWYRQAPGQQLELVAAISDVGNTIYADSMKGRFTISRGNADNTVYLQMNRLKPEDTATYYCNAGTFSTSXGDSYYRGYDFWGQGTQVTVSS

SEQ ID NO: 169 - protein, 119 aa, synthetic construct
QVQLQESGGGLVQAGGSLRLSCAASGSIRSINAMAMAWYRQAPGKQRELVASISNSGSIGYADSVKGRFTISRDNGKNSVYLQMNSLKPEDTAVYLCNGRTGWPRYDLWGQGTQVTVSS

SEQ ID NO: 170 - protein, 118 aa, synthetic construct
QVQLQESGGGLVXAGGSLRLSCAASGSIRSINDMAWYRQAPGKQRELVASISDSGSIDYADSVKGRFTISRDNGKSTVYLQMNSLKPEDTAIYLCNGRTGWPRYDLWGQGTQVTVSSX

SEQ ID NO: 171 - protein, 119 aa, synthetic construct
QVQLQESGGGLVQSGASLRLSCAASERTLSGYAMGWFRQAPGKEREFVAAIRWSGSSTYYAESVEGRFTISRDNAKNTVYLQMNILKPEDTAVYYCAAGSDYADEYDYWGQGTQVTVSS

SEQ ID NO: 172 - protein, 120 aa, synthetic construct
QVQLQESGGGLVQPGGSLILSCVASGFAMDYLTMGWSREAPRNEREDISCIDTSGRSTDYLDSVKGRFTISRDNAKNTLYLHMNDLKPEDTAEYYCLASTSYCGAGYEYRGQGTQVTVSS

SEQ ID NO: 173 - protein, 126 aa, synthetic construct
QVQLQESGGGLVQPGGSLRLSCVESGRTFTVYDMGWFRQAPGKEREIVAAISADGATSIYQDSVKGRFTISRDNDAKNTVYLQMNSLKPEDTAVYYCAAGRSLIYNLYRANNTDYWGQGTQVTVSS

SEQ ID NO: 174 - protein, 125 aa, synthetic construct
QVQLQESGGGLVQTGGSLRLSCVASGRTFNWSAMGWFRQPPGKEREVVATISGGGTSAYYADSVKGRFTISRDGAKNTLYLQMNSLKPEDTAVYYCAADRVDYRMTLTQNGNHYWGQGTQVTVSS

SEQ ID NO: 175 - protein, 129 aa, synthetic construct
QVQLQESGGGLVQAGGSLRLSCAASARTESMYAMAWFRQAPGKEREIVAGIIQSGGTTYISDYMKGRFSIDRGNAENTVYLQMDSLKPEDTAVYSCAAKVSSGTPSDIQISQSRWYDHWGQGTQVTVSS

SEQ ID NO: 176 - protein, 123 aa, synthetic construct
QVQLQESGGGLVRAGGSLRLSCAASGHTFSNYVMGWFRQAPGKEREFVAAISWGGSNTYYADSVKGRFTISRDNAKNTVYLQMNSLKVEDTAVYYCAADRRFEGWNYYSMDYWGKGTQVTVSS

SEQ ID NO: 177 - protein, 127 aa, synthetic construct
QVQLQESGGGLVQAGGSLRLSCAASGRTVDSYAMGWFRQTPGKEREFVAASWGGGSTYYADSVKGRFTISRDNVKNTMYLQMDSLKPEDTAVYYCAAGPTYYSGPINDNWEGRYDSWGQGTQVTVSS

SEQ ID NO: 178 - protein, 118 aa, synthetic construct
QVQLQESGGGLVQPGGSLRLSCAASGRAWNEYNMGWFRQAPGKEREFVALIWWSSGNTYYADSVKGRFTISRDKVKNTVYLQMNSLKPEDTAVYYCAAGPTFNKYEYWGQGTQVTVSS

SEQ ID NO: 179 - protein, 117 aa, synthetic construct
QVQLQESGGGLVQPGGSLRLSCAASGFTFDSYAMSWVRQAPGKGLEWVSSIGSGGGSTAYTESVKGRFTISRDNAKNTLYLQMTSLKPEDTAVYYCTRSRDSHTRPAGXGTQVTVSS

SEQ ID NO: 180 - protein, 118 aa, synthetic construct
QVQLQESGGGLVQPGGSLRLSCAASGSTFSIHAMGWYRQAPGKKRELVARITRGGSTNYADSVKGRFTISVDNAKNTVYLQMNSLKPEDTAVYDCNAETGWGEWDDYWGQGTQVTVSS

SEQ ID NO: 181 - protein, 128 aa, synthetic construct
QVQLQESGGGWVQPGGSLRLSCAASGFTFGNSDMSWVRXAPGKGPEWVSTINTAGGSTYYADSVKGRFTISRDNAKNTLYLQMNSLKPEDTAVYYCATALGSSWYSRFSGDLYEYDYWGQGTQVTVSS

SEQ ID NO: 182 - protein, 126 aa, synthetic construct
QVQLQESGGGLVQAGDFLRLSCVFSGGTFGDYSMGWFRQAPGKEREFVAAIRWSGDSTQYAEAVKGRFTISRDNAKSTVYLQMSSLKPEDTAVYYCAADPSYYIGPNHTPSRYRDWGQGTQVTVSS

SEQ ID NO: 183 - protein, 114 aa, synthetic construct
QVQLQESGGGLVQVGDSLKLSCVASGTTFSTHVMAWYRQAPGKQREWVASIDSAGTTNYKDAVKGRFTISRDNVKRTVYLQMNNLKPEDSAVYLCSIPSRNAWGQGTQVTVSSX

SEQ ID NO: 184 - protein, 130 aa, synthetic construct
QVQLQESGGGLVQAGGSLRLSCAASGRTFSTYAMGWFRQAPGKEREFVAAISWSGGSTYYADSVKGRFTISRDNAKNTGYLQMNSLKPEDTAVYVCAADAEDPILYGSSWNNRRKEYDYWGQGTQVTVSS

SEQ ID NO: 185 - protein, 126 aa, synthetic construct
QVQLQESGGGLVQAGGSLRLSCAASGFTFDDYAIGWFRQAPGKEREGISCISSSDYGTYYAGSVKGRFTISSDNAKNTVYLQMNSLKPEDTAVYYCAAEAISEYDCLGINYGMDYWGKGTQVTVSS

SEQ ID NO: 186 - protein, 126 aa, synthetic construct
QVQLQESGGGLVQAGGSLRLSCAASGRTFGSHRMGWFRQAPGQEREFVAAIAWRGGTQYYTDSVKGRFTISRDNANNTVYLQMNSLKPEDAAVYYCVAGEGSSWGDITDPRRYVFWGQGTQVTVSS

SEQ ID NO: 187 - protein, 125 aa, synthetic construct
QVQLQESGGGLVQAGGSLRLSCAPSGRTFTTYVMGWFRQAPGKEREFVAHISWSGGRTYYADSVKGRFTISRDNAKNTSYLQMNSLKPEDTAVYYCAAARSDAVRIGYAEGYEYWGQGTQVTVSS

SEQ ID NO: 188 - protein, 123 aa, synthetic construct
QVQLQESGGGLVQAGGSLRLSCAASGRTFSSYAMGWFRQVPGKERDFVAAVSWGGLSTYYADSVEGRFTISRDNAKNTVFLQMNSLKPEDTAVYYCAADRAYEGLGYGEYEYWGQGTQVTVSS

SEQ ID NO: 189 - protein, 122 aa, synthetic construct
QVQLQESGGGLVXAGGSLRLSCAASGSTFSTYGMGWFRQAPGNERDFVAGITLIGEESLYAASVKGRFTISRDNAKNTVYLQMNSLKPEDTAVYYCAADSYIKASGWSPRYWGQGTQVTVSS

SEQ ID NO: 190 - protein, 125 aa, synthetic construct
QVQLQESGGGLVQAGGPLRLSCAASGRTFSGSAMGWFRQAPGKEREFVAAITWVGGFTYYADSVKGRFTISRDNAKNTVYLQMNNLSPEDTAVYSCAAQAYHKSGYYYPPLYDYWGQGTQVTVSS

SEQ ID NO: 191 - protein, 121 aa, synthetic construct
QVQLQESGGGLVQAGGSLRLSCAPSGRTFSTYAMGWFRQAPGKEREFVAGISWSGGRINYADSVKGRFTISRDNAKNTAYLQMNSLKPEDTAVYYCAARDSDYGGQEYDYWGQGTQVTVSS

SEQ ID NO: 192 - protein, 126 aa, synthetic construct
QVQLQESGGGLVQAGGSLRLSCAASGLTSSTYVIGWFRQAPGKEREFVAAIMLSGGTTIYADSLKGRCTIYKDNAKNTVYLQMNSLKLEDTAVYYCASGLIYRYSDFPRSQKYNFWGQGTQVTVSS

SEQ ID NO: 193 - protein, 119 aa, synthetic construct
QVQLQESGGGLVQPGASLRLSCATSGPTLSTYAMGWFRQAPGKEREVVATISWSGGRVTYVDSVKGRFTISRDNAKNTAYLQMNRLRPDDTAVYTCAARESDYGAYDYWGQGTQVTVSS

SEQ ID NO: 194 - protein, 115 aa, synthetic construct
QVQLQESGGGSVQPGGSLRLSCGASGFTFSSFGMHWFRQAPGKGQEWAATISSSGGTTYYSDSVKGRFTISRDNAKNTLYLQMNSLKPEDTALYYCARDVTGAVRGQGTQVTVSS

SEQ ID NO: 195 - protein, 115 aa, synthetic construct
QVQLQESGGGSVQAGGSLRLSCGASGFTFSSFGMHWFRQAPGKGQEWVATISSSGGTTYYSDSVKGRFTISRDNAKNTLYLQMNSLKPEDTALYYCARDVTGAVRGQGTQVTVSS

SEQ ID NO: 196 - protein, 129 aa, synthetic construct
QVQLQESGGGLVQAGGSLRLSCAASGRTFSNYAMAWFRQAPGTEREFVAGLSGSGGDTEFYDDSVKGRFAISRDSAKNTVWLQMNSLKPEDTAVYYCAAATHVVVHYGLVRRRGTYDYWGQGTQVTVSS

SEQ ID NO: 197 - protein, 129 aa, synthetic construct
QVQLQESGGGLVQDGGSLRLSCAASGRTFSNYAMAWFRQAPGTEREFVAGLSGSGGDTAFYDNSVMGRFTISRDSAKNTMWLQINNLKPEDTAVYYCAAATHVVVSYGLVRKRGTYDYWGQGTQVTVSS

SEQ ID NO: 198 - protein, 129 aa, synthetic construct
QVQLQESGGGLVQAGGSLRLSCAASGRTFSNYAMAWFRQAPGTEREFVAGLSGSGGDTAFYDNSVMGRFTISRDSAKNTMWLQINNLKPEDTAVYYCAAATHVVVNYGLVRRRGTYDYWGQGTQVTVSS

SEQ ID NO: 199 - protein, 129 aa, synthetic construct
QVQLQESGGGLVQAGGSLRLSCAASGRTFSNYAMAWFRQAPGTEREFVAGLSGSGGDTAFYDNSVMGRFTISRDSAKNTMWLQINNLKPEDTAVYYCAAATHVVVSYGLVRRRGTYDYWGQGTQVTVSS

SEQ ID NO: 200 - protein, 129 aa, synthetic construct
QVQLQESGGGLVQPGGSLRLSCAASGRTFSNYAMAWFRQAPGTEREFVAGLSGSGGDTAFYDNSVMGRFTISRDSAKNTMWLQINNLKPEDTAVYYCAAATHVVVSYGLVRRRGTYDYWGQGTQVTVSS

SEQ ID NO: 201 - protein, 125 aa, synthetic construct
QVQLQESGGGLVQPGGSLRLSCAASRRTFSKHAMAWFRQAPGKEREFVAVVRWSGIITYYADSVKGRFTISRDDAKNTVYLQMDSLKPEDTAVYYCAAGDYRGNGYYVPGMYDYWGQGTQVTVSS

SEQ ID NO: 202 - protein, 127 aa, synthetic construct
QVQLQESGGGLVQAGGSLRLSCAASGRTFSKSTMGWFRQTPGKEREFVAAITWTDTEVYANSVKDRFTISRDNAKNTFYLQMHSLKLEDTAVYYCAGDRYGSSRDSVFSSPTNYPYWGQGTQVTVSS

SEQ ID NO: 203 - protein, 127 aa, synthetic construct
QVQLQESGGGLVXAGGSLRLSCAASGRTFSTSTMAWFRQTPGKEREFVAAITGTDTEVYAHSVKGRFTISRDNAKNTFYLQMHSLKLEDTAVYYCAGDRYGSTRDSVFSSPTNYPYWGQGTQVTVSS

SEQ ID NO: 204 - protein, 124 aa, synthetic construct
QVQLQESGGGLVQPGGSLRLSCAASGISSSRLHVGWFRQSPGKEREFVAAISLSGGTQAYTDSVKGRFTISGDNVKNTAYLQMNSLRPEDTAVYYCAARERFIWTWSDLTYDYWGQGTQVTVSS

SEQ ID NO: 205 - protein, 125 aa, synthetic construct
QVQLQESGGGSVQAGGSLRLSCAVSGRTVSDYAMGWFRQAPGKEREFVAAMSSSETTVYPNSMKGRITISRDNAKNAVYLQLNALKPEDTAVYYCSMGPIATFQGLARQNEYHYWGQGTQVTVSS

SEQ ID NO: 206 - protein, 125 aa, synthetic construct
QVQLQESGGGSVQAGGSLRLSCAVSGRTVSDYAMGWFRQAPGKEREFVAAMSSSETTVYPNSVKGRITISRDNAKNAVYLQMNALKPEDTAVYYCSMGPIVTFQGLARQNEYHYWGQGTQVTVSS

SEQ ID NO: 207 - protein, 125 aa, synthetic construct
QVQLQESGGGSVQAGGSLRLSCAVSGRPVSDYAMGWFRQAPGKEREFVAALSASDTTVYPNSVKGRITISRDNAKNAVDLQMNALKPEDTAVYYCAMGPIGTFQGLARQNEYHYWGQGTQVTVSS

SEQ ID NO: 208 - protein, 125 aa, synthetic construct
QVQLQESGGGSVQAGGSLRLSCAVSGRTVSDYAMGWFRQAPGKEREFVVAMSSSETTVYTNSVKGRITISRDNAKNAVYLQMNALKPEDTAVYYCSMGPITTFQGLARQNEYHYWGQGTQVTVSS

SEQ ID NO: 209 - protein, 123 aa, synthetic construct
QVQLQESGGGLVQAGGSLSLSCAASGSAFRLNFMGWYRQAPGKQRELVAAINSGGTEIYHDSVKGRFTISRDNADSTVWLQMNSLKPDDTAVYYCAADRGISWSKLAGGMDYWGKGTQVTVSS

SEQ ID NO: 210 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - protein, 8 aa, synthetic construct
GRTFSSYG

SEQ ID NO: 212 - protein, 8 aa, synthetic construct
ISRDARIT

SEQ ID NO: 213 - protein, 21 aa, synthetic construct
AAAYTYSGSHYVTARSSDYHS

SEQ ID NO: 214 - protein, 8 aa, synthetic construct
GRTFSSYG

SEQ ID NO: 215 - protein, 8 aa, synthetic construct
ISRDARIT

SEQ ID NO: 216 - protein, 21 aa, synthetic construct
AAAYTYSGSHYVTARSSDYHT

SEQ ID NO: 217 - protein, 8 aa, synthetic construct
GRTFSSYG

SEQ ID NO: 218 - protein, 8 aa, synthetic construct
ISRDARIT

SEQ ID NO: 219 - protein, 21 aa, synthetic construct
AAAYTYSGSHYVTARSSDYHS

SEQ ID NO: 220 - protein, 8 aa, synthetic construct
GRTFSSYG

SEQ ID NO: 221 - protein, 8 aa, synthetic construct
ISRDARIT

SEQ ID NO: 222 - protein, 21 aa, synthetic construct
AAAYTYSGSHYVTARSSDYHS

SEQ ID NO: 223 - protein, 8 aa, synthetic construct
GRTFSSYG

SEQ ID NO: 224 - protein, 8 aa, synthetic construct
ISRGARIT

SEQ ID NO: 225 - protein, 21 aa, synthetic construct
AAAYTYSGSHYVTARSSDYHS

SEQ ID NO: 226 - protein, 8 aa, synthetic construct
GRTFSSYG

SEQ ID NO: 227 - protein, 8 aa, synthetic construct
ISRDARYT

SEQ ID NO: 228 - protein, 21 aa, synthetic construct
AAAYTYSGSHYVTARSGDYNY

SEQ ID NO: 229 - protein, 8 aa, synthetic construct
GRTFSSRG

SEQ ID NO: 230 - protein, 8 aa, synthetic construct
ISRGARNT

SEQ ID NO: 231 - protein, 21 aa, synthetic construct
AAAYIYSGSHYVTARSSDYEY

SEQ ID NO: 232 - protein, 8 aa, synthetic construct
GRTFSMHG

SEQ ID NO: 233 - protein, 8 aa, synthetic construct
ISRDARIT

SEQ ID NO: 234 - protein, 21 aa, synthetic construct
AAAYTYSGSHYVTARSSDYNY

SEQ ID NO: 235 - protein, 8 aa, synthetic construct
GRTFSSYG

SEQ ID NO: 236 - protein, 8 aa, synthetic construct
ISRGARIT

SEQ ID NO: 237 - protein, 21 aa, synthetic construct
AAAYTYSGSHYVTARSSDYHS

SEQ ID NO: 238 - protein, 8 aa, synthetic construct
GRTFSSRT

SEQ ID NO: 239 - protein, 8 aa, synthetic construct
ISRDTAYT

SEQ ID NO: 240 - protein, 21 aa, synthetic construct
AASHIYSGSHYISARSSDYMY

SEQ ID NO: 241 - protein, 8 aa, synthetic construct
GRTFSSYG

SEQ ID NO: 242 - protein, 8 aa, synthetic construct
ISRDASIT

SEQ ID NO: 243 - protein, 21 aa, synthetic construct
AAAYTYSGSHYVTARSSDYHS

SEQ ID NO: 244 - protein, 8 aa, synthetic construct
GFTFSSYG

SEQ ID NO: 245 - protein, 8 aa, synthetic construct
ISRDARIT

SEQ ID NO: 246 - protein, 21 aa, synthetic construct
AAAYTYSGSHYVTARSSDYHS

SEQ ID NO: 247 - protein, 8 aa, synthetic construct
GRTFSSYG

SEQ ID NO: 248 - protein, 8 aa, synthetic construct
ISRDARYT

SEQ ID NO: 249 - protein, 21 aa, synthetic construct
AAAYTYSGSHYVTARSGDYNY

SEQ ID NO: 250 - protein, 8 aa, synthetic construct
GFILDYVG

SEQ ID NO: 251 - protein, 8 aa, synthetic construct
ISRDARIT

SEQ ID NO: 252 - protein, 21 aa, synthetic construct
AAAYTYSGSHYVTARSSDYHT

SEQ ID NO: 253 - protein, 8 aa, synthetic construct
GRTFSSYG

SEQ ID NO: 254 - protein, 8 aa, synthetic construct
ISRDARIT

SEQ ID NO: 255 - protein, 21 aa, synthetic construct
AAAYTYSGSHYVTARSSDYHS

SEQ ID NO: 256 - protein, 8 aa, synthetic construct
GRTFSSYG

SEQ ID NO: 257 - protein, 8 aa, synthetic construct
ISRDARIT

SEQ ID NO: 258 - protein, 21 aa, synthetic construct
AAAYTYSGSHYVTARSSDYHT

SEQ ID NO: 259 - protein, 8 aa, synthetic construct
GRTFSSYG

SEQ ID NO: 260 - protein, 8 aa, synthetic construct
ISRGARIT

SEQ ID NO: 261 - protein, 21 aa, synthetic construct
AAAYTYSGSHYVTARSSDYHS

SEQ ID NO: 262 - protein, 8 aa, synthetic construct
GRTFSSYG

SEQ ID NO: 263 - protein, 8 aa, synthetic construct
ISRDARYT

SEQ ID NO: 264 - protein, 21 aa, synthetic construct
AAAYTYSGSHYVTARSGDYNY

SEQ ID NO: 265 - protein, 8 aa, synthetic construct
GPSFSSYG

SEQ ID NO: 266 - protein, 8 aa, synthetic construct
ISRDARIT

SEQ ID NO: 267 - protein, 21 aa, synthetic construct
AAAYTYSGSHYVTARSSDYHS

SEQ ID NO: 268 - protein, 8 aa, synthetic construct
GRTFSSYG

SEQ ID NO: 269 - protein, 8 aa, synthetic construct
ISRDARIT

SEQ ID NO: 270 - protein, 21 aa, synthetic construct
AAAYTYSGSHYVTARSSDYHS

SEQ ID NO: 271 - protein, 8 aa, synthetic construct
GRTFSSYG

SEQ ID NO: 272 - protein, 8 aa, synthetic construct
ISRGARNT

SEQ ID NO: 273 - protein, 21 aa, synthetic construct
AAAYTYSGSHYVTARSSDYPY

SEQ ID NO: 274 - protein, 8 aa, synthetic construct
GRTFSSYG

SEQ ID NO: 275 - protein, 8 aa, synthetic construct
ISRGARNT

SEQ ID NO: 276 - protein, 21 aa, synthetic construct
AAAYTYSGSHYVTARSSDYPY

SEQ ID NO: 277 - protein, 8 aa, synthetic construct
GRTFSSYG

SEQ ID NO: 278 - protein, 8 aa, synthetic construct
ISRGARNT

SEQ ID NO: 279 - protein, 21 aa, synthetic construct
AAAYTYSGSHYVTARSSDYHT

SEQ ID NO: 280 - protein, 8 aa, synthetic construct
GRTFSSYG

SEQ ID NO: 281 - protein, 8 aa, synthetic construct
ISRDARYT

SEQ ID NO: 282 - protein, 21 aa, synthetic construct
AAAYTYSGSHYVTARSGDYNY

SEQ ID NO: 283 - protein, 8 aa, synthetic construct
GRTFSSYG

SEQ ID NO: 284 - protein, 8 aa, synthetic construct
ISRDARIT

SEQ ID NO: 285 - protein, 21 aa, synthetic construct
AAAYTYSGSHYVTARSSDYHS

SEQ ID NO: 286 - protein, 8 aa, synthetic construct
GRTFSSYG

SEQ ID NO: 287 - protein, 8 aa, synthetic construct
ISRDARIT

SEQ ID NO: 288 - protein, 21 aa, synthetic construct
AAAYTYSGSHYVTARSSDYHS

SEQ ID NO: 289 - protein, 8 aa, synthetic construct
GRTFSSYG

SEQ ID NO: 290 - protein, 8 aa, synthetic construct
GRTFSSYG

SEQ ID NO: 291 - protein, 21 aa, synthetic construct
AAAYTYSGSHYVTARSSDYAY

SEQ ID NO: 292 - protein, 8 aa, synthetic construct
GRTFSSYG

SEQ ID NO: 293 - protein, 8 aa, synthetic construct
ISRGARIT

SEQ ID NO: 294 - protein, 21 aa, synthetic construct
AAAYTYSGSHYVTARSSDYHS

SEQ ID NO: 295 - protein, 8 aa, synthetic construct
GRTFSSYG

SEQ ID NO: 296 - protein, 8 aa, synthetic construct
ISRDARYT

SEQ ID NO: 297 - protein, 21 aa, synthetic construct
AAAYTYSGSHYVTARSGDYNY

SEQ ID NO: 298 - protein, 8 aa, synthetic construct
GSIFAIDD

SEQ ID NO: 299 - protein, 7 aa, synthetic construct
ITATNSA

SEQ ID NO: 300 - protein, 13 aa, synthetic construct
NAKQIVGFQIDEY

SEQ ID NO: 301 - protein, 8 aa, synthetic construct
GSIFAIDD

SEQ ID NO: 302 - protein, 7 aa, synthetic construct
ITATNSA

SEQ ID NO: 303 - protein, 13 aa, synthetic construct
NAKQIVGFQIDEY

SEQ ID NO: 304 - protein, 8 aa, synthetic construct
GSIFASDD

SEQ ID NO: 305 - protein, 7 aa, synthetic construct
ITATNSA

SEQ ID NO: 306 - protein, 13 aa, synthetic construct
NAKQIVGFQIDEY

SEQ ID NO: 307 - protein, 8 aa, synthetic construct
GSIFAIDD

SEQ ID NO: 308 - protein, 7 aa, synthetic construct
ITATNSA

SEQ ID NO: 309 - protein, 13 aa, synthetic construct
NAKQIVGFQIDEY

SEQ ID NO: 310 - protein, 8 aa, synthetic construct
GSIFAIDD

SEQ ID NO: 311 - protein, 7 aa, synthetic construct
ITATNSA

SEQ ID NO: 312 - protein, 13 aa, synthetic construct
NTKQIVGFQIDEY

SEQ ID NO: 313 - protein, 8 aa, synthetic construct
GSIFAIDD

SEQ ID NO: 314 - protein, 7 aa, synthetic construct
ITATNSA

SEQ ID NO: 315 - protein, 13 aa, synthetic construct
NAKQIVGFQIDEY

SEQ ID NO: 316 - protein, 8 aa, synthetic construct
GSIFAIDD

SEQ ID NO: 317 - protein, 7 aa, synthetic construct
ITATNSA

SEQ ID NO: 318 - protein, 13 aa, synthetic construct
NAKQIVGFQIDEY

SEQ ID NO: 319 - protein, 8 aa, synthetic construct
GSIFAIDD

SEQ ID NO: 320 - protein, 7 aa, synthetic construct
ITATNSA

SEQ ID NO: 321 - protein, 13 aa, synthetic construct
NAKQIVGFQIDEY

SEQ ID NO: 322 - protein, 8 aa, synthetic construct
GSIFAIDD

SEQ ID NO: 323 - protein, 7 aa, synthetic construct
ITATNSA

SEQ ID NO: 324 - protein, 13 aa, synthetic construct
NAKQIVGFQIDEY

SEQ ID NO: 325 - protein, 8 aa, synthetic construct
GSIFAIDD

SEQ ID NO: 326 - protein, 7 aa, synthetic construct
ITATNSA

SEQ ID NO: 327 - protein, 13 aa, synthetic construct
NAKQIVGFQIDEY

SEQ ID NO: 328 - protein, 8 aa, synthetic construct
GSIFAIDD

SEQ ID NO: 329 - protein, 7 aa, synthetic construct
ITATNSA

SEQ ID NO: 330 - protein, 13 aa, synthetic construct
NAKQIVGFQIDEY

SEQ ID NO: 331 - protein, 8 aa, synthetic construct
GSIFAIDD

SEQ ID NO: 332 - protein, 7 aa, synthetic construct
ITATNSA

SEQ ID NO: 333 - protein, 13 aa, synthetic construct
NAKQIVGFQIDDY

SEQ ID NO: 334 - protein, 8 aa, synthetic construct
GSIFAIDD

SEQ ID NO: 335 - protein, 7 aa, synthetic construct
ITATNSA

SEQ ID NO: 336 - protein, 13 aa, synthetic construct
NAKQIVGFQIDEY

SEQ ID NO: 337 - protein, 8 aa, synthetic construct
GSIFAIDD

SEQ ID NO: 338 - protein, 7 aa, synthetic construct
ITATNSA

SEQ ID NO: 339 - protein, 13 aa, synthetic construct
NAKQIVGFQIDEY

SEQ ID NO: 340 - protein, 8 aa, synthetic construct
GSIFAIDD

SEQ ID NO: 341 - protein, 7 aa, synthetic construct
ITATNSA

SEQ ID NO: 342 - protein, 13 aa, synthetic construct
NAKQIVGFQIDEY

SEQ ID NO: 343 - protein, 8 aa, synthetic construct
GNIVSIYN

SEQ ID NO: 344 - protein, 7 aa, synthetic construct
ITSSGDP

SEQ ID NO: 345 - protein, 9 aa, synthetic construct
KVPTEKGNY

SEQ ID NO: 346 - protein, 8 aa, synthetic construct
GSIVSIYN

SEQ ID NO: 347 - protein, 7 aa, synthetic construct
ITSSGDP

SEQ ID NO: 348 - protein, 9 aa, synthetic construct
KVPTEKGNY

SEQ ID NO: 349 - protein, 8 aa, synthetic construct
GSILSIYN

SEQ ID NO: 350 - protein, 7 aa, synthetic construct
ITPSGDP

SEQ ID NO: 351 - protein, 9 aa, synthetic construct
KVPTEKGNY

SEQ ID NO: 352 - protein, 8 aa, synthetic construct
GSIFSIYN

SEQ ID NO: 353 - protein, 7 aa, synthetic construct
ITSSGDP

SEQ ID NO: 354 - protein, 9 aa, synthetic construct
KLPTEKGNY

SEQ ID NO: 355 - protein, 8 aa, synthetic construct
GSIVSIYN

SEQ ID NO: 356 - protein, 7 aa, synthetic construct
ITSSGDP

SEQ ID NO: 357 - protein, 9 aa, synthetic construct
KVPTEKGNY

SEQ ID NO: 358 - protein, 8 aa, synthetic construct
GSIFSIYN

SEQ ID NO: 359 - protein, 7 aa, synthetic construct
ITSSGDP

SEQ ID NO: 360 - protein, 9 aa, synthetic construct
KLPTEKGNY

SEQ ID NO: 361 - protein, 8 aa, synthetic construct
GSIFSIYL

SEQ ID NO: 362 - protein, 7 aa, synthetic construct
ITSSGDP

SEQ ID NO: 363 - protein, 9 aa, synthetic construct
KLPTEKENY

SEQ ID NO: 364 - protein, 8 aa, synthetic construct
GSIVSIYN

SEQ ID NO: 365 - protein, 7 aa, synthetic construct
ITSSGDP

SEQ ID NO: 366 - protein, 9 aa, synthetic construct
KVPTEKGNY

SEQ ID NO: 367 - protein, 8 aa, synthetic construct
GSIISIYN

SEQ ID NO: 368 - protein, 7 aa, synthetic construct
ITSNGDS

SEQ ID NO: 369 - protein, 9 aa, synthetic construct
KVPTENGNY

SEQ ID NO: 370 - protein, 8 aa, synthetic construct
GSIFSIYN

SEQ ID NO: 371 - protein, 7 aa, synthetic construct
ITPSGDP

SEQ ID NO: 372 - protein, 9 aa, synthetic construct
KLPTEKGNY

SEQ ID NO: 373 - protein, 8 aa, synthetic construct
GSIFSIYN

SEQ ID NO: 374 - protein, 7 aa, synthetic construct
ITPSGDP

SEQ ID NO: 375 - protein, 9 aa, synthetic construct
KVPTEKGNY

SEQ ID NO: 376 - protein, 8 aa, synthetic construct
GSIFSIYN

SEQ ID NO: 377 - protein, 7 aa, synthetic construct
ITPSGDP

SEQ ID NO: 378 - protein, 9 aa, synthetic construct
KVPTEKGNY

SEQ ID NO: 379 - protein, 8 aa, synthetic construct
GSIFSIYN

SEQ ID NO: 380 - protein, 7 aa, synthetic construct
ITPSGDP

SEQ ID NO: 381 - protein, 9 aa, synthetic construct
KVPTEKGNY

SEQ ID NO: 382 - protein, 8 aa, synthetic construct
GSIFSIYN

SEQ ID NO: 383 - protein, 7 aa, synthetic construct
ITSSGDP

SEQ ID NO: 384 - protein, 9 aa, synthetic construct
KVPTEKGNY

SEQ ID NO: 385 - protein, 8 aa, synthetic construct
GFTFSTSG

SEQ ID NO: 386 - protein, 8 aa, synthetic construct
ISSGGGDT

SEQ ID NO: 387 - protein, 8 aa, synthetic construct
SKDPSGAT

SEQ ID NO: 388 - protein, 8 aa, synthetic construct
GFTFSTSG

SEQ ID NO: 389 - protein, 8 aa, synthetic construct
ISSGGGDT

SEQ ID NO: 390 - protein, 8 aa, synthetic construct
SKDPSGAT

SEQ ID NO: 391 - protein, 8 aa, synthetic construct
GFTFSTSG

SEQ ID NO: 392 - protein, 8 aa, synthetic construct
IGIIVGSS

SEQ ID NO: 393 - protein, 8 aa, synthetic construct
SKDVTGSS

SEQ ID NO: 394 - protein, 8 aa, synthetic construct
GFTFSTSG

SEQ ID NO: 395 - protein, 8 aa, synthetic construct
IGIIVDSS

SEQ ID NO: 396 - protein, 8 aa, synthetic construct
GFTFSSSG

SEQ ID NO: 397 - protein, 8 aa, synthetic construct
SKDPTGSS

SEQ ID NO: 398 - protein, 8 aa, synthetic construct
ISSGGDVT

SEQ ID NO: 399 - protein, 8 aa, synthetic construct
SKDPTGAT

SEQ ID NO: 400 - protein, 8 aa, synthetic construct
GFTFSTSG

SEQ ID NO: 401 - protein, 8 aa, synthetic construct
IGIIVDSS

SEQ ID NO: 402 - protein, 8 aa, synthetic construct
SKDPTGSS

SEQ ID NO: 403 - protein, 8 aa, synthetic construct
GFTFSTFG

SEQ ID NO: 404 - protein, 8 aa, synthetic construct
IGIIDDNS

SEQ ID NO: 405 - protein, 8 aa, synthetic construct
SKDTTGSS

SEQ ID NO: 406 - protein, 8 aa, synthetic construct
GFTFSTSG

SEQ ID NO: 407 - protein, 8 aa, synthetic construct
IGIIVDSS

SEQ ID NO: 408 - protein, 8 aa, synthetic construct
SKDPTGSS

SEQ ID NO: 409 - protein, 8 aa, synthetic construct
GFTFSTSG

SEQ ID NO: 410 - protein, 8 aa, synthetic construct
IGIIVDSS

SEQ ID NO: 411 - protein, 8 aa, synthetic construct
SKDVTGSS

SEQ ID NO: 412 - protein, 8 aa, synthetic construct
GFTFSTSG

SEQ ID NO: 413 - protein, 8 aa, synthetic construct
IGATVDNS

SEQ ID NO: 414 - protein, 8 aa, synthetic construct
SKDPTGSS

SEQ ID NO: 415 - protein, 8 aa, synthetic construct
GSAALIFL

SEQ ID NO: 416 - protein, 7 aa, synthetic construct
VSRGGST

SEQ ID NO: 417 - protein, 13 aa, synthetic construct
KADQNYGDSLRSS

SEQ ID NO: 418 - protein, 8 aa, synthetic construct
GSAALIFL

SEQ ID NO: 419 - protein, 7 aa, synthetic construct
VSRYGST

SEQ ID NO: 420 - protein, 13 aa, synthetic construct
KADQYYGDSLRSS

SEQ ID NO: 421 - protein, 8 aa, synthetic construct
GSAALIFL

SEQ ID NO: 422 - protein, 7 aa, synthetic construct
VSRRGST

SEQ ID NO: 423 - protein, 13 aa, synthetic construct
KADQYYGDSLRSS

SEQ ID NO: 424 - protein, 8 aa, synthetic construct
GSAALIFL

SEQ ID NO: 425 - protein, 7 aa, synthetic construct
VSRGGST

SEQ ID NO: 426 - protein, 13 aa, synthetic construct
KADQNYGDSLRSS

SEQ ID NO: 427 - protein, 8 aa, synthetic construct
GSAALIFL

SEQ ID NO: 428 - protein, 7 aa, synthetic construct
VSRYGST

SEQ ID NO: 429 - protein, 13 aa, synthetic construct
KADQYYGDSLRSS

SEQ ID NO: 430 - protein, 8 aa, synthetic construct
GSAALIFL

SEQ ID NO: 431 - protein, 7 aa, synthetic construct
VSRGGST

SEQ ID NO: 432 - protein, 13 aa, synthetic construct
KADQNYGDSLRSS

SEQ ID NO: 433 - protein, 8 aa, synthetic construct
GSAALIFL

SEQ ID NO: 434 - protein, 7 aa, synthetic construct
VSRYGST

SEQ ID NO: 435 - protein, 13 aa, synthetic construct
KADQYYGDSLRSS

SEQ ID NO: 436 - protein, 8 aa, synthetic construct
GSAALIFL

SEQ ID NO: 437 - protein, 7 aa, synthetic construct
VSRYGST

SEQ ID NO: 438 - protein, 13 aa, synthetic construct
KADQYYGDSLRSS

SEQ ID NO: 439 - protein, 8 aa, synthetic construct
GSAALIFL

SEQ ID NO: 440 - protein, 7 aa, synthetic construct
VSRYGST

SEQ ID NO: 441 - protein, 13 aa, synthetic construct
KADQYYGDSLRSS

SEQ ID NO: 442 - protein, 8 aa, synthetic construct
GFTFRSYA

SEQ ID NO: 443 - protein, 8 aa, synthetic construct
ISSSGTDT

SEQ ID NO: 444 - protein, 9 aa, synthetic construct
AKASQNYSP

SEQ ID NO: 445 - protein, 8 aa, synthetic construct
GFTISNYA

SEQ ID NO: 446 - protein, 8 aa, synthetic construct
IGSNIAST

SEQ ID NO: 447 - protein, 9 aa, synthetic construct
AKASQNHSP

SEQ ID NO: 448 - protein, 8 aa, synthetic construct
GFTFSAYA

SEQ ID NO: 449 - protein, 8 aa, synthetic construct
ISSTGTNP

SEQ ID NO: 450 - protein, 9 aa, synthetic construct
AEAYRNYSP

SEQ ID NO: 451 - protein, 8 aa, synthetic construct
GFAFSSVA

SEQ ID NO: 452 - protein, 8 aa, synthetic construct
ISSGGHDT

SEQ ID NO: 453 - protein, 9 aa, synthetic construct
AKSSRNYSP

SEQ ID NO: 454 - protein, 8 aa, synthetic construct
GFTLGSYA

SEQ ID NO: 455 - protein, 8 aa, synthetic construct
ISSSGTNT

SEQ ID NO: 456 - protein, 9 aa, synthetic construct
AKASQNHSP

SEQ ID NO: 457 - protein, 8 aa, synthetic construct
GFTLSTYA

SEQ ID NO: 458 - protein, 8 aa, synthetic construct
ISSSGAST

SEQ ID NO: 459 - protein, 9 aa, synthetic construct
AKASQNYSP

SEQ ID NO: 460 - protein, 8 aa, synthetic construct
GFTFRSYA

SEQ ID NO: 461 - protein, 8 aa, synthetic construct
ISSSGTDT

SEQ ID NO: 462 - protein, 9 aa, synthetic construct
AKASQNYSP

SEQ ID NO: 463 - protein, 8 aa, synthetic construct
GRTFSTYA

SEQ ID NO: 464 - protein, 8 aa, synthetic construct
ISWSGGRT

SEQ ID NO: 465 - protein, 19 aa, synthetic construct
AADPMDYSGSLYAQRSYDY

SEQ ID NO: 466 - protein, 8 aa, synthetic construct
GRTFVTYA

SEQ ID NO: 467 - protein, 8 aa, synthetic construct
ISWSGGRL

SEQ ID NO: 468 - protein, 19 aa, synthetic construct
AADPQDYTGRDYQRRRYDY

SEQ ID NO: 469 - protein, 8 aa, synthetic construct
GRTFSTYA

SEQ ID NO: 470 - protein, 8 aa, synthetic construct
ISWSGGRT

SEQ ID NO: 471 - protein, 19 aa, synthetic construct
AADPSDYMGSYYGARRYDY

SEQ ID NO: 472 - protein, 8 aa, synthetic construct
GRTFVTYA

SEQ ID NO: 473 - protein, 8 aa, synthetic construct
ISWSGGRL

SEQ ID NO: 474 - protein, 19 aa, synthetic construct
AADPQDYTGRDYQRRRYDY

SEQ ID NO: 475 - protein, 8 aa, synthetic construct
GRTFSTYA

SEQ ID NO: 476 - protein, 8 aa, synthetic construct
ISWSGGRT

SEQ ID NO: 477 - protein, 19 aa, synthetic construct
AADPADYTGSYYYQRSYDY

SEQ ID NO: 478 - protein, 8 aa, synthetic construct
GRTFSTYA

SEQ ID NO: 479 - protein, 8 aa, synthetic construct
ISWSGGRT

SEQ ID NO: 480 - protein, 19 aa, synthetic construct
AADPADYTGSYYYQRSYDY

SEQ ID NO: 481 - protein, 8 aa, synthetic construct
GRTFSTYA

SEQ ID NO: 482 - protein, 8 aa, synthetic construct
ISWSGGRL

SEQ ID NO: 483 - protein, 19 aa, synthetic construct
AADEEDYSGTYYYSRKYNY

SEQ ID NO: 484 - protein, 8 aa, synthetic construct
GFTFDDYT

SEQ ID NO: 485 - protein, 8 aa, synthetic construct
LSWSAGST

SEQ ID NO: 486 - protein, 20 aa, synthetic construct
AAKNPPHRWSDITKSLQYAY

SEQ ID NO: 487 - protein, 8 aa, synthetic construct
GRTFSNSV

SEQ ID NO: 488 - protein, 8 aa, synthetic construct
LMWSAGSE

SEQ ID NO: 489 - protein, 20 aa, synthetic construct
AAKNPPHRWSDIGKSPHYEY

SEQ ID NO: 490 - protein, 8 aa, synthetic construct
GRTFSNSV

SEQ ID NO: 491 - protein, 8 aa, synthetic construct
LSWSAGSE

SEQ ID NO: 492 - protein, 20 aa, synthetic construct
AAKNPPHRWSDIGKSPQYAY

SEQ ID NO: 493 - protein, 8 aa, synthetic construct
GRTFSNSV

SEQ ID NO: 494 - protein, 8 aa, synthetic construct
LMWSAGSE

SEQ ID NO: 495 - protein, 20 aa, synthetic construct
AAKNPPHRWSDIGKSPHYEY

SEQ ID NO: 496 - protein, 8 aa, synthetic construct
GRTLSNSV

SEQ ID NO: 497 - protein, 8 aa, synthetic construct
LMWSAGSE

SEQ ID NO: 498 - protein, 20 aa, synthetic construct
AAKNPPHRWSDIGKSPHYEY

SEQ ID NO: 499 - protein, 8 aa, synthetic construct
GPTFSNSV

SEQ ID NO: 500 - protein, 8 aa, synthetic construct
LSWSADTI

SEQ ID NO: 501 - protein, 20 aa, synthetic construct
AAKNPPHRYSDINNAPHYAY

SEQ ID NO: 502 - protein, 8 aa, synthetic construct
GRTINAYI

SEQ ID NO: 503 - protein, 8 aa, synthetic construct
VSGSGGST

SEQ ID NO: 504 - protein, 17 aa, synthetic construct
AATYESVYLQNPGQYDY

SEQ ID NO: 505 - protein, 8 aa, synthetic construct
GRTINAYI

SEQ ID NO: 506 - protein, 8 aa, synthetic construct
VSGSGGST

SEQ ID NO: 507 - protein, 17 aa, synthetic construct
AATYESVYLRNPGQYDY

SEQ ID NO: 508 - protein, 8 aa, synthetic construct
GRTINAYI

SEQ ID NO: 509 - protein, 8 aa, synthetic construct
VSGSGGST

SEQ ID NO: 510 - protein, 17 aa, synthetic construct
AATYESVYLQNPGQYDY

SEQ ID NO: 511 - protein, 8 aa, synthetic construct
GRTINAYI

SEQ ID NO: 512 - protein, 8 aa, synthetic construct
VSGSGGST

SEQ ID NO: 513 - protein, 17 aa, synthetic construct
AATYESVYLQNPGQYDY

SEQ ID NO: 514 - protein, 8 aa, synthetic construct
GRTINAYI

SEQ ID NO: 515 - protein, 8 aa, synthetic construct
VSGSGGST

SEQ ID NO: 516 - protein, 17 aa, synthetic construct
AATYESVYLQNPGQYDY

SEQ ID NO: 517 - protein, 8 aa, synthetic construct
GRTINAYV

SEQ ID NO: 518 - protein, 8 aa, synthetic construct
ISGSGGST

SEQ ID NO: 519 - protein, 17 aa, synthetic construct
AATYESVYLQNPGQYDY

SEQ ID NO: 520 - protein, 8 aa, synthetic construct
GRTFSTYA

SEQ ID NO: 521 - protein, 8 aa, synthetic construct
ISWSGGST

SEQ ID NO: 522 - protein, 23 aa, synthetic construct
AADAEDPILYGSSWNNRRKEYDY

SEQ ID NO: 523 - protein, 8 aa, synthetic construct
GLSFKDYS

SEQ ID NO: 524 - protein, 12 aa, synthetic construct
ISSGGSWIGDIT

SEQ ID NO: 525 - protein, 20 aa, synthetic construct
AAASYGTRYYVGSTANEYTT

SEQ ID NO: 526 - protein, 8 aa, synthetic construct
GRTFSSYG

SEQ ID NO: 527 - protein, 12 aa, synthetic construct
ISSAGSWIGDIT

SEQ ID NO: 528 - protein, 20 aa, synthetic construct
AAAYHGSRYYVGSTANEYTT

SEQ ID NO: 529 - protein, 8 aa, synthetic construct
GRTFSNYN

SEQ ID NO: 530 - protein, 12 aa, synthetic construct
ISSAGSWIGDIT

SEQ ID NO: 531 - protein, 20 aa, synthetic construct
AAAYHGSRYYVGSTANEYTT

SEQ ID NO: 532 - protein, 8 aa, synthetic construct
GRTFSSYT

SEQ ID NO: 533 - protein, 12 aa, synthetic construct
ISSGGSWIGDIT

SEQ ID NO: 534 - protein, 20 aa, synthetic construct
AAASYGTRYYVGSTANEYTT

SEQ ID NO: 535 - protein, 8 aa, synthetic construct
GRTFSSYA

SEQ ID NO: 536 - protein, 8 aa, synthetic construct
ISWSGVSI

SEQ ID NO: 537 - protein, 21 aa, synthetic construct
AGTEGAYTVAGGSTFGYDMDY

SEQ ID NO: 538 - protein, 8 aa, synthetic construct
GRTFSSYA

SEQ ID NO: 539 - protein, 8 aa, synthetic construct
ISWSGVST

SEQ ID NO: 540 - protein, 21 aa, synthetic construct
AATEGAYTVAGGSTFGYDMDY

SEQ ID NO: 541 - protein, 7 aa, synthetic construct
GVIKDYA

SEQ ID NO: 542 - protein, 8 aa, synthetic construct
ISWSGVSI

SEQ ID NO: 543 - protein, 21 aa, synthetic construct
AGTEGAYTVAGGSTFGYDMDY

SEQ ID NO: 544 - protein, 8 aa, synthetic construct
GRTFSSYA

SEQ ID NO: 545 - protein, 8 aa, synthetic construct
ISXSGVSI

SEQ ID NO: 546 - protein, 21 aa, synthetic construct
AGTEGAYTVAGGSTFGYDMDY

SEQ ID NO: 547 - protein, 8 aa, synthetic construct
GRTFSSYA

SEQ ID NO: 548 - protein, 8 aa, synthetic construct
ISWSGVSI

SEQ ID NO: 549 - protein, 21 aa, synthetic construct
AGTEGAYTVAGGSTFGYDMDY

SEQ ID NO: 550 - protein, 8 aa, synthetic construct
GRTFSTLA

SEQ ID NO: 551 - protein, 8 aa, synthetic construct
ISWSGGRT

SEQ ID NO: 552 - protein, 12 aa, synthetic construct
AARGSDYGSYDY

SEQ ID NO: 553 - protein, 8 aa, synthetic construct
GRTFSTYA

SEQ ID NO: 554 - protein, 8 aa, synthetic construct
ISWSGGRI

SEQ ID NO: 555 - protein, 12 aa, synthetic construct
AARGTDYGAWEY

SEQ ID NO: 556 - protein, 8 aa, synthetic construct
GRTFSTLA

SEQ ID NO: 557 - protein, 8 aa, synthetic construct
ISWSGGRT

SEQ ID NO: 558 - protein, 12 aa, synthetic construct
AARESDYGSYDY

SEQ ID NO: 559 - protein, 8 aa, synthetic construct
GRTFSTLA

SEQ ID NO: 560 - protein, 8 aa, synthetic construct
ISWSGGRT

SEQ ID NO: 561 - protein, 12 aa, synthetic construct
AARESDYGSYDY

SEQ ID NO: 562 - protein, 8 aa, synthetic construct
GRTFSTYA

SEQ ID NO: 563 - protein, 8 aa, synthetic construct
ISWSGGRI

SEQ ID NO: 564 - protein, 12 aa, synthetic construct
ATRGTDYGAWEY

SEQ ID NO: 565 - protein, 8 aa, synthetic construct
GRTFSTLA

SEQ ID NO: 566 - protein, 8 aa, synthetic construct
ISWSGGRT

SEQ ID NO: 567 - protein, 12 aa, synthetic construct
AARGTDYGAYDY

SEQ ID NO: 568 - protein, 8 aa, synthetic construct
GRTFSTLA

SEQ ID NO: 569 - protein, 8 aa, synthetic construct
ISWSGGRT

SEQ ID NO: 570 - protein, 12 aa, synthetic construct
AARETDYGAYDY

SEQ ID NO: 571 - protein, 8 aa, synthetic construct
QTLVGIEA

SEQ ID NO: 572 - protein, 7 aa, synthetic construct
ITLNGGT

SEQ ID NO: 573 - protein, 13 aa, synthetic construct
VAGVESDNEEPTY

SEQ ID NO: 574 - protein, 8 aa, synthetic construct
QTLVGIEA

SEQ ID NO: 575 - protein, 7 aa, synthetic construct
ITLNGGT

SEQ ID NO: 576 - protein, 13 aa, synthetic construct
VAGVESDNEEPTY

SEQ ID NO: 577 - protein, 8 aa, synthetic construct
QTLVGIEA

SEQ ID NO: 578 - protein, 7 aa, synthetic construct
ITLNGGT

SEQ ID NO: 579 - protein, 13 aa, synthetic construct
VAGVESDNEEPTY

SEQ ID NO: 580 - protein, 8 aa, synthetic construct
QTLVGIEA

SEQ ID NO: 581 - protein, 7 aa, synthetic construct
ITLNGGT

SEQ ID NO: 582 - protein, 13 aa, synthetic construct
VAGVESDNEEPTY

SEQ ID NO: 583 - protein, 8 aa, synthetic construct
QTLVGIEA

SEQ ID NO: 584 - protein, 7 aa, synthetic construct
ITLNGGT

SEQ ID NO: 585 - protein, 13 aa, synthetic construct
VAGVESDNEEPTY

SEQ ID NO: 586 - protein, 8 aa, synthetic construct
GFAFSSYW

SEQ ID NO: 587 - protein, 8 aa, synthetic construct
VDTDGGIT

SEQ ID NO: 588 - protein, 15 aa, synthetic construct
ARDRTTLEYTRTYYR

SEQ ID NO: 589 - protein, 8 aa, synthetic construct
GFAFSSYW

SEQ ID NO: 590 - protein, 8 aa, synthetic construct
VNPGGEIT

SEQ ID NO: 591 - protein, 15 aa, synthetic construct
ARDRTTIDYTRTYYR

SEQ ID NO: 592 - protein, 8 aa, synthetic construct
GFAFSTYW

SEQ ID NO: 593 - protein, 8 aa, synthetic construct
VNTGGDIT

SEQ ID NO: 594 - protein, 15 aa, synthetic construct
ARDRTTMSYTRTYYR

SEQ ID NO: 595 - protein, 8 aa, synthetic construct
GFPFSTYA

SEQ ID NO: 596 - protein, 8 aa, synthetic construct
VNSDGSST

SEQ ID NO: 597 - protein, 18 aa, synthetic construct
AKYEDCSDYVCNRDRATY

SEQ ID NO: 598 - protein, 8 aa, synthetic construct
GFTFSTYA

SEQ ID NO: 599 - protein, 8 aa, synthetic construct
INSGGLST

SEQ ID NO: 600 - protein, 18 aa, synthetic construct
AKYEDCSDYMCHRDRAGY

SEQ ID NO: 601 - protein, 8 aa, synthetic construct
GFTFSTYA

SEQ ID NO: 602 - protein, 8 aa, synthetic construct
VNSGGLST

SEQ ID NO: 603 - protein, 18 aa, synthetic construct
AKYEDCSDYMCHRDRAGY

SEQ ID NO: 604 - protein, 9 aa, synthetic construct
GHTFSRYTT

SEQ ID NO: 605 - protein, 8 aa, synthetic construct
IRWSGDIT

SEQ ID NO: 606 - protein, 19 aa, synthetic construct
ARGYSEYGVLSRNRGLYAY

SEQ ID NO: 607 - protein, 9 aa, synthetic construct
GHTFSRYTT

SEQ ID NO: 608 - protein, 8 aa, synthetic construct
IRWSGDIT

SEQ ID NO: 609 - protein, 19 aa, synthetic construct
ARGYSEYGVLSRNRGLYAY

SEQ ID NO: 610 - protein, 9 aa, synthetic construct
GHTFSSYTT

SEQ ID NO: 611 - protein, 8 aa, synthetic construct
IGWSGDIT

SEQ ID NO: 612 - protein, 19 aa, synthetic construct
ARGYSEYGVLSRNRGLYAY

SEQ ID NO: 613 - protein, 8 aa, synthetic construct
GRTFSSYT

SEQ ID NO: 614 - protein, 8 aa, synthetic construct
ISQDGGST

SEQ ID NO: 615 - protein, 18 aa, synthetic construct
AADMIKWGLRVGVDSFKY

SEQ ID NO: 616 - protein, 8 aa, synthetic construct
GRTFSSYT

SEQ ID NO: 617 - protein, 8 aa, synthetic construct
ISQDGGST

SEQ ID NO: 618 - protein, 18 aa, synthetic construct
AADMIKWGLRVGVDSFKY

SEQ ID NO: 619 - protein, 8 aa, synthetic construct
GRTFSSYT

SEQ ID NO: 620 - protein, 8 aa, synthetic construct
ISQDGGST

SEQ ID NO: 621 - protein, 18 aa, synthetic construct
AADMIKWGLRVGVDSFKY

SEQ ID NO: 622 - protein, 8 aa, synthetic construct
GRTFSNYR

SEQ ID NO: 623 - protein, 8 aa, synthetic construct
INWDGGHT

SEQ ID NO: 624 - protein, 18 aa, synthetic construct
AVRVREYGSSRYEDQFDY

SEQ ID NO: 625 - protein, 8 aa, synthetic construct
GRTFSNYR

SEQ ID NO: 626 - protein, 8 aa, synthetic construct
INWDGGHT

SEQ ID NO: 627 - protein, 18 aa, synthetic construct
AVRVREYGSSRYEDQFDY

SEQ ID NO: 628 - protein, 8 aa, synthetic construct
GRTFSNYR

SEQ ID NO: 629 - protein, 8 aa, synthetic construct
INWDGGHT

SEQ ID NO: 630 - protein, 18 aa, synthetic construct
AVRVREYGSSRYEDQFDY

SEQ ID NO: 631 - protein, 8 aa, synthetic construct
GLTFSWYR

SEQ ID NO: 632 - protein, 6 aa, synthetic construct
WYGVLT

SEQ ID NO: 633 - protein, 15 aa, synthetic construct
AASLGGNYRSYEYDY

SEQ ID NO: 634 - protein, 8 aa, synthetic construct
GRTFSWYR

SEQ ID NO: 635 - protein, 6 aa, synthetic construct
WYGVLT

SEQ ID NO: 636 - protein, 15 aa, synthetic construct
AASLGGNYRSYEYDY

SEQ ID NO: 637 - protein, 8 aa, synthetic construct
GRTFSWYR

SEQ ID NO: 638 - protein, 6 aa, synthetic construct
WYGVLT

SEQ ID NO: 639 - protein, 15 aa, synthetic construct
AASLGGNYRSYEYDY

SEQ ID NO: 640 - protein, 8 aa, synthetic construct
GSILRINL

SEQ ID NO: 641 - protein, 7 aa, synthetic construct
MSTGGST

SEQ ID NO: 642 - protein, 14 aa, synthetic construct
NAKAWQLGRLENDD

SEQ ID NO: 643 - protein, 8 aa, synthetic construct
GSILRINL

SEQ ID NO: 644 - protein, 7 aa, synthetic construct
MSTGGST

SEQ ID NO: 645 - protein, 14 aa, synthetic construct
NAKAWQLGRLENDD

SEQ ID NO: 646 - protein, 8 aa, synthetic construct
GSILRINL

SEQ ID NO: 647 - protein, 7 aa, synthetic construct
MSTGGST

SEQ ID NO: 648 - protein, 14 aa, synthetic construct
NAKAWQLGRLENDD

SEQ ID NO: 649 - protein, 8 aa, synthetic construct
GFPFSTHA

SEQ ID NO: 650 - protein, 8 aa, synthetic construct
INSGGDST

SEQ ID NO: 651 - protein, 16 aa, synthetic construct
AKYEDCSDYDCSSRAS

SEQ ID NO: 652 - protein, 8 aa, synthetic construct
GFPFSTHA

SEQ ID NO: 653 - protein, 8 aa, synthetic construct
INSGGDST

SEQ ID NO: 654 - protein, 16 aa, synthetic construct
AKYEDCSDYDCSSRAS

SEQ ID NO: 655 - protein, 8 aa, synthetic construct
GFTFSTYA

SEQ ID NO: 656 - protein, 8 aa, synthetic construct
VNSGGLST

SEQ ID NO: 657 - protein, 16 aa, synthetic construct
AKYEDCSDYVCDRAGY

SEQ ID NO: 658 - protein, 8 aa, synthetic construct
GFTFSTYA

SEQ ID NO: 659 - protein, 8 aa, synthetic construct
VNSGGLST

SEQ ID NO: 660 - protein, 16 aa, synthetic construct
AKYEDCSDYVCDRAGY

SEQ ID NO: 661 - protein, 8 aa, synthetic construct
GRTFDTYI

SEQ ID NO: 662 - protein, 8 aa, synthetic construct
LSWSDDTT

SEQ ID NO: 663 - protein, 22 aa, synthetic construct
AAKAKPPAATWSSYRNREWYDY

SEQ ID NO: 664 - protein, 8 aa, synthetic construct
GRTFDTYI

SEQ ID NO: 665 - protein, 8 aa, synthetic construct
LSWSDDTT

SEQ ID NO: 666 - protein, 22 aa, synthetic construct
AAKAKPPAATWSSYRNREWYDY

SEQ ID NO: 667 - protein, 5 aa, synthetic construct
GRTYA

SEQ ID NO: 668 - protein, 7 aa, synthetic construct
IIGGDRT

SEQ ID NO: 669 - protein, 20 aa, synthetic construct
AANRRLYNSGRGYSQSEYEY

SEQ ID NO: 670 - protein, 5 aa, synthetic construct
GRTYA

SEQ ID NO: 671 - protein, 7 aa, synthetic construct
IIGGDRT

SEQ ID NO: 672 - protein, 20 aa, synthetic construct
AANRRLYNSGRGYSQSEYEY

SEQ ID NO: 673 - protein, 8 aa, synthetic construct
GRTFSDYP

SEQ ID NO: 674 - protein, 7 aa, synthetic construct
ISNGGTA

SEQ ID NO: 675 - protein, 19 aa, synthetic construct
AAVHLLSNLRGADPHEADY

SEQ ID NO: 676 - protein, 8 aa, synthetic construct
GRTFSTYP

SEQ ID NO: 677 - protein, 7 aa, synthetic construct
TSNGGSA

SEQ ID NO: 678 - protein, 19 aa, synthetic construct
AAVHLLSSLWGADPHEADY

SEQ ID NO: 679 - protein, 8 aa, synthetic construct
GRTFSNYA

SEQ ID NO: 680 - protein, 7 aa, synthetic construct
INRSGTS

SEQ ID NO: 681 - protein, 19 aa, synthetic construct
VAGMPFSYSDYSRDSKYDY

SEQ ID NO: 682 - protein, 8 aa, synthetic construct
GRTFSNYA

SEQ ID NO: 683 - protein, 7 aa, synthetic construct
INRSGTT

SEQ ID NO: 684 - protein, 19 aa, synthetic construct
VAGMPFSYSDYPRDSKYDY

SEQ ID NO: 685 - protein, 8 aa, synthetic construct
GRTFNNYA

SEQ ID NO: 686 - protein, 7 aa, synthetic construct
ITRSDTT

SEQ ID NO: 687 - protein, 19 aa, synthetic construct
AAGMPYSYSDYPRDSKYDY

SEQ ID NO: 688 - protein, 8 aa, synthetic construct
GRNLSTYA

SEQ ID NO: 689 - protein, 8 aa, synthetic construct
ISWAGSTT

SEQ ID NO: 690 - protein, 23 aa, synthetic construct
AAPPYVTTTYYATPPSDIRDYHY

SEQ ID NO: 691 - protein, 8 aa, synthetic construct
GRNLSTYA

SEQ ID NO: 692 - protein, 8 aa, synthetic construct
ISWAGSTT

SEQ ID NO: 693 - protein, 23 aa, synthetic construct
AAPPYVTTTYYATPPSDIRDYHY

SEQ ID NO: 694 - protein, 8 aa, synthetic construct
GRTFSVYT

SEQ ID NO: 695 - protein, 6 aa, synthetic construct
MSGTRT

SEQ ID NO: 696 - protein, 17 aa, synthetic construct
AERRYGGPSLVWDEYDY

SEQ ID NO: 697 - protein, 8 aa, synthetic construct
GRTFSVYT

SEQ ID NO: 698 - protein, 6 aa, synthetic construct
MSGTRT

SEQ ID NO: 699 - protein, 17 aa, synthetic construct
AERRYGGPSLVWDEYDY

SEQ ID NO: 700 - protein, 8 aa, synthetic construct
GFTFSSNA

SEQ ID NO: 701 - protein, 8 aa, synthetic construct
ISSGGGTT

SEQ ID NO: 702 - protein, 9 aa, synthetic construct
SKSSTGSGS

SEQ ID NO: 703 - protein, 8 aa, synthetic construct
GRTFSTYA

SEQ ID NO: 704 - protein, 8 aa, synthetic construct
ISWSGGRT

SEQ ID NO: 705 - protein, 13 aa, synthetic construct
AARIDDYDRGYDY

SEQ ID NO: 706 - protein, 8 aa, synthetic construct
GRTFSTYA

SEQ ID NO: 707 - protein, 8 aa, synthetic construct
ISWSGGRT

SEQ ID NO: 708 - protein, 13 aa, synthetic construct
AARIDDYDRGYDY

SEQ ID NO: 709 - protein, 8 aa, synthetic construct
GNTFSINA

SEQ ID NO: 710 - protein, 7 aa, synthetic construct
ISDVGNT

SEQ ID NO: 711 - protein, 19 aa, synthetic construct
NAGTFYTSPGDSYYRGYDF

SEQ ID NO: 712 - protein, 8 aa, synthetic construct
GNTFSINA

SEQ ID NO: 713 - protein, 6 aa, synthetic construct
ISDVGN

SEQ ID NO: 714 - protein, 19 aa, synthetic construct
NAGTFSTSXGDSYYRGYDF

SEQ ID NO: 715 - protein, 10 aa, synthetic construct
GSIRSINAMA

SEQ ID NO: 716 - protein, 7 aa, synthetic construct
ISNSGSI

SEQ ID NO: 717 - protein, 11 aa, synthetic construct
NGRTGWPRYDL

SEQ ID NO: 718 - protein, 8 aa, synthetic construct
GSIRSIND

SEQ ID NO: 719 - protein, 7 aa, synthetic construct
ISDSGSI

SEQ ID NO: 720 - protein, 11 aa, synthetic construct
NGRTGWPRYDL

SEQ ID NO: 721 - protein, 8 aa, synthetic construct
ERTLSGYA

SEQ ID NO: 722 - protein, 8 aa, synthetic construct
IRWSGSST

SEQ ID NO: 723 - protein, 12 aa, synthetic construct
AAGSDYADEYDY

SEQ ID NO: 724 - protein, 8 aa, synthetic construct
GFAMDYLT

SEQ ID NO: 725 - protein, 8 aa, synthetic construct
IDTSGRST

SEQ ID NO: 726 - protein, 13 aa, synthetic construct
LASTSYCGAGYEY

SEQ ID NO: 727 - protein, 8 aa, synthetic construct
GRTFTVYD

SEQ ID NO: 728 - protein, 9 aa, synthetic construct
ISADGATSI

SEQ ID NO: 729 - protein, 18 aa, synthetic construct
AAGRSLIYNLYRANNTDY

SEQ ID NO: 730 - protein, 8 aa, synthetic construct
GRTFNWSA

SEQ ID NO: 731 - protein, 8 aa, synthetic construct
ISGGGTSA

SEQ ID NO: 732 - protein, 18 aa, synthetic construct
AADRVDYRMTLTQNGNHY

SEQ ID NO: 733 - protein, 8 aa, synthetic construct
ARTESMYA

SEQ ID NO: 734 - protein, 8 aa, synthetic construct
IIQSGGTT

SEQ ID NO: 735 - protein, 22 aa, synthetic construct
AAKVSSGTPSDIQISQSRWYDH

SEQ ID NO: 736 - protein, 8 aa, synthetic construct
GHTFSNYV

SEQ ID NO: 737 - protein, 8 aa, synthetic construct
ISWGGSNT

SEQ ID NO: 738 - protein, 16 aa, synthetic construct
AADRRFEGWNYYSMDY

SEQ ID NO: 739 - protein, 8 aa, synthetic construct
GRTVDSYA

SEQ ID NO: 740 - protein, 7 aa, synthetic construct
SWGGGST

SEQ ID NO: 741 - protein, 21 aa, synthetic construct
AAGPTYYSGPINDNWEGRYDS

SEQ ID NO: 742 - protein, 8 aa, synthetic construct
GRAWNEYN

SEQ ID NO: 743 - protein, 8 aa, synthetic construct
IWWSSGNT

SEQ ID NO: 744 - protein, 11 aa, synthetic construct
AAGPTFNKYEY

SEQ ID NO: 745 - protein, 8 aa, synthetic construct
GFTFDSYA

SEQ ID NO: 746 - protein, 8 aa, synthetic construct
IGSGGGST

SEQ ID NO: 747 - protein, 10 aa, synthetic construct
TRSRDSHTRP

SEQ ID NO: 748 - protein, 8 aa, synthetic construct
GSTFSIHA

SEQ ID NO: 749 - protein, 7 aa, synthetic construct
ITRGGST

SEQ ID NO: 750 - protein, 12 aa, synthetic construct
NAETGWGEWDDY

SEQ ID NO: 751 - protein, 8 aa, synthetic construct
GFTFGNSD

SEQ ID NO: 752 - protein, 8 aa, synthetic construct
INTAGGST

SEQ ID NO: 753 - protein, 21 aa, synthetic construct
ATALGSSWYSRFSGDLYEYDY

SEQ ID NO: 754 - protein, 8 aa, synthetic construct
GGTFGDYS

SEQ ID NO: 755 - protein, 8 aa, synthetic construct
IRWSGDST

SEQ ID NO: 756 - protein, 19 aa, synthetic construct
AADPSYYIGPNHTPSRYRD

SEQ ID NO: 757 - protein, 8 aa, synthetic construct
GTTFSTHV

SEQ ID NO: 758 - protein, 7 aa, synthetic construct
IDSAGTT

SEQ ID NO: 759 - protein, 7 aa, synthetic construct
SIPSRNA

SEQ ID NO: 760 - protein, 8 aa, synthetic construct
GRTFSTYA

SEQ ID NO: 761 - protein, 8 aa, synthetic construct
ISWSGGST

SEQ ID NO: 762 - protein, 23 aa, synthetic construct
AADAEDPILYGSSWNNRRKEYDY

SEQ ID NO: 763 - protein, 8 aa, synthetic construct
GFTFDDYA

SEQ ID NO: 764 - protein, 8 aa, synthetic construct
ISSSDYGT

SEQ ID NO: 765 - protein, 19 aa, synthetic construct
AAEAISEYDCLGINYGMDY

SEQ ID NO: 766 - protein, 8 aa, synthetic construct
GRTFGSHR

SEQ ID NO: 767 - protein, 8 aa, synthetic construct
IAWRGGTQ

SEQ ID NO: 768 - protein, 19 aa, synthetic construct
VAGEGSSWGDITDPRRYVF

SEQ ID NO: 769 - protein, 8 aa, synthetic construct
GRTFTTYV

SEQ ID NO: 770 - protein, 8 aa, synthetic construct
ISWSGGRT

SEQ ID NO: 771 - protein, 18 aa, synthetic construct
AAARSDAVRIGYAEGYEY

SEQ ID NO: 772 - protein, 8 aa, synthetic construct
GRTFSSYA

SEQ ID NO: 773 - protein, 8 aa, synthetic construct
VSWGGLST

SEQ ID NO: 774 - protein, 16 aa, synthetic construct
AADRAYEGLGYGEYEY

SEQ ID NO: 775 - protein, 8 aa, synthetic construct
GSTFSTYG

SEQ ID NO: 776 - protein, 8 aa, synthetic construct
ITLIGEES

SEQ ID NO: 777 - protein, 15 aa, synthetic construct
AADSYIKASGWSPRY

SEQ ID NO: 778 - protein, 8 aa, synthetic construct
GRTFSGSA

SEQ ID NO: 779 - protein, 8 aa, synthetic construct
ITWVGGFT

SEQ ID NO: 780 - protein, 18 aa, synthetic construct
AAQAYHKSGYYYPPLYDY

SEQ ID NO: 781 - protein, 8 aa, synthetic construct
GRTFSTYA

SEQ ID NO: 782 - protein, 8 aa, synthetic construct
ISWSGGRI

SEQ ID NO: 783 - protein, 14 aa, synthetic construct
AARDSDYGGQEYDY

SEQ ID NO: 784 - protein, 8 aa, synthetic construct
GLTSSTYV

SEQ ID NO: 785 - protein, 8 aa, synthetic construct
IMLSGGTT

SEQ ID NO: 786 - protein, 19 aa, synthetic construct
ASGLIYRYSDFPRSQKYNF

SEQ ID NO: 787 - protein, 8 aa, synthetic construct
GPTLSTYA

SEQ ID NO: 788 - protein, 8 aa, synthetic construct
ISWSGGRV

SEQ ID NO: 789 - protein, 12 aa, synthetic construct
AARESDYGAYDY

SEQ ID NO: 790 - protein, 8 aa, synthetic construct
GFTFSSFG

SEQ ID NO: 791 - protein, 8 aa, synthetic construct
ISSSGGTT

SEQ ID NO: 792 - protein, 8 aa, synthetic construct
ARDVTGAV

SEQ ID NO: 793 - protein, 8 aa, synthetic construct
GFTFSSFG

SEQ ID NO: 794 - protein, 8 aa, synthetic construct
ISSSGGTT

SEQ ID NO: 795 - protein, 8 aa, synthetic construct
ARDVTGAV

SEQ ID NO: 796 - protein, 8 aa, synthetic construct
GRTFSNYA

SEQ ID NO: 797 - protein, 9 aa, synthetic construct
LSGSGGDTE

SEQ ID NO: 798 - protein, 21 aa, synthetic construct
AAATHVVVHYGLVRRRGTYDY

SEQ ID NO: 799 - protein, 8 aa, synthetic construct
GRTFSNYA

SEQ ID NO: 800 - protein, 9 aa, synthetic construct
LSGSGGDTA

SEQ ID NO: 801 - protein, 21 aa, synthetic construct
AAATHVVVSYGLVRKRGTYDY

SEQ ID NO: 802 - protein, 8 aa, synthetic construct
GRTFSNYA

SEQ ID NO: 803 - protein, 9 aa, synthetic construct
LSGSGGDTA

SEQ ID NO: 804 - protein, 21 aa, synthetic construct
AAATHVVVNYGLVRRRGTYDY

SEQ ID NO: 805 - protein, 8 aa, synthetic construct
GRTFSNYA

SEQ ID NO: 806 - protein, 9 aa, synthetic construct
LSGSGGDTA

SEQ ID NO: 807 - protein, 21 aa, synthetic construct
AAATHVVVNYGLVRRRGTYDY

SEQ ID NO: 808 - protein, 8 aa, synthetic construct
GRTFSNYA

SEQ ID NO: 809 - protein, 9 aa, synthetic construct
LSGSGGDTA

SEQ ID NO: 810 - protein, 21 aa, synthetic construct
AAATHVVVSYGLVRRRGTYDY

SEQ ID NO: 811 - protein, 8 aa, synthetic construct
RRTFSKHA

SEQ ID NO: 812 - protein, 8 aa, synthetic construct
VRWSGIIT

SEQ ID NO: 813 - protein, 18 aa, synthetic construct
AAGDYRGNGYYVPGMYDY

SEQ ID NO: 814 - protein, 8 aa, synthetic construct
GRTFSKST

SEQ ID NO: 815 - protein, 7 aa, synthetic construct
ITWTDTE

SEQ ID NO: 816 - protein, 21 aa, synthetic construct
AGDRYGSSRDSVFSSPTNYPY

SEQ ID NO: 817 - protein, 8 aa, synthetic construct
GRTFSTST

SEQ ID NO: 818 - protein, 7 aa, synthetic construct
ITGTDTE

SEQ ID NO: 819 - protein, 21 aa, synthetic construct
AGDRYGSTRDSVFSSPTNYPY

SEQ ID NO: 820 - protein, 8 aa, synthetic construct
GISSSRLH

SEQ ID NO: 821 - protein, 8 aa, synthetic construct
ISLSGGTQ

SEQ ID NO: 822 - protein, 17 aa, synthetic construct
AARERFIWTWSDLTYDY

SEQ ID NO: 823 - protein, 8 aa, synthetic construct
GRTVSDYA

SEQ ID NO: 824 - protein, 7 aa, synthetic construct
MSSSETT

SEQ ID NO: 825 - protein, 19 aa, synthetic construct
SMGPIATFQGLARQNEYHY

SEQ ID NO: 826 - protein, 8 aa, synthetic construct
GRTVSDYA

SEQ ID NO: 827 - protein, 7 aa, synthetic construct
MSSSETT

SEQ ID NO: 828 - protein, 19 aa, synthetic construct
SMGPIVTFQGLARQNEYHY

SEQ ID NO: 829 - protein, 8 aa, synthetic construct
GRPVSDYA

SEQ ID NO: 830 - protein, 7 aa, synthetic construct
LSASDTT

SEQ ID NO: 831 - protein, 19 aa, synthetic construct
AMGPIGTFQGLARQNEYHY

SEQ ID NO: 832 - protein, 8 aa, synthetic construct
GRTVSDYA

SEQ ID NO: 833 - protein, 7 aa, synthetic construct
MSSSETT

SEQ ID NO: 834 - protein, 19 aa, synthetic construct
SMGPITTFQGLARQNEYHY

SEQ ID NO: 835 - protein, 8 aa, synthetic construct
GSAFRLNF

SEQ ID NO: 836 - protein, 7 aa, synthetic construct
INSGGTE

SEQ ID NO: 837 - protein, 17 aa, synthetic construct
AADRGISWSKLAGGMDY

SEQ ID NO: 838 - protein, 217 aa, synthetic construct
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 839 - protein, 218 aa, synthetic construct
PAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 840 - protein, 218 aa, synthetic construct
PAPNLLGGPDVFIFPPKIKDVLMISLSPIVTCVVVDVSEDDPDVQISWFVNNVEVHTAQTQTHREDYNSTLRVVSALPIQHQDWMSGKEFKCKVNNKDLPAPEERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 841 - protein, 862 aa, Homo sapiens
MAHTFRGCSLAFMFIITWLLIKAKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 842 - protein, 874 aa, Mus musculus
MAQTVRECSLALLFLFMWLLIKANIDVCKLGTVTVQPAPVIPLGSAANISCSLNPKQGCSHYPSSNELILLKFVNDVLVENLHGKKVHDHTGHSSTFQVTNLSLGMTLFVCKLNCSNSQKKPPVPVCGVEISVGVAPEPPQNISCVQEGENGTVACSWNSGKVTYLKTNYTLQLSGPNNLTCQKQCFSDNRQNCNRLDLGINLSPDLAESRFIVRVTAINDLGNSSSLPHTFTFLDIVIPLPPWDIRINFLNASGSRGTLQWEDEGQVVLNQLRYQPLNSTSWNMVNATNAKGKYDLRDLRPFTEYEFQISSKLHLSGGSWSNWSESLRTRTPEEEPVGILDIWYMKQDIDYDRQQISLFWKSLNPSEARGKILHYQVTLQEVTKKTTLQNTTRHTSWTRVIPRTGAWTASVSAANSKGASAPTHINIVDLCGTGLLAPHQVSAKSENMDNILVTWQPPKKADSAVREYIVEWRALQPGSITKFPPHWLRIPPDNMSALISENIKPYICYEIRVHALSESQGGCSSIRGDSKHKAPVSGPHITAITEKKERLFISWTHIPFPEQRGCILHYRIYWKERDSTAQPELCEIQYRRSQNSHPISSLQPRVTYVLWMTAVTAAGESPQGNEREFCPQGKANWKAFVISSICIAIITVGTFSIRYFRQKAFTLLSTLKPQWYSRTIPDPANSTWVKKYPILEEKIQLPTDNLLMAWPTPEEPEPLIIHEVLYHMIPVVRQPYYFKRGQGFQGYSTSKQDAMYIANPQATGTLTAETRQLVNLYKVLESRDPDSKLANLTSPLTVTPVNYLPSHEGYLPSNIEDLSPHEADPTDSFDLEHQHISLSIFASSSLRPLIFGGERLTLDRLKMGYDSLMSNEA

SEQ ID NO: 843 - protein, 15 aa, synthetic construct
EPKSCDKTHTCPPCP

SEQ ID NO: 844 - protein, 15 aa, synthetic construct
EPRGPTIKPCPPCKC

(19) United States
(12) Patent Application Publication
(10) Pub. No.: US 2026/0209362 A1
(43) Pub. Date: Jul. 23, 2026

(54) Binding Molecules Targeting IL-12RB2

(71) Applicants: VIB VZW, Gent (BE); UNIVERSITEIT GENT, Gent (BE)

(72) Inventors: Savvas Savvides, Kortrijk (BE); Ioannis Skordos, Gent (BE); Luc Van Rompaey, Keerbengen (BE); Giel Steven Tanghe, De Pinte (BE); Rudi Beyaert, Kruisem (BE); Harald Braun, Gent (BE)

(21) Appl. No.: 18/997,272

(22) PCT Filed: Jul. 21, 2023
(86) PCT No.: PCT/GR2023/000036

§ 371 Date: Jan. 21, 2025

(30) Foreign Application Priority Data
Jul. 21, 2022 (GB) 2210679.3

(51) Int. Cl. C07K 16/28; A61K 39/00; A61P 37/02

(52) U.S. Cl. CPC C07K 16/2866; A61P 37/02; A61K 2039/505; C07K 2317/33; C07K 2317/52; C07K 2317/565; C07K 2317/569; C07K 2317/732; C07K 2317/734

(57) ABSTRACT

The present invention provides binding molecules, particularly antibodies which bind to the IL-12Rβ2. The antibodies are able to bring about the killing of cells expressing IL-12Rβ2 on their surface. The invention further relates to pharmaceutical compositions comprising the binding molecules. The binding molecules and pharmaceutical compositions of the invention may be used in therapy and diagnosis. They may be employed to deplete cells expressing IL-12Rβ2. As IL-12Rβ2 is expressed narrowly, predominately on ‘type 1 immune cells’ such as CD4± Th1, CD8+ Tc1, and ILC1 cells in autoimmune and inflammatory conditions, the binding molecules of the invention may be used to treat and prevent such conditions via such cell depletion.

### CROSS REFERENCE TO RELATED APPLICATIONS

This application is a national phase entry under 35 U.S.C. § 371 of International Patent Application PCT/GR2023/000036, filed Jul. 21, 2023, designating the United States of America and published in English as International Patent Publication WO2024/018241 on Jan. 25, 2024, which claims the benefit under Article 8 of the Patent Cooperation Treaty to United Kingdom Patent Application Serial No. 2210679.3, filed Jul. 21, 2022, the entireties of which are hereby incorporated by reference.

### INCORPORATION BY REFERENCE

The ST.26 XML Sequence listing named “10488-10868-US-ST26 Sequence Listing”, created on Jul. 19, 2023, and having a size of 761,856 bytes, is hereby incorporated herein by this reference in its entirety.

### FIELD OF INVENTION

The present invention relates to binding molecules that bind to the IL-12 receptor β2 subunit (IL-12Rβ2). The present invention further relates to the use of such binding molecules in treatment and diagnosis. The binding molecules are particularly useful in depleting cells that express IL-12Rβ2.

### BACKGROUND OF INVENTION

The Interleukin-12 (IL-12) family of cytokines is unusual as the cytokines in the family are heterodimeric each consisting of two different polypeptide chains. Each member of the IL-12 family comprises an α chain, with a helical structure similar to type 1 cytokines like IL-6, and a β-chain structurally related to the extracellular region of Type 1 cytokine receptors like soluble IL-6 receptor. Different members of the IL-12 family overlap with each other structurally in the sense that they have the same chain for one of the two chains of the heterodimer. Interleukin-12 (IL-12) consists of an α chain (IL-12p35) and a β chain (IL-12p40) encoded respectively by two separate genes, IL-12A and IL-12B. Other IL-12 family members are IL-23 (consisting respectively of the α and β chains IL-23p19 and IL-12p40), IL-27 (consisting respectively of the α and β chains IL-27p28 and Ebi3) and IL-35 (consisting respectively of the α and β chains IL-12p35 and Ebi3). IL-12 therefore shares an α chain with IL-35 and a β chain with IL-23.

The IL-12 family cytokines each recruit two receptor subunits to form a tripartite signalling assembly of the cytokine and the two receptor subunits. The receptor for the cytokine is effectively the two receptor subunits, but the two receptor subunits do not exist together in the absence of the cytokine. IL-12 is thought to first bind the high affinity IL-12Rβ2 and then IL-12Rβ1 is recruited to create the tripartite signalling assembly of IL-12, IL-12Rβ1, and IL-12Rβ2. IL-23 recruits IL-12Rβ1 and IL-23Rα receptor subunits to form a tripartite signalling assembly of IL-23, IL-12Rβ1 and IL-23Rα. IL-27 recruits IL-27Rα and gp130 receptor subunits to form a tripartite signalling assembly of IL-27, IL-27Rα and gp130. IL-35 is unusual in that is able to form a tripartite signalling assembly with IL-12Rβ2 and gp130, but also tripartite signalling assemblies with IL-35 and two IL-12Rβ2 receptors subunits or two gp130 receptor subunits are reported. In addition, it can form a complex with the IL-12Rβ2 and IL-27Rα receptor chains. The different IL-12 family cytokines therefore overlap in the receptor subunits recruited to the tripartite signalling assembly. The tripartite signalling assemblies formed by IL-12 and IL-23 receptors both include the IL-12Rβ1 subunit. The tripartite signalling assemblies formed by IL-12 and IL-35 both include the IL-12Rβ2 subunit. Signalling via the assembled tripartite signalling assemblies involves Janus kinases (JAKs), with the different receptors varying in terms of which STAT proteins are involved in signalling.

The different cytokines in the IL-12 family also have different functional roles. IL-12, IL-23, and IL-27 are thought to promote immune responses. IL-35 is thought to have a role in inhibiting immune responses, tolerance induction and immune system maintenance mediated via regulatory B and T cells, such as iTr35 cells. IL-12 is considered a key cytokine in driving type 1 cell-mediated effector immunity, particularly against intracellular microbes. IL-12 promotes differentiation of CD4+ Th1 and CD8+ Tc1 cells by inducing T-bet transcription factor expression, and the production of IFN-γ. However, Tc1 cells can be induced to produce IFN-γ independently of IL-12. IL-12 has the ability to trigger IFN-γ production in group 1 innate lymphoid cells (ILC1s), which are defined to include ILCs expressing T-bet and producing IFN-γ.

### SUMMARY OF INVENTION

IL-12Rβ2 is notable in that it has a very specific expression pattern. IL-12Rβ2 is hardly expressed at all normally, but upon T cell activation, including in inflammation and a number of autoimmune disorders, IL-12Rβ2 expression is significantly upregulated, with IL-12Rβ2 then primarily found on type 1 immune cells, including CD4+ Th1, CD8+ Tc1 and ILC1 cells. The narrow and very specific expression pattern of IL-12Rβ2 offers a potential way to target and deplete cells with the IL-12Rβ2 subunit of their cell surface and in particular type 1 immune cells. The present invention provides for binding molecules, in particular antibodies, against the IL-12Rβ2 subunit for use in a method of treatment comprising depleting cells expressing the IL-12Rβ2 subunit. In an especially preferred embodiment, the cells to be depleted are ‘type 1 immune cells’ which include CD8+ Tc1 cells, ILC1 cells and in particular CD4+ Th1 cells. In a further especially preferred embodiment, the antibody displays ADCC, ADCP, and/or CDC activity allowing the depletion of IL-12Rβ2 subunit expressing cells via the such activity. The present invention also provides binding molecules, in particular antibodies, against the IL-12Rβ2 subunit. Those antibodies may be put to a variety of uses.

Accordingly, the present invention provides a binding molecule specific for a IL-12Rβ2 subunit for use in a method of treatment, the method comprising depleting cells expressing the IL-12Rβ2 subunit using the binding molecule.

The present invention further provides a method of treating comprising administering a binding molecule specific for a IL-12Rβ2 subunit to a subject, wherein the binding molecule depletes target cells expressing the IL-12Rβ2 subunit in the subject.

The present invention also provides a binding molecule that binds to IL-12Rβ2, wherein the binding molecule comprises one or more of the following VHH antigen-domains:
(a) a VHH antigen-binding domain that binds IL-12Rβ2 comprising a set of three CDRs (CDR1, CDR2, and CDR3) selected from the sets of three CDRs of Table 1 for clones 20407, 21050, 21053, 21060, 21062, 20432, 20438;
(b) a VHH antigen-binding domain that binds IL-12Rβ2 comprising a set of three CDRs (CDR1, CDR2, and CDR3) that correspond to a set of three CDRs of Table 1 selected from the sets of three CDRs of Table 1 for clones 20407, 21050, 21053, 21060, 21062, 20432, 20438 apart from a maximum of ten amino acid sequence changes;
(c) a VHH antigen-binding domain that binds IL-12Rβ2 comprising a set of three CDRs (CDR1, CDR2, and CDR3) that have at least 90% sequence identity to a set of three CDRs of Table 1 selected from the sets of three CDRs of Table 1 for clones 20407, 21050, 21053, 21060, 21062, 20432, 20438; or
(d) a VHH antigen-binding domain that can compete for binding to IL-12Rβ2 with a VHH antigen-domain of any of (a) to (c).

The present invention also provides a binding molecule that binds to an Interleukin-12 receptor β2 subunit (IL-12Rβ2), wherein the binding molecule comprises one or more of the following VHH antigen-domains:
(a) a VHH antigen-binding domain that binds IL-12Rβ2 comprising a set of three CDRs (CDR1, CDR2, and CDR3) selected from the sets of three CDRs of Table 1;
(b) a VHH antigen-binding domain that binds IL-12Rβ2 comprising a set of three CDRs (CDR1, CDR2, and CDR3) that correspond to a set of three CDRs of Table 1 apart from a maximum of ten amino acid sequence changes;
(c) a VHH antigen-binding domain that binds IL-12Rβ2 comprising a set of three CDRs (CDR1, CDR2, and CDR3) that have at least 90% sequence identity to a set of three CDRs of Table 1; or
(d) a VHH antigen-binding domain that can compete for binding to IL-12Rβ2 with a VHH antigen-domain of any of (a) to (c).

The present invention also provides a pharmaceutical composition comprising a binding molecule of the present invention and a pharmaceutically acceptable carrier.

The present invention further provides a binding molecule or pharmaceutical composition of the present invention for use in a method of treatment or diagnosis of the human or animal body.

The present invention also provides a binding molecule or pharmaceutical composition of the present invention for use in a method of treating or preventing an autoimmune or inflammatory disorder.

The present invention also provides a binding molecule specific for a IL-12Rβ2 subunit for use in a method of treatment or diagnosis of the human or animal body, optionally wherein the binding molecule is an antibody, preferably wherein the antibody comprises a single VHH domain and an Fc region.

### BRIEF DESCRIPTION OF THE FIGURES

FIG. 1: Shows the binding of selected anti-IL-12Rβ2 VHH clones to human IL-12Rβ2 expressed on HEK293T cells. A: Periplasmic extracts containing anti-IL-12Rβ2 VHHs were tested for binding to HEK293T cells transiently transfected with a human IL-12Rβ2 expression plasmid. Cells were consecutively stained with anti-IL-12Rβ2 VHH-HA (hemagglutinin tag), mouse anti-HA antibody and the anti-mouse-PE detection antibody using anti flow cytometry. The Figure corresponds to Table 3, with the Figure providing illustrative results for some of the antibodies from Table 3. B: Periplasmic extracts containing anti-IL-12Rβ2 VHHs were tested for binding to HEK293T cells with stable expression of mouse IL-12Rβ2. Cells were consecutively stained with anti-IL-12Rβ2 VHH-HA (hemagglutinin tag), mouse anti-HA antibody and the anti-mouse-PE detection antibody using flow cytometry. The Figure corresponds to Table 4, with the Figure providing illustrative results for some of the antibodies from the Table.

FIG. 2: Shows the ability of selected anti-IL-12Rβ2 VHH clones to compete with IL-12 signalling. HEK-Blue IL-12 reporter cells were incubated with different dilutions of periplasmic extracts containing anti-IL-12Rβ2 VHHs prior to addition of recombinant human IL-12. One day later, IL-12 signalling activity was determined by measuring the levels of secreted embryonic alkaline phosphatase in the cell culture medium. The Figure corresponds to Table 5, clones with various IL-12 competition properties are depicted as illustrative examples of the antibodies.

FIG. 3: Shows the ability of anti-IL-12Rβ2-VHH-FcDE antibodies to induce cell death of IL-12Rβ2-expressing cells mediated by PBMC effectors in an in vitro assay for ADCC activity. Daudi cells with stable transgenic human IL-12Rβ2 expression (‘target cells’) were opsonized with different concentrations of the indicated antibodies and subsequently co-cultured with human PBMCs as effector cells. The latter were activated for 24 hours with IL-2 prior to addition to the target cells. Cells were cultured for 4 hours at an effector-to-target ratio of 10:1 before cell death was measured using flow cytometry. The heatmap depicts the amount of cell death normalized to background cell death (“% specific lysis”). The anti-IL-12Rβ2-VHH-Fc antibodies contained either a wildtype human IgG1 Fc domain (“FcWT”) or an Fc domain with the S239D and I332E amino acid mutations enhancing ADCC activity (“FcDE”).

FIG. 4: Shows the ability of anti-IL-12Rβ2-VHH-Fc antibodies to induce cell death of IL-12Rβ2-expressing cells by NK effectors in an in vitro assay for ADCC activity. Daudi cells with stable transgenic human IL-12Rβ2 expression (‘target cells’) were opsonized with different concentrations of the indicated antibodies and subsequently co-cultured with human NK cells as effector cells. The latter were activated for 24 hours with IL-2 prior to addition to the target cells. Cells were cultured for 4 hours at an effector-to-target ratio of 1:1 before cell death was measured using flow cytometry. The graph depicts the amount of cell death normalized to background cell death (“% specific lysis”). The anti-IL-12Rβ2-VHH-Fc antibodies contained the S239D and I332E amino acid mutations in the Fc domain (“FcDE”), enhancing ADCC activity.

FIG. 5: Shows the ability of selected cross-reactive anti-IL-12Rβ2-VHH-Fc antibodies to kill human or mouse IL-12Rβ2-expressing cells in an in vitro ADCC assay. Jurkat cells with stable transgenic human or mouse IL-12Rβ2 expression (‘target cells’) were opsonized with 10 μg/ml of the indicated antibodies and subsequently co-cultured with human PBMCs as effector cells. The latter were activated for 24 hours with IL-2 prior to addition to the target cells. Cells were cultured for 4 hours at an effector-to-target ratio of 10:1 or 30:1 before cell death was measured using flow cytometry. The graph depicts the amount of cell death normalized to background cell death (‘% specific lysis’). The anti-IL-12Rβ2-VHH-Fc antibodies contained the S239D and 1332E amino acid mutations in the Fc domain (‘FcDE’), enhancing ADCC activity

FIG. 6: Shows the ability of anti-IL-12Rβ2-VHH-Fc antibodies to mediate killing of primary human CD4+ Th1 cells. A: CD4 T cells were enriched from human PBMCs and differentiated for 3 days in Th1 differentiation medium. The levels of IL-12Rβ2 expression were then analyzed by flow cytometry. B: ADCC assay with Th1 differentiated cells as target cells and autologous NK cells as effector cells at effector-to-target ratio of 5:1. The graph depicts the amount of cell death normalized to background cell death (‘% specific lysis’) based on pooled data from two independent donors. The anti-IL-12Rβ2-VHH-Fc antibodies contained the S239D and I332E amino acid mutations in the Fc domain (‘FcDE’), enhancing ADCC activity

FIG. 7: Shows the ability of selected anti-IL-12Rβ2-VHH-Fc antibodies to compete with IL-12 for IL-12R activation. Activated primary human CD4 T cells were pre-treated with 100 nM of anti-IL-12Rβ2-VHH-Fc antibodies for 30 minutes and sequentially treated with different concentrations of recombinant IL-12 for 30 minutes. Next, phospho-STAT4 within the activated CD4 T cells was measured by flow cytometry as measure for IL-12 receptor activation.

FIG. 8: IL-12Rβ2 expression is upregulated on Tbet+ Th1 cells. Primary human CD4 T cells were ex vivo differentiated for three days into T helper 1 (Th1) cells. Cells were harvested and analysed by flow cytometry for surface expression of IL-12Rβ and intracellular expression of the transcription factor Tbet. Graphs depict the fluorescence intensity for the indicated markers of CD4 T cells.

FIG. 9: IL-12Rβ2 and Tbet expression is upregulated in alloreactive CD4 T cells. A mixed lymphocyte reaction was set up by co-culturing of human PBMCs from two allogeneic donors for 7 days. Subsequently the expression of IL-12Rβ2 and Tbet was analysed in alloreactive or naïve CD4 and CD8 T cells by flow cytometry. Graphs depict the mean fluorescence intensity (MFI) of the indicated proteins.

FIG. 10: Shows the ability of anti-IL-12Rβ2-VHH-Fc antibodies to prevent expansion of alloreactive Tbet+CD4+ T cells. A one-way mixed lymphocyte reaction was set up by co-culturing of human PBMCs from two allogeneic donors for 7 days. Cells were treated at the start and at day 3 of co-culture initiation with anti-IL-12Rβ2-VHH-Fc antibodies, the negative control antibody BCII-10-FcDE, or the positive control proteins alemtuzumab and CTLA-4-Ig. Responder T cells were labelled with CFSE to track proliferation. A: Depicts the percentage of responder CD4 T cells that are activated (CD25+) and show proliferation by dilution of the CFSE fluorescent label. B: Depicts percentage of Tbet+ Th1 cells within the responder CD4 T cells.

FIG. 11: Demonstrates that selected human-mouse cross-reactive anti-IL-12Rβ2 VHH antibody does not show changes in the frequency of major lymphocyte subsets in the blood of naïve mice. Anti-IL-12Rβ2 VHH-FcDE clone 21053 and negative control antibody were administered intraperitoneally in mice at 100 and 300 μg/mouse. Blood samples were collected 48 h later from animals and the frequency of circulating T, B and NK cells (A), CD4 T and CD8 T cells (B), and regulatory T cells (C) was assessed by flow cytometry. Bar graphs depict mean±S.E.M. (* p<0.05, n=3).

### DETAILED DESCRIPTION

### Binding Molecule Formats

Any suitable format binding molecule able to bind to IL-12Rβ2 may be employed in the present invention. In a preferred embodiment the binding molecule will be able to kill a target cell expressing IL-12Rβ2 on the cell surface. The binding molecule may do so by any suitable means, for instance by virtue of having ADCC (Antibody Dependent Cellular Cytotoxicity), ADCP (Antibody Dependent Cellular Phagocytosis), and/or CDC (cell Dependent Cytotoxicity) activity or being conjugated to a molecule, such as a toxin, that can kill the target cell.

In an especially preferred embodiment, the binding molecule of the invention is an antibody. The term “antibody” as used herein is not limited to the “classical” structure of the four-chain structure of an IgG antibody in humans comprising two light and two heavy chains. However, such structure of antibodies may be employed in the invention, for example such antibodies may be used that specifically bind IL-12Rβ2 on the surface of a target cell and bring about the killing of the cell. In embodiments where a binding molecule comprises antibody-based sequences, the overall binding molecule may be simply referred to as an antibody. Hence, reference to an antibody may be used to refer to the overall molecule, even if the binding molecule comprises a constituent which itself would be viewed as an antibody, for instance, a VHH binding domain. The term “antibody” specifically includes a single chain antibody and a binding molecule comprising such a single chain antibody. A binding molecule of the present invention may be, or comprise, a single chain antibody that is specific for IL-12Rβ2. A binding molecule of the present invention may also comprise non-antibody sequences, for example it may comprise binding sites specific for IL-12Rβ2 that are not antibody based, but which still mean that the binding molecule can be used to target and kill cells expressing IL-12Rβ2 on their surface. In one embodiment, a binding molecule of the present invention does not comprise any antibody-based sequences, but instead comprises non-antibody based binding sites or binding sites specific for IL-12Rβ2, and can kill cells expressing IL-12Rβ2.

In a preferred embodiment, the binding molecule is characterised as being, or comprising, a single domain binding region for IL-12Rβ2. A single-domain antibody (sdAb) is an antibody fragment consisting of a single monomeric variable antibody domain. Like a whole antibody, sdAb is able to bind selectively to a specific antigen. sdAb may be antibody fragments that can be engineered from single monomeric variable domains of either camelids' heavy-chain antibody (VHH) or cartilaginous fishes' IgNAR (VNAR), or be developed from camelized human antibodies. Any such sdAbs may be employed. Examples of single binding domain binders (sdBs) include in particular single domain antibodies (sdAb), for example, heavy chain only antibodies (HCAb), particularly VHH domain antibodies. Especially preferred sdAbs are VHH domains. In one embodiment, a binding molecule of the present invention may comprise at least one sdAb domain. In another preferred embodiment, a binding molecule of the present may comprise VHH binding domains as sdAbs. SdAbs from organisms such as Camelids, sharks, and other cartilaginous fish that produce heavy chain-only antibodies may be employed. The single-domain variable fragments of these heavy chain-only antibodies are termed VHHs or nanobodies or sdAb. VHHs retain the immunoglobulin fold shared by antibodies, using three hypervariable loops, CDR1, CDR2 and CDR3, to bind to their targets. A VHH fragment (e.g., NANOBODY®) is a recombinant, antigen-specific, single-domain, variable fragment derived from camelid heavy chain antibodies. Single domain binders include, for instance, non-Ig engineered protein scaffolds such as darpins, affibodies, adnectins, anticalin proteins, or peptides and the like. So wherever reference is made to sdB, sdAb, HCAb, and VHH, it may be possible to also employ a darpin, affibody, adnectin, anticalin, or peptide that is able to bind IL-12Rβ2 and the term sdB encompasses such binding entities being employed. In an especially preferred embodiment, a binding molecule of the present invention comprises any one of the above mentioned sdAbs and an Fc region. In one embodiment, it consists essentially of those components. In one embodiment, it consists of those components.

In an especially preferred embodiment, a binding molecule of the present invention is, or comprises, a VHH domain antibody specific for an IL-12Rβ2 subunit. The present application sets out examples of preferred VHH domains and those VHH domains may be employed in any suitable binding molecule format set out herein. In another especially preferred embodiment, a binding molecule of the present invention comprises a VHH binding domain specific for IL-12Rβ2 and an Fc region. In one particularly preferred embodiment, the binding molecule is a VHH-FC binding molecule comprising a VHH with a Fc region with no CH1 region. As well as the specific CDR sets, VHH domains, and binding molecules set out herein, the invention provides variants of any thereof, as well as employing CDR sets, VHH domains, or binding molecules that can compete for binding with any of the specific molecules set out herein.

### IL-12Rβ2 Receptor Subunit as a Target

The present invention provides and employs binding molecules that are able to bind to the IL-12Rβ2 receptor subunit to kill target cells expressing the IL-12Rβ2 receptor subunit. In a preferred embodiment, the binding molecule will be able to bring about the lysis of a target cell expressing a IL-12Rβ2 subunit on the surface of the cell. In an especially preferred embodiment, the binding molecule will be able to bind to the IL-12Rβ2 subunit and bring about the death of the target cell by ADCC (antibody dependent cellular cytotoxicity), ADCP (antibody dependent cellular phagocytosis) and/or CDC (cell dependent cytotoxicity).

In a particularly preferred embodiment, a binding molecule is able to bind to a human IL-12Rβ2 receptor subunit. In one preferred embodiment, a binding molecule is able to bind to the mouse IL-12Rβ2 receptor subunit. In one preferred embodiment, a binding molecule is able bind to the human and mouse IL-12Rβ2 receptor subunits. In an alternative embodiment, a binding molecule is able bind to the human IL-12Rβ2 receptor subunit, but not the mouse IL-12Rβ2 receptor. The amino acid sequence of the human and mouse IL-12Rβ2 receptor subunits are provided below:

SEQ ID NO: 841, sequence of the human IL-12Rβ2
(NP_001550.1, interleukin-12 receptor subunit
beta-2 isoform a precursor [Homo sapiens]):
MAHTFRGCSLAFMFIITWLLIKAKIDACKRGDVTVKPSHVILLGSTVNIT
CSLKPRQGCFHYSRRNKLILYKFDRRINFHHGHSLNSQVTGLPLGTTLFV
CKLACINSDEIQICGAEIFVGVAPEQPQNLSCIQKGEQGTVACTWERGRD
THLYTEYTLQLSGPKNLTWQKQCKDIYCDYLDFGINLTPESPESNFTAKV
TAVNSLGSSSSLPSTFTFLDIVRPLPPWDIRIKFQKASVSRCTLYWRDEG
LVLLNRLRYRPSNSRLWNMVNVTKAKGRHDLLDLKPFTEYEFQISSKLHL
YKGSWSDWSESLRAQTPEEEPTGMLDVWYMKRHIDYSRQQISLFWKNLSV
SEARGKILHYQVTLQELTGGKAMTQNITGHTSWTTVIPRTGNWAVAVSAA
NSKGSSLPTRINIMNLCEAGLLAPRQVSANSEGMDNILVTWQPPRKDPSA
VQEYVVEWRELHPGGDTQVPLNWLRSRPYNVSALISENIKSYICYEIRVY
ALSGDQGGCSSILGNSKHKAPLSGPHINAITEEKGSILISWNSIPVQEQM
GCLLHYRIYWKERDSNSQPQLCEIPYRVSQNSHPINSLQPRVTYVLWMTA
LTAAGESSHGNEREFCLQGKANWMAFVAPSICIAIIMVGIFSTHYFQQKV
FVLLAALRPQWCSREIPDPANSTCAKKYPIAEEKTQLPLDRLLIDWPTPE
DPEPLVISEVLHQVTPVFRHPPCSNWPQREKGIQGHQASEKDMMHSASSP
PPPRALQAESRQLVDLYKVLESRGSDPKPENPACPWTVLPAGDLPTHDGY
LPSNIDDLPSHEAPLADSLEELEPQHISLSVFPSSSLHPLTFSCGDKLTL
DQLKMRCDSLML
SEQ ID NO: 842, sequence of mouse IL-12Rβ2 (NP_
032380.1, interleukin-12 receptor subunit beta-2
isoform 1 precursor [Mus musculus]):
MAQTVRECSLALLFLFMWLLIKANIDVCKLGTVTVQPAPVIPLGSAANIS
CSLNPKQGCSHYPSSNELILLKFVNDVLVENLHGKKVHDHTGHSSTFQVT
NLSLGMTLFVCKLNCSNSQKKPPVPVCGVEISVGVAPEPPQNISCVQEGE
NGTVACSWNSGKVTYLKTNYTLQLSGPNNLTCQKQCFSDNRQNCNRLDLG
INLSPDLAESRFIVRVTAINDLGNSSSLPHTFTFLDIVIPLPPWDIRINF
LNASGSRGTLQWEDEGQVVLNQLRYQPLNSTSWNMVNATNAKGKYDLRDL
RPFTEYEFQISSKLHLSGGSWSNWSESLRTRTPEEEPVGILDIWYMKQDI
DYDRQQISLFWKSLNPSEARGKILHYQVTLQEVTKKTTLQNTTRHTSWTR
VIPRTGAWTASVSAANSKGASAPTHINIVDLCGTGLLAPHQVSAKSENMD
NILVTWQPPKKADSAVREYIVEWRALQPGSITKFPPHWLRIPPDNMSALI
SENIKPYICYEIRVHALSESQGGCSSIRGDSKHKAPVSGPHITAITEKKE
RLFISWTHIPFPEQRGCILHYRIYWKERDSTAQPELCEIQYRRSQNSHPI
SSLQPRVTYVLWMTAVTAAGESPQGNEREFCPQGKANWKAFVISSICIAI
ITVGTFSIRYFRQKAFTLLSTLKPQWYSRTIPDPANSTWVKKYPILEEKI
QLPTDNLLMAWPTPEEPEPLIIHEVLYHMIPVVRQPYYFKRGQGFQGYST
SKQDAMYIANPQATGTLTAETRQLVNLYKVLESRDPDSKLANLTSPLTVT
PVNYLPSHEGYLPSNIEDLSPHEADPTDSFDLEHQHISLSIFASSSLRPL
IFGGERLTLDRLKMGYDSLMSNEA

In another embodiment, a binding molecule will bind to a IL-12Rβ2 receptor subunit when it is present part of a tripartite signalling complex with IL-12Rβ1 and IL-12. In another embodiment, a binding molecule will bind to an IL-12Rβ2 receptor subunit when it is part of a tripartite signalling complex with IL-35 and gp130. In one embodiment, a binding molecule will be able to bind to an IL-12Rβ2 receptor subunit when the receptor subunit is part of a bipartite signalling complex with IL-12 and IL-12Rβ1 as well as bind to the IL-12Rβ2 receptor subunit when it is part of a tripartite signalling complex with IL-35 and gp130.

In a preferred embodiment, a binding molecule does not compete with IL-12 for binding to a IL-12Rβ2 receptor subunit. Hence, in such embodiments, the binding molecule of the present invention does not inhibit the ability of IL-12 to form a tripartite signalling assembly with the IL-12Rβ2 and IL-12Rβ1 receptor subunits. In an alternative embodiment, a binding molecule of the present invention is able to compete with IL-12 for binding to the IL-12Rβ2 receptor subunit. Hence, in one embodiment a binding molecule of the present invention may be used to inhibit the action of IL-12.

The “valency” of a binding molecule as used herein denotes the number of antigen-binding sites that the binding molecule comprises. In a preferred embodiment, a binding molecule comprises a single antigen-binding domain for IL-12Rβ2 and so has a valency of one for IL-12Rβ2. In one embodiment, that antigen-binding site is the only one of the binding molecule. In a particularly preferred embodiment, a binding molecule of the present invention comprises a single antigen-binding site for IL-12Rβ2, with the antigen-binding site being a VHH domain antibody specific for IL-12Rβ2. In an alternative embodiment, a binding molecule of the present invention may comprise more than one antigen-binding site specific for IL-12Rβ2, for example, two, three, four, five, six or more such antigen-binding sites.

The strength of binding of an individual antigen-binding site to an IL-12Rβ2 polypeptide may be referred to as the “affinity” of the binding site for its target, the IL-12Rβ2 polypeptide. Whilst the overall strength of binding of a binding molecule is often also referred to as the affinity of the binding molecule, where the binding molecule has more than one binding site, the strength of binding may be referred to using the term avidity, which reflects the overall strength of binding when all of the binding sites of the binding molecule are taken into account. A binding molecule of the present invention may be said to specifically bind an IL-12Rβ2 receptor subunit. Specific binding may constitute binding to an IL-12Rβ2, but not significantly binding to other polypeptides. In one embodiment, an IL-12Rβ2 binding domain may have a K_{D} affinity value for IL-12Rβ2 which is about 400 nM or smaller, 200 nM or smaller such as about 100 nM, 50 nM, 20 nM, 10 nM, 1 nM, 500 μM, 250 μM, 200 μM, 100 μM or smaller. In one embodiment, the K_{D} is 50 μM or smaller. In one embodiment, the K_{D} of an individual antigen-binding site of a binding molecule of the present invention may be less than 1 μM, less than 750 nM, less than 500 nM, less than 250 nM, less than 200 nM, less than 150 nM, less than 100 nM, less than 75 nM, less than 50 nM, less than 10 nM, less than 1 nM, less than 0.1 nM, less than 10 μM, less than 1 μM, or less than 0.1 μM. In some embodiments, the K_{D} is from about 0.1 μM to about 1 μM. It may be an individual antigen-binding domain has such K_{D}. It may be that such a K_{D} for IL-12Rβ2 is displayed by the overall binding molecule of the invention. A K_{D} affinity value K_{D} is the equilibrium dissociation constant, a calculated ratio of Koff/Kon, between the antibody and its antigen. The association constant (Kon) is used to characterise how quickly the antibody binds to its target. The dissociation constant (Koff) is used to measure how quickly an antibody dissociates from its target.

Binding, including the presence or absence of binding, can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE™ analysis or Octet™ analysis (forteBIO)), and other methods such as indirect binding assays, competitive binding assays fluorescence resonance energy transfer (FRET), gel electrophoresis and chromatography (e.g., gel filtration). Binding to an IL-12Rβ2 may be measured using cells expressing an IL-12Rβ2 on their surface as discussed later in relation to assays and also in the Examples of the present application.

### Constant Regions

In one particularly preferred embodiment, a binding molecule of the present invention will comprise a constant region. A VHH domain antibody may include, for example, the CH1 and CH2 domains of the constant region, but not a CH1 domain. Hence, in one preferred embodiment a binding molecule of the present invention will comprise at least one VHH binding domain and an Fc region, but not a CH1 region. Fc domain as employed herein generally refers to CH2-CH3, unless the context clearly indicates otherwise, where CH2 is the heavy chain CH2 domain, CH3 is the heavy chain CH3 domain.

In one embodiment, a binding molecule of the present invention will be able to bind to at least one Fc receptor type. In another preferred embodiment, binding of the invention is able to bring about the death of a target cell. In a particularly preferred embodiment, the binding molecule is able to induce ADCC (Antibody Dependent Cell Cytotoxicity). In another preferred embodiment, the binding molecule is able to induce ADCP (Antibody Dependent Cellular Phagocytosis). In another embodiment, the binding molecule is able to induce CDC (Cell Dependent Cytotoxicity). In one embodiment, the binding molecule itself does not kill the target cell, rather it has ADCC, ADCP, and/or CDC activity and so triggers cell killing by effector cells. In a further particularly preferred embodiment the target cell in which cytotoxicity is induced is a type 1 immune cell. Examples of a type 1 immune cell include CD8+ Tc1, ILC1 cells and CD4+ Th1 cells. Particularly preferred target cells are CD4+ Th1 cells. The Th1 cell targeted will express the IL-12Rβ2 receptor subunit.

In a further preferred embodiment, a binding molecule of the present invention will comprise sequence modifications in the constant region of the antibody that enhance the ability of the antibody to bring about ADCC, ADCP, and/or CDC. In one embodiment an antibody of the present invention comprises a polypeptide comprising a VHH domain and an Fc region. In a preferred embodiment, a binding molecule of the invention will comprise an Fc region having amino acid mutations enhancing ADCC activity. Examples of possible sequence modifications include S239D, 1332E and A330L. Those mutations may be singly present, or two may be present, or all three may be present. Particularly preferred constant region modifications are S239D and/or 1332E. In an especially preferred embodiment, the Fc region comprises both the S239D and 1332E modifications, which may be referred to as an FcDE region. Hence, any of the specific VHH domains and sets of three CDRs set out herein may be provided as part of a binding molecule comprising such a constant region, as may variant and competing sequences set out herein.

In a further preferred embodiment, a binding molecule may comprise an Fc region which has reduced glycosylation. In a particularly preferred embodiment, the Fc region of the binding molecule is afucosylated. In one embodiment, the binding molecule may comprise any of the sequence modifications discussed herein for enhancing ADCC and also be afucosylated. Hence, in one preferred embodiment, the Fc region comprises the S239D and 1332E sequence modifications and is afucosylated.

Any of the constant regions mentioned above, may be employed with any of the specific CDR sets or VHH domains set out herein as well as variant and competing versions to those specific CDR sets and VHH. In an especially preferred embodiment, they are provided with a single VHH domain and an Fc region as discussed above.

### Illustrative Preferred Binding Molecules for IL-12Rβ2

In a particularly preferred embodiment, the binding molecule comprises at least one VHH binding-domain. Table 1 of the present application provides examples of particularly preferred VHH antibodies, as well as particularly preferred CDR “sets” of three CDRs which may be employed in a binding molecule. As well as the specific VHH binding domains and CDR sets of Table 1, variants and competing VHH binding domains are also provided, or may be employed, as discussed further below. VHH domain antibodies comprise three CDRs, CDR1, CDR2, and CDR3. They do not typically comprise a light chain. Reference to a “set of CDRs” in relation to a VHH domain antibody refers to the CDR1, CDR2, and CDR3 of that VHH domain. Table 1 provides examples of VHH binding domains and CDR sets that are provided. For example, the first antibody describe in Table 1 is a VHH binding domain of SEQ ID NO: 1, with the CDR1, CDR2, and CDR3 of the antibody provided as SEQ ID Nos: 211, 212, and 213 respectively. The CDR1, CDR, and CDR3 sequences of SEQ ID NOs: 211, 212, and 213 may be therefore referred to as a “set” of CDR sequences.

Also representing particularly preferred VHH domains are humanised versions of those shown in Table 1, with the CDR sequences either remaining unchanged or representing variant CDR sequences as defined herein.

The present invention provides a binding molecule comprising, or consisting of, a VHH domain as set out in Table 1, hence a VHH domain having, or comprising, the sequence of any one of SEQ ID NOs 1 to 209. The present invention also provides a binding molecule comprising, or consisting of, a variant or competing VHH binding domain of any those set out in Table 1. The present invention further provides a binding molecule comprising a humanised version of one of the VHH domains of Table 1, so a VHH domain where the framework regions have been modified or substituted so that they are human sequences. Variants and competing VHH domains of those set out in Table 1 may also be humanised.

The present invention also provides a binding molecule comprising a “set” of CDRs from Table 1, so the sets of CDR1, CDR2, and CDR3 provided in Table 1 which have the various sequences set out as SEQ ID NOs: 211 to 837. The present invention also provides a binding molecule comprising a VHH binding domain where the CDRs are a set of three CDRs from Table 1, but the framework regions of the VHH binding domain are human.

Binding molecules with variant VHH domains or sets of variant CDR sequences are also provided and may be employed in the present invention. Variants may be defined, for example, in terms of having a particular level of sequence identity or number of sequence changes in comparison to a specific VHH domain or set of CDR sequences from Table 1. The sequence identity may be over the entire length of a sequence, such as over the entire length of a VHH domain or just over the length of the set of the CDR sequences.

Sequence identity can be defined in terms of over the entire length of the polypeptide in question. It may also be defined over the length of the CDR set or the length of a VHH domain. Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987, Sequence Analysis Primer, Gribskov, M. and Devereux, J., eds., M Stockton Press, New York, 1991, the BLAST™ software available from NCBI (Altschul, S. F. et al., 1990, J. Mol. Biol. 215:403-410; Gish, W. & States, D. J. 1993, Nature Genet. 3:266-272. Madden, T. L. et al., 1996, Meth. Enzymol. 266:131-141; Altschul, S. F. et al., 1997, Nucleic Acids Res. 25:3389-3402; Zhang, J. & Madden, T. L. 1997, Genome Res. 7:649-656). “Identity”, as used herein, indicates that at any particular position in the aligned sequences, the amino acid residue is identical between the sequences. “Similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine;
aspartate and glutamate (amino acids having acidic side chains);
asparagine and glutamine (amino acids having amide side chains); and
cysteine and methionine (amino acids having sulphur-containing side chains).

In one embodiment, a variant VHH binding domain may have at least 80% amino acid identity, for example 85% or greater, such as 90% or greater, in particular 95%, 96%, 97%, 98% or 99% or greater identity to one of the specific VHH binding domains set out in Table 1. In one embodiment a sequence may have at least 95% sequence identity to at least one of those sequences. In another embodiment, a variant VHH binding domain may have such percentages values in relation to the degree of amino acid sequence similarity that they display to the specific sequence. The variant will be still able to bind IL-12Rβ2. A variant will be able to still kill target cells expressing IL-12Rβ2.

In another embodiment, a binding molecule of the invention may comprise a variant set of CDRs which are a variant of one of the specific CDR sets of Table 1. In one embodiment, only one of the three CDRs shows sequence variation in comparison to the corresponding CDR of the set of three CDRs. In another embodiment, two of the CDRs show sequence variation in comparison to the specific set of three CDRs. In another embodiment, all three CDRs may show sequence variation compared to the specific CDRs of the set. In one embodiment, the sequence variation is only in the CDR3 of the CDR set. In one embodiment, the level of sequence identity over the total length of the three CDRs in comparison to the set of three specific CDR sequences from Table 1 is at least 80%. In another embodiment, it is at least 85%. In a further embodiment, the level of identity is at least 90%. In a preferred embodiment, the level of sequence identity is at least 95%. The variant will still be able to bind IL-12Rβ2. A variant will be able to still kill target cells expressing IL-12Rβ2.

In one embodiment, a variant may have a set of three CDRs comprising from one to twenty, such as from one to ten, for example as one, two, three, four, five or up to those values of amino acid sequence changes or at least those values, or up to those values compared to the set of CDRs from Table 1, so long as the variant is still able to bind IL-12Rβ2. A variant will be able to still kill target cells expressing IL-12Rβ2.

In another embodiment, a variant of the present invention may have at least five, six, seven, eight, nine, ten, eleven or twelve amino acid sequence changes compared to the CDRs of one of the specific antibodies set out herein, for example it may have that number of sequence changes in a set of CDRs making up a VHH domain. A binding molecule of the present invention may have that number of sequence changes in a set of three CDRs compared to the sequence of the set of CDRs identified in Table 1. In one embodiment, a set of three CDRs may have from five to ten, ten to fifteen, or fifteen to twenty amino acid sequence changes compared to a specific set of three CDRs set out herein.

Variant binding molecules will typically retain the ability to specifically bind IL-12Rβ2. They may also retain one of the other functions set out herein. A variant will be able to still kill target cells expressing IL-12Rβ2. A variant will retain the ability to bind IL-12Rβ2. In one embodiment, a variant will retain ADCC, ADCP, and/or CDC.

As well as variant CDR sets, variants of the specific VHH domain sequences set out herein are also provided. Such variants may have any of the levels of sequence identity, sequence similarity, or number of amino acid changes mentioned above in relation to CDR sets, but instead in respect of the entire VHH domain sequence.

In one embodiment, the binding molecules, are mutated to provide improved affinity for IL-12Rβ2. Such variants can be obtained by a number of affinity maturation protocols including mutating the CDRs (Yang et al., J. Mol. Biol., 254, 392-403, 1995), chain shuffling (Marks et al., Bio/Technology, 10, 779-783, 1992), use of mutator strains of E. coli (Low et al J. Mol. Biol., 250, 359-368, 1996), DNA shuffling (Patten et al Curr. Opin. Biotechnol., 8, 724-733, 1997), phage display (Thompson et al., J. Mol. Biol., 256, 77-88, 1996), and sexual PCR (Crameri et al Nature, 391, 288-291, 1998). Vaughan et al discusses these methods of affinity maturation (Vaughan et al., Nat. Biotech., 16, 535-539, 1998). Where not specifically for VHH domains such approaches may be adapted for them. Improving the affinity of binding of individual binding sites will typically also improve the overall avidity for the target where the binding molecule has more than one binding site.

The present invention also provides, and may employ, binding molecules which are able to compete with the specific binding molecules set out herein. The assays section of the present application sets out various binding and competition assays that may be performed and such assays may be used to confirm a given binding molecule is one able to compete with one of the specific binding molecules set out herein. Hence, the present invention also provides a binding molecule that is able to compete for binding with one of the VHH binding domains of Table 1. The present invention also provides a binding molecule that is able to compete for binding to IL-12Rβ2 with a binding molecule having a VHH binding-domain comprising a set of three CDRs from Table 1. In one embodiment, variant antibodies may be identified by identifying such antibodies that are able to cross-block specific antibodies set out herein. Cross-blocking binding molecules, in particular antibodies, can be identified using any suitable method in the art, for example by using competition ELISA or BIAcore assays where binding of the cross-blocking antibody to antigen prevents the binding of an antibody of the present invention or vice versa. Such cross-blocking assays may use cells expressing IL-12Rβ2 as a target. In one embodiment, flow cytometry is used to assess binding to cells expressing IL-12Rβ2.

The skilled person may generate binding molecules using any suitable method known in the art. Antigen polypeptides, for use in generating antibodies for example for use to immunize a host or for use in panning, such as in phage display, may be prepared by processes well known in the art from genetically engineered host cells comprising expression systems or they may be recovered from natural biological sources. In one embodiment, the host may be immunised with a cell expressing an IL-12Rβ2. In a particularly preferred embodiment, a VHH domain of the present invention is obtained by immunising a camelid and in particular a llama.

In one example, the antigen-binding sites, and in particular the variable regions, of the antibodies according to the invention are humanised. Humanised (which include CDR-grafted antibodies) as employed herein refers to molecules having one or more complementarity determining regions (CDRs) from a non-human species and a framework region from a human immunoglobulin molecule (see, e.g., U.S. Pat. No. 5,585,089; WO 91/09967 which are incorporated by reference). It will be appreciated that it may only be necessary to transfer the specificity determining residues of the CDRs rather than the entire CDR (see for example, Kashmiri et al., 2005, Methods, 36, 25-34). In a preferred embodiment though, the whole CDR or CDRs is/are transplanted. Humanised antibodies may optionally further comprise one or more framework residues derived from the non-human species from which the CDRs were derived. As used herein, the term “humanised antibody molecule” refers to an antibody molecule wherein one or more CDRs (including, if desired, one or more modified CDRs) from a donor antibody (e.g., a murine monoclonal antibody) are grafted into a framework of an acceptor antibody (e.g., a human antibody). For a review, see Vaughan et al, Nature Biotechnology, 16, 535-539, 1998.

When the CDRs or specificity determining residues are grafted, any appropriate acceptor variable region framework sequence may be used having regard to the class/type of the donor antibody from which the CDRs are derived, including mouse, primate, and human framework regions. Suitably, the humanised antibody according to the present invention has a variable domain comprising human acceptor framework regions as well as one or more of the CDRs provided herein. Examples of human frameworks which can be used in the present invention are KOL, NEWM, REI, EU, TUR, TEI, LAY and POM. For example, KOL and NEWM can be used for the heavy chain, REI can be used for the light chain and EU, LAY and POM can be used for both the heavy chain and the light chain. Alternatively, human germline sequences may be used; these are available at:
http://www2.mrc-lmb.cam.ac.uk/vbase/list2.php.

In a humanised binding molecule, in particular an antibody, of the present invention, the acceptor framework does not necessarily need to be derived from the same antibody and may, if desired, comprise composite chains having framework regions derived from different chains. The framework regions need not have exactly the same sequence as those of the acceptor antibody. For instance, unusual residues may be changed to more frequently occurring residues for that acceptor chain class or type. Alternatively, selected residues in the acceptor framework regions may be changed so that they correspond to the residue found at the same position in the donor antibody (see Reichmann et al 1998, Nature, 332, 323-324). Such changes should be kept to the minimum necessary to recover the affinity of the donor antibody. A protocol for selecting residues in the acceptor framework regions which may need to be changed is set forth in WO 91/09967. Derivatives of frameworks may have 1, 2, 3, 4, 5, 6, 7, 8, 9, 10 or more amino acids replaced with an alternative amino acid, for example with a donor residue. Donor residues are residues from the donor antibody, i.e., the antibody from which the CDRs were originally derived, in particular the residue in a corresponding location from the donor sequence is adopted. Donor residues may be replaced by a suitable residue derived from a human receptor framework (acceptor residues).

The Kabat et al numbering system is referred to herein. This system is set forth in Kabat et al., 1987, in Sequences of Proteins of Immunological Interest, US Department of Health and Human Services, NIH, USA (hereafter “Kabat et al. (supra)”). This numbering system is used in the present specification except where otherwise indicated. The Kabat residue designations do not always correspond directly with the linear numbering of the amino acid residues. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard Kabat numbered sequence. The CDRs of the heavy chain variable domain are typically located at residues 31-35 (CDR-H1), residues 50-65 (CDR-H2) and residues 95-102 (CDR-H3) according to the Kabat numbering system. However, according to Chothia (Chothia, C. and Lesk, A. M. J. Mol. Biol., 196, 901-917 (1987)), the loop equivalent to CDR-H1 extends from residue 26 to residue 32. Thus, unless indicated otherwise “CDR-H1” as employed herein is intended to refer to residues 26 to 35, as described by a combination of the Kabat numbering system and Chothia's topological loop definition. The CDRs of the light chain variable domain are typically located at residues 24-34 (CDR-L1), residues 50-56 (CDR-L2) and residues 89-97 (CDR-L3) according to the Kabat numbering system.

The skilled person is able to test variants of CDRs or humanised sequences in any suitable assay such as those described herein to confirm activity is maintained.

Further provided, and which may be employed, are binding molecules that bind the same epitope on IL-12Rβ2 as one of the specific antibodies set out herein. For instance, the binding molecule may be an antibody that binds to the same epitope.

An example of a preferred VHH is SEQ ID NO: 52. An example of a preferred set of three CDRs is SEQ ID NOs: 364, 365, and 366. An example of a preferred VHH is SEQ ID NO: 68. An example of a preferred set of three CDRs are those of SEQ ID NOs: 412, 413, and 414. An example of a preferred VHH is SEQ ID NO: 72. An example of a preferred set of three CDRs are those of SEQ ID NOs: 424, 425, and 426. An example of a preferred VHH is SEQ ID NO: 102. An example of a preferred set of three CDRs are those of SEQ ID NOs: 514, 515, and 516.

An example of a preferred VHH is SEQ ID NO: 111. An example of a preferred set of three CDRs are those of SEQ ID NOs: 541, 542, and 543. An example of a preferred VHH is SEQ ID NO: 128. An example of a preferred set of three CDRs are those of SEQ ID NOs: 592, 593, and 594. An example of a preferred VHH is SEQ ID NO: 131. An example of a preferred set of three CDRs are those of SEQ ID NOs: 601, 602, and 603. An example of a preferred VHH is SEQ ID NO: 132. An example of a preferred set of three CDRs are those of SEQ ID NOs: 604, 605, and 606. An example of a preferred VHH is SEQ ID NO: 146. An example of a preferred set of three CDRs are those of SEQ ID NOs: 646, 647, and 648.

An example of a preferred VHH is SEQ ID NO: 148. An example of a preferred set of three CDRs are those of SEQ ID NOs: 652, 653, and 654. An example of a preferred VHH is SEQ ID NO: 150. An example of a preferred set of three CDRs are those of SEQ ID NOs: 658, 659, and 660.

An example of a preferred VHH is SEQ ID NO: 151. An example of a preferred set of three CDRs are those of SEQ ID NOs: 661, 662, and 663. An example of a preferred VHH is SEQ ID NO: 175. An example of a preferred set of three CDRs are those of SEQ ID NOs: 733, 734, and 735.

An example of a preferred VHH is SEQ ID NO: 159. An example of a preferred set of three CDRs are those of SEQ ID NOs: 685, 686, and 687.

An example of a preferred VHH is SEQ ID NO: 196. An example of a preferred set of three CDRs are those of SEQ ID NOs: 796, 797, and 798. An example of a preferred VHH is SEQ ID NO: 204. An example of a preferred set of three CDRs are those of SEQ ID NOs: 820, 821, and 822.

An example of a preferred VHH is SEQ ID NO: 209. An example of a preferred set of three CDRs are those of SEQ ID NOs: 835, 836, and 837. An example of a preferred VHH is SEQ ID NO: 49. An example of a preferred set of three CDRs are those of SEQ ID NOs: 355, 356, and 357.

Also preferred are variants of any of the preferred VHHs set out above. Also preferred are variants of any of the preferred sets of 3 CDRs set out above. In a preferred embodiment, the binding molecule also comprises an Fc region with sequence modification to enhance ADCC activity as discussed above.

In one embodiment, a variant sequence will retain at least 50% of the ability to bind IL-12Rβ2 of the specific sequence, particularly to bind IL-12Rβ2 on the cell surface. In one embodiment, a variant will retain at least 60%, 70%, or 80% of the ability to bind. In another embodiment, it will retain at least 90% of the ability to bind in comparison to the specific sequence. In another embodiment, it will retain at least 95% of the ability to bind. In one embodiment, a variant will display at least such percentage values in comparison to the cell killing shown by a specific sequence. In another embodiment, it will show such percentage values in relation to ADCC, ADCP, and/or CDC activity. In one embodiment, a competing sequence may display such values.

### Cell Killing and Depletion

In an especially preferred embodiment, a binding molecule of the present invention may be used to deplete or kill target cells with IL-12Rβ2 on the cell surface.

The binding molecule may kill the target cell by virtue of activating the immune system to kill the target cell. In one embodiment, the binding molecule will have Antibody Dependent Cellular Cytotoxicity (ADCC) activity. Hence, the binding molecule will be able to trigger the lysis of the target cell by other immune cells. In another embodiment, the binding molecule will also, or alternatively display Antibody Dependent Cellular Phagocytosis (ADCP) activity. Hence, opsonisation of the target cell with the binding molecule will bring about phagocytosis and subsequent killing of the target cell by other immune cells such as neutrophils and macrophages. In another embodiment, the binding molecule will have Complement Dependent Cytotoxicity (CDC) activity, bringing about the lysis of the target cell via the activation of the complement system. The binding molecule displaying ADCC, ADCP, and/or CDC will typically mean that the binding molecule is able to bind an Fc receptor or receptors. As discussed herein, the binding molecule may have constant region modifications or be afucosylated in order to promote such functions. In another embodiment, rather than activate the immune system to kill the target cell the binding molecule itself will kill the target cell after binding IL-12Rβ2 on the cell surface. In one embodiment, the binding molecule may be conjugated to a molecule that is able to kill the target cell. In one embodiment, the binding molecule is conjugated to a toxin that is able to kill the target cell. The binding molecule may be conjugated to a radioisotope to kill the target cell.

### Conjugates, Fusion Proteins, Effector Molecules, and Labels

In a preferred embodiment, a binding molecule, particularly an antibody, of the present invention may exert its effect by binding IL-12Rβ2 and bringing about killing of the target cell without any need for a further effector molecule as part of the binding molecule. In an especially preferred embodiment, the binding molecule specific for IL-12Rβ2 will display ADCC, ADCP, and/or CDC and so will be able to bring about killing of the target cell that way via effector cells with Fc receptors binding to the binding molecule via those receptors and then killing the target cell.

In an alternative embodiment, a binding molecule of the present invention may additionally, or instead be able to kill target cells by virtue of being conjugated to an effector molecule that is able to kill cells. Where it is desired to obtain a binding molecule, particularly an antibody, according to the present invention linked to an effector molecule or label, this may be prepared by standard chemical or recombinant DNA procedures in which the binding molecule is linked either directly or via a coupling agent to the effector molecule. Techniques for conjugating such effector molecules to antibodies are well known in the art (see, Hellstrom et al., Controlled Drug Delivery, 2nd Ed., Robinson et al., eds., 1987, pp. 623-53; Thorpe et al., 1982, Immunol. Rev., 62:119-58 and Dubowchik et al., 1999, Pharmacology and Therapeutics, 83, 67-123). Particular chemical procedures include, for example, those described in WO 93/06231, WO 92/22583, WO 89/00195, WO 89/01476 and WO 03/031581. Alternatively, where the effector or label molecule is a protein or polypeptide the linkage may be achieved using recombinant DNA procedures, for example as described in WO 86/01533 and EP0392745.

In another embodiment, a binding molecule, particularly an antibody, of the invention is not conjugated to an effector molecule. In one embodiment, a binding molecule, particularly an antibody, of the invention is not conjugated to a toxin. In another embodiment, a binding molecule, particularly an antibody, of the invention is not conjugated to a radioisotope. In another embodiment, it is not conjugated to an agent for imaging.

The binding molecules provided may be used to detect IL-12Rβ2, for instance via flow cytometry. In such embodiments, the binding molecule may be itself labelled or can be recognised by another reagent to bring about such labelling. The binding molecules may be used to identify the presence of cells expressing IL-12Rβ2. In one embodiment, such labelled molecules are employed to determine the level of cells expressing IL-12Rβ2 after, or during, the cell depletion methods of the present invention.

### Assays

In one embodiment, an assay may be employed to determine if a given binding molecule, has a particular property or properties, or the level of an activity of interest a binding molecule has. Such assays may detect or measure the activity of interest. One or more of the assays described in the Examples of the present application may be employed to assess a particular binding molecule and whether it has a desired property or properties. The assays in the Examples may be employed, for instance, to determine the ability of a binding molecule to bind to IL-12Rβ2, kill target cells expressing IL-12Rβ2 on their surface, compete with IL-12, and/or compete with another binding molecule.

Any suitable method for measuring binding may be employed, such as those used in the Examples of the present application. The ability to bind IL-12Rβ2 may be assessed by employing techniques like surface plasmon resonance using IL-12Rβ2, or a portion thereof, bound to a chip. A binding molecule of the present invention will be typically able to bind to IL-12Rβ2 when present on the cell surface. Hence, in a particularly preferred embodiment, the ability to bind IL-12Rβ2 on the surface of cells will be measured. In a preferred embodiment, flow cytometry will be employed to measure such binding.

In one embodiment, the ability of a candidate binding molecule to bind to IL-12Rβ2 is assessed in an assay comprising: (a) contacting a candidate binding molecule with a cell expressing IL-12Rβ2 on its surface; and (b) detecting any binding of the candidate binding molecule to the cells with IL-12Rβ2 on their surface. In a particularly preferred embodiment, the method is a flow cytometry method. In one embodiment, the cells express human IL-12Rβ2 on their surface. In one embodiment, the cells employed are HEK293T cells transiently transfected with a human IL-12Rβ2 expression plasmid. In another embodiment, the cells used are a stable cell line expressing IL-12Rβ2. In one embodiment, the cells are a stable HEK293T cell line expressing IL-12Rβ2 on their surface. In a further preferred embodiment, either the candidate binding molecule is itself labelled or is detected using a secondary antibody. In one embodiment, the ability of a candidate binding molecule to bind to IL-12Rβ2 is assessed in a flow cytometry assay comprising: (a) contacting a candidate binding molecule with a stable HEK293T cell line expressing IL-12Rβ2 on its surface; and (b) detecting by flow cytometry any binding of the candidate binding molecule to the cells, wherein the candidate binding molecule comprises HA, with binding to the cell detected using a mouse anti-HA antibody and an anti-mouse PE antibody.

Competition assays may also be used to determine whether a candidate binding molecule is able to compete for binding to IL-12Rβ2 with another binding molecule. In one embodiment, a variant binding molecule has the ability to compete for binding to IL-12Rβ2 with one of the specific binding molecules of the present invention. In one embodiment, an assay to assess competition may comprise: (a) contacting a candidate binding molecule, a labelled binding molecule of the invention, and a cell expressing IL-12Rβ2 on its surface; (b) determining the amount of binding of the labelled binding molecule of the present invention to the cells compared to the amount of binding for the same assay performed without the candidate binding molecule. In one embodiment, rather than a competition assay, the ability of a candidate binding molecule and a specific binding molecule of the present invention to bind to cells expressing IL-12Rβ2 is measured individually using the same assay and the two results obtained are compared. Any of the assays and methods set out herein to measure binding to cells may also be used to measure competition or to compare binding of two binding molecules to IL-12Rβ2.

In one preferred embodiment, a binding molecule of the present invention does not compete with IL-12 for binding to IL-12Rβ2 or does not significantly do so. Hence, an assay may be used to determine the ability of a given binding molecule to compete with IL-12 for binding. In one embodiment, the assay may comprise: (a) contacting HEK-Blue IL-12 reporter cells with IL-12 in the presence and absence of the binding molecule; and (b) measuring secreted embryonic alkaline phosphatase in the cell culture medium to determine if the presence of the binding molecule decreases the amount of secreted embryonic alkaline phosphatase indicating that the binding molecule is competing with II-12 for binding to the IL-12 receptor. In one preferred embodiment, the assay is performed with different dilutions of the binding molecule to determine the effect of increasing binding molecule concentration on the ability of IL-12 to bind to its receptor.

In one embodiment, the ability of a binding molecule of the present invention to kill a target cell is assessed. The ability to kill a target cell expressing IL-12Rβ2 via ADCC may be assessed. Any of the target cells expressing IL-12Rβ2 used in the Examples of the present case may be, for instance, employed such as Daudi or HEK293T cells expressing human IL-12Rβ2. Effector cells such as PBMC or NK cells may be employed, for example, in such assays after the target cells have been opsonised with a candidate binding molecule. A preferred assay method comprises: (a) contacting Daudi cells with stable transgenic human IL-12Rβ2 expression with a binding molecule; (b) adding PBMCs that have been activated with IL-2; and (c) measuring cell lysis. In one embodiment, the method is performed with human NK cells rather than PBMCs as the effector cells. In a further embodiment, rather than Daudi cells Jurkat cells with stable human or mouse IL-12Rβ2 expression may be used as the target cells.

In one embodiment, a binding molecule may be assessed for its ability to mediate killing of human CD4+ Th1 cells, for instance using the assays described in the Examples of the present application. In one embodiment, a variant may retain such activity.

In one embodiment, the ability of a binding molecule to compete with IL-12 for IL-12R activation will be measured, for instance by using primary human CD4 T cells as described in the Examples of the present application and measurement of phosphorylation of STAT4. In one embodiment, a variant will be able to compete with IL-12 in such an assay. In another it will not.

In one embodiment, the ability of a binding molecule to prevent expansion of alloreactive Tbet+CD4+ T cells may be measured, for instance using the assay set out in the Examples of the present application. In one embodiment, a variant will retain such activity.

In another embodiment, toxicity in naïve mice will be measured by administration of a test binding molecule intraperitoneally to mice followed 48 hours later by measurement of T, B, and NK cell levels and/or regulatory T cell levels.

The efficacy of a binding molecule, in particular of an antibody, may be assessed in an in vivo system, such as in an animal model of a disease condition. In one embodiment, an animal model of any of the conditions mentioned herein may be employed to assess a binding molecule of the present invention. Such animal models may be used to assess whether a given binding molecule is able to treat or prevent the condition in question or to reduce the degree of severity of the disease.

### Therapy & Diagnosis

The present invention provides a way to kill target cells expressing IL-12Rβ2. In one preferred embodiment, the invention provides a binding molecule specific for IL-12Rβ2 for use in a method of treatment comprising killing target cells expressing IL-12Rβ2. In one embodiment, the method comprises administering the binding molecule to a subject to deplete cells expressing IL-12Rβ2. In one embodiment, cells expressing IL-12Rβ2 will be depleted in a tissue, organ, or population of cells prior to transplant to a subject or post transplantation.

In one embodiment, the subject has an autoimmune or inflammatory disease. In one embodiment, the disorder to be treated is characterized by a type 1 immune response. In a preferred embodiment, the subject has an autoimmune disorder. In one embodiment, the autoimmune disorder is an organ specific autoimmune disorder. In one embodiment, the disorder is an autoimmune disorder of the central nervous system (CNS). In another embodiment, the disorder is an autoimmune disorder affecting the bowel.

An example of a preferred disorder to be treated is arthritis. In a preferred embodiment, the arthritis is rheumatoid arthritis. In another particularly preferred embodiment, the disorder is multiple sclerosis. In a further preferred embodiment, the disorder to be treated is inflammatory bowel disease (IBD). In another preferred embodiment, the disorder is psoriasis. In a further preferred embodiment, the condition is Type 1 diabetes.

CD8+ Tc1 and CD4+ Th1 type one immune cells are characterised by markers such as IL-12Rβ2, IL-27Rα/WSX-1, IFN-γR2, IL-18R, CCR5, and CXCR3, and the expression of the transcriptional regulators, STAT4 and T-bet. ILC1 cells are characterised by markers such as CD127, CD161, IL-1R1, IL-18R, IL-12Rβ2, and the expression of the transcriptional regulators, STAT4 and T-bet. In one embodiment, the subject may be first diagnosed with a condition characterised by a type 1 biased immune response or having a flare-up of such a condition. In one embodiment, the binding molecule specific for IL-12Rβ2 may be first used to detect the presence of a high level of cells expressing IL-12Rβ2. The binding molecule may be also used to monitor the course of treatment, for instance as a means to determine the depletion of IL-12Rβ2 expressing cells from the subject. Any of the binding molecules specific for IL-12Rβ2 herein may be labelled to provide a way to detect and/or quantify the level of cells expressing IL-12Rβ2. In one embodiment, part of the diagnosis of any of the conditions mentioned herein may comprise measuring the level of cells expressing IL-12Rβ2 on their surface using the binding molecules discussed herein to measure the level of such cells. Measurement of the level of such cells using the binding molecules against IL-12Rβ2 may also be used to determine the suitability of a subject for cell depletion using such binding molecules.

Also provided is a binding molecule, in particular an antibody, of the present invention for use as a medicament. In another embodiment a binding molecule, in particular an antibody, of the present invention is provided for use in a method of therapy of the human or animal body. Please note that, in the various therapeutic uses set out herein where reference is made to a binding molecule or an antibody of the present invention, a pharmaceutical composition comprising it may be also employed and vice versa unless stated otherwise, as may be a composition encoding a binding molecule, in particular an antibody, of the invention. A binding molecule, in particular an antibody, of the present invention may also be used in in vitro diagnosis, for example such diagnosis performed on a sample from a subject.

As discussed further below, a binding molecule, in particular an antibody, of the present invention may be employed to treat a condition. As used herein, the terms “treat” or “treatment” refer to both therapeutic treatment and prophylactic or preventative measures, wherein the object is to prevent or slow down (lessen) an undesired physiological change or disorder. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented. The invention may be used to treat such disorders in the sense that the condition is ameliorated, for example one or more symptoms of the disorder are lessened or eliminated. The present invention also provides methods of treatment for such disorders comprising administering a binding molecule specific for IL-12Rβ2 to deplete target cells expressing IL-12Rβ2. The present invention also provides for the use of a binding molecule specific for IL-12Rβ2 as discussed herein in the manufacture of a medicament to treat or prevent any of the conditions mentioned herein,

### Pharmaceutical Compositions

In one embodiment, the present invention provides a pharmaceutical composition comprising: (a) a binding molecule specific for IL-12Rβ2; and (b) a pharmaceutically acceptable carrier, diluent, and/or excipient. The particularly preferred binding molecule for any of the pharmaceutical compositions of the present invention is an antibody of the present invention. In one embodiment, a pharmaceutical composition of the present invention comprises binding molecule of the present invention as well as a carrier, a stabilizer, an excipient, a diluent, a solubilizer, a surfactant, an emulsifier, a preservative and/or adjuvant. In one embodiment, a pharmaceutical composition of the present invention is in solid or liquid form. In one embodiment, the pharmaceutical composition may be in the form of a powder, a tablet, a solution or an aerosol. In one embodiment, a pharmaceutical composition of the present invention is provided in a frozen form. In one embodiment, a pharmaceutical composition of the present invention is provided in lyophilized form.

A pharmaceutical composition of the present invention will usually be supplied as a sterile, pharmaceutical composition. A pharmaceutical composition of the present invention may additionally comprise a pharmaceutically acceptable adjuvant. In another embodiment, no such adjuvant is present in a pharmaceutical composition of the present invention. The present invention also provides a process for preparation of a pharmaceutical or medicament composition comprising adding and mixing binding molecule of the present invention together with one or more of a pharmaceutically acceptable excipient, diluent or carrier.

Pharmaceutically acceptable carriers in therapeutic compositions may additionally contain liquids such as water, saline, glycerol and ethanol. Such carriers may be used, for example, so that the pharmaceutical compositions to be formulated as tablets, pills, dragées, capsules, liquids, gels, syrups, slurries and suspensions, for ingestion by the patient. The term “pharmaceutically acceptable excipient” as used herein typically refers to a pharmaceutically acceptable formulation carrier, solution or additive to enhance the desired characteristics of the compositions of the present invention. Excipients are well known in the art and include buffers (e.g., citrate buffer, phosphate buffer, acetate buffer, and bicarbonate buffer), amino acids, urea, alcohols, ascorbic acid, phospholipids, proteins (e.g., serum albumin), EDTA, sodium chloride, liposomes, mannitol, sorbitol, and glycerol. Solutions or suspensions can be encapsulated in liposomes or biodegradable microspheres. Suitable carriers may be large, slowly metabolised macromolecules such as proteins, polypeptides, liposomes, polysaccharides, polylactic acids, polyglycolic acids, polymeric amino acids, amino acid copolymers and inactive virus particles. Pharmaceutically acceptable salts can be used, for example mineral acid salts, such as hydrochlorides, hydrobromides, phosphates and sulphates, or salts of organic acids, such as acetates, propionates, malonates, and benzoates.

In certain embodiments, the pharmaceutical composition may contain formulation materials for the purpose of modifying, maintaining or preserving certain characteristics of the composition such as the pH, osmolarity, viscosity, clarity, color, isotonicity, odour, sterility, stability, rate of dissolution or release, adsorption or penetration. A thorough discussion of pharmaceutically acceptable carriers is available in Remington's Pharmaceutical Sciences (Mack Publishing Company, N.J. 1991). Additional pharmaceutical compositions include formulations involving the binding molecule, in particular antibody, of the present invention in sustained or controlled delivery formulations. Techniques for formulating a variety of sustained- or controlled-delivery means are known to those skilled in the art. A binding molecule, in particular antibody, of the present invention may also be entrapped in microcapsules prepared, for example, by coacervation techniques or by interfacial polymerization, in colloidal drug delivery systems, or in macroemulsions. Such techniques are also disclosed in Remington's Pharmaceutical Sciences.

A subject will be typically administered a therapeutically effective amount of a pharmaceutical composition and hence of a binding molecule, in particular an antibody, of the present invention. The term “therapeutically effective amount” typically refers to an amount of a therapeutic agent needed to treat, ameliorate or prevent a targeted disease or condition, or to exhibit a detectable therapeutic or preventative effect. The precise therapeutically effective amount for a human subject will depend upon the severity of the disease state, the general health of the subject, the age, weight and gender of the subject, diet, time and frequency of administration, drug combination(s), reaction sensitivities, and tolerance/response to therapy. This amount can be determined by routine experimentation and is within the judgement of the clinician. Generally, a therapeutically effective amount will be from 0.01 mg/kg to 50 mg/kg, for example 0.1 mg/kg to 20 mg/kg per day. Alternatively, the dose may be 1 to 500 mg per day, such as 10 to 100, 200, 300 or 400 mg per day. In one embodiment, the amount in a given dose is at least enough to bring about a particular function, such as to treat, prevent, or ameliorate a disorder of interest.

In one embodiment, a binding molecule, in particular an antibody, of the present invention may be given in combination with another treatment for the condition being treated. For example, a binding molecule, in particular an antibody, of the present invention may be provided simultaneously, sequentially, or separately with such a further agent. In another embodiment, an antibody of the present invention may be provided in the same pharmaceutical composition as a second therapeutic agent.

In one preferred embodiment, the therapeutic agent of the invention, when in a pharmaceutical preparation, may be present in unit dose forms. Suitable doses may be calculated for patients according to their weight, for example suitable doses may be in the range of 0.01 to 20 mg/kg, for example 0.1 to 20 mg/kg, for example 1 to 20 mg/kg, for example 10 to 20 mg/kg or for example 1 to 15 mg/kg, for example 10 to 15 mg/kg. To effectively treat conditions of use in the present invention in a human, suitable doses may be within the range of 0.001 to 10 mg, 0.01 to 1000 mg, for example 0.1 to 1000 mg, for example 0.1 to 500 mg, for example 500 mg, for example 0.1 to 100 mg, or 0.1 to 80 mg, or 0.1 to 60 mg, or 0.1 to 40 mg, or for example 1 to 100 mg, or 1 to 50 mg, of a dual targeting protein of this invention, which may be administered parenterally, for example subcutaneously, intravenously or intramuscularly. Such a dose may be, if necessary, repeated at appropriate time intervals selected as appropriate by a physician. A binding molecule, and in particular an antibody, of the present invention may be, for instance, lyophilized for storage and reconstituted in a suitable carrier prior to use. Lyophilization and reconstitution techniques can be employed.

The binding molecules, in particular antibodies, and pharmaceutical compositions of this invention may be administered by any number of routes including, but not limited to, oral, intravenous, intramuscular, intra-arterial, intramedullary, intrathecal, intraventricular, transdermal, transcutaneous (for example, see WO 98/20734), subcutaneous, intraperitoneal, intranasal, enteral, topical, sublingual, intravaginal or rectal routes. Hyposprays may also be used to administer the pharmaceutical compositions of the invention. Direct delivery of the compositions will generally be accomplished by injection, subcutaneously, intraperitoneally, intravenously, or intramuscularly, or delivered to the interstitial space of a tissue. In one preferred embodiment, administration is via intravenous administration. In another preferred embodiment, administration is via subcutaneous administration, for example via subcutaneous injection. The compositions can also be administered into a specific tissue of interest. In some embodiments, administration is via site-specific or targeted local delivery techniques. Examples of site-specific or targeted local delivery techniques include various implantable depot sources of the antibody molecule or local delivery catheters, such as infusion catheters, indwelling catheters, or needle catheters, synthetic grafts, adventitial wraps, shunts and stents or other implantable devices, site specific carriers, direct injection, or direct application.

Dosage treatment may be a single dose schedule or a multiple dose schedule. Where the product is for injection or infusion, it may take the form of a suspension, solution, or emulsion in an oily or aqueous vehicle and it may contain formulary agents, such as suspending, preservative, stabilising and/or dispersing agents. Alternatively, the pharmaceutical may be in dry form, for reconstitution before use with an appropriate sterile liquid. In one embodiment, a pharmaceutical composition comprising an antibody of the present invention is provided in lyophilised form. If a composition is to be administered by a route using the gastrointestinal tract, the composition will typically need to contain agents which protect the binding molecule, in particular antibody, from degradation but which release the binding molecule once it has been absorbed from the gastrointestinal tract. In another embodiment, a nebulisable formulation according to the present invention may be provided, for example, as single dose units (e.g., sealed plastic containers or vials) packed in foil envelopes. Each vial contains a unit dose in a volume, e.g., 2 mL, of solvent/solution buffer.

A pharmaceutical composition of the present invention may be provided in a receptacle that provides means for administration to a subject. In one embodiment, a pharmaceutical composition of the present invention may be provided in a prefilled syringe. The present invention therefore provides such a loaded syringe. It also provides an auto-injector loaded with a pharmaceutical composition of the present invention.

In one embodiment the formulation is provided as a formulation for topical administrations including inhalation. Suitable inhalable preparations include inhalable powders, metering aerosols containing propellant gases or inhalable solutions free from propellant gases. Inhalable powders according to the invention containing the active substance may consist solely of the abovementioned active substances or of a mixture of the abovementioned active substances with physiologically acceptable excipient. These inhalable powders may include monosaccharides (e.g., glucose or arabinose), disaccharides (e.g., lactose, saccharose, maltose), oligo- and polysaccharides (e.g., dextranes), polyalcohols (e.g., sorbitol, mannitol, xylitol), salts (e.g., sodium chloride, calcium carbonate) or mixtures of these with one another. Mono- or disaccharides are suitably used, the use of lactose or glucose, particularly but not exclusively in the form of their hydrates.

Particles for deposition in the lung require a particle size less than 10 microns, such as 1-9 microns for example from 1 to 5 μm. The particle size of the active ingredient such as the antibody or fragment is of primary importance. The propellant gases which can be used to prepare the inhalable aerosols are known in the art. Suitable propellant gases are selected from among hydrocarbons such as n-propane, n-butane or isobutane and halohydrocarbons such as chlorinated and/or fluorinated derivatives of methane, ethane, propane, butane, cyclopropane or cyclobutane. The above mentioned propellent gases may be used on their own or in mixtures thereof. Particularly suitable propellent gases are halogenated alkane derivatives selected from among TG 11, TG 12, TG 134a and TG227. Of the above-mentioned halogenated hydrocarbons, TG134a (1,1,1,2-tetrafluoroethane) and TG227 (1,1,1,2,3,3,3-heptafluoropropane) and mixtures thereof are particularly suitable. The propellent-gas-containing inhalable aerosols may also contain other ingredients such as cosolvents, stabilisers, surface-active agents (surfactants), antioxidants, lubricants and means for adjusting the pH. All these ingredients are known in the art. The propellant-gas-containing inhalable aerosols according to the invention may contain up to 5% by weight of active substance. Aerosols according to the invention contain, for example, 0.002 to 5% by weight, 0.01 to 3% by weight, 0.015 to 2% by weight, 0.1 to 2% by weight, 0.5 to 2% by weight or 0.5 to 1% by weight of active ingredient.

Alternatively topical administrations to the lung may also be by administration of a liquid solution or suspension formulation, for example employing a device such as a nebulizer, for example, a nebulizer connected to a compressor (e.g., the Pari LC-Jet Plus (R) nebulizer connected to a Pari Master (R) compressor manufactured by Pari Respiratory Equipment, Inc., Richmond, Va.).

Nebulisable formulation according to the present invention may be provided, for example, as single dose units (e.g., sealed plastic containers or vials) packed in foil envelopes. Each vial contains a unit dose in a volume, e.g., 2 mL of solvent/solution buffer. The present invention also provides a syringe loaded with a composition comprising a binding molecule, in particular an antibody, of the invention. In one embodiment, a pre-filled syringe loaded with a unit dose of an antibody is provided. In another embodiment, an autoinjector loaded with a binding molecule, in particular an antibody, of the invention is provided. In a further embodiment, an IV bag loaded with a pharmaceutical composition of the invention is provided.

It is also envisaged that an antibody of the present invention may be administered by use of gene therapy. In order to achieve this, DNA sequences encoding the binding molecule, in particular antibody, under the control of appropriate DNA components are introduced into a patient such that the binding molecule, in particular antibody chains and so antibody, are expressed from the DNA sequences and assembled in situ.

Once formulated, the compositions of the invention can be administered directly to the subject. By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on. In a preferred embodiment, the subject to be treated is a mammal. The subjects to be treated can be animals. However, in one or more embodiments the compositions are adapted for administration to humans. In a particularly preferred embodiment, the subject is human. In another embodiment, the subject is an animal model of one of the conditions recited herein. In one embodiment, the subject has experimental autoimmune encephalomyelitis (EAE), for example is a rodent with EAE. In another embodiment, the subject is a rodent with arthritis, for example has collagen-induced arthritis (CIA)

### Kits

The present invention also extends to a kit comprising a binding molecule of the invention, in particular an antibody, of the invention, optionally with instructions for administration. In yet another embodiment, the kit further comprises one or more reagents for performing one or more functional assays. In another embodiment, a kit containing single-chambered or multi-chambered pre-filled syringe is provided which is pre-filled with a pharmaceutical composition of the invention. The invention also provides a kit for a single-dose administration unit which comprises a pharmaceutical composition of the invention. In another embodiment, the kit comprises packaging.

### Further Embodiments

The following represent further numbered embodiments, but do not at present represent claims.
[1] A binding molecule specific for an IL-12Rβ2 subunit for use in a method of treatment, the method comprising depleting cells expressing the IL-12Rβ2 subunit using the binding molecule.
[2] The binding molecule for use of [1], wherein the binding molecule has ADCC, ADCP, and/or CDC activity.
[3] The binding molecule for use of [1] or [2], wherein:
   (a) the binding molecule does compete for binding with IL-12; or
   (b) the binding molecule does not compete for binding with IL-12.
[4] The binding molecule for use of any one of [1] to [3], wherein the binding molecule is an antibody.
[5] The binding molecule for use of any one of the preceding claims, wherein the binding molecule is, or comprises, a VHH domain antibody.
[6] The binding molecule for use of any one of [1] to [5], wherein the method is for treating an autoimmune or inflammatory disorder, or a pathology where IL-12 via its IL-12 receptor complex including the IL-12Rβ2 contributes to the pathology.
[7] The binding molecule for use of any one [1] to [6], wherein the method is for treating or preventing arthritis, multiple sclerosis, autoimmune uveitis, inflammatory bowel disease, graft versus host disease or Type 1 diabetes.
[8] The binding molecule for use of [1] to [7], wherein the method comprises depleting ‘type 1 immune cells’ such as CD4+ Th1, CD8+ Tc1, ILC1 cells.
[9] A method of treating comprising administering a binding molecule specific for an IL-12Rβ2 subunit to a subject, wherein the binding molecule depletes cells expressing the IL-12Rβ2 subunit in the subject.
[10] The method of [9], wherein the binding molecule has ADCC, ADCP, and/or CDC activity.
[11] The method of [10], wherein:
   (a) the binding molecule does compete for binding with IL-12; or
   (b) the binding molecule does not compete for binding with IL-12.
[12] The method of any one of [9] to wherein the binding molecule is an antibody.
[13] The method of any one of [9] to [12], wherein the binding molecule is, or comprises, a VHH domain antibody.
[14] The method of any one of [9] to [13], wherein the method is for treating an autoimmune or inflammatory disorder, or a pathology where IL-12 via its IL-12 receptor complex including the IL-12Rβ2 contributes to the pathology.
[15] The method of any one of [9] to [14], wherein the method is for treating or preventing arthritis, multiple sclerosis, autoimmune uveitis, inflammatory bowel disease, graft versus host disease or Type 1 diabetes.
[16] The method of any one of [9] to [15], wherein the method comprises depleting type 1 immune cells such as CD4+ Th1, CD8+ Tc1, ILC1 cells.
[17] A binding molecule that binds to IL-12Rβ2, wherein the binding molecule comprises one or more of the following VHH antigen-domains:
   (a) a VHH antigen-binding domain that binds IL-12Rβ2 comprising a set of three CDRs (CDR1, CDR2, and CDR3) selected from the sets of three CDRs of Table 1;
   (b) a VHH antigen-binding domain that binds IL-12Rβ2 comprising a set of three CDRs (CDR1, CDR2, and CDR3) that correspond to a set of three CDRs of Table 1 apart from a maximum of ten amino acid sequence changes;
   (c) a VHH antigen-binding domain that binds IL-12Rβ2 comprising a set of three CDRs (CDR1, CDR2, and CDR3) that have at least 90% sequence identity to a set of three CDRs of Table 1; or
   (d) a VHH antigen-binding domain that can compete for binding to IL-12Rβ2 with a VHH antigen-domain of any of (a) to (c).
[18] The binding molecule of [17], wherein the binding molecule comprises one or more of the following VHH antigen-domains:
   (a) a VHH antigen-binding domain that binds IL-12Rβ2 and is selected from the VHH antigen-binding domain having the sequence of one of the VHH antigen-binding domains of Table 1;
   (b) a VHH antigen-binding domain that binds IL-12Rβ2 and has at least 80% sequence identity to one of the VHH antigen-binding domains of Table 1;
   (c) a VHH antigen-binding domain that binds IL-12Rβ2 and is a humanized version of one of VHH antigen-binding domains of Table 1; or
   (d) a VHH antigen-binding domain that can compete for binding to IL-12Rβ2 with an a VHH antigen-domain of any of (a) to (c).
[19] The binding molecule of or [18], wherein the binding molecule comprises a single a VHH antigen-binding domain and an Fc domain.
[20] The binding molecule of any one of to [19], wherein the binding molecule has ADCC, ADCP, and/or CDC activity.
[21] The binding molecule of any one of to [20], wherein the binding molecule is able to kill cells expressing IL-12Rβ2.
[22] The binding molecule of any one of to [21], wherein:
   (a) the binding molecule does not compete for binding to the IL-12Rβ2 with IL-12; or
   (b) the binding molecule does compete for binding to the IL-12Rβ2 with IL-12.
[23] A pharmaceutical composition comprising a binding molecule according to any one of to [22] and a pharmaceutically acceptable carrier.
[24] A binding molecule of any one of to or pharmaceutical composition according to claim 23 for use in a method of treatment or diagnosis of the human or animal body.
[25] A binding molecule of any one of claims to or pharmaceutical composition according to [6] for use in a method of treating or preventing an autoimmune or inflammatory disorder.
[26] The binding molecule or pharmaceutical composition for the use of [25], wherein the method of treating the autoimmune or inflammatory disorder comprises depleting cells expressing the IL-12Rβ2 receptor subunit.
[27] A binding molecule specific for an IL-12Rβ2 subunit for use in a method of treatment or diagnosis of the human or animal body, optionally wherein the binding molecule is an antibody, preferably wherein the antibody comprises a single VHH domain and an Fc region.

### EXAMPLES

The invention will be further understood with reference to the following non-limiting Examples.

### Example 1: Generation of Anti-IL-12Rβ2 Antibodies

A: Llama Immunization with Recombinant IL-12Rβ2

A male and female llama were subcutaneously injected on days 0, 14, 28 and 42, each time with a mixture of recombinant human IL-12Rβ2 fused to hIgG1 Fc-His6 at the C-terminus (hIL-12Rβ2-Fc) (R&D Systems, Cat. No. 1959-B2B-050), recombinant mouse IL-12Rβ2 fused to hIgG1 Fc-His6 at the C-terminus (mIL-12Rβ2-Fc) (R&D Systems, Cat. No. 7406-MR-050), tagless human IL-12Rβ2 (produced in-house), and tagless mouse IL-12Rβ2 (produced in-house) in combination with Gerbu adjuvant P. At four and eight days after the last immunization (4 d.p.i. and 8 d.p.i.) 100 ml of anticoagulated blood was collected from each llama for lymphocyte preparation.

### B: Selection of VHH Binding to IL-12Rβ2

Individual libraries of VHH regions of llama heavy chain-only antibodies were constructed from each llama's lymphocytes to screen for the presence of antigen-specific VHHs. To that end, total RNAs from peripheral blood lymphocytes from 4 d.p.i. & 8 d.p.i. were pooled per animal and used as template for cDNA synthesis. The VHH encoding sequences were then amplified by PCR and cloned into the pMECS phagemid vector. Phagmid libraries were separately panned in solution on random biotinylated human or mouse IL-12Rβ2 (produced in-house) for 3 rounds. Colonies from each panning set were analyzed by ELISA for the presence of antigen-specific VHHs in their periplasmic extracts (P.E.). The screening ELISA was performed using the same human and mouse biotinylated IL-12Rβ2, with streptavidin-coated blocked wells as negative control. Based on the sequence data of the positive colonies, the number of unique full length VHHs were determined and categorized in different CDR3 groups. 209 unique anti-IL-12Rβ2 VHHs were identified in 62 CDR3 groups, of which 19 showed signs of human/mouse cross-reactivity based on ELISA results. The results obtained are provided in Table 1 and Table 2 below.

Dissociation off-rate constants of the human-specific and human-mouse cross-reactive anti-IL-12Rβ2 VHH clones (based on ELISA binding study) were analyzed using Bio-Layer Interferometry (BLI). Biotinylated human or mouse recombinant IL-12Rβ2 proteins (10 μg/ml) were immobilized on a Fortebio streptavidin-coated biosensor tips. Next, 200 μl VHH-containing P.E. were mixed with 2 μl of 10% Tween20-PBS in a 96-well plate. pMECS vector P.E. was used as negative control. The P.E. plate was loaded into a Fortebio Octet Red and brought into contact with antigen-coated Octet tips. Consequently, the binding profile for each clone was determined. Using the Fortebio Data Analysis Software, the blanks were subtracted and the curves were aligned. Based on these curves, the off-rate constants were calculated using a 1:1 binding model. The Data obtained is shown in Table 2.

### C: Binding of Anti-IL-12Rβ2 VHH Clones to Cells Expressing IL-12Rβ2

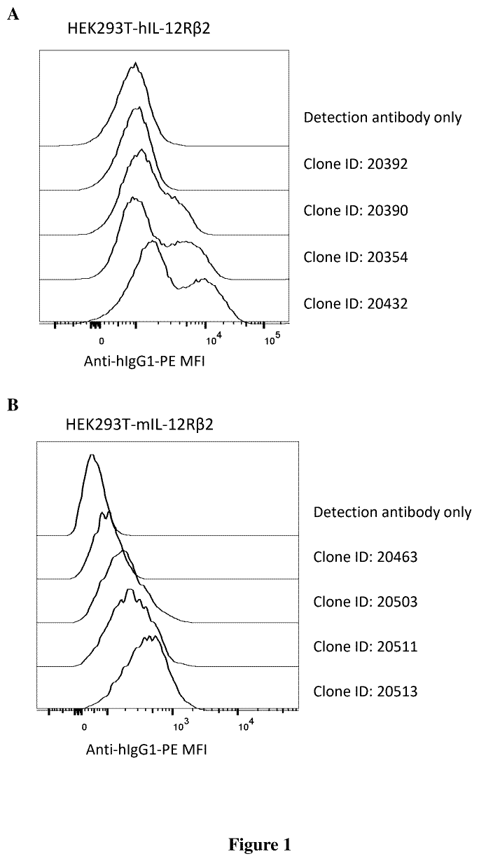

Anti-IL-12Rβ2 VHH clones which showed binding to human-only or human and mouse IL-12Rβ2 in the ELISA screening were next assessed for their ability to bind human and mouse cell membrane-expressed IL-12Rβ2. This was analyzed using HEK293T cells transiently transfected with a pcDNA3 expression plasmid coding for the human or mouse IL-12Rβ2 receptor (Genscript, clone OHu21785 & OMu01776). Surface expression was confirmed by flow cytometry using anti-IL-12Rβ2 (R&D, FAB1959A). 48 hours after transfection cells were harvested and reseeded at 100000 cells/well of a 96-plate, washed with FACS buffer, and incubated with different dilutions of anti-IL-12Rβ2 VHH-containing P.E. in FACS buffer at 4° C. for 40 minutes. After washing the cells with FACS buffer, cells were incubated with a mouse anti-HA antibody (Biolegend, clone HA.11), to bind the C-terminal hemagglutinin (HA) tag fused to the VHHs, for 40 minutes at 4° C. Cells were washed again with FACS buffer and stained with anti-mouse IgG-PE detection antibody (Biolegend, 405307) for 30 minutes at 4° C. Stained cells were analyzed on an LSR HTS (BD Biosciences). Dead cells were excluded from the analysis based on forward and side scatter properties. Representative graphs of selected VHHs are shown in FIG. 1, all data can be found in Table 3.

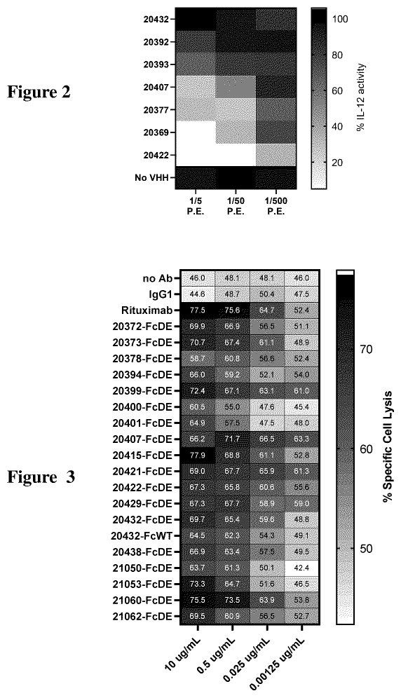

The ability of mouse-specific anti-IL-12Rβ2 VHH clones to bind mouse cell membrane-expressed IL-12Rβ2 was also tested. One VHH clone per CDR3 family was selected for the analysis. This was analyzed using HEK293T cells transduced with pLVX-EFla-mIL-12Rβ2-IRES-ZsGreen (in-house constructed, mIL-12Rβ2 cDNA: NM_008354) derived lentiviral particles and subsequently sorted for ZsGreen-positive cells. The staining procedure was similar as described above. Representative graphs of selected VHHs are shown in FIG. 2, all data can be found in Table 4.

D: Competition of Anti-IL-12Rβ2 VHH with IL-12 Signalling

Anti-IL-12Rβ2 VHH showing the ability to bind to human-only or human and mouse IL-12Rβ2 in the ELISA screening, were assessed for their ability to compete with IL-12 cytokine signalling. That was determined using the HEK-Blue IL-12 reporter cell line (Invivogen, #hkb-IL-12). The reporter cell line overexpresses IL-12Rβ1, IL-12Rβ2 and a STAT4-dependent secreted embryonic alkaline phosphatase (SEAP) reporter gene. Cell culture was performed according to the manufacturer's protocol. Cells were seeded at 50000 cells/well of a 96-well plate, and pre-incubated with three dilutions of anti-IL-12Rβ2 VHH-containing P.E. (⅕- 1/50- 1/500) for 30 minutes at room temperature. Subsequently, cells were stimulated with 2.6 ng/ml human recombinant IL-12 (in-house produced). After 24 hours culturing at 37° C. in a CO₂ incubator the SEAP levels were measured by adding culture supernatans to Quanti-Blue substrate (Invivogen). Colorimetric changes were measured at O.D. 650 nm using an iMark Microplate Absorbance Reader. The inhibitory capacity of anti-IL-12Rβ2 VHHs was determined as percent decreased activity compared to IL-12 stimulated cells treated with P.E. that does not contain a VHH. VHH clones were considered to show good ability to inhibit IL-12 signalling when they showed more than 50% reduction of IL-12 activity. CDR3 families were designated as IL-12 competing if 50% or more of the clones show 50% or more reduced IL-12 activity. The results obtained are summarized in Table 5. 16 out of 32 tested CDR3 groups were designated as IL-12 competing.

### E: Anti-IL-12Rβ2 Antibody Generation

18 VHH clones of the human-specific and human-mouse cross-reactive anti-IL-12Rβ2 clones with varying binding and IL-12 competition characteristics were selected and used to generate VHHs fused to a human IgG1 domain (Table 6). 5 VHH clones of the mouse-specific anti-IL-12Rβ2 clones were used to generate VHH fused to mouse IgG2a Fc domain.

TABLE 7

| | | | | | | | P.E. cell binding | |
|---|---|---|---|---|---|---|---|---|
| | | | | | P.E. binding ELISA | | hIL-12RB2 | |
| | | | SEQ | CDR3 | hIL-12RB2 | mIL-12RB2 | | % |
| | Clone | Clone | ID | group | hIL12RB2/ | mIL12RB2/ | | cells |
| | name | ID | NO: | ID | control | control | dMFI | bound |
| Human- | 3CRHS54 | 20422 | 52 | 3 | 7.85 | 1.25 | 870 | 41.5 |
| specific | 3CRMS9 | 20415 | 49 | 3 | 5.08 | 1.09 | 941 | 42.1 |
| | 3CRHS128 | 20432 | 68 | 4 | 3.65 | 0.98 | 1381 | 50.5 |
| | 3CRHS | 20373 | 72 | 5 | 8.64 | 0.81 | 735 | 39.5 |
| | 3CAHS124 | 20400 | 102 | 9 | 5.67 | 1.00 | 1095 | 46.3 |
| | 3CAHS142 | 20401 | 111 | 11 | 4.98 | 1.07 | 812 | 40.9 |
| | 3CANS142 | 20438 | 128 | 14 | 3.58 | 0.93 | 1053 | 43.7 |
| | 3CRHS106 | 20429 | 131 | 15 | 10.18 | 1.03 | 1011 | 43.3 |
| | 3CRH25 | 20378 | 132 | 16 | 5.16 | 1.04 | 520 | 34.8 |
| | 3CAHS102 | 20399 | 146 | 20 | 3.54 | 1.02 | 994 | 44.7 |
| | 3CAHS171 | 20407 | 148 | 21 | 3.27 | 0.96 | 1183 | 47.5 |
| | 3CRHSS1 | 20421 | 150 | 22 | 12.00 | 0.95 | 1139 | 46 |
| | 3CAHS41 | 20394 | 151 | 23 | 8.77 | 1.35 | 971 | 43.3 |
| | 2CRH82 | 20372 | 175 | 37 | 16.13 | 1.77 | 744 | 40.1 |
| Cross- | 3CAHM11 | 21050 | 159 | 26 | 3.04 | 38.79 | 684 | 37.6 |
| reactive | 3CRHM14 | 21053 | 196 | 57 | 3.11 | 25.12 | 589 | 35.8 |
| | 3CRHM99 | 21062 | 204 | 60 | 4.24 | 5.24 | 458 | 24.4 |
| | 3CRHM73 | 21060 | 209 | 62 | 6.90 | 26.28 | 297 | 19.5 |
| | | | | | | | | P.E. IL-12 |
| | | P.E. cell binding | | | | | | competition |
| | | mIL-12RB2 | | P.E. Off-rate constant ranking | | | | IL-12 |
| | | | % | hIL-12RB2 | | mIL-12RB2 | | competing |
| | Clone | | cells | | k_{d} | | k_{d} | CDR3 |
| | name | dMFI | bound | Response | (s⁻¹) | Response | (s⁻¹) | group? |
| Human- | 3CRHS54 | N/A | N/A | 0.2035 | 2.72E−03 | N/A | N/A | yes |
| specific | 3CRMS9 | N/A | N/A | 0.2219 | 3.40E−03 | N/A | N/A | yes |
| | 3CRHS128 | N/A | N/A | 0.2945 | 2.74E−03 | N/A | N/A | no |
| | 3CRHS | N/A | N/A | 0.4911 | 3.37E−03 | N/A | N/A | yes |
| | 3CAHS124 | N/A | N/A | 0.2928 | 3.24E−03 | N/A | N/A | no |
| | 3CAHS142 | N/A | N/A | 0.3518 | 3.24E−03 | N/A | N/A | no |
| | 3CANS142 | N/A | N/A | 0.3385 | 3.02E−03 | N/A | N/A | yes |
| | 3CRHS106 | N/A | N/A | 0.2975 | 3.55E−03 | N/A | N/A | yes |
| | 3CRH25 | N/A | N/A | 0.523 | 2.05E−03 | N/A | N/A | yes |
| | 3CAHS102 | N/A | N/A | 0.3227 | 2.67E−03 | N/A | N/A | yes |
| | 3CAHS171 | N/A | N/A | 0.1948 | 4.31E−03 | N/A | N/A | yes |
| | 3CRHSS1 | N/A | N/A | 0.3271 | 4.30E−03 | N/A | N/A | yes |
| | 3CAHS41 | N/A | N/A | 0.5086 | 4.41E−03 | N/A | N/A | no |
| | 2CRH82 | N/A | N/A | 0.4264 | 2.74E−03 | N/A | N/A | no |
| Cross- | 3CAHM11 | 194 | 7.51 | 1.3968 | 5.19E−03 | 1.0262 | 4.62E−03 | no |
| reactive | 3CRHM14 | 312 | 18.6 | 1.1937 | 5.32E−03 | 0.9568 | 4.83E−03 | no |
| | 3CRHM99 | 251 | 10.7 | 1.4457 | 6.34E−03 | 0.8117 | 6.71E−03 | no |
| | 3CRHM73 | 257 | 10.4 | 1.4487 | 6.05E−03 | 0.9443 | 6.05E−03 | no |

For this purpose, VHH cDNAs were subcloned into a mammalian expression vector comprising the cDNA encoding the CH2 and CH3 of human IgG1 or the CH2 and CH3 of mouse IgG2a. Mutations were introduced into the human IgG1 or mouse IgG2a sequences to enhance the Fc-mediated effector functions (Lazar et al. 2006., PNAS USA., 14; 103(11):4005-4010-DOI: 10.1073pnas.0508123103). Particularly, amino acid substitutions S239D and 1332E (Eu numbering, Edelman al., 1969. PNAS USA, 63(1): 78-8-PMID: 5257969,) were introduced into the human IgG1 immunoglobulin heavy chain constant region, or the corresponding amino acid substitutions were introduced into the mouse IgG2a immunoglobulin heavy chain. indicated as ‘FcDE’ (Table 8).

Antibody molecules were subsequently produced by transient transfection in HEK293T cells and purified from cell supernatants by protein A affinity chromatography.

TABLE 1
Amino acid sequence of anti-IL-12Rβ2 VHHs.

| | | | Protein Sequence |
|---|---|---|---|
| | | | (The first SEQ ID NO in each entry provides the overall sequence of |
| | | CDR3 | the VHH. The next three SEQ ID NOs indicated provided the CDR1, |
| Clone | Clone | group | CDR2, and CDR3 sequences of the VHH. The three sequences of the |
| Name | ID | ID | CDR1, CDR2, and CDR3 given for each entry represent a CDR set). |
| 2CAM8 | 20469 | 1 | QVQLQESGGGLVQPGGSLRLSCAASGRTFSSYGMGWFRQAPGKKR |
| | | | EFVAAISRDARITDYADSVKGRFTISRDNAKNTAYLEMNSLNPED |
| | | | TAVYFCAAAYTYSGSHYVTARSSDYHSWGQGTQVTVSS |
| | | | SEQ ID NO: 1 |
| | | | CDR1: GRTFSSYG - SEQ ID NO: 211/ |
| | | | CDR2: ISRDARIT - SEQ ID NO: 212/ |
| | | | CDR3: AAAYTYSGSHYVTARSSDYHS - SEQ ID NO: 213 |
| 2CAM30 | 20478 | 1 | QVQLQESGGGLVQAGGSLTLSCAASGMPISFNVLGWFRQIPGKER |
| | | | DFVAAISRDARITDYADSVKGRFTISRDNVKNTVYLQMNSLNPED |
| | | | TAVYFCAAAYTYSGSHYVTARSSDYHSWGQGTQVTVSS |
| | | | SEQ ID NO: 2 |
| | | | CDR1: GRTFSSYG - SEQ ID NO: 214/ |
| | | | CDR2: ISRDARIT - SEQ ID NO: 215/ |
| | | | CDR3: AAAYTYSGSHYVTARSSDYHT - SEQ ID NO: 216 |
| 2CAM36 | 20480 | 1 | QVQLQESGGGLVQAGGSLRLSCAASGRTFSSYGMGWFRQAPGKER |
| | | | EFVAAISRDARITDYADSVKGRFTISRDNAKNTAYLEMNSLNPED |
| | | | TAVYFCAAAYTYSGSHYVTARSSDYHSWGQGTQVTVSS |
| | | | SEQ ID NO: 3 |
| | | | CDR1: GRTFSSYG - SEQ ID NO: 217/ |
| | | | CDR2: ISRDARIT - |
| | | | SEQ ID NO: 218/ |
| | | | CDR3: AAAYTYSGSHYVTARSSDYHS - SEQ ID NO: 219 |
| 2CAM38 | 20481 | 1 | QVQLQESGGGLVQAGGSLRLSCAASGRTFSSYGMGWFRQAPGKER |
| | | | DFVAAISRDARITDYADSVKGRFTISRDNVKNTVYLQMNSLNPED |
| | | | TAVYFCAAAYTYSGSHYVTARSSDYHSWGQGTQVTVSS |
| | | | SEQ ID NO: 4 |
| | | | CDR1: GRTFSSYG - SEQ ID NO: 220/ |
| | | | CDR2: ISRDARIT - SEQ ID NO: 221/ |
| | | | CDR3: AAAYTYSGSHYVTARSSDYHS - SEQ ID NO: 222 |
| 2CAM48 | 20488 | 1 | QVQLQESGGGLMQAGDSLRLSCAASGRTFSSYGMGWFRQAPGKER |
| | | | EFVAAISRGARITDYADSVKGRFTISRDNAKNTAYLQMNSLNPED |
| | | | TAVYFCAAAYTYSGSHYVTARSSDYHSWGQGTQVTVSS |
| | | | SEQ ID NO: 5 |
| | | | CDR1: GRTFSSYG - SEQ ID NO: 223/ |
| | | | CDR2: ISRGARIT - SEQ ID NO: 224/ |
| | | | CDR3: AAAYTYSGSHYVTARSSDYHS - SEQ ID NO: 225 |
| 2CAM57 | 20491 | 1 | QVQLQESGGGLVQAGGSLRLSCAASGRTFSSYGMGWFRQAPGKER |
| | | | DFVAAISRDARYTDYADSVKGRFTISKDNAKNTAYLQMNSLNPED |
| | | | TAVYFCAAAYTYSGSHYVTARSGDYNYWGQGTQVTVSS |
| | | | SEQ ID NO: 6 |
| | | | CDR1: GRTFSSYG - SEQ ID NO: 226/ |
| | | | CDR2: I SRDARYT - SEQ ID NO: 227/ |
| | | | CDR3: AAAYTYSGSHYVTARSGDYNY - SEQ ID NO: 228 |
| 2CAM60 | 20492 | 1 | QVQLQESGGGLVQAGGSLRLSCAASGRTFSSRGMGXFRQAPGKER |
| | | | DFVAAISRGARNTDYADSVKGRFTISRDNAKNTAYLQMNSLNPGD |
| | | | TAVYFCAAAYIYSGSHYVTARSSDYEYWGQGTQVTVSS |
| | | | SEQ ID NO: 7 |
| | | | CDR1: GRTFSSRG - SEQ ID NO: 229/ |
| | | | CDR2: ISRGARNT - SEQ ID NO: 230/ |
| | | | CDR3: AAAYIYSGSHYVTARSSDYEY - SEQ ID NO: 231 |
| 2CAM69 | 20495 | 1 | QVQLQESGGGLVQAGGSLRLSCAASGRTFSMHGMGWFRQAPGKER |
| | | | EFVAAISRDARITDYADSVKGRFTISRDNAKNTAYLQMNSLNPED |
| | | | TAVYFCAAAYTYSGSHYVTARSSDYNYWGQGTQVTVSS |
| | | | SEQ ID NO: 8 |
| | | | CDR1: GRTFSMHG - SEQ ID NO: 232/ |
| | | | CDR2: ISRDARIT - SEQ ID NO: 233/ |
| | | | CDR3: AAAYTYSGSHYVTARSSDYNY - SEQ ID NO 234: |
| 2CAM72 | 20497 | 1 | QVQLQESGGGLVQAGDSLRLSCAASGRTFSSYGMGWFRQAPGKER |
| | | | EFVAAISRGARITDYADSVKGRFTISRDNAKNTAYLQMNSLNPED |
| | | | TAVYFCAAAYTYSGSHYVTARSSDYHSWGQGTQVTVSS |
| | | | SEQ ID NO: 9 |
| | | | CDR1: GRTFSSYG - SEQ ID NO: 235/ |
| | | | CDR2: ISRGARIT - SEQ ID NO: 236/ |
| | | | CDR3: AAAYTYSGSHYVTARSSDYHS - SEQ ID NO: 237 |
| 2CAM73 | 20498 | 1 | QVQLQESGGGLVQAGGSLRLSCASSGRTFSSRTVGWFRQAPGKER |
| | | | YFVAAISRDTAYTDYADSVKGRFTISRGNAKNTVYLQMNSLNPED |
| | | | TAVYYCAASHIYSGSHYISARSSDYMYWGQGTQVTVSS |
| | | | SEQ ID NO: 10 |
| | | | CDR1: GRTFSSRT - |
| | | | SEQ ID NO: 238/ |
| | | | CDR2: ISRDTAYT - |
| | | | SEQ ID NO: 239/ |
| | | | CDR3: AASHIYSGSHYISARSSDYMY - SEQ ID NO: 240 |
| 2CAM75 | 20499 | 1 | QVQLQESGGGLVQAGGSLRLSCAASGRTFSSYGMGWFRQAPGKER |
| | | | EFVAAISRDASITDYADSVKGRFTISRDNAKNTAYLEMNSLNPED |
| | | | TAVYFCAAAYTYSGSHYVTARSSDYHSWGQGTQVTVSS |
| | | | SEQ ID NO: 11 |
| | | | CDR1: - GRTFSSYG - SEQ ID NO: 241/ |
| | | | CDR2: ISRDASIT - SEQ ID NO: 242/ |
| | | | CDR3: AAAYTYSGSHYVTARSSDYHS - SEQ ID NO: 243 |
| 2CAM81 | 20500 | 1 | QVQLQESGGGLVQPGGSLRLSCAASGFTFSSYGMGWFRQAPGKER |
| | | | EFVAAISRDARITDYADSVKGRFTISRDNAKNTAYLEMNSLNPED |
| | | | TAVYFCAAAYTYSGSHYVTARSSDYHSWGQGTQVTVSS |
| | | | SEQ ID NO: 12 |
| | | | CDR1: GFTFSSYG - SEQ ID NO: 244/ |
| | | | CDR2: ISRDARIT - SEQ ID NO: 245/ |
| | | | CDR3: AAAYTYSGSHYVTARSSDYHS - SEQ ID NO: 246 |
| 2CAM91 | 20502 | 1 | QVQLQESGGGLVQTGGSLKLSCAASGRTFSSYGMGWFRQAPGKER |
| | | | DFVAAISRDARYTDYADSVKGRFTISKDNAKNTAYLQMNSLNPED |
| | | | TAVYFCAAAYTYSGSHYVTARSGDYNYWGXGTQVTVSS |
| | | | SEQ ID NO: 13 |
| | | | CDR1: GRTFSSYG - SEQ ID NO: 247/ |
| | | | CDR2: ISRDARYT - SEQ ID NO: 248/ |
| | | | CDR3: AAAYTYSGSHYVTARSGDYNY - SEQ ID NO: 249 |
| 3CAM3 | 20504 | 1 | QVQLQESGGGLVQAGGSLKLSCAATGFILDYVGVGWFRQAPGKER |
| | | | DFVAAISRDARITDYADSVKGRFTISRDNAKNTAYLQMNSLNPED |
| | | | TAVYFCAAAYTYSGSHYVTARSSDYHTWGQGTQVTVSS |
| | | | SEQ ID NO: 14 |
| | | | CDR1: GFILDYVG - SEQ ID NO: 250/ |
| | | | CDR2: ISRDARIT - SEQ ID NO: 251/ |
| | | | CDR3: AAAYTYSGSHYVTARSSDYHT - SEQ ID NO: 252 |
| 3CAM4 | 20505 | 1 | QVQLQESGGGSVQAGGSLRLSCAASGRTFSSYGMGWFRQAPGKER |
| | | | EFVAAISRDARITDYADSVKGRFTISRDNAKNTAYLEMNSLNPED |
| | | | TAVYFCAAAYTYSGSHYVTARSSDYHSWGQGTQVTVSS |
| | | | SEQ ID NO: 15 |
| | | | CDR1: GRTFSSYG - SEQ ID NO: 253/ |
| | | | CDR2: ISRDARIT - SEQ ID NO: 254/ |
| | | | CDR3: AAAYTYSGSHYVTARSSDYHS - SEQ ID NO: 255 |
| 3CAM30 | 20509 | 1 | QVQLQESGGGLVQAGGSLRLSCAAAGRTFSSYGMGWFRQAPGKER |
| | | | DFVAAISRDARITDYADSVKGRFTISRDNAKNTAYLQMNSLNPED |
| | | | TAVYFCAAAYTYSGSHYVTARSSDYHTWGQGTQDTVSS |
| | | | SEQ ID NO: 16 |
| | | | CDR1: GRTFSSYG - SEQ ID NO: 256/ |
| | | | CDR2: ISRDARIT - SEQ ID NO: 257/ |
| | | | CDR3: AAAYTYSGSHYVTARSSDYHT - SEQ ID NO: 258 |
| 3CAM31 | 20510 | 1 | QVQLQESGGGLVQAGGSLRLSCAASGRTFSSYGMGWFRQAPGKER |
| | | | EFVAAISRGARITDYADSVKGRFTISRDNAKNTAYLQMNSLNPED |
| | | | TAVYFCAAAYTYSGSHYVTARSSDYHSWGQGTQVTVSS |
| | | | SEQ ID NO: 17 |
| | | | CDR1: GRTFSSYG - SEQ ID NO: 259/ |
| | | | CDR2: ISRGARIT - SEQ ID NO: 260/ |
| | | | CDR3: AAAYTYSGSHYVTARSSDYHS - SEQ ID NO: 261 |
| 3CAM36 | 20511 | 1 | QVQLQESGGGLVQAGESLTLSCTASGRTFSSYGMGWFRQAPGKER |
| | | | DFVAAISRDARYTDYADSVKGRFTISKDNAKNTAYLQMNSLNPED |
| | | | TAVYFCAAAYTYSGSHYVTARSGDYNYWGQGTQVTVSS |
| | | | SEQ ID NO: 18 |
| | | | CDR1: GRTFSSYG - SEQ ID NO: 262/ |
| | | | CDR2: ISRDARYT - SEQ ID NO: 263/ |
| | | | CDR3: AAAYTYSGSHYVTARSGDYNY - SEQ ID NO: 264 |
| 3CAM70 | 20514 | 1 | QVQLQESGGGLVQAGDSLRLSCIASGPSFSSYGMGWFRQAPGKER |
| | | | EFVAAISRDARITDYADSVKGRFTISRDNAKNTAYLEMNSLNPED |
| | | | TAVYFCAAAYTYSGSHYVTARSSDYHSWGQGTQVTVSS |
| | | | SEQ ID NO: 19 |
| | | | CDR1: GPSFSSYG - SEQ ID NO: 265/ |
| | | | CDR2: ISRDARIT - SEQ ID NO: 266/ |
| | | | CDR3: AAAYTYSGSHYVTARSSDYHS - SEQ ID NO: 267 |
| 3CAM85 | 20516 | 1 | QVQLQESGGGLVQAGGSLRLSCAASGRTFSSYGMSWFRQAPGKER |
| | | | DFVAAISRDARITDYADSVKGRFTISRDNVKNTVYLQMNSLNPED |
| | | | TAVYFCAAAYTYSGSHYVTARSSDYHSWGQGTQVTVSS |
| | | | SEQ ID NO: 20 |
| | | | CDR1: GRTFSSYG - SEQ ID NO:268/ |
| | | | CDR2: ISRDARIT - SEQ ID NO: 269/ |
| | | | CDR3: AAAYTYSGSHYVTARSSDYHS - SEQ ID NO: 270 |
| 2CAMS53 | 20536 | 1 | QVQLQESGGGMVQAGGSLRLSCAASGRTFSSYGMGWFRQAPGKER |
| | | | EFVAAISRGARNTDYADSVKGRFTISRDNAKNTAYLQMNSLNPED |
| | | | TAVYFCAAAYTYSGSHYVTARSSDYPYWGQGTQVTVSS |
| | | | SEQ ID NO: 21 |
| | | | CDR1: GRTFSSYG - SEQ ID NO: 271/ |
| | | | CDR2: ISRGARNT - SEQ ID NO: 272/ |
| | | | CDR3: AAAYTYSGSHYVTARSSDYPY - SEQ ID NO: 273 |
| 2CAMS83 | 20538 | 1 | QVQLQESGGGLVQPGDSLRLSCAASGRTFSSYGMGWFRQAPGKER |
| | | | EFVAAISRGARNTDYADSVKGRFTISRDNAKNTAYLQMNSLNPED |
| | | | TAVYFCAAAYTYSGSHYVTARSSDYPYWGQGTQVTVSS |
| | | | SEQ ID NO: 22 |
| | | | CDR1: GRTFSSYG - SEQ ID NO: 274/ |
| | | | CDR2: ISRGARNT - SEQ ID NO: 275/ |
| | | | CDR3: AAAYTYSGSHYVTARSSDYPY - SEQ ID NO: 276 |
| 2CAMS87 | 20539 | 1 | QVQLQESGGGLVQAGGSLRLSCAASGRTFSSYGMGWFRQAPGKER |
| | | | EFVAAISRGARNTDYADSVKGRFTISRDNAKNTAFLQMNSLNPED |
| | | | TAVYFCAAAYTYSGSHYVTARSSDYHTWGQGTQVTVSS |
| | | | SEQ ID NO: 23 |
| | | | CDR1: GRTFSSYG - SEQ ID NO: 277/ |
| | | | CDR2: ISRGARNT - SEQ ID NO: 278/ |
| | | | CDR3: AAAYTYSGSHYVTARSSDYHT - SEQ ID NO: 279 |
| 3CAMS5 | 20540 | 1 | QVQLQESGGGLVQAGDSLRISCAASGRTFSSYGMGWFRQAPGKER |
| | | | DFVAAISRDARYTDYADSVKGRFTISKDNAKNTAYLQMNSLNPED |
| | | | TAVYFCAAAYTYSGSHYVTARSGDYNYWGQGTQVTVSS |
| | | | SEQ ID NO: 24 |
| | | | CDR1: GRTFSSYG - SEQ ID NO: 280/ |
| | | | CDR2: ISRDARYT - SEQ ID NO:281/ |
| | | | CDR3: AAAYTYSGSHYVTARSGDYNY - SEQ ID NO: 282 |
| 3CAMS9 | 20541 | 1 | QVQLQESGGGLVQAGGSLRLSCAASGRTFSSYGMGWFRQAPGKER |
| | | | EFVAAISRDARITDYADSVKGRFTISRDNAKNTAYLEMNSLNPED |
| | | | TAVYSCAAAYTYSGSHYVTARSSDYHSWGQGTQVTVSS |
| | | | SEQ ID NO: 25 |
| | | | CDR1: GRTFSSYG - |
| | | | SEQ ID NO: 283/ |
| | | | CDR2: ISRDARIT - SEQ ID NO: 284/ |
| | | | CDR3: AAAYTYSGSHYVTARSSDYHS - SEQ ID NO: 285 |
| 3CAMS19 | 20544 | 1 | QVQLQESGGGLVQAGGPLRLSCAASGRTFSSYGMGWFRQAPGKER |
| | | | DFVAAISRDARITDYADSVKGRFTISRDNVKNTVYLQMNSLNPED |
| | | | TAVYFCAAAYTYSGSHYVTARSSDYHSWGQGTQVTVSS |
| | | | SEQ ID NO: 26 |
| | | | CDR1: GRTFSSYG - SEQ ID NO: 286/ |
| | | | CDR2: ISRDARIT - SEQ ID NO: 287/ |
| | | | CDR3: AAAYTYSGSHYVTARSSDYHS - SEQ ID NO: 288 |
| 3CAMS47 | 20547 | 1 | QVQLQESGGGLVQAGGSLRLSCAASGRTFSSYGMGWFRQAPGKER |
| | | | DFVAAISRGARNADYADSVKGRFTISRDNAKNTAYLQMNSLNPED |
| | | | TAVYFCAAAYTYSGSHYVTARSSDYAYWGQGTQVTVSS |
| | | | SEQ ID NO: 27 |
| | | | CDR1: GRTFSSYG - SEQ ID NO: 289/ |
| | | | CDR2: GRTFSSYG - SEQ ID NO: 290/ |
| | | | CDR3: AAAYTYSGSHYVTARSSDYAY - SEQ ID NO: 291 |
| 3CAMS50 | 20548 | 1 | QVQLQESGGGLVQAGGSLRLSCADSGRTFSSYGMGWFRQAPGKER |
| | | | EFVAAISRGARITDYADSVKGRFTISRDNAKNTAYLQMNSLNPED |
| | | | TAVYFCAAAYTYSGSHYVTARSSDYHSWGQGTQVTVSS |
| | | | SEQ ID NO: 28 |
| | | | CDR1: GRTFSSYG - SEQ ID NO: 292/ |
| | | | CDR2: ISRGARIT - SEQ ID NO: 293/ |
| | | | CDR3: AAAYTYSGSHYVTARSSDYHS - SEQ ID NO: 294 |
| 3CAMS56 | 20550 | 1 | QVQLQESGGGSVQLGGSLRLSCAASGRTFSSYGMGWFRXAPGKER |
| | | | DFVAAISRDARYTDYADSVKGRFTISKDNAKNTAYLQMNSLNPED |
| | | | TAVYFCAAAYTYSGSHYVTARSGDYNYWGQGTQVTVSS |
| | | | SEQ ID NO: 29 |
| | | | CDR1: GRTFSSYG - SEQ ID NO: 295/ |
| | | | CDR2: ISRDARYT - SEQ ID NO: 296/ |
| | | | CDR3: AAAYTYSGSHYVTARSGDYNY - SEQ ID NO: 297 |
| 2CRM12 | 20442 | 2 | QVQLQESGGGLVQPGGSLRLSCTASGSIFAIDDMYWYRQAPGKQR |
| | | | EFVAYITATNSANYLDSVNGRFTISRDNAKNSVYLQMNSLKPEDT |
| | | | AVYYCNAKQIVGFQIDEYWGQGTQVTVSS |
| | | | SEQ ID NO: 30 |
| | | | CDR1: GSIFAIDD - SEQ ID NO: 298/ |
| | | | CDR2: ITATNSA - SEQ ID NO: 299/ |
| | | | CDR3: NAKQIVGFQIDEY - SEQ ID NO: 300 |
| 2CRM18 | 20444 | 2 | QVQLQESGGGLVQAGGSLRLSCTASGSIFAIDDMYWYRQAPGKQR |
| | | | EFVAYITATNSANYLDSVNGRFTISRDNAKNSVYLQMNSLKPEDT |
| | | | AVYYCNAKQIVGFQIDEYWGQGTQVTVSS |
| | | | SEQ ID NO: 31 |
| | | | CDR1: GSIFAIDD - SEQ ID NO: 301/ |
| | | | CDR2: ITATNSA - SEQ ID NO: 302/ |
| | | | CDR3: NAKQIVGFQIDEY - SEQ ID NO: 303 |
| 2CRM32 | 20446 | 2 | QVQLQESGGGLVQPGGSLRLSCTASGSIFASDDMYWYRQAPGKQR |
| | | | EFVAYITATNSANYLDSVNGRFTISRDNAKNSVYLQMNSLKPEDT |
| | | | AVYYCNAKQIVGFQIDEYWGQGTQVTVSS |
| | | | SEQ ID NO: 32 |
| | | | CDR1: GSIFASDD - SEQ ID NO: 304/ |
| | | | CDR2: ITATNSA - SEQ ID NO: 305/ |
| | | | CDR3: NAKQIVGFQIDEY - SEQ ID NO: 306 |
| 2CRM93 | 20454 | 2 | QVQLQESGGGLVQAGDSLRLSCTASGSIFAIDDMYWYRQAPGKQR |
| | | | EFVAYITATNSANYLDSVNGRFTISRDNAENSVYLQMNSLKPEDT |
| | | | VVYYCNAKQIVGFQIDEYWGQGTQVTVSS |
| | | | SEQ ID NO: |
| | | | SEQ ID NO: 33 |
| | | | CDR1: GSIFAIDD - SEQ ID NO: 307/ |
| | | | CDR2: ITATNSA - SEQ ID NO: 308/ |
| | | | CDR3: NAKQIVGFQIDEY - SEQ ID NO: 309 |
| 3CRM6 | 20455 | 2 | QVQLQESGGGLVQPGGSLRLSCTASGSIFAIDDMYWYRQAPGKQR |
| | | | EFVAYITATNSANYLDSVNGRFTISRDNAKNSVYLQMNSLKPEDT |
| | | | AVYYCNTKQIVGFQIDEYWGQGTQVTVSS |
| | | | SEQ ID NO: 34 |
| | | | CDR1: GSIFAIDD - SEQ ID NO: 310/ |
| | | | CDR2: ITATNSA - SEQ ID NO: 311/ |
| | | | CDR3: NTKQIVGFQIDEY - SEQ ID NO: 312 |
| 3CRM14 | 20456 | 2 | QVQLQESGGGLVQPGGSLTLSCTASGSIFAIDDMYWYRQAPGKQR |
| | | | EFVAYITATNSANYLDSVNGRFTISRDNAKNSVYLQMNSLKPEDT |
| | | | AVYYCNAKQIVGFQIDEYWGQGTQVTVSS |
| | | | SEQ ID NO:35 |
| | | | CDR1: GSIFAIDD - SEQ ID NO: 313/ |
| | | | CDR2: ITATNSA - SEQ ID NO: 314/ |
| | | | CDR3: - NAKQIVGFQIDEY |
| | | | SEQ ID NO: 315 |
| 3CRM45 | 20458 | 2 | QVQLQESGGGLVQAGGSLRLSCTASGSIFAIDDMYWYRQAPGKER |
| | | | EFVAYITATNSANYLDSVNGRFTISRDNAKNSVYLQMNSLKPEDT |
| | | | AVYYCNAKQIVGFQIDEYWGQGTQVTVSS |
| | | | SEQ ID NO:36 |
| | | | CDR1: GSIFAIDD - SEQ ID NO: 316/ |
| | | | CDR2: ITATNSA - SEQ ID NO: 317/ |
| | | | CDR3: NAKQIVGFQIDEY - SEQ ID NO: 318 |
| 3CRM86 | 20464 | 2 | QVQLQESGGGLVQAGGSLRLFCTASGSIFAIDDMYWYRQAPGKQR |
| | | | EFVAYITATNSANYLDSVNGRFTISRDNAKNSVYLQMNSLKPEDT |
| | | | AVYYCNAKQIVGFQIDEYWGQGTQVTVSS |
| | | | SEQ ID NO:37 |
| | | | CDR1: GSIFAIDD - SEQ ID NO: 319/ |
| | | | CDR2: ITATNSA - SEQ ID NO: 320/ |
| | | | CDR3: NAKQIVGFQIDEY - SEQ ID NO: 321 |
| 2CRMS10 | 20517 | 2 | QVQLQESGGGLAQPGGSLRLSCTASGSIFAIDDMYWYRQAPGKQR |
| | | | EFVAYITATNSANYLDSVNGRFTISRDNAKNSVYLQMNSLKPEDT |
| | | | AVYYCNAKQIVGFQIDEYWGQGTQVTVSS |
| | | | SEQ ID NO: 38 |
| | | | CDR1: GSIFAIDD - SEQ ID NO: 322/ |
| | | | CDR2: ITATNSA - SEQ ID NO: 323/ |
| | | | CDR3: NAKQIVGFQIDEY - SEQ ID NO: 324 |
| 2CRMS15 | 20518 | 2 | QVQLQESGGGLVQPGGSLRLSCTASGSIFAIDDMYWYRQAPGRQR |
| | | | EFVAYITATNSANYLDSVNGRFTISRDNAKNSVYLQMNSLKPEDT |
| | | | AVYYCNAKQIVGFQIDEYWGQGTQVTVSS |
| | | | SEQ ID NO: 39 |
| | | | CDR1: GSIFAIDD - SEQ ID NO: 325/ |
| | | | CDR2: ITATNSA - SEQ ID NO: 326/ |
| | | | CDR3: NAKQIVGFQIDEY - SEQ ID NO: 327 |
| 2CRMS16 | 20519 | 2 | QVQLQESGGGLVQAGESLRLSCTASGSIFAIDDMYWYRQAPGKQR |
| | | | EFVAYITATNSANYLDSVNGRFTISRDNAKNSVYLQMNSLKPEDT |
| | | | AVYYCNAKQIVGFQIDEYWGQGTQVTVSS |
| | | | SEQ ID NO: 40 |
| | | | CDR1: GSIFAIDD - SEQ ID NO: 328/ |
| | | | CDR2: ITATNSA - SEQ ID NO: 329/ |
| | | | CDR3: NAKQIVGFQIDEY - SEQ ID NO: 330 |
| 2CRMS58 | 20522 | 2 | QVQLQESGGGLVQPGGSLRLSCTASGSIFAIDDMYWYRQAPGKQR |
| | | | EFVAYITATNSANYLDSVNGRFTISRDNAKNSVYLQMNSLKPEDT |
| | | | AVYYCNAKQIVGFQIDDYWGQGTQVTVSS |
| | | | SEQ ID NO:41 |
| | | | CDR1: GSIFAIDD - SEQ ID NO: 331/ |
| | | | CDR2: ITATNSA - SEQ ID NO: 332/ |
| | | | CDR3: NAKQIVGFQIDDY - SEQ ID NO: 333 |
| 3CRMS14 | 20524 | 2 | QVQLQESGGGLVQAGGSLRLSCTASGSIFAIDDMYWYRQAPGKQR |
| | | | EFVAYITATNSANYLDPVNGRFTISRDNAKNSVYLQMNSLKPEDT |
| | | | AVYYCNAKQIVGFQIDEYWGQGTQVTVSS |
| | | | SEQ ID NO: 42 |
| | | | CDR1: GSIFAIDD - SEQ ID NO: 334/ |
| | | | CDR2: ITATNSA - SEQ ID NO: 335/ |
| | | | CDR3: NAKQIVGFQIDEY - SEQ ID NO: 336 |
| 3CRMS26 | 20525 | 2 | QVQLQESGGGLVQAGGSLRLSCAASGSIFAIDDMYWYRQAPGKQR |
| | | | EFVAYITATNSANYLDSVNGRFTISRDNAKNSVYLQMNSLKPEDT |
| | | | AVYYCNAKQIVGFQIDEYWGQGTQVTVSS |
| | | | SEQ ID NO: 43 |
| | | | CDR1: GSIFAIDD - SEQ ID NO: 337/ |
| | | | CDR2: ITATNSA - SEQ ID NO: 338/ |
| | | | CDR3: NAKQIVGFQIDEY - SEQ ID NO: 339 |
| 3CRMS94 | 20529 | 2 | QVQLQESGGGSVQAGDSLRLSCTASGSIFAIDDMYWYRQAPGKQR |
| | | | EFVAYITATNSANYLDSVNGRFTISRDNAKNSVYLQMNSLKPEDT |
| | | | AVYYCNAKQIVGFQIDEYWGQGTQVTVSS |
| | | | SEQ ID NO: 44 |
| | | | CDR1: GSIFAIDD - SEQ ID NO: 340/ |
| | | | CDR2: ITATNSA - SEQ ID NO: 341/ |
| | | | CDR3: NAKQIVGFQIDEY - SEQ ID NO:342 |
| 3CRH18 | 20377 | 3 | QVQLQESGGGLVQPGGSLRLSCTASGNIVSIYNMDWYRQAPGNQR |
| | | | EWVASITSSGDPYYDGSAKGRFTISRDNAKMTVYLQMNSLRPEDT |
| | | | AVYYCKVPTEKGNYWGQGTQVTVSS |
| | | | SEQ ID NO: 45 |
| | | | CDR1: GNIVSIYN - SEQ ID NO: 343/ |
| | | | CDR2: ITSSGDP - SEQ ID NO: 344/ |
| | | | CDR3: KVPTEKGNY - SEQ ID NO: 345 |
| 3CRH27 | 20379 | 3 | QVQLQESGGGLVQAGGSLRLSCTASGSIVSIYNMDWYRQAPGNER |
| | | | EWVASITSSGDPYYDGSAKGRFTISRDNAKMTVYLQMNSLKPEDT |
| | | | AVYYCKVPTEKGNYWGQGTQVTVSS |
| | | | SEQ ID NO: 46 |
| | | | CDR1: GSIVSIYN - SEQ ID NO: 346/ |
| | | | CDR2: ITSSGDP - SEQ ID NO: 347/ |
| | | | CDR3: KVPTEKGNY - SEQ ID NO: 348 |
| 3CRH68 | 20388 | 3 | QVQLQESGGGLVQPGGSLRLSCTASGSILSIYNMDWYRQVPGNQR |
| | | | EWVASITPSGDPYYDGSVKGRFTISRDNAKMMVYLQMNSLKPEDT |
| | | | AVYYCKVPTEKGNYWGQGTQVTVSS |
| | | | SEQ ID NO: 47 |
| | | | CDR1: GSILSIYN - SEQ ID NO: 349/ |
| | | | CDR2: ITPSGDP - |
| | | | SEQ ID NO: 350/ |
| | | | CDR3: KVPTEKGNY - SEQ ID NO: 351 |
| 3CRHS3 | 20414 | 3 | QVQLQESGGGLVQPGGSLRLSCTASGSIFSIYNMDWYRQAPGNQR |
| | | | EWVASITSSGDPYYDASVKGRFTISRDSAKMTVYLQMNSLKPEDT |
| | | | AVYYCKLPTEKGNYWGQGTQVTVSS |
| | | | SEQ ID NO: 48 |
| | | | CDR1: GSIFSIYN - SEQ ID NO: 352/ |
| | | | CDR2: ITSSGDP - SEQ ID NO: 353/ |
| | | | CDR3: KLPTEKGNY - SEQ ID NO: 354 |
| 3CRHS9 | 20415 | 3 | QVQLQESGGGLVQPGGSLRLSCTASGSIVSIYNMDWYRQAPGNER |
| | | | EWVASITSSGDPYYDGSAKGRFTISRDNAKMTVYLQMNSLKPEDT |
| | | | AVYYCKVPTEKGNYWGQGTQVTVSS |
| | | | SEQ ID NO: 49 |
| | | | CDR1: GSIVSIYN - SEQ ID NO: 355/ |
| | | | CDR2: ITSSGDP - SEQ ID NO: 356/ |
| | | | CDR3: KVPTEKGNY - SEQ ID NO: 357 |
| 3CRHS29 | 20417 | 3 | QVQLQESGGGLVQPGGSLRLSCTASGSIFSIYNMDWYRQAPGNQR |
| | | | EWVASITSSGDPYYDGPVKGRFTISRDNAKMTAYLQMNSLKPEDT |
| | | | AVYYCKLPTEKGNYWGXGTQVTVSS |
| | | | SEQ ID NO: 50 |
| | | | CDR1: GSIFSIYN - SEQ ID NO: 358/ |
| | | | CDR2: ITSSGDP - SEQ ID NO:359/ |
| | | | CDR3: KLPTEKGNY - SEQ ID NO: 360 |
| 3CRHS38 | 20420 | 3 | QVQLQESGGGLVXPGGSLRLSCTASGSIFSIYLMSWYRQAPGNQR |
| | | | EWVASITSSGDPYYDESAKGRFTISRDNAKMTGYLQMNSLKPEDT |
| | | | AVYYCKLPTEKENYWGQGTQVTVSS |
| | | | SEQ ID NO: 51 |
| | | | CDR1: GSIFSIYL - SEQ ID NO: 361/ |
| | | | CDR2: ITSSGDP - SEQ ID NO:362/ |
| | | | CDR3: KLPTEKENY - SEQ ID NO: 363 |
| 3CRHS54 | 20422 | 3 | QVQLQESGGGLVQPGGSLRLSCTASGSIVSIYNMDWYRQAPGNER |
| | | | EWVASITSSGDPYYDGSAKGRFTISRDNAKMTVYLQMNSLEPEDT |
| | | | AVYFCKVPTEKGNYWGQGTQVTVSS |
| | | | SEQ ID NO: 52 |
| | | | CDR1: GSIVSIYN - SEQ ID NO: 364/ |
| | | | CDR2: ITSSGDP - SEQ ID NO: 365/ |
| | | | CDR3: KVPTEKGNY - SEQ ID NO: 366 |
| 3CRHS55 | 20423 | 3 | QVQLQESGGGLVQAGGSLNLSCTASGSIISIYNMDWYRQAPGNQR |
| | | | EWVASITSNGDSYYDGSAKGRFTISRDNAKMTVYLQMNSLKPEDT |
| | | | AVYYCKVPTENGNYWGQGTQVTVSS |
| | | | SEQ ID NO: 53 |
| | | | CDR1: GSIISIYN - SEQ ID NO: 367/ |
| | | | CDR2: ITSNGDS - SEQ ID NO: 368/ |
| | | | CDR3: KVPTENGNY - |
| | | | SEQ ID NO: 369 |
| 3CRHS90 | 20426 | 3 | QVQLQESGGGLVQPGGSLRLSCTASGSIFSIYNMDWYRXAPGNQR |
| | | | EWVASITPSGDPYYDGPVKGRFTISRDNARMTVYLQMNSLKPEDT |
| | | | AVYYCKLPTEKGNYWGQGTQVTVSS |
| | | | SEQ ID NO:54 |
| | | | CDR1: GSIFSIYN - SEQ ID NO: 370/ |
| | | | CDR2: ITPSGDP - SEQ ID NO: 371/ |
| | | | CDR3: KLPTEKGNY - SEQ ID NO: 372 |
| 3CRHS100 | 20428 | 3 | QVQLQESGGGLVQPGGSLRLSCTASGSIFSIYNMDWYRQAPGNQR |
| | | | EWVASITPSGDPYYDDSAKGRFTISRDNAKMTVYLQMNSLKPEDT |
| | | | AVYYCKVPTEKGNYWGQGTQVTVSS |
| | | | SEQ ID NO: 55 |
| | | | CDR1: GSIFSIYN - SEQ ID NO: 373/ |
| | | | CDR2: ITPSGDP - SEQ ID NO: 374/ |
| | | | CDR3: KVPTEKGNY - SEQ ID NO: 375 |
| 3CRHS148 | 20436 | 3 | QVQLQESGGGLVQPGGSLRLSCIASGSIFSIYNMDWYRQAPGNQR |
| | | | EWVASITPSGDPYYDDSAKGRFTISRDNAKMTVYLQMNSLKPEDT |
| | | | AVYYCKVPTEKGNYWGQGTQVTVSS |
| | | | SEQ ID NO: 56 |
| | | | CDR1: GSIFSIYN - SEQ ID NO: 376/ |
| | | | CDR2: ITPSGDP - SEQ ID NO: 377/ |
| | | | CDR3: KVPTEKGNY - |
| | | | SEQ ID NO: 378 |
| 3CRHS173 | 20440 | 3 | QVQLQESGGGLVQPGGSLRLSCTASGSTVSIYNMDWYRQAPGNER |
| | | | EWVASITSSGDPYYDGSAKGRFTISRDNAKMTVYLQMNSLEPEDT |
| | | | AVYFCKVPTERGNYWGXGTQVTVSS |
| | | | SEQ ID NO: 57 |
| | | | CDR1: GSIFSIYN - |
| | | | SEQ ID NO: 379/ |
| | | | CDR2: ITPSGDP - SEQ ID NO: 380/ |
| | | | CDR3: KVPTEKGNY - |
| | | | SEQ ID NO: 381 |
| 3CRHM61 | 21059 | 3 | QVQLQESGGGLVXPGGSLRLSCTASGSIFSIYNMDWYRQAPGNQR |
| | | | EWVASITSSGDPYYDGSAKGRFTISRDNAKMTVYLQMNSLKPEDT |
| | | | AVYYCKVPTEKGNYWGQGTQVTVSS |
| | | | SEQ ID NO: 58 |
| | | | CDR1: GSIFSIYN - SEQ ID NO: 382/ |
| | | | CDR2: ITSSGDP - SEQ ID NO: 383/ |
| | | | CDR3: KVPTEKGNY - SEQ ID NO: 384 |
| 2CRH33 | 20363 | 4 | QVQLQESGGGSVQPGWSLRLTCAASGFTFSTSGMHWFRQAPGKGL |
| | | | EWVSSISSGGGDTYYTDSVKGRITISRDNAKNTLYLQLNDLKSED |
| | | | TAVYYCSKDPSGATRGQGTQVTVSS |
| | | | SEQ ID NO: 59 |
| | | | CDR1: GFTFSTSG - SEQ ID NO: 385/ |
| | | | CDR2: ISSGGGDT - SEQ ID NO: 386/ |
| | | | CDR3: SKDPSGAT - SEQ ID NO: 387 |
| 2CRH60 | 20367 | 4 | QVQLQESGGGSVQPGGSLRLTCAASGFTFSTSGMHWFRQAPGKGL |
| | | | EWVSSISSGGGDTYYTDSVKGRITISRDNAKNTLYLQLNDLKSED |
| | | | TAVYYCSKDPSGATRGQGTQVTVSS |
| | | | SEQ ID NO: 60 |
| | | | CDR1: GFTFSTSG - |
| | | | SEQ ID NO: 388/ |
| | | | CDR2: ISSGGGDT - SEQ ID NO: 389/ |
| | | | CDR3: SKDPSGAT - |
| | | | SEQ ID NO: 390 |
| 2CRH80 | 20371 | 4 | QVQLQESGGGLVQPGGSLRLSCAASGFTFSTSGMHWFRQAPGKGL |
| | | | EWVSSIGIIVGSSYYASSVKGRFTISRDNAKNTLYLHMNSLKSED |
| | | | TAVYYCSKDVTGSSRGQGTQVTVSS |
| | | | SEQ ID NO: 61 |
| | | | CDR1: GFTFSTSG - SEQ ID NO: 391/ |
| | | | CDR2: IGIIVGSS - SEQ ID NO: 392/ |
| | | | CDR3: SKDVTGSS - SEQ ID NO: 393 |
| 3CRH14 | 20374 | 4 | QVQLQESGGGLVQPGGSLRLSCAASGFTFSTSGMHWFRQAPGKGL |
| | | | EWVSSIGIIVDSSYYADSVKGRFTISRDNAKNTLFLQMNSLKSED |
| | | | TAVYYCSKDPTGSSRGQGTQVTVSS |
| | | | SEQ ID NO:62 |
| | | | CDR1: GFTFSTSG - SEQ ID NO: 394/ |
| | | | CDR2: IGIIVDSS - SEQ ID NO: 395/ |
| | | | CDR3: SKDPTGSS - SEQ ID NO: 397 |
| 3CRH15 | 20375 | 4 | QVQLQESGGGFVXPGGSLRLSCAASGFTFSSSGMHWFRQAPGKGL |
| | | | EWVSTISSGGDVTYYLDSVKGRITISRDNAKNTLYLQMNDLKSED |
| | | | TAVYYCSKDPTGATRGQGTQVTVSS |
| | | | SEQ ID NO: 63 |
| | | | CDR1: GFTFSSSG - SEQ ID NO: 396/ |
| | | | CDR2: ISSGGDVT - SEQ ID NO: 398/ |
| | | | CDR3: SKDPTGAT - SEQ ID NO: 399 |
| 3CRH43 | 20382 | 4 | QVQLQESGGGSVQPGGSLRLSCAASGFTFSTSGMHWFRQAPGKGL |
| | | | EWVSSIGIIVDSSYYASSVKGRFTISRDNAKNIVYLQMNSLKSED |
| | | | TAVYYCSKDPTGSSRGQGTQVTVSS |
| | | | SEQ ID NO:64 |
| | | | CDR1: GFTFSTSG - SEQ ID NO: 400/ |
| | | | CDR2: IGIIVDSS - SEQ ID NO: 401/ |
| | | | CDR3: SKDPTGSS - SEQ ID NO: 402 |
| 3CRH45 | 20383 | 4 | QVQLQESGGGLVQPGGSLRLSCAASGFTFSTFGMHWFRQAPGKGL |
| | | | EWVSSIGIIDDNSYYASSVKGRFTISRDNAKNTLYLQMNSLKSED |
| | | | TAVYYCSKDTTGSSRGQGTQVTVSS |
| | | | SEQ ID NO: 65 |
| | | | CDR1: GFTFSTFG - SEQ ID NO: 403/ |
| | | | CDR2: IGIIDDNS - SEQ ID NO: 404/ |
| | | | CDR3: SKDTTGSS - SEQ ID NO: 405 |
| 3CRHS1 | 20412 | 4 | QVQLQESGGGSVQPGGSLRLSCAASGFTFSTSGMHWFRQAPGKGL |
| | | | EWVSSIGIIVDSSYYASSVKGRFTISRDNAKNTLYLQMNSLKSED |
| | | | TAVYYCSKDPTGSSRGQGTQVTVSS |
| | | | SEQ ID NO: 66 |
| | | | CDR1: GFTFSTSG - SEQ ID NO: 406/ |
| | | | CDR2: IGIIVDSS - SEQ ID NO: 407/ |
| | | | CDR3: SKDPTGSS - SEQ ID NO: 408 |
| 3CRHS109 | 20430 | 4 | QVQLQESGGGLVQPGGSLRLSCAASGFTFSTSGMHWFRQAPGKGL |
| | | | EWVSSIGIIVDSSYYASSVKGRFTISRDNAKNTLYLHMNSLKSED |
| | | | TAVYYCSKDVTGSSRGQGTQVTVSS |
| | | | SEQ ID NO: 67 |
| | | | CDR1: GFTFSTSG - SEQ ID NO: 409/ |
| | | | CDR2: IGIIVDSS - SEQ ID NO: 410/ |
| | | | CDR3: SKDVTGSS - SEQ ID NO: 411 |
| 3CRHS128 | 20432 | 4 | QVQLQESGGGLVQPGGSLRLSCAASGFTFSTSGMHWFRQAPGKGL |
| | | | EWVSSIGATVDNSYYASSVKGRFTISRDNAKNTLYLLMNSLKSED |
| | | | TAVYYCSKDPTGSSRGQGTQVTVSS |
| | | | SEQ ID NO: 68 |
| | | | CDR1: GFTFSTSG - SEQ ID NO: 412/ |
| | | | CDR2: IGATVDNS - SEQ ID NO: 413/ |
| | | | CDR3: SKDPTGSS - SEQ ID NO: 414 |
| 2CRH10 | 20362 | 5 | QVQLQESGGGLVQAGGSLVLSCAASGSAALIFLMSWYRQAPGKQR |
| | | | ELVAEVSRGGSTTYADSVEGRFHISRDNAKNIVDLQMNSLKPEDT |
| | | | AVYYCKADQNYGDSLRSSWGRGTQVTVSS |
| | | | SEQ ID NO: 69 |
| | | | CDR1: GSAALIFL - SEQ ID NO: 415/ |
| | | | CDR2: VSRGGST - SEQ ID NO: 416/ |
| | | | CDR3: KADQNYGDSLRSS - SEQ ID NO: 417 |
| 2CRH53 | 20366 | 5 | QVQLQESGGGLVQAGGSLVLSCAASGSAALIFLMSWYRQAPGKQR |
| | | | ELVAEVSRYGSTTYADSVEGRFTISRDNAKDIVDLQMNSLKPEDT |
| | | | AVYYCKADQYYGDSLRSSWGRGTQVTVSS |
| | | | SEQ ID NO: 70 |
| | | | CDR1: GSAALIFL |
| | | | SEQ ID NO:418/ |
| | | | CDR2: VSRYGST - SEQ ID NO: 419/ |
| | | | CDR3: KADQYYGDSLRSS - SEQ ID NO: 420 |
| 2CRH73 | 20369 | 5 | QVQLQESGGGLVQAGGSLRLTCTASGSAALIFLMSWYRQAPGKQR |
| | | | ELVAEVSRRGSTTYADSVEGRFTISRDNAKNIVDLQMNSLKPEDT |
| | | | AVYYCKADQYYGDSLRSSWGRGTQVTVSS |
| | | | SEQ ID NO: 71 |
| | | | CDR1: GSAALIFL - SEQ ID NO: 421/ |
| | | | CDR2: VSRRGST - SEQ ID NO: 422/ |
| | | | CDR3: KADQYYGDSLRSS - SEQ ID NO: 423 |
| 3CRH5 | 20373 | 5 | QVQLQESGGGLVQAGGSLVLSCAASGSAALIFLMSWYRQAPGKQR |
| | | | ELVAEVSRGGSTTYADSVEGRFTISRDNAKNIVDLQMNSLKPEDT |
| | | | AVYYCKADQNYGDSLRSSWGQGTQVTVSS |
| | | | SEQ ID NO: 72 |
| | | | CDR1: GSAALIFL - SEQ ID NO: 424/ |
| | | | CDR2: VSRGGST - SEQ ID NO:425/ |
| | | | CDR3: KADQNYGDSLRSS - SEQ ID NO: 426 |
| 3CRH17 | 20376 | 5 | QVQLQESGGGLVQAGGSLVLSCAASGSAALIFLMSWYRQAPGKQR |
| | | | ELVAEVSRYGSTTYADSVEGRFTISRDNAKNIVDLQMNSLKPEDT |
| | | | AVYYCKADQYYGDSLRSSWGRGTQVTVSS |
| | | | SEQ ID NO: 73 |
| | | | CDR1: GSAALIFL - SEQ ID NO: 427/ |
| | | | CDR2: VSRYGST - SEQ ID NO: 428/ |
| | | | CDR3: KADQYYGDSLRSS - SEQ ID NO: 429 |
| 3CRH38 | 20381 | 5 | QVQLQESGGGLVQAGGSLVLSCAASGSAALIFLMSWYRQAPGKQR |
| | | | ELVAEVSRGGSTTYADSVEGRFTISRDNAKNIVDLQMNSLKPEDT |
| | | | AVYYCKADQNYGDSLRSSWGRGTQVTVSS |
| | | | SEQ ID NO: 74 |
| | | | CDR1: GSAALIFL - SEQ ID NO: 430/ |
| | | | CDR2: VSRGGST - SEQ ID NO: 431/ |
| | | | CDR3: KADQNYGDSLRSS - SEQ ID NO: 432 |
| 3CRH67 | 20387 | 5 | QVQLQESGGGLVQAGGSLVLSCAASGSAALIFLMSWYRQAPGKQR |
| | | | ELVAEVSRYGSTTYADSVEGRFTISRDNAKNIVDLQMNSLKPEDT |
| | | | AAYYCKADQYYGDSLRSSWGRGTQVTVSS |
| | | | SEQ ID NO: 75 |
| | | | CDR1: GSAALIFL - |
| | | | SEQ ID NO: 433/ |
| | | | CDR2: VSRYGST - SEQ ID NO: 434/ |
| | | | CDR3: KADQYYGDSLRSS - SEQ ID NO: 435 |
| 3CRHS2 | 20413 | 5 | QVQLQESGGGLVQLGGSLTLSCAASGSAALIFLMSWYRQAPGKQR |
| | | | ELVAEVSRYGSTTYADSVEGRFTISRDNAKNIVDLQMNSLKPEDT |
| | | | AVYYCKADQYYGDSLRSSWGRGTQVTVSS |
| | | | SEQ ID NO: 76 |
| | | | CDR1: GSAALIFL - SEQ ID NO: 436/ |
| | | | CDR2: VSRYGST - SEQ ID NO: 437/ |
| | | | CDR3: KADQYYGDSLRSS - SEQ ID NO: 438 |
| 3CRHS184 | 20441 | 5 | QVQLQESGGGLVQAGGSLVLSCAASGSAALIFLMSWYRQAPGKQR |
| | | | ELVAEVSRYGSTTYADSVEGRFTISRDNAKNIVDLQMNSLKPEDT |
| | | | AVYYCKADQYYGDSLRSSWGRGTQLTVSS |
| | | | SEQ ID NO: 77 |
| | | | CDR1: GSAALIFL - SEQ ID NO: 439/ |
| | | | CDR2: VSRYGST - SEQ ID NO: 440/ |
| | | | CDR3: KADQYYGDSLRSS - SEQ ID NO: 441 |
| 2CRM39 | 20447 | 6 | QVQLQESGGGLVQPGGSLRLSCAASGFTFRSYAMSWVRQAPGKGL |
| | | | EWVSSISSSGTDTNYADSVKGRLTISRDNVKNTLYLQMNSLKPED |
| | | | TAVYYCAKASQNYSPPGQGTQVTVSS |
| | | | SEQ ID NO: 78 |
| | | | CDR1: GFTFRSYA - SEQ ID NO: 442/ |
| | | | CDR2: ISSSGTDT - SEQ ID NO: 443/ |
| | | | CDR3: AKASQNYSP - SEQ ID NO: 444 |
| 2CRM61 | 20450 | 6 | QVQLQESGGGLVQPGGSLRLSCAASGFTISNYAMSWVRQAPGKGL |
| | | | EWVSSIGSNIASTNYADSVKGRFTISRDNAKNTLYLQMNSLKPED |
| | | | TAVYYCAKASQNHSPPGQGTQVTVSS |
| | | | SEQ ID NO: 79 |
| | | | CDR1: GFTISNYA - SEQ ID NO: 445/ |
| | | | CDR2: IGSNIAST - SEQ ID NO 446:/ |
| | | | CDR3: AKASQNHSP - |
| | | | SEQ ID NO: 447 |
| 2CRM83 | 20451 | 6 | QVQLQESGGGLVQPGGSLRLSCAASGFTFSAYAMSWVRQAPGKGL |
| | | | EWVSSISSTGTNPNYADSVKGRFTISRDNAKNTLYLQMNSLKPED |
| | | | TAVYYCAEAYRNYSPPGQGTQVTVSS |
| | | | SEQ ID NO: 80 |
| | | | CDR1: GFTFSAYA - SEQ ID NO: 448/ |
| | | | CDR2: ISSTGTNP - SEQ ID NO:449/ |
| | | | CDR3: AEAYRNYSP - SEQ ID NO: 450 |
| 2CRM84 | 20452 | 6 | QVQLQESGGGLVQAGGSLRLSCAASGFAFSSVAMSWVRQAPGKGL |
| | | | EWVSSISSGGHDTNYANSVKGRFTISRDNAKNTLYLQMNSLKPED |
| | | | TAVYYCAKSSRNYSPPGQGTQVTVSS |
| | | | SEQ ID NO: 81 |
| | | | CDR1: GFAFSSVA - |
| | | | SEQ ID NO: 451/ |
| | | | CDR2: ISSGGHDT - SEQ ID NO: 452/ |
| | | | CDR3: AKSSRNYSP - SEQ ID NO: 453 |
| 3CRM63 | 20460 | 6 | QVQLQESGGGLVQAGGSLRLSCAASGFTLGSYAMSWVRQAPGKGL |
| | | | EWVSSISSSGTNTNYADSVKGRFTISRDNAKNTLYLQMNSLKPED |
| | | | TAVYYCAKASQNHSPPGQGTPVTVSS |
| | | | SEQ ID NO: 82 |
| | | | CDR1: GFTLGSYA - SEQ ID NO: 454/ |
| | | | CDR2: ISSSGTNT - SEQ ID NO: 455/ |
| | | | CDR3: AKASQNHSP - SEQ ID NO: 456 |
| 3CRMS90 | 20527 | 6 | QVQLQESGGGLVQAGGSLRLSCAASGFTLSTYAMSWVRQAPGKGL |
| | | | EWVSSISSSGASTNYADSVKGRFTISRDNAKNTLYLQMNSLKPED |
| | | | TAVYYCAKASQNYSPPGQGTQVTVSS |
| | | | SEQ ID NO: 83 |
| | | | CDR1: GFTLSTYA - SEQ ID NO: 457/ |
| | | | CDR2: ISSSGAST - |
| | | | SEQ ID NO: 458/ |
| | | | CDR3: AKASQNYSP - SEQ ID NO: 459 |
| 3CRMS92 | 20528 | 6 | QVQLQESGGGLVQPGGSLRLSCAASGFTFRSYAMSWVRXAPGKGL |
| | | | EWVSSISSSGTDTNYADSVKGRFTISRDNVKNTLYLQMNSLKPED |
| | | | TAVYYCAKASQNYSPPGQGTQVTVSS |
| | | | SEQ ID NO: 84 |
| | | | CDR1: GFTFRSYA - SEQ ID NO: 460/ |
| | | | CDR2: ISSSGTDT - SEQ ID NO: 461/ |
| | | | CDR3: AKASQNYSP - SEQ ID NO: 462 |
| 2CAM2 | 20466 | 7 | QVQLQESGGGLVQAGDSLRLSCAPSGRTFSTYAMGWFRQAPGKER |
| | | | EFVAGISWSGGRTYYADSVKGRFTISRDNAKNTVYLQMNSLKPED |
| | | | TAVYYCAADPMDYSGSLYAQRSYDYWGQGTQVTVSS |
| | | | SEQ ID NO: 85 |
| | | | CDR1: GRTFSTYA - SEQ ID NO: 463/ |
| | | | CDR2: ISWSGGRT - SEQ ID NO: 464/ |
| | | | CDR3: AADPMDYSGSLYAQRSYDY - SEQ ID NO: 465 |
| 2CAM11 | 20471 | 7 | QVQLQESGGGLVQAGGSLRLSCAASGRTFVTYAMGWFRQAPGKER |
| | | | EFVAAISWSGGRLYYADSAKGRFTISRDNAKNTVYLQMNSLKPED |
| | | | TAVYYCAADPQDYTGRDYQRRRYDYWGQGTQVTVSS |
| | | | SEQ ID NO: 86 |
| | | | CDR1: GRTFVTYA - SEQ ID NO: 466/ |
| | | | CDR2: ISWSGGRL - SEQ ID NO: 467/ |
| | | | CDR3: AADPQDYTGRDYQRRRYDY - SEQ ID NO: 468 |
| 3CAM79 | 20515 | 7 | QVQLQESGGGLVQAGGSLRLSCAASGRTFSTYAMGWFRQAPGKER |
| | | | EFVAAISWSGGRTYYADSVKGRFAISRDNAKNTAYLQMNSLKPED |
| | | | TAVYYCAADPSDYMGSYYGARRYDYWGQGTQVTVSS |
| | | | SEQ ID NO: 87 |
| | | | CDR1: - GRTFSTYA |
| | | | SEQ ID NO: 469/ |
| | | | CDR2: ISWSGGRT - SEQ ID NO: 470/ |
| | | | CDR3: AADPSDYMGSYYGARRYDY - SEQ ID NO: 471 |
| 2CAMS19 | 20532 | 7 | QVQLQESGGGLVQAGGSLRLSCAASGRTFVTYAMGWFRQAPGKER |
| | | | EFVAAISWSGGRLYYADSVKGRFTISRDNAKNTVYLQMNSLKPED |
| | | | TAVYYCAADPQDYTGRDYQRRRYDYWGQGTQVTVSS |
| | | | SEQ ID NO: 88 |
| | | | CDR1: GRTFVTYA - SEQ ID NO: 472/ |
| | | | CDR2: ISWSGGRL - SEQ ID NO: 473/ |
| | | | CDR3: AADPQDYTGRDYQRRRYDY - SEQ ID NO: 474 |
| 3CAMS12 | 20542 | 7 | QVQLQESGGGLVQAGGSLRLSCAASGRTFSTYAMGWFRQAPGKER |
| | | | EFVAAISWSGGRTYYADSVKGRFTISRDNAKNTAYLQMSSLIPED |
| | | | TAVYYCAADPADYTGSYYYQRSYDYWGQGTQVTVSS |
| | | | SEQ ID NO: 89 |
| | | | CDR1: GRTFSTYA - SEQ ID NO: 475/ |
| | | | CDR2: ISWSGGRT - SEQ ID NO: 476/ |
| | | | CDR3: AADPADYTGSYYYQRSYDY - SEQ ID NO: 477 |
| 3CAMS73 | 20553 | 7 | QVQLQESGGGLVQPGGSLRLSCAASGRTFSTYAMGWFRQAPGKER |
| | | | EFVAAISWSGGRTYYADSVKGRFTISRDNAKNTAYLQMSSLIPED |
| | | | TAVYYCAADPADYTGSYYYQRSYDYWGQGTQVTVSS |
| | | | SEQ ID NO: 90 |
| | | | CDR1: GRTFSTYA - SEQ ID NO: 478/ |
| | | | CDR2: ISWSGGRT - SEQ ID NO:479/ |
| | | | CDR3: AADPADYTGSYYYQRSYDY - SEQ ID NO: 480 |
| 3CAMS80 | 20554 | 7 | QVQLQESGGGLVQAGGSLRLSCAASGRTFSTYAMGWFRQAPGKER |
| | | | EFVAAISWSGGRLYYADSVKGRFTISRDNAKNTAYLQMNSLKPED |
| | | | TAVYYCAADEEDYSGTYYYSRKYNYWGQGTQVTVSS |
| | | | SEQ ID NO: 91 |
| | | | CDR1: GRTFSTYA - SEQ ID NO: 481/ |
| | | | CDR2: ISWSGGRL - SEQ ID NO: 482/ |
| | | | CDR3: AADEEDYSGTYYYSRKYNY - SEQ ID NO: 483 |
| 2CAM16 | 20474 | 8 | QVQLQESGGGLVQAGGSLRLSCAASGFTFDDYTIGWFRQAPGKER |
| | | | EFVAALSWSAGSTFFTDSVKDRFSISRDNAKNTVLLQMNSLKPED |
| | | | TAVYYCAAKNPPHRWSDITKSLQYAYWGQGTQVTVSS |
| | | | SEQ ID NO: 92 |
| | | | CDR1: GFTFDDYT - SEQ ID NO: 484/ |
| | | | CDR2: LSWSAGST - SEQ ID NO: 485/ |
| | | | CDR3: AAKNPPHRWSDITKSLQYAY - SEQ ID NO: 486 |
| 2CAM33 | 20479 | 8 | QVQLQESGGGLVQTGGSLRLSCAASGRTFSNSVMGWFRQAPGKER |
| | | | EFVAALMWSAGSEFYTDSVKGRFSISGDGAKNTVYLQMNSLKAED |
| | | | TAVYYCAAKNPPHRWSDIGKSPHYEYWGQGTQVTVSS |
| | | | SEQ ID NO: 93 |
| | | | CDR1: GRTFSNSV - SEQ ID NO: 487/ |
| | | | CDR2: LMWSAGSE - |
| | | | SEQ ID NO: 488/ |
| | | | CDR3: AAKNPPHRWSDIGKSPHYEY - SEQ ID NO: 489 |
| 2CAM41 | 20483 | 8 | QVQLQESGGGLVQAGGSLRLSCAASGRTFSNSVIGWFRQAPGKER |
| | | | EFVAALSWSAGSEFYVDSFKGRFSISRDNAKNTVYLQMNSLKPED |
| | | | TAVYYCAAKNPPHRWSDIGKSPQYAYWGQGTQVTVSS |
| | | | SEQ ID NO: 94 |
| | | | CDR1: GRTFSNSV - SEQ ID NO: 490/ |
| | | | CDR2: LSWSAGSE - SEQ ID NO: 491/ |
| | | | CDR3: AAKNPPHRWSDIGKSPQYAY - SEQ ID NO: 492 |
| 2CAM42 | 20484 | 8 | QVQLQESGGGLVQPGGSLRLSCAASGRTFSNSVMGWFRQAPGKER |
| | | | EFVAALMWSAGSEFYTDSVKGRFSISGDGAKNTVYLQMNSLKAED |
| | | | TAVYYCAAKNPPHRWSDIGKSPHYEYWGQGTQVTVSS |
| | | | SEQ ID NO: 95 |
| | | | CDR1: GRTFSNSV - SEQ ID NO: 493/ |
| | | | CDR2: LMWSAGSE - SEQ ID NO: 494/ |
| | | | CDR3: AAKNPPHRWSDIGKSPHYEY - SEQ ID NO: 495 |
| 2CAM71 | 20496 | 8 | QVQLQESGGGLVQAGGSLRLSCAASGRTLSNSVMGWFRQAPGKER |
| | | | EFVAALMWSAGSEFYTDSVKGRFSISGDGAKNTVYLQMNSLKAED |
| | | | TAVYYCAAKNPPHRWSDIGKSPHYEYWGQGTQVTVSS |
| | | | SEQ ID NO: 96 |
| | | | CDR1: GRTLSNSV - SEQ ID NO: 496/ |
| | | | CDR2: LMWSAGSE - SEQ ID NO: 497/ |
| | | | CDR3: AAKNPPHRWSDIGKSPHYEY - SEQ ID NO: 498 |
| 3CAMS61 | 20551 | 8 | QVQLQESGGGLVXAGGSLRLSCAASGPTFSNSVIGWFRQAPGKER |
| | | | EFVAALSWSADTIFYADSVKDRFSISRDNAKNTVHLQMNSLKPED |
| | | | TAVYYCAAKNPPHRYSDINNAPHYAYWAQGTQVTVSS |
| | | | SEQ ID NO: 97 |
| | | | CDR1: GPTFSNSV - SEQ ID NO: 499/ |
| | | | CDR2: LSWSADTI - SEQ ID NO:500/ |
| | | | CDR3: AAKNPPHRYSDINNAPHYAY - SEQ ID NO: 501 |
| 3CAHS39 | 20393 | 9 | QVQLQESGGGLVQAGGSLRLSCAASGRTINAYIMGWFRQAPGKER |
| | | | EFVAAVSGSGGSTYYADSVKGRFTISRDNAKNTVNLQMDNLRSED |
| | | | TDVYYCAATYESVYLQNPGQYDYWGQGTQVTVSS |
| | | | SEQ ID NO: 98 |
| | | | CDR1: GRTINAYI - SEQ ID NO: 502/ |
| | | | CDR2: VSGSGGST - SEQ ID NO: 503/ |
| | | | CDR3: AATYESVYLQNPGQYDY - SEQ ID NO: 504 |
| 3CAHS43 | 20395 | 9 | QVQLQESGGGSVQAGGSLRLSCAASGRTINAYIMGWFRQAPGKER |
| | | | EFVAAVSGSGGSTYYADSVKGRFTISRDNAKNTVNLQMDNLRSED |
| | | | TDVYYCAATYESVYLRNPGQYDYWGQGTQVTVSS |
| | | | SEQ ID NO: 99 |
| | | | CDR1: GRTINAYI - SEQ ID NO:505/ |
| | | | CDR2: VSGSGGST - SEQ ID NO: 506/ |
| | | | CDR3: - AATYESVYLRNPGQYDY |
| | | | SEQ ID NO: 507 |
| 3CAHS52 | 20396 | 9 | QVQLQESGGGSVQAGGSLRLSCAASGRTINAYIMGWFRQAPGKER |
| | | | EFVAAVSGSGGSTYYADSVKGRFTISRDNAKNTVNLQMDNLRSED |
| | | | TDVYYCAATYESVYLQNPGQYDYWGQGTQVTVSS |
| | | | SEQ ID NO: 100 |
| | | | CDR1: GRTINAYI - SEQ ID NO:508/ |
| | | | CDR2: VSGSGGST - SEQ ID NO: 509/ |
| | | | CDR3: AATYESVYLQNPGQYDY - SEQ ID NO: 510 |
| 3CAHS53 | 20397 | 9 | QVQLQESGGGLVEPGGSLRLSCAASGRTINAYIMGWFRQAPGKER |
| | | | EFVAAVSGSGGSTYYADSVKGRFTISRDSAKNTVNLQMDNLRSED |
| | | | TDVYYCAATYESVYLQNPGQYDYWGQGTQVTVSS |
| | | | SEQ ID NO: 101 |
| | | | CDR1: GRTINAYI - SEQ ID NO: 511/ |
| | | | CDR2: VSGSGGST - SEQ ID NO: 512/ |
| | | | CDR3: AATYESVYLQNPGQYDY - SEQ ID NO: 513 |
| 3CAHS124 | 20400 | 9 | QVQLQESGGGLVQPGGSLRLSCAASGRTINAYIMGWFRQAPGKER |
| | | | EFVAAVSGSGGSTYYADSVKGRFTISRDNAKNTVNLQMDNLRSED |
| | | | TDVYYCAATYESVYLQNPGQYDYWGQGTQVTVSS |
| | | | SEQ ID NO: 102 |
| | | | CDR1: GRTINAYI - SEQ ID NO: 514/ |
| | | | CDR2: VSGSGGST - SEQ ID NO: 515/ |
| | | | CDR3: AATYESVYLONPGQYDY - SEQ ID NO: 516 |
| 3CAHS150 | 20403 | 9 | QVQLQESGGGSVQAGGSLRLSCAASGRTINAYVMGWFRQAPGKER |
| | | | EFVAAISGSGGSTYYADSVKGRFTISRDNAKNTVNLQMDNLRSED |
| | | | TDVYYCAATYESVYLQNPGQYDYWGQGTQVTVSS |
| | | | SEQ ID NO: 103 |
| | | | CDR1: GRTINAYV - SEQ ID NO: 517/ |
| | | | CDR2: ISGSGGST - SEQ ID NO: 518/ |
| | | | CDR3: AATYESVYLQNPGQYDY - SEQ ID NO: 519 |
| 2CAM6 | 20468 | 10 | QVQLQESGGGLVQAGGSLRLSCVASGRTFSSYGMGWFRQAPGKER |
| | | | EFVTGISSAGSWIGDITSYADSVKGRFTISRDNAKNILYLQMNSL |
| | | | KPEDTAVYYCAAAYHGSRYYVGSTANEYTTWGQGTQVTVSS |
| | | | SEQ ID NO: 104 |
| | | | CDR1: GRTFSTYA - SEQ ID NO: 520/ |
| | | | CDR2: ISWSGGST - SEQ ID NO: 521/ |
| | | | CDR3: AADAEDPILYGSSWNNRRKEYDY - SEQ ID NO: 522 |
| 2CAM20 | 20476 | 10 | QVQLQESGGGSVQAGGSLRLSCAVFGLSFKDYSMSWFRQAPGKER |
| | | | EFVAGISSGGSWIGDITSYADSVKGRFTISRDNAKNMIYLQMNSL |
| | | | KPDDTGVYYCAAASYGTRYYVGSTANEYTTWGQGTQVTVSS |
| | | | SEQ ID NO: 105 |
| | | | CDR1: GLSFKDYS - SEQ ID NO: 523/ |
| | | | CDR2: ISSGGSWIGDIT - SEQ ID NO:524/ |
| | | | CDR3: AAASYGTRYYVGSTANEYTT - SEQ ID NO: 525 |
| 2CAM62 | 20493 | 10 | QVQLQESGGGLVQAEGSLRLSCVASGRTFSSYGMGWFRQAPGKER |
| | | | EFVTGISSAGSWIGDITSYADSVKGRFTISRDNAKNILYLQMNSL |
| | | | KPEDTAVYYCAAAYHGSRYYVGSTANEYTTWGQGTQVTVSS |
| | | | SEQ ID NO: 106 |
| | | | CDR1: GRTFSSYG - SEQ ID NO: 526/ |
| | | | CDR2: ISSAGSWIGDIT - SEQ ID NO: 527/ |
| | | | CDR3: AAAYHGSRYYVGSTANEYTT - SEQ ID NO: 528 |
| 2CAMS7 | 20530 | 10 | QVQLQESGGGLVQAGDSLRLSCAASGRTFSNYNMGWLRQAPGKER |
| | | | EFVTGISSAGSWIGDITSYADSVKGRFTISRDNAKNILYLQMNSL |
| | | | KPEDTAVYYCAAAYHGSRYYVGSTANEYTTWGQGTQVTVSS |
| | | | SEQ ID NO: 107 |
| | | | CDR1: GRTFSNYN - SEQ ID NO: 529/ |
| | | | CDR2: ISSAGSWIGDIT - SEQ ID NO: 530/ |
| | | | CDR3: AAAYHGSRYYVGSTANEYTT - SEQ ID NO: 531 |
| 2CAMS26 | 20533 | 10 | QVQLQESGGGLVQAGGSLTLSCTASGRTFSSYTTGWFRQAPGKER |
| | | | EFVAGISSGGSWIGDITSYADSVKGRFTISRDNAKNMIYLQMNSL |
| | | | KPDDTGVYYCAAASYGTRYYVGSTANEYTTWGQGTQVTVSS |
| | | | SEQ ID NO: 108 |
| | | | CDR1: GRTFSSYT - SEQ ID NO: 532/ |
| | | | CDR2: ISSGGSWIGDIT - SEQ ID NO: 533/ |
| | | | CDR3: AAASYGTRYYVGSTANEYTT - SEQ ID NO: 534 |
| 2CAH27 | 20354 | 11 | QVQLQESGGGSVQSGGSLRLSCVASGRTFSSYAMGWFRQGPGKER |
| | | | EFVAAISWSGVSIYYADSVKGRFTISRDNAKNTVYLQMNSLEPED |
| | | | TAVYYCAGTEGAYTVAGGSTFGYDMDYWGKGTQVTVSS |
| | | | SEQ ID NO: 109 |
| | | | CDR1: GRTFSSYA - SEQ ID NO: 535/ |
| | | | CDR2: ISWSGVSI - SEQ ID NO: 536/ |
| | | | CDR3: AGTEGAYTVAGGSTFGYDMDY - SEQ ID NO: 537 |
| 3CAH48 | 20360 | 11 | QVQLQESGGGLVQAGGSLRLSCAASGRTFSSYAMGWFRQGPGKER |
| | | | EFVAAISWSGVSTYYADSVKGRFTISRDNAKNTVYLQMNSLRPED |
| | | | TAVYYCAATEGAYTVAGGSTFGYDMDYWGKGTQVTVSS |
| | | | SEQ ID NO: 110 |
| | | | CDR1: GRTFSSYA - SEQ ID NO:538/ |
| | | | CDR2: ISWSGVST - SEQ ID NO: 539/ |
| | | | CDR3: AATEGAYTVAGGSTFGYDMDY - SEQ ID NO: 540 |
| 3CAHS142 | 20401 | 11 | QVQLQESGGGLVQAGESLTLSCIASGVIKDYAMGWFRQAPGKERE |
| | | | FVAAISWSGVSIYYADSVKGRFTISRDNAKNTVYLQMNSLEPEDT |
| | | | AVYYCAGTEGAYTVAGGSTFGYDMDYWGKGTQVTVSS |
| | | | SEQ ID NO: 111 |
| | | | CDR1: GVIKDYA - SEQ ID NO: 541/ |
| | | | CDR2: ISWSGVSI - SEQ ID NO: 542/ |
| | | | CDR3: AGTEGAYTVAGGSTFGYDMDY - SEQ ID NO: 543 |
| 3CAHS154 | 20405 | 11 | QVQLQESGGGLVQAGGSLRLSCVASGRTFSSYAMGWFRQGPGKER |
| | | | EFVAAISXSGVSIYYADSVKGRFTISRDNAKNTVYLQMNSLEPED |
| | | | TAAYYCAGTEGAYTVAGGSTFGYDMDYWGKGTQVTVSS |
| | | | SEQ ID NO: 112 |
| | | | CDR1: GRTFSSYA - SEQ ID NO: 544/ |
| | | | CDR2: ISXSGVSI - SEQ ID NO: 545/ |
| | | | CDR3: AGTEGAYTVAGGSTFGYDMDY - SEQ ID NO: 546 |
| 3CAHS170 | 20406 | 11 | QVQLQESGGGLVQAGGSLRLSCVASGRTFSSYAMGWFRQGPGKER |
| | | | EFVAAISWSGVSIYYADSVKGRFTISRDNAKNTVYLQMNSLEPED |
| | | | TAVYYCAGTEGAYTVAGGSTFGYDMDYWGKGTQVTVSS |
| | | | SEQ ID NO: 113 |
| | | | CDR1: GRTFSSYA - SEQ ID NO: 547/ |
| | | | CDR2: ISWSGVSI - SEQ ID NO: 548/ |
| | | | CDR3: AGTEGAYTVAGGSTFGYDMDY - SEQ ID NO: 549 |
| 2CAM3 | 20467 | 12 | QVQLQESGGGLVQAGGSLRLSCAPSGRTFSTLAMAWFRQAPGKER |
| | | | EVVATISWSGGRTYYADSVKGRFTISRDNAKNTAYLQMNSLRPDD |
| | | | TAVYYCAARGSDYGSYDYWGQGTQVTVSS |
| | | | SEQ ID NO: 114 |
| | | | CDR1: GRTFSTLA - SEQ ID NO: 550/ |
| | | | CDR2: ISWSGGRT - SEQ ID NO: 551/ |
| | | | CDR3: AARGSDYGSYDY - SEQ ID NO: 552 |
| 2CAM27 | 20477 | 12 | QVQLQESGGGLVQAGGSLRLSCAPSGRTFSTYAMGWFRQALGKER |
| | | | EVVATISWSGGRIYYTDSVKGRFTISRDNAKNTAYLQMNSLRPDD |
| | | | TAVYYCAARGTDYGAWEYWGQGTQVTVSS |
| | | | SEQ ID NO: 115 |
| | | | CDR1: GRTFSTYA - SEQ ID NO: 553/ |
| | | | CDR2: ISWSGGRI - SEQ ID NO: 554/ |
| | | | CDR3: AARGTDYGAWEY - SEQ ID NO: 555 |
| 2CAM46 | 20486 | 12 | QVQLQESGGGLVQAGGSLRLSCAPSGRTFSTLAMAWFRQAPGKER |
| | | | EVVATISWSGGRTYYADSVKGRFTISRDNAKNTAFLQMNSLRPDD |
| | | | TAVYYCAARESDYGSYDYWGQGTQVTVSS |
| | | | SEQ ID NO: 116 |
| | | | CDR1: GRTFSTLA - |
| | | | SEQ ID NO:556/ |
| | | | CDR2: ISWSGGRT - SEQ ID NO: 557/ |
| | | | CDR3: AARESDYGSYDY - SEQ ID NO: 558 |
| 2CAM50 | 20489 | 12 | QVQLQESGGGLVQPGGPLRLSCAPSGRTFSTLAMAWFRQAPGKER |
| | | | EVVATISWSGGRTYYADSVKGRFTISRDNAKNTAFLQMNSLRPDD |
| | | | TAVYYCAARESDYGSYDYWGQGTQVTVSS |
| | | | SEQ ID NO: 117 |
| | | | CDR1: GRTFSTLA - SEQ ID NO: 559/ |
| | | | CDR2: ISWSGGRT - SEQ ID NO: 560/ |
| | | | CDR3: - AARESDYGSYDY |
| | | | SEQ ID NO: 561 |
| 3CAM6 | 20506 | 12 | QVQLQESGGGLVQVGGSLRLSCAPSGRTFSTYAMGWFRQALGKER |
| | | | EVVATISWSGGRIYYTDSVKGRFTISRDNAKNTAYLQMNSLRPDD |
| | | | ADVYYCATRGTDYGAWEYWGQGTQVTVSS |
| | | | SEQ ID NO: 118 |
| | | | CDR1: GRTFSTYA - SEQ ID NO:562/ |
| | | | CDR2: ISWSGGRI - SEQ ID NO: 563/ |
| | | | CDR3: ATRGTDYGAWEY - |
| | | | SEQ ID NO: 564 |
| 3CAM57 | 20512 | 12 | QVQLQESGGGLVQAGGSLRLSCTPSGRTFSTLAMGWFRQAPGKER |
| | | | EVVATISWSGGRTYYASSVKSRFTISRDNAKNTAYLQMNSLRPDD |
| | | | TAVYYCAARGTDYGAYDYWGQGTQVTVSS |
| | | | SEQ ID NO: 119 |
| | | | CDR1: GRTFSTLA - SEQ ID NO: 565/ |
| | | | CDR2: ISWSGGRT - SEQ ID NO: 566/ |
| | | | CDR3: AARGTDYGAYDY - SEQ ID NO: 567 |
| 3CAMS81 | 20555 | 12 | QVQLQESGGGLVQAGGSLRLSCAPSGRTFSTLAMGWFRQAPGKER |
| | | | EVVATISWSGGRTYYADSVKGRFTISRNNAKNTAYLQMNNLRPDD |
| | | | TAVYYCAARETDYGAYDYWGQGTQVTVSS |
| | | | SEQ ID NO: 120 |
| | | | CDR1: GRTFSTLA - SEQ ID NO: 568/ |
| | | | CDR2: ISWSGGRT - SEQ ID NO: 569/ |
| | | | CDR3: AARETDYGAYDY - SEQ ID NO: 570 |
| 2CRHS4 | 20411 | 13 | QVQLQESGGGLVQPGGSLRLSCVSSQTLVGIEAMGWYRQAPGKQR |
| | | | ELVALITLNGGTFYADSVKGRFTISRDVAGHMLYLEMNSLNPEDT |
| | | | AVYFCVAGVESDNEEPTYWGQGTQVTVSS |
| | | | SEQ ID NO: 121 |
| | | | CDR1: QTLVGIEA - SEQ ID NO: 571/ |
| | | | CDR2: ITLNGGT - SEQ ID NO: 572/ |
| | | | CDR3: VAGVESDNEEPTY - SEQ ID NO: 573 |
| 3CRHS67 | 20424 | 13 | QVQLQESGGGLVQAGGSLTLSCVSSQTLVGIEAMGWYRQAPGKQR |
| | | | ELVALITLNGGTFYADSVKGRFTISRDVAEHMLYLEMNSLNPEDT |
| | | | AVYFCVAGVESDNEEPTYWGQGTQVTVSS |
| | | | SEQ ID NO: 122 |
| | | | CDR1: QTLVGIEA - SEQ ID NO: 574/ |
| | | | CDR2: ITLNGGT - SEQ ID NO: 575/ |
| | | | CDR3: VAGVESDNEEPTY - SEQ ID NO: 576 |
| 3CRHS142 | 20434 | 13 | QVQLQESGGGLVQAGGSLRLSCVSSQTLVGIEAMGWYRQAPGKQR |
| | | | ELVALITLNGGTFYADSVKGRFTISRDVAEHMLYLEMNSLNPEDT |
| | | | AVYFCVAGVESDNEEPTYWGQGTQVTVSS |
| | | | SEQ ID NO: 123 |
| | | | CDR1: QTLVGIEA - SEQ ID NO: 577/ |
| | | | CDR2: ITLNGGT - SEQ ID NO: 578/ |
| | | | CDR3: VAGVESDNEEPTY - |
| | | | SEQ ID NO: 579 |
| 3CRHS156 | 20437 | 13 | QVQLQESGGGLVQSGGSLRLSCVSSQTLVGIEAMGWYRQAPGKQR |
| | | | ELVALITLNGGTFYADSVKGRFAISRDVAEHMLYLEMNSLNPEDT |
| | | | AVYFCVAGVESDNEEPTYWGQGTQVTVSS |
| | | | SEQ ID NO: 124 |
| | | | CDR1: QTLVGIEA - SEQ ID NO: 580/ |
| | | | CDR2: ITLNGGT - SEQ ID NO: 581/ |
| | | | CDR3: VAGVESDNEEPTY - SEQ ID NO: 582 |
| 3CRHS166 | 20439 | 13 | QVQLQESGGGLVQSGGSLRLSCVSSQTLVGIEAMGWYRQAPGKQR |
| | | | ELVALITLNGGTFYADSVKGRFTISRDVAEHMLYLEMNSLNPEDT |
| | | | AVYFCVAGVESDNEEPTYWGQGTQVTVSS |
| | | | SEQ ID NO: 125 |
| | | | CDR1: QTLVGIEA - SEQ ID NO: 583/ |
| | | | CDR2: ITLNGGT - SEQ ID NO: 584/ |
| | | | CDR3: VAGVESDNEEPTY - SEQ ID NO: 585 |
| 2CRH41 | 20365 | 14 | QVQLQESGGGLVQPGGSLRLSCTASGFAFSSYWMYWVRQSPEKGF |
| | | | EWVSVVDTDGGITYYADSVKGRFTISRDNAKNTLYLQMNSLKPQD |
| | | | TALYYCARDRTTLEYTRTYYRTGQGTQVTVSS |
| | | | SEQ ID NO: 126 |
| | | | CDR1: GFAFSSYW - SEQ ID NO: 586/ |
| | | | CDR2: VDTDGGIT - SEQ ID NO: 587/ |
| | | | CDR3: ARDRTTLEYTRTYYR - SEQ ID NO: 588 |
| 3CRH59 | 20385 | 14 | QVQLQESGGGLVQPGGSLRLSCTASGFAFSSYWMYWVRQTPGKGF |
| | | | EWVSVVNPGGEITYYGDSMEDRFTISRDNAKNTLYLQMNSLKPED |
| | | | TGLYYCARDRTTIDYTRTYYRGGQGTQVTVSS |
| | | | SEQ ID NO: 127 |
| | | | CDR1: GFAFSSYW - SEQ ID NO: 589/ |
| | | | CDR2: VNPGGEIT - SEQ ID NO: 590/ |
| | | | CDR3: ARDRTTIDYTRTYYR - SEQ ID NO: 591 |
| 3CRHS163 | 20438 | 14 | QVQLQESGGGLVQPGGSLRLSCTASGFAFSTYWMYWIRQSPEKGF |
| | | | EWVSVVNTGGDITYYADSVKGRFTISRDNAKNTLYLQMNSLKPED |
| | | | TALYYCARDRTTMSYTRTYYRGGQGTQVTVSS |
| | | | SEQ ID NO: 128 |
| | | | CDR1: GFAFSTYW - SEQ ID NO: 592/ |
| | | | CDR2: VNTGGDIT - SEQ ID NO: 593/ |
| | | | CDR3: ARDRTTMSYTRTYYR - SEQ ID NO: 594 |
| 3CRHS35 | 20419 | 15 | QVQLQESGGGLVQAGKSLTLACAASGFPFSTYAMSWVRXAPGKGL |
| | | | EWVSAVNSDGSSTDYADSVKGRFTISRDNAKNMVYLQMNSLKPED |
| | | | TARYYCAKYEDCSDYVCNRDRATYWGQGTQVTVSS |
| | | | SEQ ID NO: 129 |
| | | | CDR1: GFPFSTYA - SEQ ID NO: 595/ |
| | | | CDR2: VNSDGSST |
| | | | SEQ ID NO: 596/ |
| | | | CDR3: AKYEDCSDYVCNRDRATY - SEQ ID NO: 597 |
| 3CRHS99 | 20427 | 15 | QVQLQESGGGLVQPGGSLRLSCKASGFTFSTYAMSWVRQAPGKGL |
| | | | EWVSAINSGGLSTDYADSVEGRFTISRDDAKNMLFLQMNSLKAED |
| | | | TARYYCAKYEDCSDYMCHRDRAGYWGQGTQVTVSS |
| | | | SEQ ID NO: 130 |
| | | | CDR1: GFTFSTYA - SEQ ID NO: 598/ |
| | | | CDR2: INSGGLST - SEQ ID NO: 599/ |
| | | | CDR3: AKYEDCSDYMCHRDRAGY - SEQ ID NO: 600 |
| 3CRHS106 | 20429 | 15 | QVQLQESGGGLVQPGGSLRLSCKASGFTFSTYAMSWVRQAPGKGL |
| | | | EWVSAVNSGGLSTDYADSVEGRFTISRDDAKNMLFLQMNSLKPED |
| | | | TARYYCAKYEDCSDYMCHRDRAGYWGQGTQVTVSS |
| | | | SEQ ID NO: 131 |
| | | | CDR1: GFTFSTYA - SEQ ID NO: 601/ |
| | | | CDR2: VNSGGLST - SEQ ID NO: 602/ |
| | | | CDR3: AKYEDCSDYMCHRDRAGY - SEQ ID NO: 603 |
| 3CRH25 | 20378 | 16 | QVQLQESGGGLVQAGDSLRLSCTASGHTFSRYTTIGWFRQAPGKD |
| | | | REFVAAIRWSGDITDYADAVKGRFTISRDNVKNTVYLQMNSLNPE |
| | | | DTAVYYCARGYSEYGVLSRNRGLYAYWGQGTQVTVSS |
| | | | SEQ ID NO: 132 |
| | | | CDR1: GHTFSRYTT - SEQ ID NO: 604/ |
| | | | CDR2: IRWSGDIT - SEQ ID NO: 605/ |
| | | | CDR3: ARGYSEYGVLSRNRGLYAY - SEQ ID NO: 606 |
| 3CRH64 | 20386 | 16 | QVQLQESGGGLVQAGGSLRLSCTASGHTFSRYTTIGWFRQAPGKD |
| | | | REFVAAIRWSGDITDYADAVKGRFTISRDNVKNTVYLQTNSLNPE |
| | | | DTAVYYCARGYSEYGVLSRNRGLYAYWGQGTQVTVSS |
| | | | SEQ ID NO: 133 |
| | | | CDR1: GHTFSRYTT - SEQ ID NO: 607/ |
| | | | CDR2: IRWSGDIT - SEQ ID NO:608/ |
| | | | CDR3: ARGYSEYGVLSRNRGLYAY - SEQ ID NO: 609 |
| 3CRHS77 | 20425 | 16 | QVQLQESGGGLVQAGGSLRLSCTASGHTFSSYTTIGWFRQAPGKE |
| | | | REFVAAIGWSGDITDYADAVKGRFTISRHNVENTVYLQMNSLNPE |
| | | | DTAVYYCARGYSEYGVLSRNRGLYAYWGQGTQVTVSS |
| | | | SEQ ID NO: 134 |
| | | | CDR1: GHTFSSYTT - SEQ ID NO: 610/ |
| | | | CDR2: IGWSGDIT - SEQ ID NO:611/ |
| | | | CDR3: ARGYSEYGVLSRNRGLYAY - SEQ ID NO: 612 |
| 2CAM14 | 20473 | 17 | QVQLQESGGGLVQAGDSLRLSCAASGRTFSSYTMGWFRQAPGKER |
| | | | EFVARISQDGGSTYYADSMKGRFTVARDNAKNTVYLQMNSLKPED |
| | | | TAVYYCAADMIKWGLRVGVDSFKYWGQGTQVTVSS |
| | | | SEQ ID NO: 135 |
| | | | CDR1: GRTFSSYT - SEQ ID NO: 613/ |
| | | | CDR2: ISQDGGST - SEQ ID NO:614/ |
| | | | CDR3: - AADMIKWGLRVGVDSFKY |
| | | | SEQ ID NO: 615 |
| 2CAM88 | 20501 | 17 | QVQLQESGGGLVPPGDSLRLSCAASGRTFSSYTMGWFRQAPGKER |
| | | | EFVARISQDGGSTYYADSMKGRFTVARDNAKNTVYLQMNSLKPED |
| | | | TAVYYCAADMIKWGLRVGVDSFKYWGQGTQVTVSS |
| | | | SEQ ID NO: 136 |
| | | | CDR1: GRTFSSYT - SEQ ID NO: 616/ |
| | | | CDR2: ISQDGGST - SEQ ID NO: 617/ |
| | | | CDR3: AADMIKWGLRVGVDSFKY - SEQ ID NO: 618 |
| 3CAMS39 | 20546 | 17 | QVQLQESGGGLVXAGDSLRLSCAASGRTFSSYTMGWFRQAPRKER |
| | | | EFVARISQDGGSTYYADSMKGRFTVARDNAKNTVYLQMNSLKPED |
| | | | TAVYYCAADMIKWGLRVGVDSFKYWGQGTQVTVSS |
| | | | SEQ ID NO: 137 |
| | | | CDR1: GRTFSSYT - SEQ ID NO: 619/ |
| | | | CDR2: ISQDGGST - SEQ ID NO: 620/ |
| | | | CDR3: AADMIKWGLRVGVDSFKY - SEQ ID NO: 621 |
| 2CRM16 | 20443 | 18 | QVQLQESGGGLVQAGGSLRLSCAASGRTFSNYRMGWFRQAPGKER |
| | | | EFVAGINWDGGHTYYTDSVKGRFTISRDNAKNTVYLQMNSLKPED |
| | | | TAVYQCAVRVREYGSSRYEDQFDYWGQGTQVTVSS |
| | | | SEQ ID NO: 138 |
| | | | CDR1: GRTFSNYR - SEQ ID NO: 622/ |
| | | | CDR2: INWDGGHT - SEQ ID NO: 623/ |
| | | | CDR3: AVRVREYGSSRYEDQFDY - SEQ ID NO: 624 |
| 2CRM42 | 20449 | 18 | QVQLQESGGGLVQPGESLRLSCQASGRTFSNYRMGWFRQAPGKER |
| | | | EFVAGINWDGGHTYYTDSVKGRFTISRDNAKNTVYLQMNSLKPED |
| | | | TAVYQCAVRVREYGSSRYEDQFDYWGQGTQVTVSS |
| | | | SEQ ID NO: 139 |
| | | | CDR1: GRTFSNYR - SEQ ID NO: 625/ |
| | | | CDR2: INWDGGHT - SEQ ID NO: 626/ |
| | | | CDR3: AVRVREYGSSRYEDQFDY - SEQ ID NO: 627 |
| 2CRM88 | 20453 | 18 | QVQLQESGGGLVQAGGSLRLSCQASGRTFSNYRMGWFRQAPGKER |
| | | | EFVAGINWDGGHTYYTDSVKGRFTISRDNAKNTVYLQMNSLKPED |
| | | | TAVYQCAVRVREYGSSRYEDQFDYWGQGTQVTVSS |
| | | | SEQ ID NO: 140 |
| | | | CDR1: GRTFSNYR - SEQ ID NO: 628/ |
| | | | CDR2: INWDGGHT - SEQ ID NO: 629/ |
| | | | CDR3: AVRVREYGSSRYEDQFDY - SEQ ID NO: 630 |
| 2CAM9 | 20470 | 19 | QVQLQESGGGLVQAGMSLRLSCAASGLTFSWYRMGWFRQAPGKER |
| | | | EFVAGWYGVLTQYRDSVKGRFTISRDDAKNTVYLQMNSLKPEDTA |
| | | | VYYCAASLGGNYRSYEYDYWGQGTQVTVSS |
| | | | SEQ ID NO: 141 |
| | | | CDR1: GLTFSWYR - SEQ ID NO: 631/ |
| | | | CDR2: WYGVLT - SEQ ID NO: 632/ |
| | | | CDR3: AASLGGNYRSYEYDY - SEQ ID NO: 633 |
| 2CAM12 | 20472 | 19 | QVQLQESGGGLVQAGGSLRLSCAASGRTFSWYRMGWFRQAPGKER |
| | | | EFVAGWYGVLTQYRDSVKGRFTISRDDAKNTVYLQMNSLKPEDTA |
| | | | VYYCAASLGGNYRSYEYDYWGQGTQVTVSS |
| | | | SEQ ID NO: 142 |
| | | | CDR1: GRTFSWYR - SEQ ID NO: 634/ |
| | | | CDR2: WYGVLT - SEQ ID NO: 635/ |
| | | | CDR3: AASLGGNYRSYEYDY - SEQ ID NO: 636 |
| 3CAMS30 | 20545 | 19 | QVQLQESGGGLVXPGGSLRLSCAASGRTFSWYRMGWFRQAPGKER |
| | | | EFVAGWYGVLTQYRDSVKGRFTISRDDAKNTVYLQMNSLKPEDTA |
| | | | VYYCAASLGGNYRSYEYDYWGQGTQVTVSS |
| | | | SEQ ID NO: 143 |
| | | | CDR1: GRTFSWYR - SEQ ID NO: 637/ |
| | | | CDR2: WYGVLT - SEQ ID NO:638/ |
| | | | CDR3: AASLGGNYRSYEYDY - SEQ ID NO: 639 |
| 2CAH71 | 20355 | 20 | QVQLQESGGGLVQAGGSLRLSCAASGSILRINLMGWYRQVPGKQR |
| | | | ELVAEMSTGGSTTYVDSVKGRFTISRDNAKNTVYLQMNSLKPEDT |
| | | | AVYYCNAKAWQLGRLENDDWGQGTQVTVSS |
| | | | SEQ ID NO: 144 |
| | | | CDR1: GSILRINL - SEQ ID NO: 640/ |
| | | | CDR2: MSTGGST - SEQ ID NO: 641/ |
| | | | CDR3: NAKAWQLGRLENDD - SEQ ID NO: 642 |
| 3CAH32 | 20357 | 20 | QVQLQESGGGLVQPGGSLRLSCAASGSILRINLMGWYRQVPGKQR |
| | | | ELVAEMSTGGSTTYVDSVKGRFTISRDNAKNTVYLQMNSLKPEDT |
| | | | AVYYCNAKAWQLGRLENDDWGQGTQVTVSS |
| | | | SEQ ID NO: 145 |
| | | | CDR1: GSILRINL - SEQ ID NO:643/ |
| | | | CDR2: MSTGGST - SEQ ID NO:644/ |
| | | | CDR3: NAKAWQLGRLENDD - SEQ ID NO: 645 |
| 3CAHS102 | 20399 | 20 | QVQLQESGGGLVQPGGSLRLSCAASGSILRINLMGWYRQVPGKQR |
| | | | ELVAEMSTGGSTTYVDSVEGRFTISRDNAKNTVYLQMNSLKPEDT |
| | | | AVYYCNAKAWQLGRLENDDWGQGTQVTVSS |
| | | | SEQ ID NO: 146 |
| | | | CDR1: GSILRINL - SEQ ID NO: 646/ |
| | | | CDR2: MSTGGST - SEQ ID NO: 647/ |
| | | | CDR3: NAKAWQLGRLENDD - SEQ ID NO: 648 |
| 3CAHS79 | 20398 | 21 | QVQLQESGGGLVQPGGSLRLTCVASGFPFSTHAMSWVRQPPGKGR |
| | | | EWISAINSGGDSTDYADSVMGRFTISRDNAKNTVYLQMNSLKSED |
| | | | TAVYYCAKYEDCSDYDCSSRASWGQGTQVTVSS |
| | | | SEQ ID NO: 147 |
| | | | CDR1: GFPFSTHA - SEQ ID NO: 649/ |
| | | | CDR2: INSGGDST - SEQ ID NO: 650/ |
| | | | CDR3: AKYEDCSDYDCSSRAS - SEQ ID NO: 651 |
| 3CAHS171 | 20407 | 21 | QVQLQESGGGLVQPGGSLRLTCVASGFPFSTHAMSWVRQPPGKGR |
| | | | EWISAINSGGDSTDYADSVMGRFTISRDNAKNTVYLQMNSLKSED |
| | | | TAAYYCAKYEDCSDYDCSSRASWGQGTQVTVSS |
| | | | SEQ ID NO: 148 |
| | | | CDR1: GFPFSTHA - SEQ ID NO: 652/ |
| | | | CDR2: INSGGDST - SEQ ID NO: 653/ |
| | | | CDR3: AKYEDCSDYDCSSRAS - SEQ ID NO: 654 |
| 3CRHS31 | 20418 | 22 | QVQLQESGGGLVQAGGSLRLSCAASGFTFSTYAMSWVRQAPGKGL |
| | | | EWVSSVNSGGLSTDYADSVEGRFTISRDDAKNMLFLQMNSLKPED |
| | | | TARYYCAKYEDCSDYVCDRAGYWGQGTQVTVSS |
| | | | SEQ ID NO: 149 |
| | | | CDR1: GFTFSTYA - SEQ ID NO: 655/ |
| | | | CDR2: VNSGGLST - SEQ ID NO: 656/ |
| | | | CDR3: AKYEDCSDYVCDRAGY - SEQ ID NO: 657 |
| 3CRHS51 | 20421 | 22 | QVQLQESGGGLVQPGGSLRLSCAASGFTFSTYAMSWVRQAPGKGL |
| | | | EWVSAVNSGGLSTDYADSVEGRFTISRDDAKNMLFLQMNRLKPED |
| | | | TARYYCAKYEDCSDYVCDRAGYWGQGTQVTVSS |
| | | | SEQ ID NO: 150 |
| | | | CDR1: GFTFSTYA |
| | | | SEQ ID NO: 658/ |
| | | | CDR2: VNSGGLST - SEQ ID NO: 659/ |
| | | | CDR3: AKYEDCSDYVCDRAGY - SEQ ID NO: 660 |
| 3CAHS41 | 20394 | 23 | QVQLQESGGGLVQAGDSLRLSCAASGRTFDTYIVGWFRQAPGKER |
| | | | EFVSALSWSDDTTIRDDSLRGRFTISRDNAKNTVYLLMNSLKPED |
| | | | TAVYYCAAKAKPPAATWSSYRNREWYDYWGQGTQVTVSS |
| | | | SEQ ID NO: 151 |
| | | | CDR1: GRTFDTYI - SEQ ID NO: 661/ |
| | | | CDR2: LSWSDDTT - SEQ ID NO: 662/ |
| | | | CDR3: AAKAKPPAATWSSYRNREWYDY - SEQ ID NO: 663 |
| 3CAHS180 | 20410 | 23 | QVQLQESGGGLVQAGDSLRLSCAASGRTFDTYIVGWFRQAPGKER |
| | | | EFVSALSWSDDTTIRDDSLRGRFTLSRDNAKNTVYLLMNSLKPED |
| | | | TAVYYCAAKAKPPAATWSSYRNREWYDYWGQGTQVTVSS |
| | | | SEQ ID NO: 152 |
| | | | CDR1: GRTFDTYI - SEQ ID NO: 664/ |
| | | | CDR2: LSWSDDTT - SEQ ID NO: 665/ |
| | | | CDR3: AAKAKPPAATWSSYRNREWYDY - SEQ ID NO: 666 |
| 2CRM40 | 20448 | 24 | QVQLQESGGGLVQTGGSLRLSCAASGRTYAMAWFRQAPGKEREFA |
| | | | AVIIGGDRTYYANSVKDRFTISRDNAKETVYLQMNNLKPEDTAVY |
| | | | YCAANRRLYNSGRGYSQSEYEYWGQGTQVTVSS |
| | | | SEQ ID NO: 153 |
| | | | CDR1: GRTYA - SEQ ID NO: 667/ |
| | | | CDR2: IIGGDRT - SEQ ID NO: 668/ |
| | | | CDR3: AANRRLYNSGRGYSQSEYEY - SEQ ID NO: 669 |
| 3CRMS53 | 20526 | 24 | QVQLQESGGGLVQAGGSLRLSCAASGRTYAMAWFRXAPGKEREFA |
| | | | AVIIGGDRTYYANSVKDRFTISRDNAKETVYLQMNNLKPEDTAVY |
| | | | YCAANRRLYNSGRGYSQSEYEYWGQGTQVTVSS |
| | | | SEQ ID NO: 154 |
| | | | CDR1: GRTYA - SEQ ID NO:670/ |
| | | | CDR2: IIGGDRT - SEQ ID NO: 671/ |
| | | | CDR3: AANRRLYNSGRGYSQSEYEY - SEQ ID NO: 672 |
| 3CAM10 | 20507 | 25 | QVQLQESGGGLVQAGGSLRLSCAASGRTFSDYPMAWFRQAPGKER |
| | | | EFVAGISNGGTAYYADSVKGRFTISRDNTRNTVYLQMNRLKVEDT |
| | | | AVYYCAAVHLLSNLRGADPHEADYWGQGTQVTVSS |
| | | | SEQ ID NO: 155 |
| | | | CDR1: GRTFSDYP - SEQ ID NO: 673/ |
| | | | CDR2: ISNGGTA - SEQ ID NO: 674/ |
| | | | CDR3: AAVHLLSNLRGADPHEADY - SEQ ID NO: 675 |
| 3CAMS55 | 20549 | 25 | QVQLQESGGGLVQAGGSLRLSCAASGRTFSTYPMAWFRXIPGKER |
| | | | EFVAGTSNGGSAYYADSVKGRFTISRDNTKNTVYLQMNSLKAEDT |
| | | | AVYYCAAVHLLSSLWGADPHEADYWGQGTQVTVSS |
| | | | SEQ ID NO: 156 |
| | | | CDR1: GRTFSTYP - SEQ ID NO: 676/ |
| | | | CDR2: TSNGGSA - SEQ ID NO: 677/ |
| | | | CDR3: AAVHLLSSLWGADPHEADY - SEQ ID NO: 678 |
| 2CAHS51 | 20392 | 26 | QVQLQESGGGLVQAGGSLRLSCAASGRTFSNYAMGWFRQAPGEER |
| | | | EFVAAINRSGTSLYHDSVMGRFTISRDNAKNTVALEMNSLKPEDT |
| | | | AVYYCVAGMPFSYSDYSRDSKYDYXGQGTQVTVSS |
| | | | SEQ ID NO: 157 |
| | | | CDR1: GRTFSNYA - SEQ ID NO: 679/ |
| | | | CDR2: INRSGTS - SEQ ID NO: 680/ |
| | | | CDR3: VAGMPFSYSDYSRDSKYDY - SEQ ID NO: 681 |
| 3CAMS63 | 20552 | 26 | QVQLQESGGGLVQAGGSLRLSCAASGRTFSNYAMGWFRQAPGKER |
| | | | EFVAAINRSGTTLYVDSVKGRFTISRDNAKNTVALQMNSLKPEDT |
| | | | AVYYCVAGMPFSYSDYPRDSKYDYWGQGTQVTVSS |
| | | | SEQ ID NO: 158 |
| | | | CDR1: GRTFSNYA - SEQ ID NO: 682/ |
| | | | CDR2: INRSGTT - SEQ ID NO: 683/ |
| | | | CDR3: VAGMPFSYSDYPRDSKYDY - SEQ ID NO: 684 |
| 3CAHM11 | 21050 | 26 | QVQLQESGGGLVQAGGSLRLSCAASGRTFNNYAMAWFRQAPGKER |
| | | | EFIAAITRSDTTLYIDSVKGRFTISRDNAKNTVALQMNSLKPEDT |
| | | | AVYYCAAGMPYSYSDYPRDSKYDYWGRGTQVTVSS |
| | | | SEQ ID NO: 159 |
| | | | CDR1: GRTFNNYA - SEQ ID NO: 685/ |
| | | | CDR2: ITRSDTT - SEQ ID NO: 686/ |
| | | | CDR3: AAGMPYSYSDYPRDSKYDY - SEQ ID NO: 687 |
| 2CAM47 | 20487 | 27 | QVQLQESGGGVVQEGASLRLSCAASGRNLSTYAMGWIRQAPGKER |
| | | | EFVAAISWAGSTTYYADSVKGRFIISRDNAKNTLNLQMNSLKPED |
| | | | TAVYYCAAPPYVTTTYYATPPSDIRDYHYWGQGTQVTVSS |
| | | | SEQ ID NO: 160 |
| | | | CDR1: GRNLSTYA - SEQ ID NO: 688/ |
| | | | CDR2: ISWAGSTT - SEQ ID NO: 689/ |
| | | | CDR3: AAPPYVTTTYYATPPSDIRDYHY - SEQ ID NO: 690 |
| 3CAM11 | 20508 | 27 | QVQLQESGGGLVQAGGSLRLSCAASGRNLSTYAMGWIRQAPGKER |
| | | | EFVAAISWAGSTTYYADSVKGRFIISRDNAKNTLNLQMNSLKPED |
| | | | TAVYYCAAPPYVTTTYYATPPSDIRDYHYWGQGTQVTVSS |
| | | | SEQ ID NO: 161 |
| | | | CDR1: GRNLSTYA - SEQ ID NO: 691/ |
| | | | CDR2: ISWAGSTT - |
| | | | SEQ ID NO: 692/ |
| | | | CDR3: AAPPYVTTTYYATPPSDIRDYHY - SEQ ID NO: 693 |
| 2CAM56 | 20490 | 28 | QVQLQESGGGEVQAGGSLRLSCVASGRTFSVYTTGWFRQAPGKER |
| | | | ELVAGMSGTRTYYADSVKGRFTISRDNAKNTVYLQMNSLKPDDTA |
| | | | VYYCAERRYGGPSLVWDEYDYRGQGTQVTVSS |
| | | | SEQ ID NO: 162 |
| | | | CDR1: GRTFSVYT - SEQ ID NO: 694/ |
| | | | CDR2: MSGTRT - SEQ ID NO: 695/ |
| | | | CDR3: AERRYGGPSLVWDEYDY - SEQ ID NO: 696 |
| 2CAM67 | 20494 | 28 | QVQLQESGGGEVQAGGSLRLSCVASGRTFSVYTTGWFRQAPGKER |
| | | | EFVAGMSGTRTYYADSVKGRFTISRDNAKNTVYLQMNSLKPDDTA |
| | | | VYYCAERRYGGPSLVWDEYDYRGQGTQVTVSS |
| | | | SEQ ID NO:163 |
| | | | CDR1: GRTFSVYT - SEQ ID NO: 697/ |
| | | | CDR2: MSGTRT - SEQ ID NO: 698/ |
| | | | CDR3: AERRYGGPSLVWDEYDY |
| | | | SEQ ID NO: 699 |
| 3CRH28 | 20380 | 29 | QVQLQESGGGLVQPGGSLTLSCAASGFTFSSNAMSWVRQAPGKGL |
| | | | EWVSSISSGGGTTYYTDSVKGRFTTARDNAKNTLYLRMNSLKPED |
| | | | TAVYYCSKSSTGSGSRGQGTQVTVSS |
| | | | SEQ ID NO: 164 |
| | | | CDR1: GFTFSSNA - SEQ ID NO: 700/ |
| | | | CDR2: ISSGGGTT - SEQ ID NO: 701/ |
| | | | CDR3: SKSSTGSGS - SEQ ID NO: 702 |
| 2CAMS80 | 20537 | 30 | QVQLQESGGGLVQTGASLRLSCAASGRTFSTYAMGWFRQAPGKER |
| | | | EFVAHISWSGGRTNYADSVKGRFTISRDNAKNTAYLQMNSLKPED |
| | | | TAVYYCAARIDDYDRGYDYWGQGTQVTVSS |
| | | | SEQ ID NO: 165 |
| | | | CDR1: GRTFSTYA - SEQ ID NO: 703/ |
| | | | CDR2: ISWSGGRT - SEQ ID NO: 704/ |
| | | | CDR3: AARIDDYDRGYDY - SEQ ID NO: 705 |
| 3CAMS14 | 20543 | 30 | QVQLQESGGGLVQPGGSLRLSCAASGRTFSTYAMGWFRQAPGKER |
| | | | EFVAHISWSGGRTYYADSVKGRFTISRDNAXNTAYLQMNSLKPED |
| | | | TAVYYCAARIDDYDRGYDYWGQGTQVTVSS |
| | | | SEQ ID NO: 166 |
| | | | CDR1: GRTFSTYA - SEQ ID NO: 706/ |
| | | | CDR2: ISWSGGRT - SEQ ID NO: 707/ |
| | | | CDR3: AARIDDYDRGYDY - SEQ ID NO: 708 |
| 3CRH83 | 20390 | 31 | QVQLQESGGGLVQAGGSLKLSCAASGNTFSINAMAWYRQAPGQEL |
| | | | ELVAAISDVGNTIYADSMKGRFTISRGNADNTVYLQMNSLKPEDT |
| | | | ATYYCNAGTFYTSPGDSYYRGYDFWGQGTQVTVSS |
| | | | SEQ ID NO: 167 |
| | | | CDR1: GNTFSINA - SEQ ID NO: 709/ |
| | | | CDR2: ISDVGNT - SEQ ID NO: 710/ |
| | | | CDR3: NAGTFYTSPGDSYYRGYDF - SEQ ID NO: 711 |
| 3CRM70 | 20462 | 31 | QVQLQESGGGLVQAGGSLKLSCAASGNTFSINAMAWYRQAPGQQL |
| | | | ELVAAISDVGNTIYADSMKGRFTISRGNADNTVYLQMNRLKPEDT |
| | | | ATYYCNAGTESTSXGDSYYRGYDFWGQGTQVTVSS |
| | | | SEQ ID NO: 168 |
| | | | CDR1: GNTFSINA - SEQ ID NO: 712/ |
| | | | CDR2: ISDVGN - SEQ ID NO: 713/ |
| | | | CDR3: NAGTFSTSXGDSYYRGYDF - SEQ ID NO: 714 |
| 3CRM23 | 20457 | 32 | QVQLQESGGGLVQAGGSLRLSCAASGSIRSINAMAMAWYRQAPGK |
| | | | QRELVASISNSGSIGYADSVKGRFTISRDNGKNSVYLQMNSLKPE |
| | | | DTAVYLONGRTGWPRYDLWGQGTQVTVSS |
| | | | SEQ ID NO: 169 |
| | | | CDR1: GSIRSINAMA - SEQ ID NO: 715/ |
| | | | CDR2: ISNSGSI - SEQ ID NO: 716/ |
| | | | CDR3: NGRTGWPRYDL - SEQ ID NO: 717 |
| 3CRMS13 | 20523 | 32 | QVQLQESGGGLVXAGGSLRLSCAASGSIRSINDMAWYRQAPGKQR |
| | | | ELVASISDSGSIDYADSVKGRFTISRDNGKSTVYLQMNSLKPEDT |
| | | | AIYLCNGRTGWPRYDLWGQGTQVTVSSX |
| | | | SEQ ID NO: 170 |
| | | | CDR1: GSIRSIND - SEQ ID NO: 718/ |
| | | | CDR2: ISDSGSI - SEQ ID NO: 719/ |
| | | | CDR3: NGRTGWPRYDL - SEQ ID NO: 720 |
| 3CAH28 | 20356 | 33 | QVQLQESGGGLVQSGASLRLSCAASERTLSGYAMGWFRQAPGKER |
| | | | EFVAAIRWSGSSTYYAESVEGRFTISRDNAKNTVYLQMNILKPED |
| | | | TAVYYCAAGSDYADEYDYWGQGTQVTVSS |
| | | | SEQ ID NO: 171 |
| | | | CDR1: ERTLSGYA - SEQ ID NO: 721/ |
| | | | CDR2: IRWSGSST - SEQ ID NO: 722/ |
| | | | CDR3: AAGSDYADEYDY - SEQ ID NO: 723 |
| 3CAH60 | 20361 | 34 | QVQLQESGGGLVQPGGSLILSCVASGFAMDYLTMGWSREAPRNER |
| | | | EDISCIDTSGRSTDYLDSVKGRFTISRDNAKNTLYLHMNDLKPED |
| | | | TAEYYCLASTSYCGAGYEYRGQGTQVTVSS |
| | | | SEQ ID NO:172 |
| | | | CDR1: GFAMDYLT - SEQ ID NO: 724/ |
| | | | CDR2: IDTSGRST - SEQ ID NO: 725/ |
| | | | CDR3: - LASTSYCGAGYEY |
| | | | SEQ ID NO: 726 |
| 2CRH62 | 20368 | 35 | QVQLQESGGGLVQPGGSLRLSCVESGRTFTVYDMGWFRQAPGKER |
| | | | EIVAAISADGATSIYQDSVKGRFTISRDNDAKNTVYLQMNSLKPE |
| | | | DTAVYYCAAGRSLIYNLYRANNTDYWGQGTQVTVSS |
| | | | SEQ ID NO: 173 |
| | | | CDR1: GRTFTVYD - SEQ ID NO: 727/ |
| | | | CDR2: ISADGATSI - SEQ ID NO: 728/ |
| | | | CDR3: AAGRSLIYNLYRANNTDY - SEQ ID NO: 729 |
| 2CRH76 | 20370 | 36 | QVQLQESGGGLVQTGGSLRLSCVASGRTFNWSAMGWFRQPPGKER |
| | | | EVVATISGGGTSAYYADSVKGRFTISRDGAKNTLYLQMNSLKPED |
| | | | TAVYYCAADRVDYRMTLTQNGNHYWGQGTQVTVSS |
| | | | SEQ ID NO: 174 |
| | | | CDR1: GRTFNWSA - SEQ ID NO: 730/ |
| | | | CDR2: ISGGGTSA - SEQ ID NO: 731/ |
| | | | CDR3: AADRVDYRMTLTQNGNHY - SEQ ID NO: 732 |
| 2CRH82 | 20372 | 37 | QVQLQESGGGLVQAGGSLRLSCAASARTESMYAMAWFRQAPGKER |
| | | | EIVAGIIQSGGTTYISDYMKGRFSIDRGNAENTVYLQMDSLKPED |
| | | | TAVYSCAAKVSSGTPSDIQISQSRWYDHWGQGTQVTVSS |
| | | | SEQ ID NO: 175 |
| | | | CDR1: ARTESMYA - SEQ ID NO: 733/ |
| | | | CDR2: IIQSGGTT - SEQ ID NO: 734/ |
| | | | CDR3: AAKVSSGTPSDIQISQSRWYDH - SEQ ID NO: 735 |
| 3CRH73 | 20389 | 38 | QVQLQESGGGLVRAGGSLRLSCAASGHTFSNYVMGWFRQAPGKER |
| | | | EFVAAISWGGSNTYYADSVKGRFTISRDNAKNTVYLQMNSLKVED |
| | | | TAVYYCAADRRFEGWNYYSMDYWGKGTQVTVSS |
| | | | SEQ ID NO:176 |
| | | | CDR1: GHTFSNYV - SEQ ID NO: 736/ |
| | | | CDR2: ISWGGSNT - SEQ ID NO: 737/ |
| | | | CDR3: AADRRFEGWNYYSMDY - |
| | | | SEQ ID NO: 738 |
| 3CRH93 | 20391 | 39 | QVQLQESGGGLVQAGGSLRLSCAASGRTVDSYAMGWFRQTPGKER |
| | | | EFVAASWGGGSTYYADSVKGRFTISRDNVKNTMYLQMDSLKPEDT |
| | | | AVYYCAAGPTYYSGPINDNWEGRYDSWGQGTQVTVSS |
| | | | SEQ ID NO: 177 |
| | | | CDR1: GRTVDSYA - SEQ ID NO: 739/ |
| | | | CDR2: SWGGGST - SEQ ID NO: 740/ |
| | | | CDR3: AAGPTYYSGPINDNWEGRYDS - |
| | | | SEQ ID NO: 741 |
| 2CRM28 | 20445 | 40 | QVQLQESGGGLVQPGGSLRLSCAASGRAWNEYNMGWFRQAPGKER |
| | | | EFVALIWWSSGNTYYADSVKGRFTISRDKVKNTVYLQMNSLKPED |
| | | | TAVYYCAAGPTFNKYEYWGQGTQVTVSS |
| | | | SEQ ID NO: 178 |
| | | | CDR1: GRAWNEYN - |
| | | | SEQ ID NO: 742/ |
| | | | CDR2: IWWSSGNT - SEQ ID NO: 743/ |
| | | | CDR3: AAGPTFNKYEY - SEQ ID NO: 744 |
| 3CRM46 | 20459 | 41 | QVQLQESGGGLVQPGGSLRLSCAASGFTFDSYAMSWVRQAPGKGL |
| | | | EWVSSIGSGGGSTAYTESVKGRFTISRDNAKNTLYLQMTSLKPED |
| | | | TAVYYCTRSRDSHTRPAGXGTQVTVSS |
| | | | SEQ ID NO: 179 |
| | | | CDR1: GFTFDSYA - |
| | | | SEQ ID NO: 745/ |
| | | | CDR2: IGSGGGST - SEQ ID NO: 746/ |
| | | | CDR3: TRSRDSHTRP - |
| | | | SEQ ID NO: 747 |
| 3CRM66 | 20461 | 42 | QVQLQESGGGLVQPGGSLRLSCAASGSTFSIHAMGWYRQAPGKKR |
| | | | ELVARITRGGSTNYADSVKGRFTISVDNAKNTVYLQMNSLKPEDT |
| | | | AVYDCNAETGWGEWDDYWGQGTQVTVSS |
| | | | SEQ ID NO: 180 |
| | | | CDR1: GSTFSIHA - SEQ ID NO: 748/ |
| | | | CDR2: ITRGGST - SEQ ID NO: 749/ |
| | | | CDR3: NAETGWGEWDDY - |
| | | | SEQ ID NO: 750 |
| 3CRM84 | 20463 | 43 | QVQLQESGGGWVQPGGSLRLSCAASGFTFGNSDMSWVRXAPGKGP |
| | | | EWVSTINTAGGSTYYADSVKGRFTISRDNAKNTLYLQMNSLKPED |
| | | | TAVYYCATALGSSWYSRFSGDLYEYDYWGQGTQVTVSS |
| | | | SEQ ID NO: 181 |
| | | | CDR1: GFTFGNSD - SEQ ID NO: 751/ |
| | | | CDR2: INTAGGST - SEQ ID NO: 752/ |
| | | | CDR3: ATALGSSWYSRFSGDLYEYDY - SEQ ID NO: 753 |
| 3CRM95 | 20465 | 44 | QVQLQESGGGLVQAGDFLRLSCVFSGGTFGDYSMGWFRQAPGKER |
| | | | EFVAAIRWSGDSTQYAEAVKGRFTISRDNAKSTVYLQMSSLKPED |
| | | | TAVYYCAADPSYYIGPNHTPSRYRDWGQGTQVTVSS |
| | | | SEQ ID NO: 182 |
| | | | CDR1: GGTFGDYS - SEQ ID NO: 754/ |
| | | | CDR2: IRWSGDST - SEQ ID NO: 755/ |
| | | | CDR3: AADPSYYIGPNHTPSRYRD - SEQ ID NO: 756 |
| 2CAM18 | 20475 | 45 | QVQLQESGGGLVQVGDSLKLSCVASGTTFSTHVMAWYRQAPGKQR |
| | | | EWVASIDSAGTTNYKDAVKGRFTISRDNVKRTVYLQMNNLKPEDS |
| | | | AVYLCSIPSRNAWGQGTQVTVSSX |
| | | | SEQ ID NO: 183 |
| | | | CDR1: GTTFSTHV - SEQ ID NO:757/ |
| | | | CDR2: IDSAGTT - SEQ ID NO: 758/ |
| | | | CDR3: SIPSRNA - SEQ ID NO: 759 |
| 2CAM39 | 20482 | 46 | QVQLQESGGGLVQAGGSLRLSCAASGRTFSTYAMGWFRQAPGKER |
| | | | EFVAAISWSGGSTYYADSVKGRFTISRDNAKNTGYLQMNSLKPED |
| | | | TAVYVCAADAEDPILYGSSWNNRRKEYDYWGQGTQVTVSS |
| | | | SEQ ID NO: 184 |
| | | | CDR1: GRTFSTYA - SEQ ID NO: 760/ |
| | | | CDR2: ISWSGGST - SEQ ID NO: 761/ |
| | | | CDR3: AADAEDPILYGSSWNNRRKEYDY - SEQ ID NO: 762 |
| 2CAM45 | 20485 | 47 | QVQLQESGGGLVQAGGSLRLSCAASGFTFDDYAIGWFRQAPGKER |
| | | | EGISCISSSDYGTYYAGSVKGRFTISSDNAKNTVYLQMNSLKPED |
| | | | TAVYYCAAEAISEYDCLGINYGMDYWGKGTQVTVSS |
| | | | SEQ ID NO: 185 |
| | | | CDR1: GFTFDDYA - SEQ ID NO:763/ |
| | | | CDR2: ISSSDYGT - SEQ ID NO:764/ |
| | | | CDR3: AAEAISEYDCLGINYGMDY - SEQ ID NO: 765 |
| 3CAM1 | 20503 | 48 | QVQLQESGGGLVQAGGSLRLSCAASGRTFGSHRMGWFRQAPGQER |
| | | | EFVAAIAWRGGTQYYTDSVKGRFTISRDNANNTVYLQMNSLKPED |
| | | | AAVYYCVAGEGSSWGDITDPRRYVFWGQGTQVTVSS |
| | | | SEQ ID NO: 186 |
| | | | CDR1: GRTFGSHR - SEQ ID NO: 766/ |
| | | | CDR2: IAWRGGTQ - SEQ ID NO: 767/ |
| | | | CDR3: VAGEGSSWGDITDPRRYVF - SEQ ID NO: 768 |
| 3CAM65 | 20513 | 49 | QVQLQESGGGLVQAGGSLRLSCAPSGRTFTTYVMGWFRQAPGKER |
| | | | EFVAHISWSGGRTYYADSVKGRFTISRDNAKNTSYLQMNSLKPED |
| | | | TAVYYCAAARSDAVRIGYAEGYEYWGQGTQVTVSS |
| | | | SEQ ID NO: 187 |
| | | | CDR1: GRTFTTYV - SEQ ID NO: 769/ |
| | | | CDR2: ISWSGGRT - SEQ ID NO: 770/ |
| | | | CDR3: AAARSDAVRIGYAEGYEY - SEQ ID NO: 771 |
| 3CAHS153 | 20404 | 50 | QVQLQESGGGLVQAGGSLRLSCAASGRTFSSYAMGWFRQVPGKER |
| | | | DFVAAVSWGGLSTYYADSVEGRFTISRDNAKNTVFLQMNSLKPED |
| | | | TAVYYCAADRAYEGLGYGEYEYWGQGTQVTVSS |
| | | | SEQ ID NO:188 |
| | | | CDR1: GRTFSSYA - SEQ ID NO: 772/ |
| | | | CDR2: VSWGGLST - SEQ ID NO: 773/ |
| | | | CDR3: AADRAYEGLGYGEYEY - SEQ ID NO: 774 |
| 3CRHS133 | 20433 | 51 | QVQLQESGGGLVXAGGSLRLSCAASGSTFSTYGMGWFRQAPGNER |
| | | | DFVAGITLIGEESLYAASVKGRFTISRDNAKNTVYLQMNSLKPED |
| | | | TAVYYCAADSYIKASGWSPRYWGQGTQVTVSS |
| | | | SEQ ID NO: 189 |
| | | | CDR1: GSTFSTYG - SEQ ID NO: 775/ |
| | | | CDR2: ITLIGEES - SEQ ID NO: 776/ |
| | | | CDR3: AADSYIKASGWSPRY - SEQ ID NO: 777 |
| 2CRMS29 | 20521 | 52 | QVQLQESGGGLVQAGGPLRLSCAASGRTFSGSAMGWFRQAPGKER |
| | | | EFVAAITWVGGFTYYADSVKGRFTISRDNAKNTVYLQMNNLSPED |
| | | | TAVYSCAAQAYHKSGYYYPPLYDYWGQGTQVTVSS |
| | | | SEQ ID NO: 190 |
| | | | CDR1: GRTFSGSA - SEQ ID NO: 778/ |
| | | | CDR2: ITWVGGFT - SEQ ID NO: 779/ |
| | | | CDR3: AAQAYHKSGYYYPPLYDY - SEQ ID NO: 780 |
| 2CAMS15 | 20531 | 53 | QVQLQESGGGLVQAGGSLRLSCAPSGRTFSTYAMGWFRQAPGKER |
| | | | EFVAGISWSGGRINYADSVKGRFTISRDNAKNTAYLQMNSLKPED |
| | | | TAVYYCAARDSDYGGQEYDYWGQGTQVTVSS |
| | | | SEQ ID NO: 191 |
| | | | CDR1: GRTFSTYA - SEQ ID NO: 781/ |
| | | | CDR2: ISWSGGRI - SEQ ID NO: 782/ |
| | | | CDR3: AARDSDYGGQEYDY - SEQ ID NO: 783 |
| 2CAMS29 | 20534 | 54 | QVQLQESGGGLVQAGGSLRLSCAASGLTSSTYVIGWFRQAPGKER |
| | | | EFVAAIMLSGGTTIYADSLKGRCTIYKDNAKNTVYLQMNSLKLED |
| | | | TAVYYCASGLIYRYSDFPRSQKYNFWGQGTQVTVSS |
| | | | SEQ ID NO: 192 |
| | | | CDR1: GLTSSTYV - SEQ ID NO: 784/ |
| | | | CDR2: IMLSGGTT - SEQ ID NO: 785/ |
| | | | CDR3: ASGLIYRYSDFPRSQKYNF - SEQ ID NO: 786 |
| 2CAMS52 | 20535 | 55 | QVQLQESGGGLVQPGASLRLSCATSGPTLSTYAMGWFRQAPGKER |
| | | | EVVATISWSGGRVTYVDSVKGRFTISRDNAKNTAYLQMNRLRPDD |
| | | | TAVYTCAARESDYGAYDYWGQGTQVTVSS |
| | | | SEQ ID NO: 193 |
| | | | CDR1: GPTLSTYA - SEQ ID NO: 787/ |
| | | | CDR2: ISWSGGRV - SEQ ID NO: 788/ |
| | | | CDR3: AARESDYGAYDY - SEQ ID NO: 789 |
| 3CAH34 | 20358 | 56 | QVQLQESGGGSVQPGGSLRLSCGASGFTFSSFGMHWFRQAPGKGQ |
| | | | EWAATISSSGGTTYYSDSVKGRFTISRDNAKNTLYLQMNSLKPED |
| | | | TALYYCARDVTGAVRGQGTQVTVSS |
| | | | SEQ ID NO: 194 |
| | | | CDR1: GFTFSSFG - SEQ ID NO: 790/ |
| | | | CDR2: ISSSGGTT - SEQ ID NO: 791/ |
| | | | CDR3: ARDVTGAV - SEQ ID NO: 792 |
| 3CAHS172 | 20408 | 56 | QVQLQESGGGSVQAGGSLRLSCGASGFTFSSFGMHWFRQAPGKGQ |
| | | | EWVATISSSGGTTYYSDSVKGRFTISRDNAKNTLYLQMNSLKPED |
| | | | TALYYCARDVTGAVRGQGTQVTVSS |
| | | | SEQ ID NO: 195 |
| | | | CDR1: GFTFSSFG - SEQ ID NO: 793/ |
| | | | CDR2: ISSSGGTT - SEQ ID NO: 794/ |
| | | | CDR3: ARDVTGAV - SEQ ID NO: 795 |
| 3CRHM14 | 21053 | 57 | QVQLQESGGGLVQAGGSLRLSCAASGRTFSNYAMAWFRQAPGTER |
| | | | EFVAGLSGSGGDTEFYDDSVKGRFAISRDSAKNTVWLQMNSLKPE |
| | | | DTAVYYCAAATHVVVHYGLVRRRGTYDYWGQGTQVTVSS |
| | | | SEQ ID NO:196 |
| | | | CDR1: GRTFSNYA - SEQ ID NO: 796/ |
| | | | CDR2: LSGSGGDTE - SEQ ID NO: 797/ |
| | | | CDR3: AAATHVVVHYGLVRRRGTYDY - SEQ ID NO: 798 |
| 3CRHM18 | 21054 | 57 | QVQLQESGGGLVQDGGSLRLSCAASGRTFSNYAMAWFRQAPGTER |
| | | | EFVAGLSGSGGDTAFYDNSVMGRFTISRDSAKNTMWLQINNLKPE |
| | | | DTAVYYCAAATHVVVSYGLVRKRGTYDYWGQGTQVTVSS |
| | | | SEQ ID NO: 197 |
| | | | CDR1: GRTFSNYA - SEQ ID NO: 799/ |
| | | | CDR2: LSGSGGDTA - SEQ ID NO: 800/ |
| | | | CDR3: AAATHVVVSYGLVRKRGTYDY - SEQ ID NO: 801 |
| 3CRHM35 | 21057 | 57 | QVQLQESGGGLVQAGGSLRLSCAASGRTFSNYAMAWFRQAPGTER |
| | | | EFVAGLSGSGGDTAFYDNSVMGRFTISRDSAKNTMWLQINNLKPE |
| | | | DTAVYYCAAATHVVVNYGLVRRRGTYDYWGQGTQVTVSS |
| | | | SEQ ID NO: 198 |
| | | | CDR1: GRTFSNYA - SEQ ID NO: 802/ |
| | | | CDR2: LSGSGGDTA - SEQ ID NO: 803/ |
| | | | CDR3: AAATHVVVNYGLVRRRGTYDY - SEQ ID NO: 804 |
| 3CRHM49 | 21058 | 57 | QVQLQESGGGLVQAGGSLRLSCAASGRTFSNYAMAWFRQAPGTER |
| | | | EFVAGLSGSGGDTAFYDNSVMGRFTISRDSAKNTMWLQINNLKPE |
| | | | DTAVYYCAAATHVVVSYGLVRRRGTYDYWGQGTQVTVSS |
| | | | SEQ ID NO: 199 |
| | | | CDR1: GRTFSNYA - SEQ ID NO:805/ |
| | | | CDR2: LSGSGGDTA - SEQ ID NO: 806/ |
| | | | CDR3: AAATHVVVNYGLVRRRGTYDY - SEQ ID NO: 807 |
| 3CRHM200 | 21064 | 57 | QVQLQESGGGLVQPGGSLRLSCAASGRTFSNYAMAWFRQAPGTER |
| | | | EFVAGLSGSGGDTAFYDNSVMGRFTISRDSAKNTMWLQINNLKPE |
| | | | DTAVYYCAAATHVVVSYGLVRRRGTYDYWGQGTQVTVSS |
| | | | SEQ ID NO: 200 |
| | | | CDR1: GRTFSNYA - SEQ ID NO: 808/ |
| | | | CDR2: LSGSGGDTA - SEQ ID NO: 809/ |
| | | | CDR3: AAATHVVVSYGLVRRRGTYDY - SEQ ID NO: 810 |
| 3CRHM85 | 21061 | 58 | QVQLQESGGGLVQPGGSLRLSCAASRRTFSKHAMAWFRQAPGKER |
| | | | EFVAVVRWSGIITYYADSVKGRFTISRDDAKNTVYLQMDSLKPED |
| | | | TAVYYCAAGDYRGNGYYVPGMYDYWGQGTQVTVSS |
| | | | SEQ ID NO: 201 |
| | | | CDR1: RRTFSKHA - SEQ ID NO: 811/ |
| | | | CDR2: VRWSGIIT - SEQ ID NO: 812/ |
| | | | CDR3: AAGDYRGNGYYVPGMYDY - SEQ ID NO: 813 |
| 3CRHM20 | 21055 | 59 | QVQLQESGGGLVQAGGSLRLSCAASGRTFSKSTMGWFRQTPGKER |
| | | | EFVAAITWTDTEVYANSVKDRFTISRDNAKNTFYLQMHSLKLEDT |
| | | | AVYYCAGDRYGSSRDSVFSSPTNYPYWGQGTQVTVSS |
| | | | SEQ ID NO: 202 |
| | | | CDR1: GRTFSKST - SEQ ID NO: 814/ |
| | | | CDR2: ITWTDTE - SEQ ID NO: 815/ |
| | | | CDR3: AGDRYGSSRDSVFSSPTNYPY - SEQ ID NO: 816 |
| 3CRHM33 | 21056 | 59 | QVQLQESGGGLVXAGGSLRLSCAASGRTFSTSTMAWFRQTPGKER |
| | | | EFVAAITGTDTEVYAHSVKGRFTISRDNAKNTFYLQMHSLKLEDT |
| | | | AVYYCAGDRYGSTRDSVFSSPTNYPYWGQGTQVTVSS |
| | | | SEQ ID NO: 203 |
| | | | CDR1: GRTFSTST - SEQ ID NO: 817/ |
| | | | CDR2: ITGTDTE - SEQ ID NO: 818/ |
| | | | CDR3: AGDRYGSTRDSVFSSPTNYPY |
| | | | SEQ ID NO: 819 |
| 3CRHM99 | 21062 | 60 | QVQLQESGGGLVQPGGSLRLSCAASGISSSRLHVGWFRQSPGKER |
| | | | EFVAAISLSGGTQAYTDSVKGRFTISGDNVKNTAYLQMNSLRPED |
| | | | TAVYYCAARERFIWTWSDLTYDYWGQGTQVTVSS |
| | | | SEQ ID NO: 204 |
| | | | CDR1: GISSSRLH - SEQ ID NO: 820/ |
| | | | CDR2: ISLSGGTQ - SEQ ID NO: 821/ |
| | | | CDR3: AARERFIWTWSDLTYDY - SEQ ID NO: 822 |
| 3CAHM40 | 21051 | 61 | QVQLQESGGGSVQAGGSLRLSCAVSGRTVSDYAMGWFRQAPGKER |
| | | | EFVAAMSSSETTVYPNSMKGRITISRDNAKNAVYLQLNALKPEDT |
| | | | AVYYCSMGPIATFQGLARQNEYHYWGQGTQVTVSS |
| | | | SEQ ID NO: 205 |
| | | | CDR1: GRTVSDYA - SEQ ID NO: 823/ |
| | | | CDR2: MSSSETT - SEQ ID NO: 824/ |
| | | | CDR3: SMGPIATFOGLARQNEYHY - |
| | | | SEQ ID NO: 825 |
| 3CRHM12 | 21052 | 61 | QVQLQESGGGSVQAGGSLRLSCAVSGRTVSDYAMGWFRQAPGKER |
| | | | EFVAAMSSSETTVYPNSVKGRITISRDNAKNAVYLQMNALKPEDT |
| | | | AVYYCSMGPIVTFOGLARQNEYHYWGQGTQVTVSS |
| | | | SEQ ID NO: 206 |
| | | | CDR1: GRTVSDYA - SEQ ID NO: 826/ |
| | | | CDR2: MSSSETT - SEQ ID NO: 827/ |
| | | | CDR3: SMGPIVTFQGLARQNEYHY - SEQ ID NO: 828 |
| 3CRHM133 | 21063 | 61 | QVQLQESGGGSVQAGGSLRLSCAVSGRPVSDYAMGWFRQAPGKER |
| | | | EFVAALSASDTTVYPNSVKGRITISRDNAKNAVDLQMNALKPEDT |
| | | | AVYYCAMGPIGTFQGLARQNEYHYWGQGTQVTVSS |
| | | | SEQ ID NO: 207 |
| | | | CDR1: GRPVSDYA - SEQ ID NO: 829/ |
| | | | CDR2: LSASDTT - SEQ ID NO: 830/ |
| | | | CDR3: AMGPIGTFQGLARQNEYHY - SEQ ID NO: 831 |
| 3CRHM240 | 21065 | 61 | QVQLQESGGGSVQAGGSLRLSCAVSGRTVSDYAMGWFRQAPGKER |
| | | | EFVVAMSSSETTVYTNSVKGRITISRDNAKNAVYLQMNALKPEDT |
| | | | AVYYCSMGPITTFQGLARQNEYHYWGQGTQVTVSS |
| | | | SEQ ID NO: 208 |
| | | | CDR1: GRTVSDYA - SEQ ID NO: 832/ |
| | | | CDR2: MSSSETT - SEQ ID NO: 833/ |
| | | | CDR3: SMGPITTFOGLARQNEYHY - SEQ ID NO: 834 |
| 3CRHM73 | 21060 | 62 | QVQLQESGGGLVQAGGSLSLSCAASGSAFRLNFMGWYRQAPGKQR |
| | | | ELVAAINSGGTEIYHDSVKGRFTISRDNADSTVWLQMNSLKPDDT |
| | | | AVYYCAADRGISWSKLAGGMDYWGKGTQVTVSS |
| | | | SEQ ID NO: 209 |
| | | | CDR1: GSAFRLNF - SEQ ID NO:835/ |
| | | | CDR2: INSGGTE - SEQ ID NO: 836/ |
| | | | CDR3: AADRGISWSKLAGGMDY - SEQ ID NO: 837 |

TABLE 2
Binding characteristics of anti-IL-12Rβ2 VHHs

| IL- | | | ELISA binding | | Off-rate constant ranking | | | Off-rate constant ranking | | |
|---|---|---|---|---|---|---|---|---|---|---|
| 12RB2 | SEQ | CDR3 | hIL- | mIL- | on human IL-12RB2 | | | on mouse IL-12RB2 | | |
| clone | ID | group | 12RB2/ | 12RB2/ | | k_{d} | k_{d} SE | | k_{d} | k_{d} SE |
| ID | NO: | ID | control | control | Response | (s⁻¹) | (s⁻¹) | Response | (s⁻¹) | (s⁻¹) |
| 20377 | 45 | 3 | 5.8352 | 1.8046 | −0.1904 | N/A | N/A | N/A | N/A | N/A |
| 20379 | 46 | 3 | 10.1835 | 1.4364 | 0.138 | 2.64E− | 4.52E−05 | N/A | N/A | N/A |
| | | | | | | 03 | | | | |
| 20388 | 47 | 3 | 4.6612 | 1.0267 | 0.4256 | 4.44E− | 7.20E−05 | N/A | N/A | N/A |
| | | | | | | 03 | | | | |
| 20414 | 48 | 3 | 6.3872 | 1.0478 | 0.3266 | 2.25E− | 2.64E−05 | N/A | N/A | N/A |
| | | | | | | 03 | | | | |
| 20415 | 49 | 3 | 5.0769 | 1.0862 | 0.2219 | 3.40E− | 5.30E−05 | N/A | N/A | N/A |
| | | | | | | 03 | | | | |
| 20417 | 50 | 3 | 4.0680 | 1.2710 | 0.5677 | 4.13E− | 6.36E−05 | N/A | N/A | N/A |
| | | | | | | 03 | | | | |
| 20420 | 51 | 3 | 7.9327 | 0.8310 | 0.0757 | N/A | N/A | N/A | N/A | N/A |
| 20422 | 52 | 3 | 7.8473 | 1.2582 | 0.2035 | 2.72E− | 3.61E−05 | N/A | N/A | N/A |
| | | | | | | 03 | | | | |
| 20423 | 53 | 3 | 3.7556 | 1.1139 | 0.2043 | 2.96E− | 4.12E−05 | N/A | N/A | N/A |
| | | | | | | 03 | | | | |
| 20426 | 54 | 3 | 5.0313 | 1.0627 | 0.1146 | 3.11E− | 4.89E−05 | N/A | N/A | N/A |
| | | | | | | 03 | | | | |
| 20428 | 55 | 3 | 5.8810 | 1.0547 | 0.2091 | 3.22E− | 4.98E−05 | N/A | N/A | N/A |
| | | | | | | 03 | | | | |
| 20436 | 56 | 3 | 3.3647 | 0.7403 | 0.0184 | N/A | N/A | N/A | N/A | N/A |
| 20440 | 57 | 3 | 5.1779 | 1.3355 | 0.3133 | 3.88E− | 6.21E−05 | N/A | N/A | N/A |
| | | | | | | 03 | | | | |
| 21059 | 58 | 3 | 5.61688312 | 7.935876623 | 1.4762 | 4.57E− | 4.86E−05 | 0.9256 | 6.83E− | 7.50E−05 |
| | | | | | | 03 | | | 03 | |
| 20363 | 59 | 4 | 6.4440 | 0.9705 | 0.6207 | 4.91E− | 7.23E−05 | N/A | N/A | N/A |
| | | | | | | 03 | | | | |
| 20367 | 60 | 4 | 5.3064 | 0.8662 | 0.6606 | 5.33E− | 8.03E−05 | N/A | N/A | N/A |
| | | | | | | 03 | | | | |
| 20371 | 61 | 4 | 5.0143 | 1.2297 | 0.53 | 3.53E− | 5.36E−05 | N/A | N/A | N/A |
| | | | | | | 03 | | | | |
| 20374 | 62 | 4 | 5.5076 | 1.1890 | 0.4381 | 3.28E− | 4.16E−05 | N/A | N/A | N/A |
| | | | | | | 03 | | | | |
| 20375 | 63 | 4 | 4.8188 | 1.7664 | 0.6085 | 4.09E− | 4.20E−05 | N/A | N/A | N/A |
| | | | | | | 03 | | | | |
| 20382 | 64 | 4 | 4.7506 | 0.8282 | 0.2961 | 2.43E− | 3.09E−05 | N/A | N/A | N/A |
| | | | | | | 03 | | | | |
| 20383 | 65 | 4 | 4.5841 | 1.3929 | 0.4269 | 3.61E− | 4.41E−05 | N/A | N/A | N/A |
| | | | | | | 03 | | | | |
| 20412 | 66 | 4 | 6.7434 | 1.0148 | 0.3005 | 2.83E− | 3.03E−05 | N/A | N/A | N/A |
| | | | | | | 03 | | | | |
| 20430 | 67 | 4 | 3.1772 | 1.0731 | 0.745 | 3.74E− | 3.20E−02 | N/A | N/A | N/A |
| | | | | | | 02 | | | | |
| 20432 | 68 | 4 | 3.6535 | 0.9840 | 0.2945 | 2.74E− | 2.61E−05 | N/A | N/A | N/A |
| | | | | | | 03 | | | | |
| 20362 | 69 | 5 | 6.2154 | 0.8859 | 0.4965 | 5.24E− | 7.75E−05 | N/A | N/A | N/A |
| | | | | | | 03 | | | | |
| 20366 | 70 | 5 | 5.5091 | 0.9673 | 0.6524 | 5.98E− | 9.39E−05 | N/A | N/A | N/A |
| | | | | | | 03 | | | | |
| 20369 | 71 | 5 | 4.3683 | 0.6056 | 0.5473 | 5.36E− | 8.20E−05 | N/A | N/A | N/A |
| | | | | | | 03 | | | | |
| 20373 | 72 | 5 | 8.6372 | 0.8076 | 0.4911 | 3.37E− | 4.57E−05 | N/A | N/A | N/A |
| | | | | | | 03 | | | | |
| 20376 | 73 | 5 | 9.7967 | 2.8484 | 0.5297 | 4.46E− | 7.46E−05 | N/A | N/A | N/A |
| | | | | | | 03 | | | | |
| 20381 | 74 | 5 | 11.7982 | 1.8528 | 0.475 | 4.13E− | 6.43E−05 | N/A | N/A | N/A |
| | | | | | | 03 | | | | |
| 20387 | 75 | 5 | 10.0298 | 2.1143 | 0.6168 | 4.95E− | 8.01E−05 | N/A | N/A | N/A |
| | | | | | | 03 | | | | |
| 20413 | 76 | 5 | 5.5935 | 1.0081 | 0.5003 | 4.04E− | 6.48E−05 | N/A | N/A | N/A |
| | | | | | | 03 | | | | |
| 20441 | 77 | 5 | 4.1670 | 1.0635 | 0.515 | 3.85E− | 5.62E−05 | N/A | N/A | N/A |
| | | | | | | 03 | | | | |
| 20393 | 98 | 9 | 4.7324 | 1.1381 | 0.6121 | 5.08E− | 7.67E−05 | N/A | N/A | N/A |
| | | | | | | 03 | | | | |
| 20395 | 99 | 9 | 8.2259 | 1.3098 | 0.6048 | 4.85E− | 7.61E−05 | N/A | N/A | N/A |
| | | | | | | 03 | | | | |
| 20396 | 100 | 9 | 10.0463 | 1.2165 | 0.5079 | 4.22E− | 6.46E−05 | N/A | N/A | N/A |
| | | | | | | 03 | | | | |
| 20397 | 101 | 9 | 6.5759 | 1.1013 | 0.4699 | 4.79E− | 7.35E−05 | N/A | N/A | N/A |
| | | | | | | 03 | | | | |
| 20400 | 102 | 9 | 5.6730 | 1.0024 | 0.2928 | 3.24E− | 3.74E−05 | N/A | N/A | N/A |
| | | | | | | 03 | | | | |
| 20403 | 103 | 9 | 4.5369 | 0.9877 | 0.3097 | 3.69E− | 5.17E−05 | N/A | N/A | N/A |
| | | | | | | 03 | | | | |
| 20354 | 109 | 11 | 3.8378 | 1.0471 | 0.482 | 4.94E− | 7.62E−05 | N/A | N/A | N/A |
| | | | | | | 03 | | | | |
| 20360 | 110 | 11 | 9.5609 | 1.1069 | 0.7592 | 4.43E− | 7.65E−05 | N/A | N/A | N/A |
| | | | | | | 03 | | | | |
| 20401 | 111 | 11 | 4.9800 | 1.0711 | 0.3518 | 3.24E− | 4.68E−05 | N/A | N/A | N/A |
| | | | | | | 03 | | | | |
| 20405 | 112 | 11 | 5.9543 | 0.9622 | 0.4941 | 3.31E− | 4.93E−05 | N/A | N/A | N/A |
| | | | | | | 03 | | | | |
| 20406 | 113 | 11 | 5.8666 | 0.9445 | 0.5147 | 3.79E− | 5.83E−05 | N/A | N/A | N/A |
| | | | | | | 03 | | | | |
| 20411 | 121 | 13 | 12.4911 | 1.0731 | 0.2794 | 3.11E− | 3.60E−05 | N/A | N/A | N/A |
| | | | | | | 03 | | | | |
| 20424 | 122 | 13 | 18.2707 | 1.1876 | 0.4162 | 4.43E− | 6.30E−05 | N/A | N/A | N/A |
| | | | | | | 03 | | | | |
| 20434 | 123 | 13 | 23.6784 | 0.9986 | 0.2047 | 2.75E− | 3.67E−05 | N/A | N/A | N/A |
| | | | | | | 03 | | | | |
| 20437 | 124 | 13 | 24.5167 | 1.0279 | 0.2822 | 2.97E− | 3.50E−05 | N/A | N/A | N/A |
| | | | | | | 03 | | | | |
| 20439 | 125 | 13 | 23.1873 | 1.0568 | 0.1491 | 2.47E− | 2.88E−05 | N/A | N/A | N/A |
| | | | | | | 03 | | | | |
| 20365 | 126 | 14 | 3.8040 | 1.6422 | 0.4928 | 4.96E− | 7.84E−05 | N/A | N/A | N/A |
| | | | | | | 03 | | | | |
| 20385 | 127 | 14 | 15.8810 | 1.8948 | 0.4874 | 4.53E− | 7.31E−05 | N/A | N/A | N/A |
| | | | | | | 03 | | | | |
| 20438 | 128 | 14 | 3.5752 | 0.9273 | 0.3385 | 3.02E− | 4.82E−05 | N/A | N/A | N/A |
| | | | | | | 03 | | | | |
| 20419 | 129 | 15 | 5.7347 | 1.1225 | 0.1869 | 3.43E− | 5.31E−05 | N/A | N/A | N/A |
| | | | | | | 03 | | | | |
| 20427 | 130 | 15 | 3.1275 | 0.9076 | 0.2589 | 4.05E− | 6.86E−05 | N/A | N/A | N/A |
| | | | | | | 03 | | | | |
| 20429 | 131 | 15 | 10.1809 | 1.0294 | 0.2975 | 3.55E− | 5.84E−05 | N/A | N/A | N/A |
| | | | | | | 03 | | | | |
| 20378 | 132 | 16 | 5.1638 | 1.0401 | 0.523 | 2.05E− | 2.50E−05 | N/A | N/A | N/A |
| | | | | | | 03 | | | | |
| 20386 | 133 | 16 | 4.9408 | 1.4119 | 0.7256 | 4.28E− | 7.78E−05 | N/A | N/A | N/A |
| | | | | | | 03 | | | | |
| 20425 | 134 | 16 | 6.3683 | 1.0157 | 0.5509 | 4.26E− | 6.66E−05 | N/A | N/A | N/A |
| | | | | | | 03 | | | | |
| 20355 | 144 | 20 | 4.2784 | 3.0805 | 0.6696 | 4.71E− | 7.05E−05 | 1.674 | 9.09E− | 1.39E−04 |
| | | | | | | 03 | | | 03 | |
| 20357 | 145 | 20 | 4.3115 | 0.8415 | 0.6067 | 4.19E− | 6.04E−05 | N/A | N/A | N/A |
| | | | | | | 03 | | | | |
| 20399 | 146 | 20 | 3.5366 | 1.0193 | 0.3227 | 2.67E− | 3.30E−05 | N/A | N/A | N/A |
| | | | | | | 03 | | | | |
| 20398 | 147 | 21 | 3.7139 | 1.0360 | 0.2677 | 4.34E− | 7.28E−05 | N/A | N/A | N/A |
| | | | | | | 03 | | | | |
| 20407 | 148 | 21 | 3.2659 | 0.9607 | 0.1948 | 4.31E− | 6.96E−05 | N/A | N/A | N/A |
| | | | | | | 03 | | | | |
| 20418 | 149 | 22 | 10.9450 | 1.1606 | 0.2343 | 4.29E− | 6.92E−05 | N/A | N/A | N/A |
| | | | | | | 03 | | | | |
| 20421 | 150 | 22 | 11.9960 | 0.9464 | 0.3271 | 4.30E− | 6.44E−05 | N/A | N/A | N/A |
| | | | | | | 03 | | | | |
| 20394 | 151 | 23 | 8.7743 | 1.3452 | 0.6086 | 4.41E− | 6.74E−05 | N/A | N/A | N/A |
| | | | | | | 03 | | | | |
| 20410 | 152 | 23 | 4.7793 | 1.1894 | 0.3759 | 2.65E− | 3.08E−05 | N/A | N/A | N/A |
| | | | | | | 03 | | | | |
| 20392 | 157 | 26 | 2.8006 | 5.5334 | 0.5951 | 4.86E− | 7.76E−05 | 1.4505 | 7.29E− | 1.18E−04 |
| | | | | | | 03 | | | 03 | |
| 21050 | 159 | 26 | 3.04235463 | 38.78822685 | 1.3968 | 5.19E− | 6.82E−05 | 1.0262 | 4.62E− | 5.29E−05 |
| | | | | | | 03 | | | 03 | |
| 20380 | 164 | 29 | 3.6420 | 0.9746 | 0.4004 | 4.28E− | 4.83E−05 | N/A | N/A | N/A |
| | | | | | | 03 | | | | |
| 20390 | 167 | 31 | 8.4631 | 13.9567 | 0.6934 | 5.28E− | 8.70E−05 | 1.3473 | 7.22E− | 1.37E−04 |
| | | | | | | 03 | | | 03 | |
| 20356 | 171 | 33 | 7.5656 | 0.3998 | 0.5756 | 4.83E− | 7.64E−05 | N/A | N/A | N/A |
| | | | | | | 03 | | | | |
| 20361 | 172 | 34 | 3.9076 | 1.8965 | 0.5903 | 5.17E− | 8.30E−05 | N/A | N/A | N/A |
| | | | | | | 03 | | | | |
| 20368 | 173 | 35 | 3.5139 | 0.8679 | 0.6427 | 6.22E− | 7.57E−05 | N/A | N/A | N/A |
| | | | | | | 03 | | | | |
| 20370 | 174 | 36 | 4.5738 | 0.9372 | 0.4791 | 3.54E− | 5.71E−05 | N/A | N/A | N/A |
| | | | | | | 03 | | | | |
| 20372 | 175 | 37 | 16.1286 | 1.7731 | 0.4264 | 2.74E− | 3.73E−05 | N/A | N/A | N/A |
| | | | | | | 03 | | | | |
| 20389 | 176 | 38 | 6.4494 | 1.1839 | 0.5649 | 5.20E− | 8.45E−05 | N/A | N/A | N/A |
| | | | | | | 03 | | | | |
| 20391 | 177 | 39 | 3.4475 | 1.3844 | 0.4265 | 4.86E− | 7.36E−05 | N/A | N/A | N/A |
| | | | | | | 03 | | | | |
| 20404 | 188 | 50 | 3.3166 | 1.0764 | 0.1634 | 3.02E− | 4.07E−05 | N/A | N/A | N/A |
| | | | | | | 03 | | | | |
| 20433 | 189 | 51 | 5.2092 | 0.9324 | −0.0271 | N/A | N/A | N/A | N/A | N/A |
| 20358 | 194 | 56 | 5.0066 | 1.1189 | 0.6903 | 6.36E− | 1.05E−04 | N/A | N/A | N/A |
| | | | | | | 03 | | | | |
| 20408 | 195 | 56 | 3.4877 | 1.1088 | 0.3875 | 3.83E− | 5.59E−05 | N/A | N/A | N/A |
| | | | | | | 03 | | | | |
| 21053 | 196 | 57 | 3.10555556 | 25.11767677 | 1.1937 | 5.32E− | 6.81E−05 | 0.9568 | 4.83E− | 5.44E−05 |
| | | | | | | 03 | | | 03 | |
| 21054 | 197 | 57 | 3.61088211 | 44.96949711 | 1.142 | 5.48E− | 8.25E−05 | 0.6986 | 6.18E− | 8.58E−05 |
| | | | | | | 03 | | | 03 | |
| 21057 | 198 | 57 | 4.84248555 | 35.48482659 | 1.359 | 5.76E− | 7.08E−05 | 1.0391 | 5.35E− | 4.51E−05 |
| | | | | | | 03 | | | 03 | |
| 21058 | 199 | 57 | 3.20228137 | 41.15057034 | 1.5377 | 6.19E− | 8.28E−05 | 1.1531 | 5.83E− | 6.12E−05 |
| | | | | | | 03 | | | 03 | |
| 21064 | 200 | 57 | 3.57674752 | 5.370088606 | 1.3753 | 5.71E− | 7.92E−05 | 1.0074 | 5.46E− | 5.45E−05 |
| | | | | | | 03 | | | 03 | |
| 21061 | 201 | 58 | 3.51651584 | 8.004751131 | 1.316 | 3.43E− | 4.15E−05 | 0.8162 | 4.74E− | 5.16E−05 |
| | | | | | | 03 | | | 03 | |
| 21055 | 202 | 59 | 8.22853894 | 20.18476407 | 1.1786 | 4.86E− | 7.10E−05 | 0.5861 | 5.90E− | 7.08E−05 |
| | | | | | | 03 | | | 03 | |
| 21056 | 203 | 59 | 5.81620718 | 19.43859649 | 1.2583 | 4.27E− | 4.94E−05 | 0.7914 | 5.74E− | 7.99E−05 |
| | | | | | | 03 | | | 03 | |
| 21062 | 204 | 60 | 4.23619602 | 5.238039787 | 1.4457 | 6.34E− | 8.66E−05 | 0.8117 | 6.71E− | 9.34E−05 |
| | | | | | | 03 | | | 03 | |
| 21051 | 205 | 61 | 3.72582571 | 11.91245523 | 1.3176 | 5.78E− | 8.28E−05 | 0.8086 | 6.44E− | 7.24E−05 |
| | | | | | | 03 | | | 03 | |
| 21052 | 206 | 61 | 7.28119181 | 38.33798883 | 1.3237 | 5.47E− | 7.71E−05 | 0.8373 | 5.99E− | 6.17E−05 |
| | | | | | | 03 | | | 03 | |
| 21063 | 207 | 61 | 3.45797444 | 3.915184774 | 1.1256 | 3.89E− | 8.08E−05 | 0.97 | 6.67E− | 7.34E−05 |
| | | | | | | 03 | | | 03 | |
| 21065 | 208 | 61 | 3.77832462 | 5.718958269 | 1.4977 | 4.57E− | 6.41E−05 | 1.0529 | 4.83E− | 3.84E−05 |
| | | | | | | 03 | | | 03 | |
| 21060 | 209 | 62 | 6.89585731 | 26.27790564 | 1.4487 | 6.05E− | 1.10E−04 | 0.9443 | 6.05E− | 6.61E−05 |
| | | | | | | 03 | | | 03 | |
| 20469 | 1 | 1 | 1.04516807 | 22.00105042 | N/A | N/A | N/A | N/A | N/A | N/A |
| 20478 | 2 | 1 | 1.01683502 | 8.782267116 | N/A | N/A | N/A | N/A | N/A | N/A |
| 20480 | 3 | 1 | 0.97934596 | 18.0636833 | N/A | N/A | N/A | N/A | N/A | N/A |
| 20481 | 4 | 1 | 0.98347911 | 15.76287658 | N/A | N/A | N/A | N/A | N/A | N/A |
| 20488 | 5 | 1 | 0.97328244 | 12.90534351 | N/A | N/A | N/A | N/A | N/A | N/A |
| 20491 | 6 | 1 | 1.00321199 | 14.39293362 | N/A | N/A | N/A | N/A | N/A | N/A |
| 20492 | 7 | 1 | 0.96365768 | 15.49003517 | N/A | N/A | N/A | N/A | N/A | N/A |
| 20495 | 8 | 1 | 0.92402973 | 15.82824112 | N/A | N/A | N/A | N/A | N/A | N/A |
| 20497 | 9 | 1 | 0.93181818 | 13.4237013 | N/A | N/A | N/A | N/A | N/A | N/A |
| 20498 | 10 | 1 | 0.9938713 | 6.618998979 | N/A | N/A | N/A | N/A | N/A | N/A |
| 20499 | 11 | 1 | 0.98455598 | 10.40057915 | N/A | N/A | N/A | N/A | N/A | N/A |
| 20500 | 12 | 1 | 0.98206751 | 24.03902954 | N/A | N/A | N/A | N/A | N/A | N/A |
| 20502 | 13 | 1 | 1.02181818 | 14.42181818 | N/A | N/A | N/A | N/A | N/A | N/A |
| 20504 | 14 | 1 | 1.26729191 | 14.46189918 | N/A | N/A | N/A | N/A | N/A | N/A |
| 20505 | 15 | 1 | 1.06575015 | 20.18768679 | N/A | N/A | N/A | N/A | N/A | N/A |
| 20509 | 16 | 1 | 1.31343284 | 10.00995025 | N/A | N/A | N/A | N/A | N/A | N/A |
| 20510 | 17 | 1 | 1.19694534 | 16.44292605 | N/A | N/A | N/A | N/A | N/A | N/A |
| 20511 | 18 | 1 | 1.20242915 | 25.64473684 | N/A | N/A | N/A | N/A | N/A | N/A |
| 20514 | 19 | 1 | 1.03088578 | 17.83974359 | N/A | N/A | N/A | N/A | N/A | N/A |
| 20516 | 20 | 1 | 1.22914349 | 21.82536151 | N/A | N/A | N/A | N/A | N/A | N/A |
| 20536 | 21 | 1 | 0.84227297 | 3.377588954 | N/A | N/A | N/A | N/A | N/A | N/A |
| 20538 | 22 | 1 | 1.07364222 | 4.775084382 | N/A | N/A | N/A | N/A | N/A | N/A |
| 20539 | 23 | 1 | 1.03455818 | 4.886264217 | N/A | N/A | N/A | N/A | N/A | N/A |
| 20540 | 24 | 1 | 0.83380637 | 8.571901608 | N/A | N/A | N/A | N/A | N/A | N/A |
| 20541 | 25 | 1 | 0.91497741 | 5.660372987 | N/A | N/A | N/A | N/A | N/A | N/A |
| 20544 | 26 | 1 | 0.91931943 | 9.31064764 | N/A | N/A | N/A | N/A | N/A | N/A |
| 20547 | 27 | 1 | 1.01688139 | 5.121723678 | N/A | N/A | N/A | N/A | N/A | N/A |
| 20548 | 28 | 1 | 0.95096406 | 8.977386337 | N/A | N/A | N/A | N/A | N/A | N/A |
| 20550 | 29 | 1 | 0.95387542 | 21.20114123 | N/A | N/A | N/A | N/A | N/A | N/A |
| 20442 | 30 | 2 | 0.99285714 | 6.675 | N/A | N/A | N/A | N/A | N/A | N/A |
| 20444 | 31 | 2 | 0.98512586 | 4.684210526 | N/A | N/A | N/A | N/A | N/A | N/A |
| 20446 | 32 | 2 | 1.00967352 | 12.02176542 | N/A | N/A | N/A | N/A | N/A | N/A |
| 20454 | 33 | 2 | 0.99626401 | 5.04234122 | N/A | N/A | N/A | N/A | N/A | N/A |
| 20455 | 34 | 2 | 1.39805825 | 11.50364078 | N/A | N/A | N/A | N/A | N/A | N/A |
| 20456 | 35 | 2 | 1.24088398 | 12.13149171 | N/A | N/A | N/A | N/A | N/A | N/A |
| 20458 | 36 | 2 | 1.46722288 | 28.40882002 | N/A | N/A | N/A | N/A | N/A | N/A |
| 20464 | 37 | 2 | 1.28791469 | 5.436018957 | N/A | N/A | N/A | N/A | N/A | N/A |
| 20517 | 38 | 2 | 0.96842105 | 5.471578947 | N/A | N/A | N/A | N/A | N/A | N/A |
| 20518 | 39 | 2 | 1.014 | 4.431 | N/A | N/A | N/A | N/A | N/A | N/A |
| 20519 | 40 | 2 | 0.97291876 | 5.436308927 | N/A | N/A | N/A | N/A | N/A | N/A |
| 20522 | 41 | 2 | 1.00992282 | 5.209481808 | N/A | N/A | N/A | N/A | N/A | N/A |
| 20524 | 42 | 2 | 0.92957043 | 9.623876124 | N/A | N/A | N/A | N/A | N/A | N/A |
| 20525 | 43 | 2 | 0.73466003 | 5.868988391 | N/A | N/A | N/A | N/A | N/A | N/A |
| 20529 | 44 | 2 | 0.85766234 | 13.24519481 | N/A | N/A | N/A | N/A | N/A | N/A |
| 20447 | 78 | 6 | 1.40320856 | 11.75721925 | N/A | N/A | N/A | N/A | N/A | N/A |
| 20450 | 79 | 6 | 2.181532 | 17.97481637 | N/A | N/A | N/A | N/A | N/A | N/A |
| 20451 | 80 | 6 | 1.26490826 | 33.4896789 | N/A | N/A | N/A | N/A | N/A | N/A |
| 20452 | 81 | 6 | 0.9894535 | 7.515819751 | N/A | N/A | N/A | N/A | N/A | N/A |
| 20460 | 82 | 6 | 2.43291995 | 16.57384442 | N/A | N/A | N/A | N/A | N/A | N/A |
| 20527 | 83 | 6 | 0.83396843 | 9.932498639 | N/A | N/A | N/A | N/A | N/A | N/A |
| 20528 | 84 | 6 | 0.87739965 | 4.038394415 | N/A | N/A | N/A | N/A | N/A | N/A |
| 20466 | 85 | 7 | 1.03043968 | 8.483652762 | N/A | N/A | N/A | N/A | N/A | N/A |
| 20471 | 86 | 7 | 0.99144385 | 5.70802139 | N/A | N/A | N/A | N/A | N/A | N/A |
| 20532 | 88 | 7 | 0.81204906 | 3.80988456 | N/A | N/A | N/A | N/A | N/A | N/A |
| 20542 | 89 | 7 | 0.849018 | 4.275777414 | N/A | N/A | N/A | N/A | N/A | N/A |
| 20553 | 90 | 7 | 0.99012346 | 15.17901235 | N/A | N/A | N/A | N/A | N/A | N/A |
| 20554 | 91 | 7 | 1.0521251 | 17.0521251 | N/A | N/A | N/A | N/A | N/A | N/A |
| 20515 | 87 | 7 | 1.31122995 | 12.01069519 | N/A | N/A | N/A | N/A | N/A | N/A |
| 20474 | 92 | 8 | 1.01222222 | 6.434444444 | N/A | N/A | N/A | N/A | N/A | N/A |
| 20479 | 93 | 8 | 1.06973995 | 9.407801418 | N/A | N/A | N/A | N/A | N/A | N/A |
| 20483 | 94 | 8 | 1.00681818 | 4.676136364 | N/A | N/A | N/A | N/A | N/A | N/A |
| 20484 | 95 | 8 | 1.01172333 | 4.862837046 | N/A | N/A | N/A | N/A | N/A | N/A |
| 20496 | 96 | 8 | 1.01267281 | 19.54608295 | N/A | N/A | N/A | N/A | N/A | N/A |
| 20551 | 97 | 8 | 0.99837486 | 6.589382449 | N/A | N/A | N/A | N/A | N/A | N/A |
| 20468 | 104 | 10 | 0.95486726 | 9.24159292 | N/A | N/A | N/A | N/A | N/A | N/A |
| 20476 | 105 | 10 | 0.95571302 | 7.26129183 | N/A | N/A | N/A | N/A | N/A | N/A |
| 20493 | 106 | 10 | 1.05094787 | 14.51895735 | N/A | N/A | N/A | N/A | N/A | N/A |
| 20530 | 107 | 10 | 0.8471464 | 3.804466501 | N/A | N/A | N/A | N/A | N/A | N/A |
| 20533 | 108 | 10 | 0.99389624 | 3.957273652 | N/A | N/A | N/A | N/A | N/A | N/A |
| 20489 | 117 | 12 | 0.99546485 | 7.879818594 | N/A | N/A | N/A | N/A | N/A | N/A |
| 20467 | 114 | 12 | 1.03747323 | 14.24946467 | N/A | N/A | N/A | N/A | N/A | N/A |
| 20486 | 116 | 12 | 0.97311272 | 6.852119959 | N/A | N/A | N/A | N/A | N/A | N/A |
| 20512 | 119 | 12 | 1.30695444 | 47.38489209 | N/A | N/A | N/A | N/A | N/A | N/A |
| 20555 | 120 | 12 | 1.05729971 | 9.414136215 | N/A | N/A | N/A | N/A | N/A | N/A |
| 20477 | 115 | 12 | 1.01963048 | 20.89145497 | N/A | N/A | N/A | N/A | N/A | N/A |
| 20506 | 118 | 12 | 0.96958318 | 19.94630116 | N/A | N/A | N/A | N/A | N/A | N/A |
| 20473 | 135 | 17 | 0.97093649 | 36.32077503 | N/A | N/A | N/A | N/A | N/A | N/A |
| 20501 | 136 | 17 | 0.99773243 | 62.28684807 | N/A | N/A | N/A | N/A | N/A | N/A |
| 20546 | 137 | 17 | 0.96155872 | 3.29699842 | N/A | N/A | N/A | N/A | N/A | N/A |
| 20443 | 138 | 18 | 0.94671107 | 4.122398002 | N/A | N/A | N/A | N/A | N/A | N/A |
| 20449 | 139 | 18 | 0.9416126 | 6.092678406 | N/A | N/A | N/A | N/A | N/A | N/A |
| 20453 | 140 | 18 | 0.87344913 | 3.731182796 | N/A | N/A | N/A | N/A | N/A | N/A |
| 20470 | 141 | 19 | 1.09122807 | 8.267836257 | N/A | N/A | N/A | N/A | N/A | N/A |
| 20472 | 142 | 19 | 0.90007294 | 3.377826404 | N/A | N/A | N/A | N/A | N/A | N/A |
| 20545 | 143 | 19 | 1.10365059 | 20.136897 | N/A | N/A | N/A | N/A | N/A | N/A |
| 20448 | 153 | 24 | 0.99335233 | 5.864197531 | N/A | N/A | N/A | N/A | N/A | N/A |
| 20526 | 154 | 24 | 1.01657798 | 4.393164142 | N/A | N/A | N/A | N/A | N/A | N/A |
| 20507 | 155 | 25 | 1.13565099 | 40.8997955 | N/A | N/A | N/A | N/A | N/A | N/A |
| 20549 | 156 | 25 | 1.15866667 | 3.196 | N/A | N/A | N/A | N/A | N/A | N/A |
| 20552 | 158 | 26 | 1.01258327 | 9.783123612 | N/A | N/A | N/A | N/A | N/A | N/A |
| 20487 | 160 | 27 | 0.96853933 | 4.907865169 | N/A | N/A | N/A | N/A | N/A | N/A |
| 20508 | 161 | 27 | 1.31309524 | 6.595238095 | N/A | N/A | N/A | N/A | N/A | N/A |
| 20490 | 162 | 28 | 0.91787853 | 5.205303678 | N/A | N/A | N/A | N/A | N/A | N/A |
| 20494 | 163 | 28 | 0.87037037 | 7.743209877 | N/A | N/A | N/A | N/A | N/A | N/A |
| 20537 | 165 | 30 | 1.0588785 | 4.682242991 | N/A | N/A | N/A | N/A | N/A | N/A |
| 20543 | 166 | 30 | 0.95454545 | 5.593073593 | N/A | N/A | N/A | N/A | N/A | N/A |
| 20462 | 168 | 31 | 1.47449585 | 6.004744958 | N/A | N/A | N/A | N/A | N/A | N/A |
| 20457 | 169 | 32 | 0.96950531 | 5.666365485 | N/A | N/A | N/A | N/A | N/A | N/A |
| 20523 | 170 | 32 | 0.87307601 | 4.522140658 | N/A | N/A | N/A | N/A | N/A | N/A |
| 20445 | 178 | 40 | 1.00065274 | 13.47650131 | N/A | N/A | N/A | N/A | N/A | N/A |
| 20459 | 179 | 41 | 1.39109507 | 29.07821901 | N/A | N/A | N/A | N/A | N/A | N/A |
| 20461 | 180 | 42 | 1.37651332 | 12.72639225 | N/A | N/A | N/A | N/A | N/A | N/A |
| 20463 | 181 | 43 | 1.44044665 | 13.88337469 | N/A | N/A | N/A | N/A | N/A | N/A |
| 20465 | 182 | 44 | 1.41347053 | 6.715622077 | N/A | N/A | N/A | N/A | N/A | N/A |
| 20475 | 183 | 45 | 1.51266985 | 3.221079692 | N/A | N/A | N/A | N/A | N/A | N/A |
| 20482 | 184 | 46 | 1.00117233 | 8.018757327 | N/A | N/A | N/A | N/A | N/A | N/A |
| 20485 | 185 | 47 | 0.97371429 | 10.37257143 | N/A | N/A | N/A | N/A | N/A | N/A |
| 20503 | 186 | 48 | 1.20054201 | 3.304878049 | N/A | N/A | N/A | N/A | N/A | N/A |
| 20513 | 187 | 49 | 1.24615385 | 5.984615385 | N/A | N/A | N/A | N/A | N/A | N/A |
| 20521 | 190 | 52 | 1.04589788 | 5.849110729 | N/A | N/A | N/A | N/A | N/A | N/A |
| 20531 | 191 | 53 | 0.98110355 | 4.501133787 | N/A | N/A | N/A | N/A | N/A | N/A |
| 20534 | 192 | 54 | 1.0608339 | 6.244702666 | N/A | N/A | N/A | N/A | N/A | N/A |
| 20535 | 193 | 55 | 0.96878049 | 6.405853659 | N/A | N/A | N/A | N/A | N/A | N/A |

TABLE 3
Binding of anti-IL-12Rβ2 VHH clones to cells transiently expressing human or mouse IL-12Rβ2

| | | | | binding to human or mouse IL-12RB2-expressing cells | | | |
|---|---|---|---|---|---|---|---|
| | SEQ | | CDR3 | hIL- | hIL-12RB2 | mIL- | mIL-12RB2 |
| | ID | Clone | group | 12RB2 | % cells | 12RB2 | % cells |
| Clone name | NO: | ID | ID | dMFI | bound | dMFI | bound |
| 3CRH18 | 45 | 20377 | 3 | 918 | 41.7 | N/A | N/A |
| 3CRH27 | 46 | 20379 | 3 | 812 | 38.9 | N/A | N/A |
| 3CRH68 | 47 | 20388 | 3 | 787 | 38.2 | N/A | N/A |
| 3CRHS3 | 48 | 20414 | 3 | 1317 | 47.2 | N/A | N/A |
| 3CRHS9 | 49 | 20415 | 3 | 1041 | 42.1 | N/A | N/A |
| 3CRHS29 | 50 | 20417 | 3 | 829 | 37.2 | N/A | N/A |
| 3CRHS38 | 51 | 20420 | 3 | 1220 | 45.8 | N/A | N/A |
| 3CRHS54 | 52 | 20422 | 3 | 970 | 41.5 | N/A | N/A |
| 3CRHS55 | 53 | 20423 | 3 | 961 | 40.5 | N/A | N/A |
| 3CRHS90 | 54 | 20426 | 3 | 1188 | 45.8 | N/A | N/A |
| 3CRHS100 | 55 | 20428 | 3 | 1132 | 43.2 | N/A | N/A |
| 3CRHS148 | 56 | 20436 | 3 | 1031 | 42 | N/A | N/A |
| 3CRHS173 | 57 | 20440 | 3 | 1360 | 48.4 | N/A | N/A |
| 3CRHM61 | 58 | 21059 | 3 | 946 | 40.3 | 114 | 0.56 |
| 2CRH33 | 59 | 20363 | 4 | 1122 | 44.8 | N/A | N/A |
| 2CRH60 | 60 | 20367 | 4 | 1104 | 45.3 | N/A | N/A |
| 2CRH80 | 61 | 20371 | 4 | 809 | 40.1 | N/A | N/A |
| 3CRH14 | 62 | 20374 | 4 | 864 | 41.7 | N/A | N/A |
| 3CRH15 | 63 | 20375 | 4 | 764 | 38.1 | N/A | N/A |
| 3CRH43 | 64 | 20382 | 4 | 1156 | 45.5 | N/A | N/A |
| 3CRH45 | 65 | 20383 | 4 | 742 | 37.6 | N/A | N/A |
| 3CRHS1 | 66 | 20412 | 4 | 1084 | 44.5 | N/A | N/A |
| 3CRHS109 | 67 | 20430 | 4 | 1261 | 46.5 | N/A | N/A |
| 3CRHS128 | 68 | 20432 | 4 | 1481 | 50.5 | N/A | N/A |
| 2CRH10 | 69 | 20362 | 5 | 595 | 30.8 | N/A | N/A |
| 2CRH53 | 70 | 20366 | 5 | 686 | 35.3 | N/A | N/A |
| 2CRH73 | 71 | 20369 | 5 | 707 | 36.7 | N/A | N/A |
| 3CRH5 | 72 | 20373 | 5 | 835 | 39.5 | N/A | N/A |
| 3CRH17 | 73 | 20376 | 5 | 583 | 32.5 | N/A | N/A |
| 3CRH38 | 74 | 20381 | 5 | 537 | 31.8 | N/A | N/A |
| 3CRH67 | 75 | 20387 | 5 | 645 | 35.1 | N/A | N/A |
| 3CRHS2 | 76 | 20413 | 5 | 1146 | 44.5 | N/A | N/A |
| 3CRHS184 | 77 | 20441 | 5 | 625 | 33.3 | N/A | N/A |
| 3CAHS39 | 98 | 20393 | 9 | 918 | 39 | N/A | N/A |
| 3CAHS43 | 99 | 20395 | 9 | 1348 | 47.6 | N/A | N/A |
| 3CAHS52 | 100 | 20396 | 9 | 1108 | 44.3 | N/A | N/A |
| 3CAHS53 | 101 | 20397 | 9 | 1456 | 49.3 | N/A | N/A |
| 3CAHS124 | 102 | 20400 | 9 | 1195 | 46.3 | N/A | N/A |
| 3CAHS150 | 103 | 20403 | 9 | 1268 | 47.4 | N/A | N/A |
| 2CAH27 | 109 | 20354 | 11 | 668 | 36.7 | N/A | N/A |
| 3CAH48 | 110 | 20360 | 11 | 801 | 39.4 | N/A | N/A |
| 3CAHS142 | 111 | 20401 | 11 | 912 | 40.9 | N/A | N/A |
| 3CAHS154 | 112 | 20405 | 11 | 773 | 35.5 | N/A | N/A |
| 3CAHS170 | 113 | 20406 | 11 | 809 | 38.3 | N/A | N/A |
| 2CRHS4 | 121 | 20411 | 13 | 254 | 3.55 | N/A | N/A |
| 3CRHS67 | 122 | 20424 | 13 | 443 | 7.11 | N/A | N/A |
| 3CRHS142 | 123 | 20434 | 13 | 210 | 1.02 | N/A | N/A |
| 3CRHS156 | 124 | 20437 | 13 | 222 | 1.51 | N/A | N/A |
| 3CRHS166 | 125 | 20439 | 13 | 266 | 2.11 | N/A | N/A |
| 2CRH41 | 126 | 20365 | 14 | 583 | 31.6 | N/A | N/A |
| 3CRH59 | 127 | 20385 | 14 | 1170 | 46.1 | N/A | N/A |
| 3CRHS163 | 128 | 20438 | 14 | 1153 | 43.7 | N/A | N/A |
| 3CRHS35 | 129 | 20419 | 15 | 1195 | 45.7 | N/A | N/A |
| 3CRHS99 | 130 | 20427 | 15 | 980 | 42.1 | N/A | N/A |
| 3CRHS106 | 131 | 20429 | 15 | 1111 | 43.3 | N/A | N/A |
| 3CRH25 | 132 | 20378 | 16 | 620 | 34.8 | N/A | N/A |
| 3CRH64 | 133 | 20386 | 16 | 645 | 31 | N/A | N/A |
| 3CRHS77 | 134 | 20425 | 16 | 867 | 37.7 | N/A | N/A |
| 2CAH71 | 144 | 20355 | 20 | 566 | 35.3 | N/A | N/A |
| 3CAH32 | 145 | 20357 | 20 | 742 | 40 | N/A | N/A |
| 3CAHS102 | 146 | 20399 | 20 | 1094 | 44.7 | N/A | N/A |
| 3CAHS79 | 147 | 20398 | 21 | 1231 | 46.1 | N/A | N/A |
| 3CAHS171 | 148 | 20407 | 21 | 1283 | 47.5 | N/A | N/A |
| 3CRHS31 | 149 | 20418 | 22 | 977 | 42.8 | N/A | N/A |
| 3CRHS51 | 150 | 20421 | 22 | 1239 | 46 | N/A | N/A |
| 3CAHS41 | 151 | 20394 | 23 | 1071 | 43.3 | N/A | N/A |
| 3CAHS180 | 152 | 20410 | 23 | 1009 | 42.6 | N/A | N/A |
| 2CAHS51 | 157 | 20392 | 26 | 248 | 1.47 | N/A | N/A |
| 3CAHM11 | 159 | 21050 | 26 | 784 | 37.6 | 294 | 7.51 |
| 3CRH28 | 164 | 20380 | 29 | 1111 | 44.6 | N/A | N/A |
| 3CRH83 | 167 | 20390 | 31 | 602 | 28.3 | N/A | N/A |
| 3CAH28 | 171 | 20356 | 33 | 37 | 0.15 | N/A | N/A |
| 3CAH60 | 172 | 20361 | 34 | 18 | 0.1 | N/A | N/A |
| 2CRH62 | 173 | 20368 | 35 | 891 | 41.9 | N/A | N/A |
| 2CRH76 | 174 | 20370 | 36 | 668 | 36.6 | N/A | N/A |
| 2CRH82 | 175 | 20372 | 37 | 844 | 40.1 | N/A | N/A |
| 3CRH73 | 176 | 20389 | 38 | 918 | 41.2 | N/A | N/A |
| 3CRH93 | 177 | 20391 | 39 | 580 | 31.5 | N/A | N/A |
| 3CAHS153 | 188 | 20404 | 50 | 789 | 36.8 | N/A | N/A |
| 3CRHS133 | 189 | 20433 | 51 | 48 | 0.24 | N/A | N/A |
| 3CAH34 | 194 | 20358 | 56 | 620 | 34.6 | N/A | N/A |
| 3CAHS172 | 195 | 20408 | 56 | 1018 | 43 | N/A | N/A |
| 3CRHM14 | 196 | 21053 | 57 | 689 | 35.8 | 412 | 18.6 |
| 3CRHM18 | 197 | 21054 | 57 | 767 | 36.7 | 369 | 15.3 |
| 3CRHM35 | 198 | 21057 | 57 | 826 | 37.3 | 425 | 16 |
| 3CRHM49 | 199 | 21058 | 57 | 759 | 36 | 421 | 17.1 |
| 3CRHM200 | 200 | 21064 | 57 | 694 | 35.3 | 470 | 17.2 |
| 3CRHM85 | 201 | 21061 | 58 | 676 | 31.2 | 157 | 0.74 |
| 3CRHM20 | 202 | 21055 | 59 | 556 | 28.7 | 86 | 0.18 |
| 3CRHM33 | 203 | 21056 | 59 | 721 | 36 | 86 | 0.21 |
| 3CRHM99 | 204 | 21062 | 60 | 558 | 24.4 | 351 | 10.7 |
| 3CAHM40 | 205 | 21051 | 61 | 544 | 32.7 | 105 | 1.02 |
| 3CRHM12 | 206 | 21052 | 61 | 495 | 30.4 | 103 | 1.21 |
| 3CRHM133 | 207 | 21063 | 61 | 721 | 33.7 | 133 | 0.59 |
| 3CRHM240 | 208 | 21065 | 61 | 635 | 33.7 | 43 | 0.33 |
| 3CRHM73 | 209 | 21060 | 62 | 397 | 19.5 | 357 | 10.4 |

TABLE 4
Binding of anti-IL-12Rβ2 VHH clones to
cells expressing mouse IL-12Rβ2

| | | | | binding to mouse IL-12RB2- | |
|---|---|---|---|---|---|
| | SEQ | | CDR3 | expressing cells | |
| Clone | ID | Clone | group | mIL-12RB2 | mIL-12RB2% |
| name | NO: | ID | ID | dMFI | cells bound |
| 3CRH83 | 167 | 20390 | 31 | 21 | 3.47 |
| 2CRM28 | 178 | 20445 | 40 | 100 | 19.5 |
| 2CRM40 | 153 | 20448 | 24 | 166 | 32.8 |
| 2CRM42 | 139 | 20449 | 18 | 22 | 2.93 |
| 2CRM83 | 80 | 20451 | 6 | 122 | 24.9 |
| 3CRM23 | 169 | 20457 | 32 | 53 | 7.71 |
| 3CRM45 | 36 | 20458 | 2 | 110 | 18.3 |
| 3CRM46 | 179 | 20459 | 41 | 39 | 4.06 |
| 3CRM66 | 180 | 20461 | 42 | 35 | 4.91 |
| 3CRM84 | 181 | 20463 | 43 | 27 | 0.96 |
| 3CRM95 | 182 | 20465 | 44 | 51 | 6.74 |
| 2CAM18 | 183 | 20475 | 45 | 13.6 | 3.19 |
| 2CAM39 | 184 | 20482 | 46 | 255 | 48.7 |
| 2CAM45 | 185 | 20485 | 47 | 51 | 6.77 |
| 2CAM62 | 106 | 20493 | 10 | 70 | 11.2 |
| 2CAM67 | 163 | 20494 | 28 | 90 | 13.5 |
| 2CAM71 | 96 | 20496 | 8 | 322 | 58.3 |
| 2CAM88 | 136 | 20501 | 17 | 93 | 14.8 |
| 3CAM1 | 186 | 20503 | 48 | 376 | 63 |
| 3CAM10 | 155 | 20507 | 25 | 212 | 41.7 |
| 3CAM11 | 161 | 20508 | 27 | 181 | 37.8 |
| 3CAM36 | 18 | 20511 | 1 | 151 | 29 |
| 3CAM57 | 119 | 20512 | 12 | 351 | 62.2 |
| 3CAM65 | 187 | 20513 | 49 | 315 | 57.9 |
| 2CRMS29 | 190 | 20521 | 52 | 58 | 8.42 |
| 2CAMS15 | 191 | 20531 | 53 | 383 | 64.8 |
| 2CAMS29 | 192 | 20534 | 54 | 99 | 14.5 |
| 2CAMS52 | 193 | 20535 | 55 | 342 | 60.9 |
| 3CAMS14 | 166 | 20543 | 30 | 278 | 51.6 |
| 3CAMS30 | 143 | 20545 | 19 | 176 | 36.5 |
| 3CAMS80 | 91 | 20554 | 7 | 189 | 36.2 |
| 3CAHM11 | 159 | 21050 | 26 | 316 | 56.9 |
| 3CAHM40 | 205 | 21051 | 61 | −1.8 | 1.97 |
| 3CRHM12 | 206 | 21052 | 61 | 4.8 | 2.2 |
| 3CRHM14 | 196 | 21053 | 57 | 136 | 26.2 |
| 3CRHM20 | 202 | 21055 | 59 | 7.4 | 2.83 |
| 3CRHM33 | 203 | 21056 | 59 | 6.1 | 2.15 |
| 3CRHM73 | 209 | 21060 | 62 | 150 | 27.3 |
| 3CRHM85 | 201 | 21061 | 65 | 3.5 | 1.7 |
| 3CRHM99 | 204 | 21062 | 60 | 309 | 57.3 |

TABLE 5
IL-12 competition activity of anti-IL-12Rβ2 VHH CDR3 families

| | | Number of clones | % IL-12 |
|---|---|---|---|
| CDR3 | Number of | with ≥50% IL-12 | competing clones/ |
| group ID | VHH clones | signaling inhibition | CDR3 group |
| 3 | 13 | 13 | 100 |
| 4 | 10 | 1 | 10 |
| 5 | 9 | 7 | 78 |
| 9 | 6 | 0 | 0 |
| 11 | 5 | 2 | 40 |
| 13 | 5 | 0 | 0 |
| 14 | 3 | 2 | 67 |
| 15 | 3 | 3 | 100 |
| 16 | 3 | 3 | 100 |
| 20 | 3 | 2 | 67 |
| 21 | 2 | 2 | 100 |
| 22 | 2 | 2 | 100 |
| 23 | 2 | 0 | 0 |
| 26 | 2 | 0 | 0 |
| 29 | 1 | 0 | 0 |
| 31 | 1 | 1 | 100 |
| 33 | 1 | 0 | 0 |
| 34 | 1 | 1 | 100 |
| 35 | 1 | 0 | 0 |
| 36 | 1 | 1 | 100 |
| 37 | 1 | 0 | 0 |
| 38 | 1 | 1 | 100 |
| 39 | 1 | 1 | 100 |
| 50 | 1 | 1 | 100 |
| 51 | 1 | 0 | 0 |
| 56 | 1 | 0 | 0 |
| 57 | 5 | 2 | 40 |
| 58 | 1 | 1 | 100 |
| 59 | 2 | 1 | 50 |
| 60 | 1 | 0 | 0 |
| 61 | 4 | 1 | 25 |
| 62 | 1 | 0 | 0 |

TABLE 6
Binding and competition characteristics of selected human-specific and human-mouse
cross-reactive anti-IL-12Rβ2 VHH clones

| | | | | | | | P.E. cell binding | |
|---|---|---|---|---|---|---|---|---|
| | | | | | P.E. binding ELISA | | hIL-12RB2 | |
| | | | SEQ | CDR3 | hIL-12RB2 | mIL-12RB2 | | % |
| | | Clone | ID | group | hIL12RB2/ | mIL12RB2/ | | cells |
| | Clone name | ID | NO: | ID | control | control | dMFI | bound |
| Human- | 3CRHSS4 | 20422 | 52 | 3 | 7.85 | 1.26 | 870 | 41.5 |
| specific | 3CRHS9 | 20415 | 49 | 3 | 5.08 | 1.09 | 941 | 42.1 |
| | SCRHS128 | 20432 | 68 | 4 | 3.65 | 0.98 | 1381 | 50.5 |
| | 3CRHS | 20373 | 72 | 5 | 8.64 | 0.81 | 735 | 39.5 |
| | 3CAHS124 | 20400 | 102 | 9 | 5.67 | 1.00 | 1095 | 46.3 |
| | 3CAHS142 | 20401 | 111 | 11 | 4.98 | 1.07 | 812 | 40.9 |
| | 3CAHS142 | 20438 | 128 | 14 | 3.58 | 0.93 | 1053 | 43.7 |
| | 3CRHS106 | 20429 | 131 | 15 | 10.18 | 1.03 | 1011 | 43.3 |
| | 3CRH25 | 20378 | 132 | 16 | 5.16 | 1.04 | 520 | 34.8 |
| | 3CAHS102 | 20399 | 146 | 20 | 3.54 | 1.02 | 994 | 44.7 |
| | 3CANS171 | 20407 | 148 | 21 | 3.27 | 0.96 | 1183 | 47.5 |
| | 3CRASS1 | 20421 | 150 | 22 | 12.00 | 0.95 | 1139 | 46 |
| | 3CAHS41 | 20394 | 151 | 23 | 8.77 | 1.35 | 971 | 43.3 |
| | 2CRH82 | 20372 | 175 | 37 | 16.13 | 1.77 | 744 | 40.1 |
| Cross- | 3CAHM11 | 21050 | 159 | 26 | 3.04 | 38.79 | 684 | 37.6 |
| reactive | 3CRHM14 | 21053 | 196 | 57 | 3.11 | 25.12 | 589 | 35.8 |
| | 3CRHM99 | 21062 | 204 | 60 | 4.24 | 5.24 | 458 | 24.4 |
| | 3CRHM73 | 21060 | 209 | 62 | 6.90 | 26.28 | 297 | 19.5 |
| | | | | | | | | P.E. IL-12 |
| | | P.E. cell binding | | | | | | competition |
| | | mIL-12RB2 | | P.E. Off-rate constant ranking | | | | IL-12 |
| | | | % | hIL-12RB2 | | mIL-12R82 | | competing |
| | | | cells | | k_{d} | | k_{d} | CDR3 |
| | Clone name | dMFI | bound | Response | (s⁻¹) | Response | (s⁻¹) | group? |
| Human- | 3CRHSS4 | N/A | N/A | 0.2035 | 2.72E−03 | N/A | N/A | yes |
| specific | 3CRHS9 | N/A | N/A | 0.2219 | 3.40E−03 | N/A | N/A | yes |
| | SCRHS128 | N/A | N/A | 0.2945 | 2.74E−03 | N/A | N/A | no |
| | 3CRHS | N/A | N/A | 0.4911 | 3.37E−03 | N/A | N/A | yes |
| | 3CAHS124 | N/A | N/A | 0.2928 | 3.24E−03 | N/A | N/A | no |
| | 3CAHS142 | N/A | N/A | 0.3518 | 3.24E−03 | N/A | N/A | no |
| | 3CAHS142 | N/A | N/A | 0.3385 | 3.02E−03 | N/A | N/A | yes |
| | 3CRHS106 | N/A | N/A | 0.2975 | 3.55E−03 | N/A | N/A | yes |
| | 3CRH25 | N/A | N/A | 0.523 | 2.05E−03 | N/A | N/A | yes |
| | 3CAHS102 | N/A | N/A | 0.3227 | 2.67E−03 | N/A | N/A | yes |
| | 3CANS171 | N/A | N/A | 0.1948 | 4.31E−03 | N/A | N/A | yes |
| | 3CRASS1 | N/A | N/A | 0.3271 | 4.30E−03 | N/A | N/A | yes |
| | 3CAHS41 | N/A | N/A | 0.6086 | 4.41E−03 | N/A | N/A | no |
| | 2CRH82 | N/A | N/A | 0.4264 | 2.74E−03 | N/A | N/A | no |
| Cross- | 3CAHM11 | 194 | 7.53 | 1.3968 | 5.19E−03 | 1.0262 | 4.62E−03 | no |
| reactive | 3CRHM14 | 312 | 18.6 | 1.1937 | 5.32E−03 | 0.9568 | 4.83E−03 | no |
| | 3CRHM99 | 251 | 10.7 | 1.4457 | 6.34E−03 | 0.8117 | 6.71E−03 | no |
| | 3CRHM73 | 257 | 10.4 | 1.4487 | 6.05E−03 | 0.9443 | 6.05E−03 | no |

TABLE 7
Binding characteristics of selected mouse-specific anti-IL-12Rβ2 VHH clones

| | | | | | | | | | | | | | | | P.E. IL-12 |
|---|---|---|---|---|---|---|---|---|---|---|---|---|---|---|---|
| | | | | | | | P.E. cell binding | | | | | | | | competition |
| | | | | | P.E. binding ELISA | | hIL-12RB2 | | mIL-12RB2 | | P.E. Off-rate ranking | | | | IL-12 |
| | | | SEQ | CDR3 | hiL12RB2 | mIL12RB2 | | % | | % | hIL-12RB2 | | mIL-128B2 | | competing |
| | | Clone | ID | group | hIL32RB2/ | mIL12RB2/ | | cells | | cells | Re- | k_{d} | Re- | k_{d} | CDR3 |
| | Clone name | ID | NO: | 10 | control | control | dMFI | bound | dMFI | bound | sponse | (s⁻¹) | sponse | (s⁻¹) | group? |
| Mouse- | 3CAM1 | 20503 | 186 | 48 | 1.20 | 3.30 | N/A | N/A | 376 | 63 | N/A | N/A | N/A | N/A | N/A |
| specific | 3CAMS7 | 20512 | 119 | 12 | 1.31 | 47.38 | N/A | N/A | 351 | 62.2 | N/A | N/A | N/A | N/A | N/A |
| | 3CAM65 | 20513 | 187 | 49 | 1.25 | 5.98 | N/A | N/A | 315 | 57.9 | N/A | N/A | N/A | N/A | N/A |
| | 2CAMS15 | 20531 | 191 | 53 | 0.98 | 4.50 | N/A | N/A | 383 | 64.8 | N/A | N/A | N/A | N/A | N/A |
| | 2CAMSSZ | 20535 | 193 | 55 | 0.97 | 5.41 | N/A | N/A | 342 | 60.9 | N/A | N/A | N/A | N/A | N/A |

TABLE 8
Sequences of heavy chain immunoglobulin used to generate
anti-IL-12Rβ2-FcDE molecules

| | Hinge (amino | |
|---|---|---|
| IgG sequence | acid sequence) | CH2—CH1 (amino acid sequence) |
| Human IgG1 WT | EPKSCDKTHTCPPCP | APELLGGPSVFLFPPKPKDTLMISRTPEVTCVVVDVSHEDP |
| SEQ ID NO: 210 | (SEQ ID NO: 843) | EVKFNWYVDGVEVHNAKTKPREEQYNSTYRVVSVLTVLHQD |
| | | WLNGKEYKCKVSNKALPAPIEKTISKAKGQPREPQVYTLPP |
| | | SRDELTKNQVSLTCLVKGFYPSDIAVEWESNGQPENNYKTT |
| | | PPVLDSDGSFFLYSKLTVDKSRWQQGNVFSCSVMHEALHNH |
| | | YTQKSLSLSPGK |
| Human IgG1 DE | EPKSCDKTHTCPPCP | 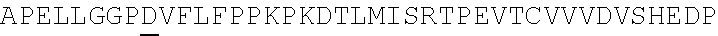 |
| (S239D/I332E) | (SEQ ID NO: 843) | EVKFNWYVDGVEVHNAKTKPREEQYNSTYRVVSVLTVLHQD |
| SEQ ID NO: 838 | | 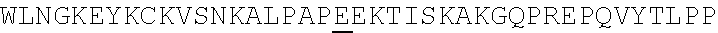 |
| | | SRDELTKNQVSLTCLVKGFYPSDIAVEWESNGQPENNYKTT |
| | | PPVLDSDGSFFLYSKLTVDKSRWQQGNVFSCSVMHEALHNH |
| | | YTQKSLSLSPGK |
| Mouse IgG2a WT | EPRGPTIKPCPPCKC | PAPNLLGGPSVFIFPPKIKDVLMISLSPIVTCVVVDVSEDD |
| SEQ ID NO 839 | (SEQ ID NO: 844) | PDVQISWFVNNVEVHTAQTQTHREDYNSTLRVVSALPIQHQ |
| | | DWMSGKEFKCKVNNKDLPAPIERTISKPKGSVRAPQVYVLP |
| | | PPEEEMTKKQVTLTCMVTDFMPEDIYVEWTNNGKTELNYKN |
| | | TEPVLDSDGSYFMYSKLRVEKKNWVERNSYSCSVVHEGLHN |
| | | HHTTKSFSRTPGK |
| Mouse IgG2a DE | EPRGPTIKPCPPCKC | 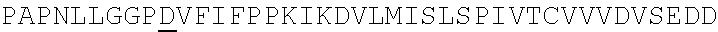 |
| SEQ ID NO: 840 | (SEQ ID NO: 844) | PDVQISWFVNNVEVHTAQTQTHREDYNSTLRVVSALPIQHQ |
| | | 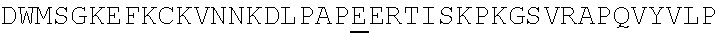 |
| | | PPEEEMTKKQVTLTCMVTDFMPEDIYVEWTNNGKTELNYKN |
| | | TEPVLDSDGSYFMYSKLRVEKKNWVERNSYSCSVVHEGLHN |
| | | HHTTKSFSRTPGK |

### Example 2: Screening Anti-IL-12Rβ2-VHH-Fc Antibodies for In Vitro ADCC Activity

To test the antibody-dependent cellular cytotoxicity (ADCC) of the selected anti-IL-12Rβ2 VHH-Fc antibodies, a Daudi (RRID: CVCL_0008) B cell line was generated with stable expression of human IL-12Rβ2 receptor as a ‘target’ cell line. Daudi-hIL-12Rβ2 cells were generated by lentiviral transduction of parental Daudi cells using pLVX-EF1α-hIL-12Rβ2-IRES-ZsGreen (in-house constructed, hIL-12Rβ2 cDNA: NM_001374259.2) derived lentivirus. Surface expression of IL-12Rβ2 was checked by flow cytometry using anti-IL-12Rβ2 antibody (Miltenyi, Clone REA333), and IL-12Rβ2 expression levels correlated with ZsGreen signal in a linear way.

Daudi-hIL-12Rβ2 target cells were labeled with 0.5 μM of CFSE (eBioscience, Cat #C34554), according to the manufacturer's protocol. Labeled cells, were rested overnight in ‘complete RPMI culture media’ (RPMI 1640 media (Gibco, 22400089) supplemented with 10% heat-inactivated FCS, 100 U/ml penicillin-streptomycin, 2 mM L-Glutamine, sodium-pyruvate and 50 μM beta-mercapto-ethanol). Peripheral blood mononuclear cells (PBMCs) were isolated from healthy donor buffy coat donations (supplied by the Red Cross Flanders Blood Service, Belgium) using a Ficoll-Paque gradient (Cytiva, Cat #17-1440-02) and cryopreserved in 90% FCS with 10% DMSO. Cryopreserved PBMCs were thawed and rested for 2 hours in complete RPMI culture media supplemented with 10 U/ml DNaseI (Roche, Cat #4536282001). After washing the PBMCs, cells were cultured overnight in complete RPMI culture media with addition of 100 IU/ml recombinant human IL-2 (Miltenyi, 130-097-744).

For the ADCC assay, CFSE-labeled Daudi-hIL-12Rβ2 target cells were opsonized for 30 minutes at 37° C. with 10, 0.5 or 0.025 μg/ml negative control human IgG1 antibody (BioXcell, Cat #BE0297), purified anti-IL-12Rβ2-VHH-Fc antibodies, or rituximab (BioXcell, Cat #SIM0008) as a positive control. Rituximab is a therapeutic monoclonal antibody targeting CD20 and mediates ADCC and CDC (complement-dependent cytotoxicity) of B cells. All anti-IL-12Rβ2-VHH-Fc antibodies included S239D and 1332E amino acid mutation in the Fc domain to enhance effector functions (indicated as ‘FcDE’). In addition, an anti-IL-12Rβ2-VHH-Fc clone 20432 with a wildtype IgG1 Fc sequence was also included in the experiment to illustrate the effect of the DE modification. The opsonized target cells were washed with RPMI culture medium and seeded at 20000 cells/well of a 96-well U-bottom plate in RPMI supplemented with 1% FCS. The FCS was heated to inactive the complement system. Then, the PBMC effector cells were harvested, washed and added to the target cells in a 10 times excess, resulting in an effector-to-target (E:T) ratio of 10:1. To assess the level of background cell death for each antibody a control was included with opsonized cells, but no effector cells (E:T of 0:1). The ADCC assay plates were centrifugated for 5′ at 300 g and incubated at 37° C. in a humidified CO₂ incubator. After 4 hours the Fixable Viability Dye eFluor780 (FVD780, eBioscience, Cat #65-0865-18) was added directly to the cell culture to stain the dead cells, and incubated for 20 minutes at 4° C. Cells were finally washed with FACS buffer and flow cytometric analysis was performed on a BD LSR HTS (BD Biosciences). Dead Daudi-hIL-12Rβ2 target cells were characterized as CFSE and FVD78 double positive. Specific lysis was calculated as follows: ((% experimental cell death—% spontaneous cell death (′E:T 0:1′))/(100-% spontaneous cell death (′E:T 0:1′))×100). The selected clones showed higher ADCC activity compared to rituximab at saturating concentration (10 μg/ml) of antibody opsonization, but showed variability in ADCC activity at 0.025 μg/ml (FIG. 3). S239D and 1332E mutations in the Fc domain of clone 20432 strongly enhanced the ADCC activity when compared to wildtype IgG1 Fc (FIG. 3) showing the added enhancement in ADCC activity resulting from the inclusion of the DE modifications.

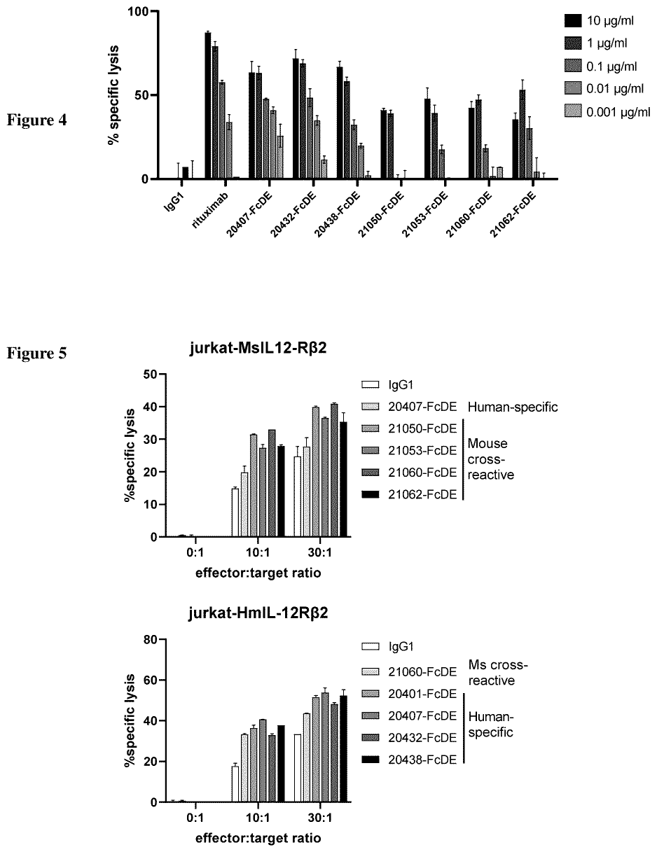

The ADCC activity of selected clones 20432, 20438, 20407, 21050, 21053, 21060 and 21062 was validated in an ADCC assay using purified natural killer (NK) cells as effector cells. The ADCC assay was similar to that described above, with some adjustments. CFSE-labeled Daudi-hIL-12Rβ2 cells were used as target cells. Cells were opsonized for 30 minutes at 37° C. with varying concentrations (10-0.0001 μg/ml) of negative control human IgG1 antibody (BioXcell, Cat #BE0297), purified anti-IL-12Rβ2-VHH-FcDE antibodies, or rituximab (BioXcell, Cat #SIM0008) as positive control. Target cells were washed and seeded at 20000 cells/well. The day before the assay, NK cells were negatively enriched using a MagniSort Human NK cell Enrichment Kit (eBioscience, Cat #8804-6819-74) from cryopreserved human PBMCs and cultured overnight in complete culture media with addition of 100 IU/ml recombinant human IL-2 (Miltenyi, 130-097-744). The NK cells were added to the target cells at effector-to-target ratio of 1:1 and incubated for 4 hours at 37° C. in a humidified CO₂ incubator. The dead cells were then stained with Fixable Viability Dye eFluor780 (FVD780, eBioscience, Cat #65-0865-18) and after washing analyzed on a BD LSR HTS flow cytometer (BD Biosciences). Dead Daudi-hIL-12Rβ2 target cells were characterized as CFSE positive and FVD78 positive. Specific lysis was calculated as follow: ((% experimental cell death-% cell death at 0.0001 μg/ml of corresponding antibody)/(100-% cell death at 0.0001 μg/ml of corresponding antibody))×100. Representative data of that obtained is given in FIG. 4.

### Example 3: Functional ADCC Cross-Reactivity of Anti-IL-12Rβ2-VHH-Fc Antibodies

The human-mouse cross reactivity of ADCC-mediated killing of selected anti-IL-12Rβ2-VHH-Fc clones was next determined using a Jurkat T cell line which was engineered to have stable expression of either human or mouse IL-12Rβ2 receptor. Jurkat-hIL-12Rβ2 or Jurkat-mIL-12Rβ2 cells were generated by lentiviral transduction of parental Jurkat cells using respectively pLVX-EF1α-hIL-12Rβ2-IRES-ZsGreen (in-house constructed, hIL-12Rβ2 cDNA: NM_001374259.2) or pLVX-EF1α-mIL-12Rβ2-IRES-ZsGreen (in-house constructed, mIL-12Rβ2 cDNA: NM_008354) derived lentivirus. The ADCC assay was performed as described in Example 2. CFSE-labeled Jurkat-hIL-12Rβ2 or Jurkat-mIL-12Rβ2 target cells were seeded at 20000 cells/well in complete RPMI with 1% heat-inactivated FCS in a 96-well U-bottom plate. PBMCs, activated overnight with 100 IU/ml recombinant human IL-2 (Miltenyi, 130-097-744), were used as effector cells and added to the target cells at effector-to-target ratios of 10:1 and 30:1. Cell death was determined 4 hours after target-effector co-culture as determined in Example 2. Specific lysis was calculated as follow: ((% experimental cell death-% spontaneous cell death (′E:T 0:1′))/(100-% spontaneous cell death (′E:T 0:1′))×100). Anti-hIL-12Rβ2-VHH-Fc clones 21050, 21053, 21060 and 21063 mediated killing of both human and mouse IL-12Rβ2-expressing Jurkat cells, while human-specific clone 20407 only killed Jurkat-hIL-12Rβ2 cells (FIG. 5).

### Example 4: Anti-IL-12Rβ2-VHH-Fc Antibodies Mediated Killing of Primary Human Th1 Cells

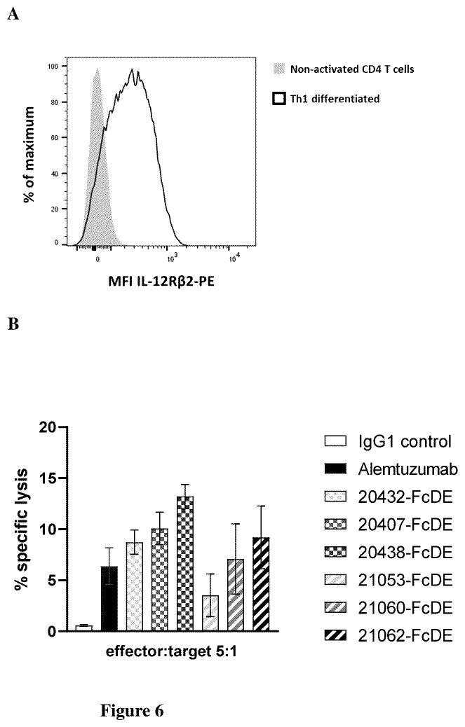

The potency of anti-IL-12Rβ2-VHH-Fc antibodies to mediate killing of primary human Th1 cells was analyzed in an ex vivo ADCC assay. Cryopreserved PBMCs were thawed and rested for 2 hours in complete RPMI culture media supplemented with 10 U/ml DNasel (Roche, Cat #4536282001). After washing the PBMCs, CD4 T cells were negatively enriched using a MagniSort Human CD4 T cell Enrichment Kit (eBioscience, Cat #8804-6811-74). CD4 T cells were subsequently cultured in Th1 differentiation media consisting of anti-CD3/CD28 Dynabeads at a bead-to-cell ratio of 2:1 with 10 ng/ml human recombinant IL-12 (in-house produced), 1 μg/ml neutralizing anti-IL-4 antibody (Biolegend, Cat #500838) and 100 IU/ml human recombinant IL-2 (Miltenyi, 130-097-744). Three days later, CD4 T cells were harvested and anti-CD3/CD28 Dynabeads were removed with a DynaMagnet. A fraction of the Th1 differentiated CD4 T cells were analyzed by flow cytometry for purity and IL-12Rβ2 expression levels (FIG. 6). The cells were then labeled with 1 μM CFSE (eBioscience, Cat #C34554) according to manufacturer's protocol. Labeled cells, were rested overnight in complete RPMI media supplemented with 100 IU/ml human recombinant IL-2. As described in example 2, autologous NK cells were negatively enriched from cryopreserved human PBMCs and cultured overnight in complete culture media with addition of 100 IU/ml recombinant human IL-2 (Miltenyi, 130-097-744). On the day of the ADCC assay, CFSE-labeled target cells were opsonized with 10 μg/ml of negative control human IgG1 antibody (BioXcell, Cat #BE0297), purified anti-IL-12Rβ2-VHH-FcDE antibodies, or alemtuzumab (R&D Systems, Cat #MAB9889-100) as positive control. Alemtuzumab is an FDA-approved therapeutic antibody against CD52 which is highly expressed on T and B cells. The opsonized target cells were seeded at 20000 cells/well together with 100000 activated NK cells (E:T 5:1), and incubated for 4 h before measuring cell death by flow cytometry as described in Example 2. Representative data is presented in FIG. 6. The human-specific anti-IL-12Rβ2-VHH-FcDE molecules induced higher levels of NK-mediated Th1 lysis compared to alemtuzumab.

### Example 5: Anti-IL-12Rβ2-VHH-Fc Antibodies can Compete with IL-12 for IL-12R Activation

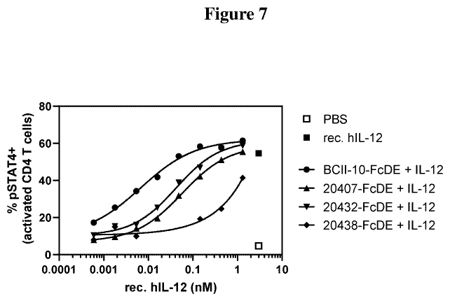

We assessed the ability of anti-IL-12Rβ2-VHH-FcDE molecules to compete with IL-12 induced pSTAT4 activation in primary human CD4 T cells. CD4 T cells were enriched from cryopreserved PBMCs and activated for 2 days as described in Example 4, however recombinant IL-12 was left out of the culture media here. Subsequently, the activated CD4 T cells were cultured for 16 hours in absence of activation beads and IL-2. Cells were seeded in 96 well plate and incubated with 100 nM of the indicated anti-IL-12Rβ2-VHH-FcDE molecules, or a control antibody (BCII-10-FcDE), for 30 minutes at 37° C. Then, the cells were stimulated with varying concentrations of recombinant human IL-12 for 30 minutes at 37° C. Cells were then fixed with paraformaldehyde (2% final concentration) for 10 minutes at room temperature, washed with FACS buffer, and incubated with BD Phosflow Perm Buffer III (BD Biosciences) for 30 minutes on ice. After washing with FACS buffer the cells were stained with anti-phospho-STAT4, anti-CD4 and anti-CD25 for 1 hour at room temperature. Dead cells were excluded from the analysis by using a Fixable Viability Dye eFluor780. Data is depicted in FIG. 7. We demonstrated that the anti-IL-12Rβ2-VHH-FcDE molecules competed with varying potencies, with 20438-FcDE being the most potent IL-12 signalling antagonist.

### Example 6: Anti-IL-12Rβ2-VHH-Fc Antibodies Prevent Alloreactive T Cell Proliferation

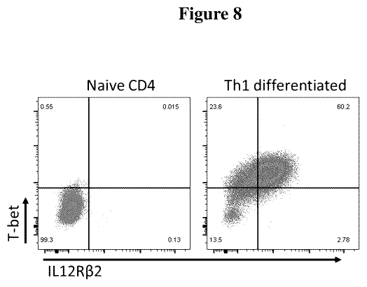

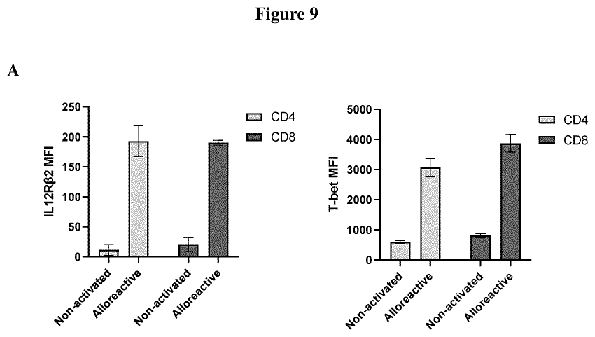

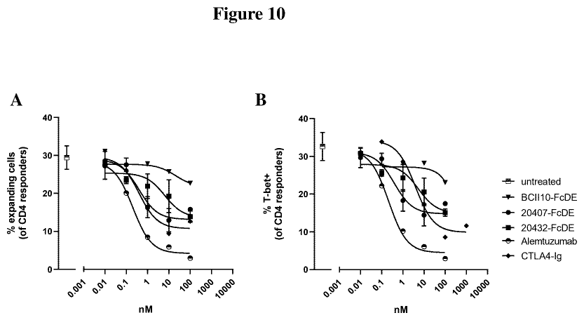

In primary human CD4 T cells, the expression of IL-12Rβ2 correlates with the expression of the Th1 lineage transcription factor Tbet, when cultured ex vivo in Th1 differentiation conditions (as described in Example 4) (FIG. 8). In line with this we demonstrate that IL-12Rb2 and Tbet protein expression is upregulated in activated alloreactive CD4 T cells in a one-way mixed lymphocyte reaction (MLR) (FIG. 9). Human PBMCs from two allogeneic donors were co-cultured for 7 days in which the ‘stimulator’ donor cells were treated with Mitomycin C to block T cell proliferation and the ‘responder’ donor cells were fluorescently labelled with CFSE to track T cell proliferation. We exploited the ex vivo MLR assay to access the ability of anti-IL-12Rβ2-VHH-FcDE molecules to prevent the expansion of alloreactive CD4 T cells. The cells were treated with varying concentrations of anti-IL-12Rβ2-VHH-FcDE molecules or control molecules at the start of the co-culture and were a second time treated on day three with the same concentration. We included alemtuzumab (R&D Systems, Cat #MAB9889-100) and recombinant CTLA-4-Ig (BioXCell, BE0099) as positive controls. We demonstrated that anti-IL-12Rβ2-VHH-FcDE molecules 20407-FcDE and 20432-FcDE dose-dependently reduce the percentage of expanding CD4 T cells and Tbet+CD4+ T cells among the CFSE+ responder cells (FIG. 10).

### Example 7: Selected Human-Mouse Cross-Reactive Anti-IL-12Rβ2 VHH Antibody does not Affect Frequency of Major Lymphocyte Subtypes in Blood of Show Acute Toxicity in Naïve Mice

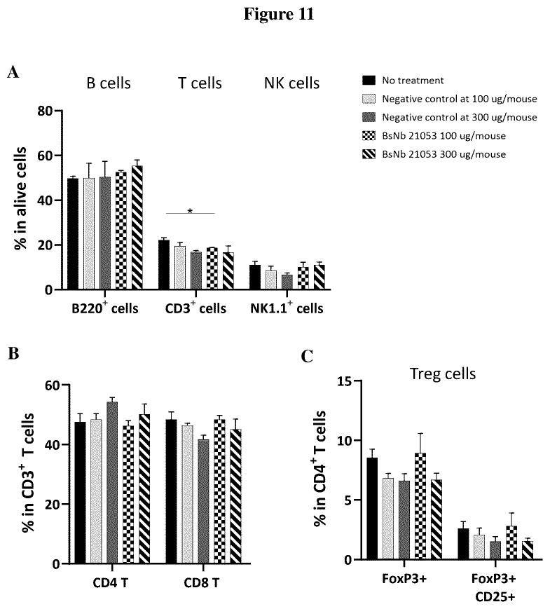

Next, we examined the tolerability of the mouse-specific anti-IL-12Rβ2 VHH-FcDE antibody 21053-IgG2aDE in mice. Mice were injected with 100 or 300 μg of antibody per mouse via intraperitoneal administration. Two days later blood was collected and analysed by flow cytometry for the homeostatic distribution of T, B and NK cells. We did not pick-up major changes in the percentages of T, B and NK cell lineages (FIG. 11).

1-8. (canceled)

9. A method of treatment, the method comprising: administering to a subject a binding molecule comprising a means for specifically binding an IL-12Rβ2 subunit, wherein the binding molecule depletes target cells expressing the IL-12Rβ2 subunit in the subject.

10. The method according to claim 9, wherein the binding molecule has Antibody Dependent Cellular Cytotoxicity, Antibody Dependent Cellular Phagocytosis, and/or Cell Dependent Cytotoxicity activity.

11. The method according to claim 10, wherein:
(a) the binding molecule does compete for IL-12Rβ2 binding with IL-12; or
(b) the binding molecule does not compete for IL-12Rβ2 binding with IL-12.

12. The method according to claim 9, wherein the means for specifically binding an IL-12Rβ2 subunit is an antibody.

13. The method according to claim 9, wherein the means for specifically binding an IL-12Rβ2 subunit is, or comprises, a VHH domain antibody.

14. The method according to claim 9, wherein the administration to the subject treats an autoimmune or inflammatory disorder, or a pathology where IL-12 via its IL-12 receptor complex including the IL-12Rβ2 contributes to the pathology.

15. The method of any one of claims 9 to 14, wherein the administration to the subject treats arthritis, multiple sclerosis, autoimmune uveitis, inflammatory bowel disease, graft versus host disease or Type 1 diabetes in the subject.

16. The method according to claim 9, wherein the method comprises depleting type 1 immune cells.

17. A binding molecule that binds to IL-12Rβ2, wherein the binding molecule comprises one or more of the following VHH antigen-domains:
(a) a VHH antigen-binding domain that binds IL-12Rβ2 comprising a set of three CDRs (CDR1, CDR2, and CDR3) selected from the sets of three CDRs for clones 20407, 21050, 21053, 21060, 21062, 20432, 20438;
(b) a VHH antigen-binding domain that binds IL-12Rβ2 comprising a set of three CDRs (CDR1, CDR2, and CDR3) that correspond to a set of three CDRs selected from the sets of three CDRs for clones 20407, 21050, 21053, 21060, 21062, 20432, 20438 apart from a maximum of ten amino acid sequence changes;
(c) a VHH antigen-binding domain that binds IL-12Rβ2 comprising a set of three CDRs (CDR1, CDR2, and CDR3) that have at least 90% sequence identity to a set of three CDRs selected from the sets of three CDRs of Table 1 for clones 20407, 21050, 21053, 21060, 21062, 20432, 20438; or
(d) a VHH antigen-binding domain that can compete for binding to IL-12Rβ2 with a VHH antigen-domain of any of (a) to (c).

18. A binding molecule that binds to an Interleukin-12 receptor β2 subunit (IL-12Rβ2), wherein the binding molecule comprises:
one or more of the following VHH antigen-domains:
(a) a VHH antigen-binding domain that binds IL-12Rβ2 comprising a set of three CDRs (CDR1, CDR2, and CDR3) selected from the sets of three CDRs of 20354, 20355, 20356, 20357, 20358, 20360, 20361, 20362, 20363, 20365, 20366, 20367, 20368, 20369, 20370, 20371, 20372, 20373, 20374, 20375, 20376, 20377, 20378, 20379, 20380, 20381, 20382, 20383, 20386, 20387, 20388, 20389, 20390, 20391, 20392, 20393, 20394, 20395, 20396, 20397, 20398, 20399, 20400, 20401, 20403, 20404, 20405, 20406, 20407, 20408, 20410, 20411, 20412, 20413, 20414, 20415, 20417, 20418, 20419, 20420, 20421, 20422, 20423, 20424, 20425, 20426, 20427, 20428, 20429, 20430, 20432, 20433, 20434, 20436, 20437, 20438, 20439, 20440, 20441, 20442, 20443, 20444, 20445, 20446, 20447, 20448, 20449, 20450, 20451, 20452, 20453, 20454, 20455, 20456, 20457, 20458, 20459, 20460, 20461, 20462, 20463, 20464, 20465, 20466, 20467, 20468, 20469, 20470, 20471, 20472, 20473, 20474, 20475, 20476, 20477, 20478, 20479, 20480, 20481, 20482, 20483, 20484, 20485, 20486, 20487, 20488, 20489, 20490, 20491, 20492, 20493, 20494, 20495, 20496, 20497, 20498, 20499, 20500, 20501, 20502, 20503, 20504, 20505, 20506, 20507, 20508, 20509, 20510, 20511, 20512, 20513, 20514, 20515, 20516, 20517, 20518, 20519, 20521, 20522, 20523, 20524, 20525, 20526, 20527, 20528, 20529, 20530, 20531, 20532, 20533, 20534, 20535, 20536, 20537, 20538, 20539, 20540, 20541, 20542, 20543, 20544, 20545, 20546, 20547, 20548, 20549, 20550, 20551, 20552, 20553, 20554, 20555, 21050, 21051, 21052, 21053, 21054, 21055, 21056, 21057, 21058, 21059, 21060, 21061, 21062, 21063, 21064, and 21065;
(b) a VHH antigen-binding domain that binds IL-12Rβ2 comprising a set of three CDRs (CDR1, CDR2, and CDR3) that correspond to a set of three CDRs of 20354, 20355, 20356, 20357, 20358, 20360, 20361, 20362, 20363, 20365, 20366, 20367, 20368, 20369, 20370, 20371, 20372, 20373, 20374, 20375, 20376, 20377, 20378, 20379, 20380, 20381, 20382, 20383, 20386, 20387, 20388, 20389, 20390, 20391, 20392, 20393, 20394, 20395, 20396, 20397, 20398, 20399, 20400, 20401, 20403, 20404, 20405, 20406, 20407, 20408, 20410, 20411, 20412, 20413, 20414, 20415, 20417, 20418, 20419, 20420, 20421, 20422, 20423, 20424, 20425, 20426, 20427, 20428, 20429, 20430, 20432, 20433, 20434, 20436, 20437, 20438, 20439, 20440, 20441, 20442, 20443, 20444, 20445, 20446, 20447, 20448, 20449, 20450, 20451, 20452, 20453, 20454, 20455, 20456, 20457, 20458, 20459, 20460, 20461, 20462, 20463, 20464, 20465, 20466, 20467, 20468, 20469, 20470, 20471, 20472, 20473, 20474, 20475, 20476, 20477, 20478, 20479, 20480, 20481, 20482, 20483, 20484, 20485, 20486, 20487, 20488, 20489, 20490, 20491, 20492, 20493, 20494, 20495, 20496, 20497, 20498, 20499, 20500, 20501, 20502, 20503, 20504, 20505, 20506, 20507, 20508, 20509, 20510, 20511, 20512, 20513, 20514, 20515, 20516, 20517, 20518, 20519, 20521, 20522, 20523, 20524, 20525, 20526, 20527, 20528, 20529, 20530, 20531, 20532, 20533, 20534, 20535, 20536, 20537, 20538, 20539, 20540, 20541, 20542, 20543, 20544, 20545, 20546, 20547, 20548, 20549, 20550, 20551, 20552, 20553, 20554, 20555, 21050, 21051, 21052, 21053, 21054, 21055, 21056, 21057, 21058, 21059, 21060, 21061, 21062, 21063, 21064, and 21065 apart from a maximum of ten amino acid sequence changes;
(c) a VHH antigen-binding domain that binds IL-12Rβ2 comprising a set of three CDRs (CDR1, CDR2, and CDR3) that have at least 90% sequence identity to a set of three CDRs of 20354, 20355, 20356, 20357, 20358, 20360, 20361, 20362, 20363, 20365, 20366, 20367, 20368, 20369, 20370, 20371, 20372, 20373, 20374, 20375, 20376, 20377, 20378, 20379, 20380, 20381, 20382, 20383, 20386, 20387, 20388, 20389, 20390, 20391, 20392, 20393, 20394, 20395, 20396, 20397, 20398, 20399, 20400, 20401, 20403, 20404, 20405, 20406, 20407, 20408, 20410, 20411, 20412, 20413, 20414, 20415, 20417, 20418, 20419, 20420, 20421, 20422, 20423, 20424, 20425, 20426, 20427, 20428, 20429, 20430, 20432, 20433, 20434, 20436, 20437, 20438, 20439, 20440, 20441, 20442, 20443, 20444, 20445, 20446, 20447, 20448, 20449, 20450, 20451, 20452, 20453, 20454, 20455, 20456, 20457, 20458, 20459, 20460, 20461, 20462, 20463, 20464, 20465, 20466, 20467, 20468, 20469, 20470, 20471, 20472, 20473, 20474, 20475, 20476, 20477, 20478, 20479, 20480, 20481, 20482, 20483, 20484, 20485, 20486, 20487, 20488, 20489, 20490, 20491, 20492, 20493, 20494, 20495, 20496, 20497, 20498, 20499, 20500, 20501, 20502, 20503, 20504, 20505, 20506, 20507, 20508, 20509, 20510, 20511, 20512, 20513, 20514, 20515, 20516, 20517, 20518, 20519, 20521, 20522, 20523, 20524, 20525, 20526, 20527, 20528, 20529, 20530, 20531, 20532, 20533, 20534, 20535, 20536, 20537, 20538, 20539, 20540, 20541, 20542, 20543, 20544, 20545, 20546, 20547, 20548, 20549, 20550, 20551, 20552, 20553, 20554, 20555, 21050, 21051, 21052, 21053, 21054, 21055, 21056, 21057, 21058, 21059, 21060, 21061, 21062, 21063, 21064, and 21065; or
(d) a VHH antigen-binding domain that can compete for binding to IL-12Rβ2 with an a VHH antigen-domain of any of (a) to (c); or
a means for specifically binding an IL-12Rβ2 subunit.

19. The binding molecule of claim 18, wherein the binding molecule comprises one or more of the following VHH antigen-domains:
(a) a VHH antigen-binding domain that binds IL-12Rβ2 and is selected from the VHH antigen-binding domain having the sequence of one of the VHH antigen-binding domains of 20354, 20355, 20356, 20357, 20358, 20360, 20361, 20362, 20363, 20365, 20366, 20367, 20368, 20369, 20370, 20371, 20372, 20373, 20374, 20375, 20376, 20377, 20378, 20379, 20380, 20381, 20382, 20383, 20386, 20387, 20388, 20389, 20390, 20391, 20392, 20393, 20394, 20395, 20396, 20397, 20398, 20399, 20400, 20401, 20403, 20404, 20405, 20406, 20407, 20408, 20410, 20411, 20412, 20413, 20414, 20415, 20417, 20418, 20419, 20420, 20421, 20422, 20423, 20424, 20425, 20426, 20427, 20428, 20429, 20430, 20432, 20433, 20434, 20436, 20437, 20438, 20439, 20440, 20441, 20442, 20443, 20444, 20445, 20446, 20447, 20448, 20449, 20450, 20451, 20452, 20453, 20454, 20455, 20456, 20457, 20458, 20459, 20460, 20461, 20462, 20463, 20464, 20465, 20466, 20467, 20468, 20469, 20470, 20471, 20472, 20473, 20474, 20475, 20476, 20477, 20478, 20479, 20480, 20481, 20482, 20483, 20484, 20485, 20486, 20487, 20488, 20489, 20490, 20491, 20492, 20493, 20494, 20495, 20496, 20497, 20498, 20499, 20500, 20501, 20502, 20503, 20504, 20505, 20506, 20507, 20508, 20509, 20510, 20511, 20512, 20513, 20514, 20515, 20516, 20517, 20518, 20519, 20521, 20522, 20523, 20524, 20525, 20526, 20527, 20528, 20529, 20530, 20531, 20532, 20533, 20534, 20535, 20536, 20537, 20538, 20539, 20540, 20541, 20542, 20543, 20544, 20545, 20546, 20547, 20548, 20549, 20550, 20551, 20552, 20553, 20554, 20555, 21050, 21051, 21052, 21053, 21054, 21055, 21056, 21057, 21058, 21059, 21060, 21061, 21062, 21063, 21064, and 21065;
(b) a VHH antigen-binding domain that binds IL-12Rβ2 and has at least 80% sequence identity to one of the VHH antigen-binding domains of 20354, 20355, 20356, 20357, 20358, 20360, 20361, 20362, 20363, 20365, 20366, 20367, 20368, 20369, 20370, 20371, 20372, 20373, 20374, 20375, 20376, 20377, 20378, 20379, 20380, 20381, 20382, 20383, 20386, 20387, 20388, 20389, 20390, 20391, 20392, 20393, 20394, 20395, 20396, 20397, 20398, 20399, 20400, 20401, 20403, 20404, 20405, 20406, 20407, 20408, 20410, 20411, 20412, 20413, 20414, 20415, 20417, 20418, 20419, 20420, 20421, 20422, 20423, 20424, 20425, 20426, 20427, 20428, 20429, 20430, 20432, 20433, 20434, 20436, 20437, 20438, 20439, 20440, 20441, 20442, 20443, 20444, 20445, 20446, 20447, 20448, 20449, 20450, 20451, 20452, 20453, 20454, 20455, 20456, 20457, 20458, 20459, 20460, 20461, 20462, 20463, 20464, 20465, 20466, 20467, 20468, 20469, 20470, 20471, 20472, 20473, 20474, 20475, 20476, 20477, 20478, 20479, 20480, 20481, 20482, 20483, 20484, 20485, 20486, 20487, 20488, 20489, 20490, 20491, 20492, 20493, 20494, 20495, 20496, 20497, 20498, 20499, 20500, 20501, 20502, 20503, 20504, 20505, 20506, 20507, 20508, 20509, 20510, 20511, 20512, 20513, 20514, 20515, 20516, 20517, 20518, 20519, 20521, 20522, 20523, 20524, 20525, 20526, 20527, 20528, 20529, 20530, 20531, 20532, 20533, 20534, 20535, 20536, 20537, 20538, 20539, 20540, 20541, 20542, 20543, 20544, 20545, 20546, 20547, 20548, 20549, 20550, 20551, 20552, 20553, 20554, 20555, 21050, 21051, 21052, 21053, 21054, 21055, 21056, 21057, 21058, 21059, 21060, 21061, 21062, 21063, 21064, and 21065;
(c) a VHH antigen-binding domain that binds IL-12Rβ2 and is a humanized version of one of VHH antigen-binding domains of 20354, 20355, 20356, 20357, 20358, 20360, 20361, 20362, 20363, 20365, 20366, 20367, 20368, 20369, 20370, 20371, 20372, 20373, 20374, 20375, 20376, 20377, 20378, 20379, 20380, 20381, 20382, 20383, 20386, 20387, 20388, 20389, 20390, 20391, 20392, 20393, 20394, 20395, 20396, 20397, 20398, 20399, 20400, 20401, 20403, 20404, 20405, 20406, 20407, 20408, 20410, 20411, 20412, 20413, 20414, 20415, 20417, 20418, 20419, 20420, 20421, 20422, 20423, 20424, 20425, 20426, 20427, 20428, 20429, 20430, 20432, 20433, 20434, 20436, 20437, 20438, 20439, 20440, 20441, 20442, 20443, 20444, 20445, 20446, 20447, 20448, 20449, 20450, 20451, 20452, 20453, 20454, 20455, 20456, 20457, 20458, 20459, 20460, 20461, 20462, 20463, 20464, 20465, 20466, 20467, 20468, 20469, 20470, 20471, 20472, 20473, 20474, 20475, 20476, 20477, 20478, 20479, 20480, 20481, 20482, 20483, 20484, 20485, 20486, 20487, 20488, 20489, 20490, 20491, 20492, 20493, 20494, 20495, 20496, 20497, 20498, 20499, 20500, 20501, 20502, 20503, 20504, 20505, 20506, 20507, 20508, 20509, 20510, 20511, 20512, 20513, 20514, 20515, 20516, 20517, 20518, 20519, 20521, 20522, 20523, 20524, 20525, 20526, 20527, 20528, 20529, 20530, 20531, 20532, 20533, 20534, 20535, 20536, 20537, 20538, 20539, 20540, 20541, 20542, 20543, 20544, 20545, 20546, 20547, 20548, 20549, 20550, 20551, 20552, 20553, 20554, 20555, 21050, 21051, 21052, 21053, 21054, 21055, 21056, 21057, 21058, 21059, 21060, 21061, 21062, 21063, 21064, and 21065; or
(d) a VHH antigen-binding domain that can compete for binding to IL-12Rβ2 with an a VHH antigen-domain of any of (a) to (c).

20. The binding molecule of claim 18, wherein the binding molecule comprises a single a VHH antigen-binding domain and an Fc domain.

21. The binding molecule of claim 18, wherein the binding molecule has Antibody Dependent Cellular Cytotoxicity, Antibody Dependent Cellular Phagocytosis, and/or Cell Dependent Cytotoxicity activity.

22. The binding molecule of claim 18, wherein the binding kills cells expressing IL-12Rβ2.

23. The binding molecule of claim 18, wherein:
(a) the binding molecule does not compete for binding to the IL-12Rβ2 with IL-12; or
(b) the binding molecule does compete for binding to the IL-12Rβ2 with IL-12.

24. The binding molecule claim 18, wherein the binding molecule is comprised in a pharmaceutical composition with a pharmaceutically acceptable carrier.

25. (canceled)

26. (canceled)

27. (canceled)

28. (canceled)
